(19) United States
(12) Patent Application Publication
(10) Pub. No.: US 2023/0053278 A1
(43) Pub. Date: Feb. 16, 2023

(54) Pharmacy assembly machine and packaging for customised polypills, and customised antipsychotic combinations for improved compliance

(71) Applicant: Steven Caffall Finch, London (GB)

(72) Inventor: Steven Caffall Finch, London (GB)

(21) Appl. No.: 17/790,731

(22) PCT Filed: Jan. 4, 2021
(86) PCT No.: PCT/GB2021/050017

§ 371 Date: Jul. 3, 2022

(30) Foreign Application Priority Data
Jan. 2, 2020 (GB) 2000032.9

(51) Int. Cl. A61J 3/07; B65B 11/52

(52) U.S. Cl. CPC A61J 3/074; B65B 11/52; A61J 2205/30; A61J 2205/10

(57) ABSTRACT

Customised polypills may be produced in the pharmacy from two or more drug packages using an assembly machine. Each drug package may comprise an array of cells containing individual doses of a single drug. The individual doses may be combined within the cells of the packages to produce an end user package with an array of cells containing the combined drugs in the form of individual polypills, which may be formed as capsules or pastilles. Label indicia of the drug packages may be combined together to form a composite label of the end user package. Serialization data may be read from the packages during assembly and sent to a remote server for authentication and supply chain management. In another aspect, patients who have a history of poor compliance with a prescribed oral antipsychotic while periodically seeking symptomatic relief from another prescribed psychoactive, are provided with both medicaments in the form of polypills to be taken in place of the solo antipsychotic when the patient is in crisis. Providing the preferred psychoactive as a combination motivates the patient to resume their antipsychotic therapy when symptoms return, which may result in better compliance and more effective management of psychosis in the community.

### FIELD OF THE INVENTION

In one broad aspect, this invention relates to systems for assembling together fixed dosage units of two or more different drugs to form customised polypills. In another broad aspect, the invention relates to antipsychotic drug combinations, and more particularly to the problem of non-compliance with a regimen of self-administered oral antipsychotics, in the treatment of chronic mental illness such as schizophrenia.

### BACKGROUND OF THE INVENTION

Polypills which are mass produced are referred to as fixed dose combinations. Fixed dose combinations may be made available for a drug combination that is commonly prescribed for a specific therapeutic indication.

For example, in the treatment of schizophrenia it is known to combine antipsychotics with antidepressants or with benzodiazepines (effective both as anxiolytics and as sedatives).

See for example: Neuropsychiatr Dis Treat. 2015; 11: 701-713; Augmentation with antidepressants in schizophrenia treatment: benefit or risk; Published online 2015 Mar. 16. doi:10.2147/NDT.562266; PMCID: PMC4370910; PMID: 25834445.

See also: Encephale. 2006 November-December; 32(6 Pt 1):1003-10; Benzodiazepines and schizophrenia, a review of the literature.

See also: Psychiatr Danub. 2017 September; 29(Suppl 3):345-348; Benzodiazepines in combination with antipsychotic drugs for schizophrenia: GABA-ergic targeted therapy.

Known combinations of antipsychotics and antidepressants include: perphenazine with fluoxetine; perphenazine with amitryptaline; olanzapine with fluoxetine; and lurasidone with D-cycloserine.

Polypills can also be customised for individuals who are prescribed multiple drugs, in order to reduce the pill burden and promote adherence to the therapeutic regimen, or to adapt the dosage or mode of delivery of the drug to the particular requirements of the patient.

For example, US2006078897 (A1) discloses capsules containing a combination of active pharmaceutical ingredients in the form of beads, pellets or minitablets having coatings selected, for example, to modify the release rate or for bioadhesion, and customized to the metabolic profile of the individual patient.

Customised polypills can be prepared by a specialised compounding pharmacy. However, this is expensive and time consuming, not only because the pharmacist must be skilled in the art of compounding but also due to the practicalities of storing and handling drugs in bulk form, including the need to carefully clean the tools that are used to combine them into individual dosage units after fulfilling each individual prescription.

Various approaches have been proposed to make customised polypills more easily available.

For example, WO2018206497 (A2) and WO2017034951 (A1) disclose systems for making customised polypills by 3D printing.

Customised polypills may also be produced using inkjet printing techniques, as disclosed in U.S. Pat. No. 7,707,964 B2.

See also: Inkjet printing of drug substances and use of porous substrates-towards individualized dosing, Journal of Pharmaceutical Sciences, 100: 3386-95, August 2011. Doi:10.1002/jps.22526. PMID 21360709.

See also: Electrodeless electrohydrodynamic drop-on-demand encapsulation of drugs into porous polymer films for fabrication of personalized dosage units, Journal of Pharmaceutical Sciences, 101: 2523-2533, July 2012. Doi:10.1002/jps.23165. PMID 22527973.

The storage and dispensing techniques of 3D printing and inkjet printing may not be equally suitable to all drugs, and may require additional cleaning, finishing or packaging steps, for example, where a substrate film must be inserted into a capsule to form the final dosage unit.

In an alternative approach, US2009149507 A1 teaches a polypill assembled from multiple fixed dosage units which may be connected by ultrasonic welding, snap-fit, adhesive or other techniques.

Following this approach, US2007193225 A1 and US2008306622 A1 disclose machines for receiving multiple tablet magazines, each containing a plurality of tablet components retained by releasable locks. The tablet components may have different active agents and different release rates. The tablet components may be loaded into the magazine by the user, or may be pre-loaded by the component vendor. The magazines are inserted into a mounting block of the machine, and then the releasable locks are actuated to release a first set of tablet components from each of the magazines. The tablet components released from the middle magazine are coated, e.g. sprayed with a bonding liquid. The tablet components are then pressed together by compression pins to form composite tablets which are discharged into a container. Another set of tablet components are then released from the magazines and assembled in the same way. RFID readers are arranged to read RFID tags on the magazines. A processor compares the sensed data with prescription information downloaded from a central database and prevents operation of the machine if the incorrect magazines are inserted.

Although such solid assembly techniques may be applicable to a wider range of drugs than printing techniques, the step of loading the tablet components into the magazines may introduce the potential for human error, while the use of a bonding agent within the assembly machine may give rise to special internal cleaning requirements. Further, packaging the polypills in bottles or other bulk containers may be less satisfactory than conventional blister packs or similar packaging that separates the individual single dosage units, in terms of consumer acceptability as well as stability in storage.

### OBJECTS OF THE INVENTION

In its first broad aspect, the present invention sets out to provide a simple and convenient way for the small, non-specialised pharmacy, or larger “hub” pharmacy or other centralised facility, to prepare customised polypills on demand. Preferably the polypills should be produced with minimal special cleaning and handling requirements and minimal opportunity for human error. Preferably the polypills should be individually packaged for the end user. Preferably the polypills should be produced subject to a robust authentication procedure to suppress counterfeit drugs.

In its second broad aspect, the invention is directed to improving compliance with a regimen of self-administered oral antipsychotics.

### SUMMARY OF THE INVENTION IN ITS FIRST BROAD ASPECT

IN ONE ASPECT, the invention provides an assembly system comprising an assembly apparatus, and a plurality of packages. The plurality of packages include at least first and second drug packages. Each package of the plurality of packages includes a plurality of cells. Each cell of the first drug package includes a single dose of a first drug, while each cell of the second drug package including a single dose of a second drug.

The assembly apparatus is configured to receive the plurality of packages including at least the first and second drug packages, and to combine together the first and second drugs of the first and second drug packages to form a plurality of single, orally ingestible bodies, wherein each single, orally ingestible body of the plurality of single, orally ingestible bodies includes the single dose of the first drug of a respective one of the cells of the first drug package, and the single dose of the second drug of a respective one of the cells of the second drug package.

Each cell of each drug package includes only one single dose of the respective drug, and the assembly apparatus is configured to combine together the first and second drugs within the cells of the packages received in the assembly apparatus.

IN ANOTHER ASPECT, the invention provides a method for forming drugs into orally ingestible bodies. The method includes providing first and second drugs, and providing a plurality of packages, the plurality of packages including at least first and second drug packages, each package of the plurality of packages including a plurality of cells.

The method further includes packaging the first drug in the first drug package so that each cell of the first drug package includes a single dose of the first drug, and packaging the second drug in the second drug package so that each cell of the second drug package includes a single dose of the second drug.

The invention further includes, after performing the abovementioned steps, combining together the first and second drugs to form a plurality of single, orally ingestible bodies, wherein each single, orally ingestible body of the plurality of single, orally ingestible bodies includes the single dose of the first drug of a respective one of the cells of the first drug package, and the single dose of the second drug of a respective one of the cells of the second drug package.

Only one said single dose is packaged in each respective cell of each respective drug package, and the first and second drugs are combined together within the cells of the plurality of packages.

IN ANOTHER ASPECT, the invention provides an assembly system comprising an assembly apparatus, and a plurality of packages. The plurality of packages include at least first and second drug packages, each package of the plurality of packages including a plurality of cells. Each cell of the first drug package includes a single dose of a first drug, while each cell of the second drug package includes a single dose of a second drug. Each single dose comprises one or more particles, the particles of all said single doses being of equal size and shape.

In this aspect of the invention, each particle has a dimension of at least 1.5 mm. Each cell of each drug package includes only one single dose of the respective drug. Each of the cells of the first drug package includes an equal number of particles, and each of the cells of the second drug package includes an equal number of particles.

The assembly apparatus is configured to receive the plurality of packages including at least the first and second drug packages, to receive a plurality of capsule caps and capsule bodies, and to combine together the first and second drugs of the first and second drug packages to form a plurality of capsules. For each capsule of the plurality of capsules, the single dose of the first drug of a respective one of the cells of the first drug package is encapsulated, together with the single dose of the second drug of a respective one of the cells of the second drug package, between a respective said capsule cap and capsule body.

IN ANOTHER ASPECT, the invention provides a method for filling drugs into capsules. The method includes providing first and second drugs, and providing a plurality of packages, the plurality of packages including at least first and second drug packages, each package of the plurality of packages including a plurality of cells. The method further includes packaging the first drug in the first drug package so that each cell of the first drug package includes a single dose of the first drug, and packaging the second drug in the second drug package so that each cell of the second drug package includes a single dose of the second drug. Each single dose comprises one or more particles, the particles of all said single doses being of equal size and shape.

In this aspect of the invention, each particle has a dimension of at least 1.5 mm. Only one said single dose is packaged in each respective cell of each respective drug package, so that each of the cells of the first drug package includes an equal number of particles, and each of the cells of the second drug package includes an equal number of particles.

The method further includes receiving in an assembly apparatus the plurality of packages including at least the first and second drug packages together with a plurality of capsule caps and capsule bodies, and operating the assembly apparatus to combine together the first and second drugs of the first and second drug packages to form a plurality of capsules. For each capsule of the plurality of capsules, the single dose of the first drug of a respective one of the cells of the first drug package is encapsulated, together with the single dose of the second drug of a respective one of the cells of the second drug package, between a respective said capsule cap and capsule body.

IN ANOTHER ASPECT, the invention provides a package for use in an assembly system, the package including a frame, the frame defining a plurality of cells separated by the frame to form a spaced array; wherein either:

(a) each cell includes at least one edible wall fixedly mounted in the frame, the at least one edible wall being configured to be detached from the frame and consumed in normal use by an end user; or

(b) each cell includes a cell wall, the cell wall extending along a cell axis between opposite, first and second ends of the cell at opposite, first and second sides of the frame; and each of the first and second ends of the cell is closed by a frangible foil or a movable closure element; and each cell contains a single dose of a drug; or

(c) each cell includes a cell wall, the cell wall extending along a cell axis between opposite, first and second ends of the cell at opposite, first and second sides of the frame; and each cell contains a part of an empty capsule, but without a complementary part required to complete the capsule.

IN ANOTHER ASPECT, the invention provides an assembly apparatus for filling capsules with drugs.

The apparatus includes a plurality of pushrods spaced apart in parallel relation, each pushrod having an end surface; and a pressure plate having a pressure surface arranged in opposed, spaced relation to the end surfaces of the pushrods to define a receiving space between the pressure plate and the end surfaces of the pushrods.

The apparatus further includes an actuation mechanism configured to cause relative movement between the pressure plate and the pushrods, in a compression stroke, along a displacement axis parallel with the pushrods; and an alignment structure configured to maintain a stack of packages in alignment with the pushrods when, in use, during the compression stroke, the pushrods are urged through the stack of packages positioned in the receiving space.

IN ANOTHER ASPECT, the invention provides an assembly apparatus for use in assembling together a plurality of packages. Each of the packages includes a frame which defines a plurality of cells separated by the frame to form a spaced array.

The apparatus includes an alignment structure for guiding the plurality of packages in a stacked configuration with the cells of each package in axial alignment with the cells of each other package of the plurality of packages, and a compression mechanism for compressing together the frames of the plurality of packages in the stacked configuration to form an assembly defining an end user package.

IN ANOTHER ASPECT, the invention provides an end user package including a plurality of cells and a plurality of single, orally ingestible bodies.

Each of the single, orally ingestible bodies is sealingly enclosed within a respective cell of the plurality of cells, each cell defining an enclosure which is openable by an end user to remove the respective single, orally ingestible body for use. Each single, orally ingestible body includes a single dose of a first drug, and a single dose of a different, second drug.

In this aspect, the invention further includes either feature (a) or feature (b).

According to feature (a), each single, orally ingestible body includes a capsule containing a plurality of spheroids, a first one or first ones of the spheroids containing the first drug but not the second drug, a second one or second ones of the spheroids containing the second drug but not the first drug.

According to feature (b), the end user package includes at least first and second drug packages. Each of the drug packages includes a respective frame, the frame defining a plurality of drug package cells separated by the respective frame to form a spaced array. Each of the drug package cells includes at least one edible wall fixedly mounted in the respective frame. The at least one edible wall of each of the drug package cells of the first drug package includes or encloses the single dose of the first drug but not the second drug, while the at least one edible wall of each of the drug package cells of the second drug package includes or encloses the single dose of the second drug but not the first drug. The frames are connected together, so that each of the drug package cells of the first drug package is in axial alignment with a respective one of the drug package cells of the second drug package to form together a respective one of the cells of the end user package. The edible walls of the respective drug package cells forming each cell of the end user package are connected together to form together the respective single, orally ingestible body contained within the respective cell of the end user package.

IN ANOTHER ASPECT, the invention provides an end user package including a plurality of capsules, each capsule including a single dose of a first drug, and a single dose of a different, second drug. Each capsule contains a plurality of particles, a first one or first ones of the particles containing the first drug but not the second drug, a second one or second ones of the particles containing the second drug but not the first drug.

The first and second ones of the particles are spheroids having a mean diameter of at least 1.5 mm, and all of the capsules contain an equal number of said first one or first ones of the particles, and all of the capsules contain an equal number of said second one or second ones of the particles.

### SUMMARY OF THE INVENTION IN ITS SECOND BROAD ASPECT

IN ANOTHER ASPECT, the invention provides a kit of medicaments for use in the treatment of psychosis in an individual with a history of non-compliance with oral medication. The kit includes a plurality of crisis oral dosage units, and a plurality of maintenance oral dosage units.

Each of the crisis oral dosage units includes a single dose of a first drug and a single dose of a different, second drug, said single doses being combined together as a single, orally ingestible body. Each of the maintenance oral dosage units includes a single dose of the first drug without the second drug.

The first drug is an antipsychotic, while the second drug is a psychoactive selected to alleviate an affective symptom associated with the psychosis.

IN ANOTHER ASPECT, the invention provides the use of a kit of medicaments as defined in the last mentioned aspect for the treatment of psychosis in an individual with a history of non-compliance with oral medication.

IN ANOTHER ASPECT, the invention provides a method for treating psychosis in an individual with a history of non-compliance with oral medication.

The method comprises providing different, first and second drugs, wherein the first drug is an antipsychotic, and the second drug is a psychoactive selected to alleviate an affective symptom associated with the psychosis; and combining together the first and second drugs to form a plurality of crisis oral dosage units, each of the crisis oral dosage units including a single dose of the first drug and a single dose of the second drug, said single doses being combined together as a single, orally ingestible body.

The method further comprises forming a plurality of maintenance oral dosage units, each of the maintenance oral dosage units including a single dose of the first drug without the second drug; and providing both the crisis oral dosage units and the maintenance oral dosage units to the individual for alternative self-administration at the discretion of the individual, wherein the maintenance oral dosage units are provided for self-administration when the individual feels well, and the crisis oral dosage units are provided for self-administration when the individual feels unwell.

IN ANOTHER ASPECT, the invention provides a method for manufacturing medicaments for treating psychosis in an individual with a history of non-compliance with oral medication.

The method comprises identifying the individual, and identifying different, first and second drugs prescribed for the identified individual, wherein the first drug is an antipsychotic, and the second drug is a psychoactive selected to alleviate an affective symptom associated with the psychosis in the identified individual.

The method further comprises combining together the first and second drugs to form a plurality of crisis oral dosage units customised for the identified individual, each of the crisis oral dosage units including a single dose of the first drug and a single dose of the second drug, said single doses being combined together as a single, orally ingestible body.

The method further comprises forming a plurality of maintenance oral dosage units, each of the maintenance oral dosage units including a single dose of the first drug without the second drug; and packaging the plurality of crisis oral dosage units and the plurality of maintenance oral dosage units, respectively as two separate components in a kit of medicaments.

### Definitions

In this specification, a combination or combination drug means a single oral dosage unit which includes two or more drugs (also referred to as active pharmaceutical ingredients or APIs) combined together.

A polypill means a combination drug which is configured as a discrete solid body.

A polypill may be configured as a capsule (i.e. a shell enclosing two or more APIs) or a tablet (i.e. a solid mass incorporating two or more APIs).

A single oral dosage unit means a body of medicament that is configured to be consumed orally as a single, discrete body. The medicament body may be a discrete solid body, for example a polypill, or alternatively could be a liquid body (in which two or more drugs may be combined together in a fixed ratio) defined by a liquid measure at the point of consumption.

Psychoactive drugs of course include antipsychotics, but for convenience, unless the context indicates otherwise, the term “psychoactive” is generally used herein to refer to a psychoactive drug prescribed to alleviate an affective symptom associated with psychosis in an individual, as distinguished from an antipsychotic prescribed to alleviate hallucinations or delusions as principal symptoms of the psychosis.

### BRIEF DESCRIPTION OF THE DRAWINGS

### Customised Polypills

The first broad aspect of the invention as directed to the production of customised polypills will be better understood by reference to the illustrative embodiments which will now be described, purely by way of example and without limitation to the scope of the claims, and with reference to the accompanying drawings, in which:

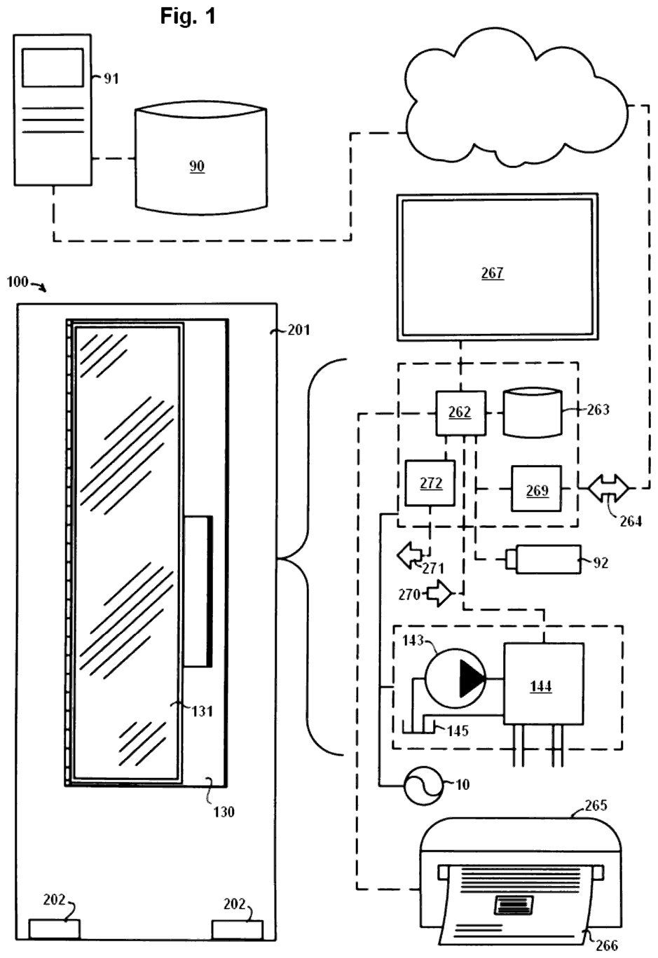

A FIRST EMBODIMENT is illustrated in FIGS. 1-58, wherein:—

FIG. 1 shows a first assembly apparatus, which hereafter will be referred to also as the FIRST MACHINE 100, in front view together with co-operating, internal and external system elements in schematic view.

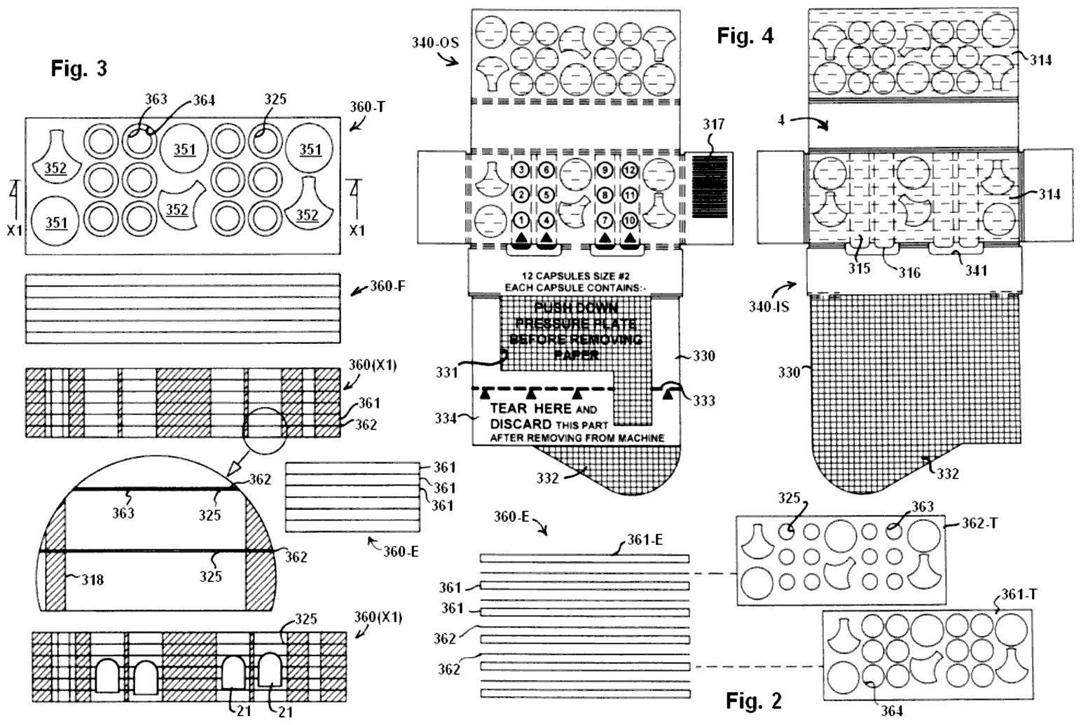

FIG. 2-9 show a CAPSULE CAP PACKAGE 304 and parts thereof, for use with the first machine, wherein:

FIG. 2 is an exploded view of the block.

FIG. 3 shows various views of the block, including sections taken at X1-X1 of FIG. 3, wherein one of the sections shows the caps inserted into the block.

FIG. 4 shows outer side and inner side views of the casing as formed from a flat sheet of card before it is folded around the block.

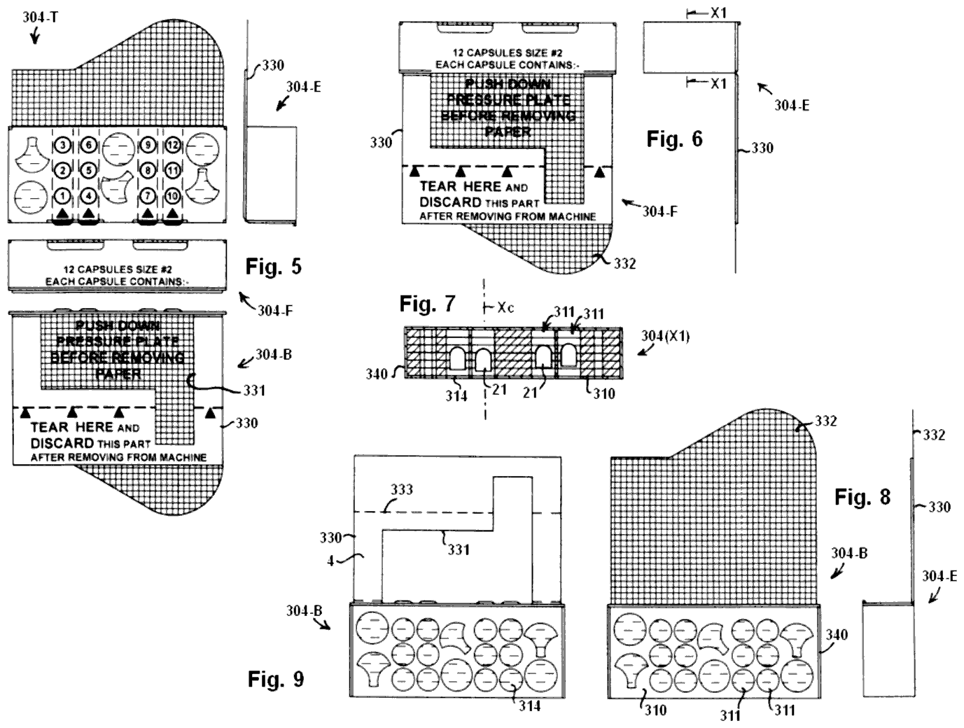

FIG. 5 shows various views of the finished cap package with the flap in a first folded position.

FIG. 6 shows front and end views of the finished cap package with the flap unfolded to the assembly position.

FIG. 7 is a section at X1-X1 of FIG. 6.

FIG. 8 shows bottom and end views of the finished cap package with the flap unfolded to a different position.

FIG. 9 is a bottom view corresponding to that of FIG. 8 with the release paper removed, revealing the adhesive rear surface of the flap.

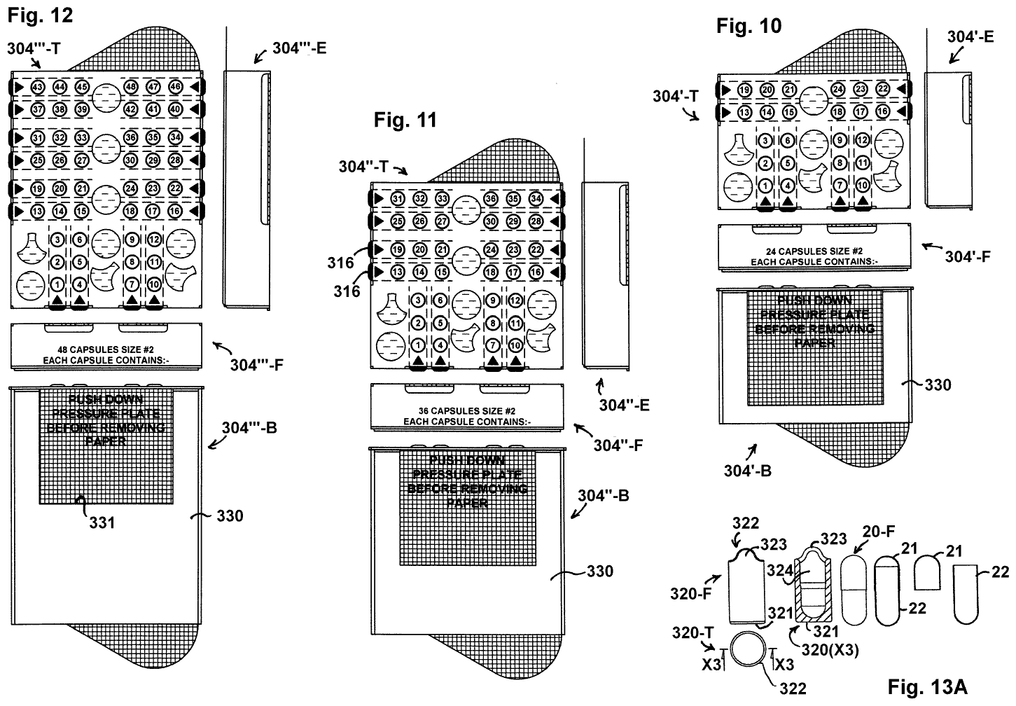

FIGS. 10, 11 and 12 show, respectively, three different cap packages, similar to that of FIGS. 2-9 but in different sizes with different numbers of cells, each cap package being shown in various views.

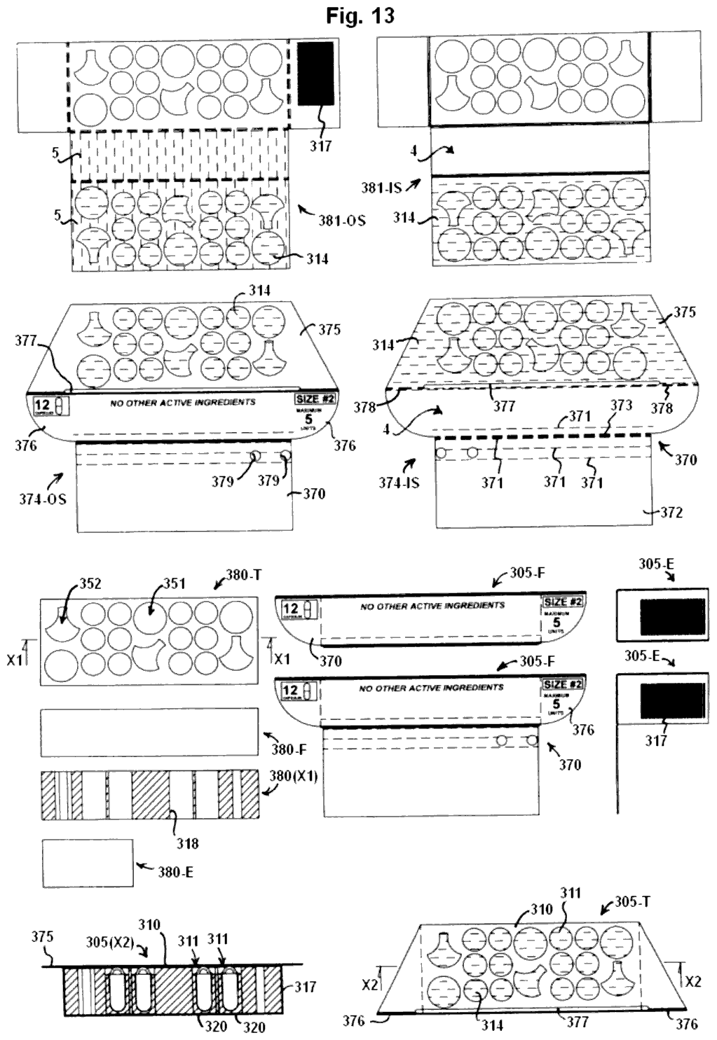

FIG. 13 shows:
various views of a CAPSULE BODY PACKAGE 305 and parts thereof, for use with the first machine, including:
   various views of the block, wherein the section suffixed (X1) is taken through the block at section line X1-X1 of FIG. 13; and,
   in the top half of the drawing, inner side and outer side views of the inner and outer casings as formed from flat sheets of card before being folded around the block; and
   in the lower right-hand quadrant of the drawing, various views of the finished package containing the carriers and capsule bodies, with the flap respectively in a folded and unfolded position; wherein the section suffixed (X2) in the bottom left-hand corner of the drawing is taken through the finished package at section line X2-X2 of FIG. 13.

FIG. 13A shows:
relatively enlarged top (open) end, side, and longitudinal sectional views of one CARRIER of the capsule body package, the longitudinal section suffixed (X3) being taken at section line X3-X3 of the top plan (i.e. open end) view; and
relatively enlarged views of one CAPSULE, of which only the capsule body is contained in the capsule body package, showing the capsule in assembled front (i.e. side) view and, in longitudinal central section, both assembled and disassembled.

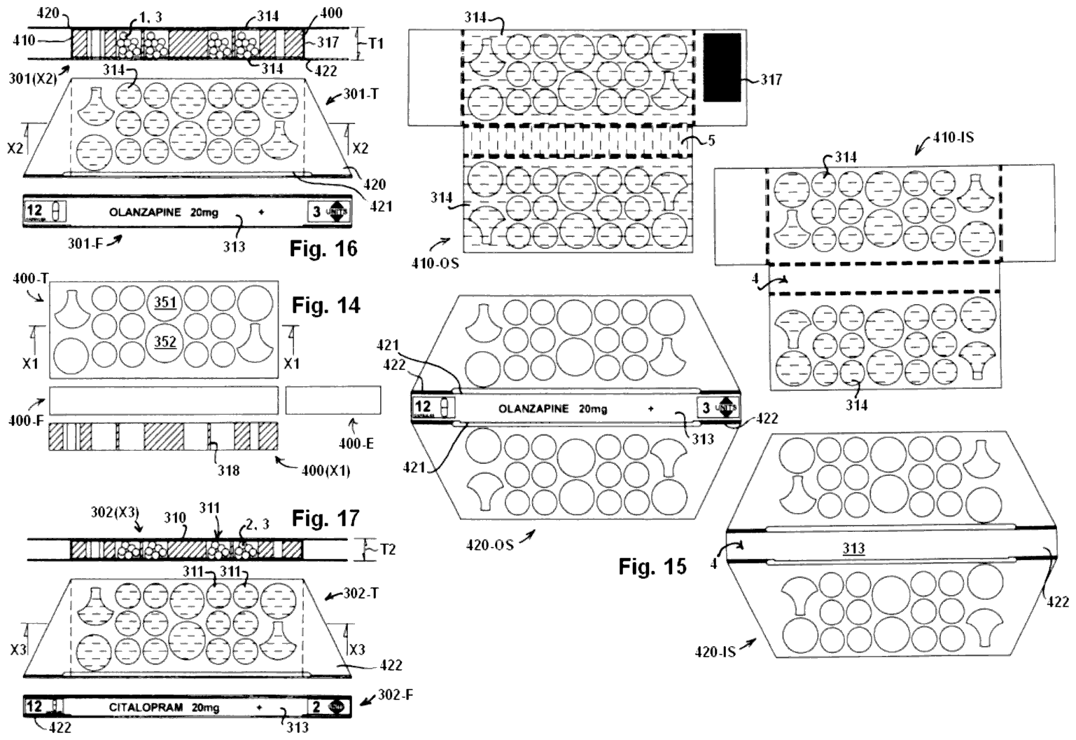

FIGS. 14-17 show first and second DRUG PACKAGES 301, 302 and parts thereof, for use with the first machine, wherein:—

FIG. 14 shows various views of the block of a first drug package 301, wherein the section suffixed (X1) is taken at section line X1-X1 of FIG. 14.

FIG. 15 shows inner side and outer side views of the inner and outer casings of the first drug package, as formed from flat sheets of card before being folded around the block.

FIG. 16 shows various views of the finished first drug package 301 containing a first drug (olanzapine, 20 mg) in spheronized form, wherein the section suffixed (X2) is taken through the finished first drug package at section line X2-X2 of FIG. 16.

FIG. 17 shows various views, corresponding to those of FIG. 16, of a similar, finished, second drug package 302 containing a second drug (citalopram, 20 mg) in spheronized form, wherein the section suffixed (X3) is taken through the finished second drug package at section line X3-X3 of FIG. 17.

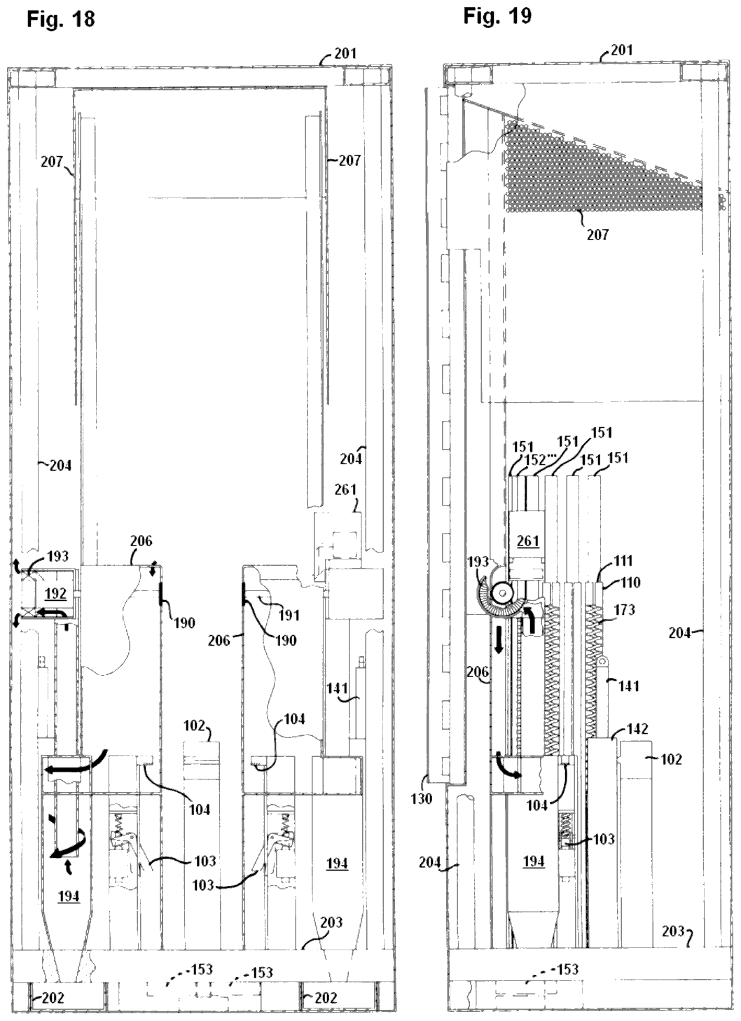

FIG. 18 is a front view of the BODY of the FIRST MACHINE, partially cut away, including various internal components but NOT INCLUDING the PUSHROD ASSEMBLY, the PEDESTAL BLOCK, or the MOVING FRAME ASSEMBLY.

FIG. 19 is a side view of the body of the first machine, corresponding to that of FIG. 18, also partially cut away.

FIG. 19A is an enlarged view of part of FIG. 19 showing one of the cutters.

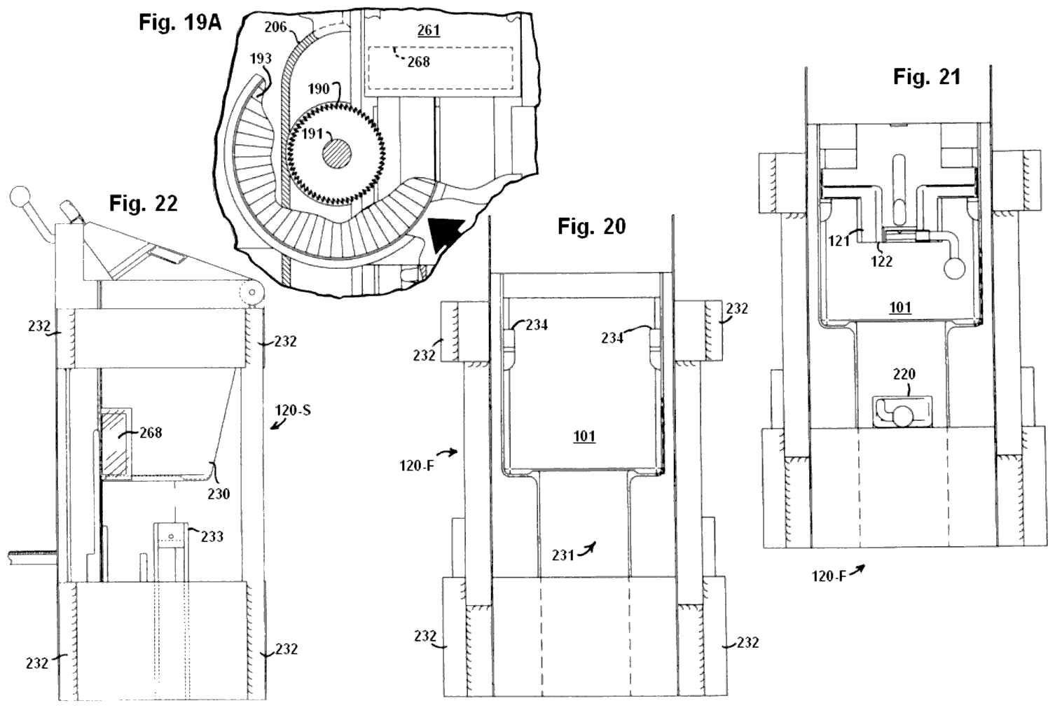

FIGS. 20-25 show the MOVING FRAME ASSEMBLY of the first machine and parts thereof, wherein:—

FIG. 20 is a front view of the moving frame without the top plate and front plate.

FIG. 21 is a front view including the top plate and front plate, both in a lowered position.

FIG. 22 is a side view with the top plate raised and the front plate (shown in part) lowered.

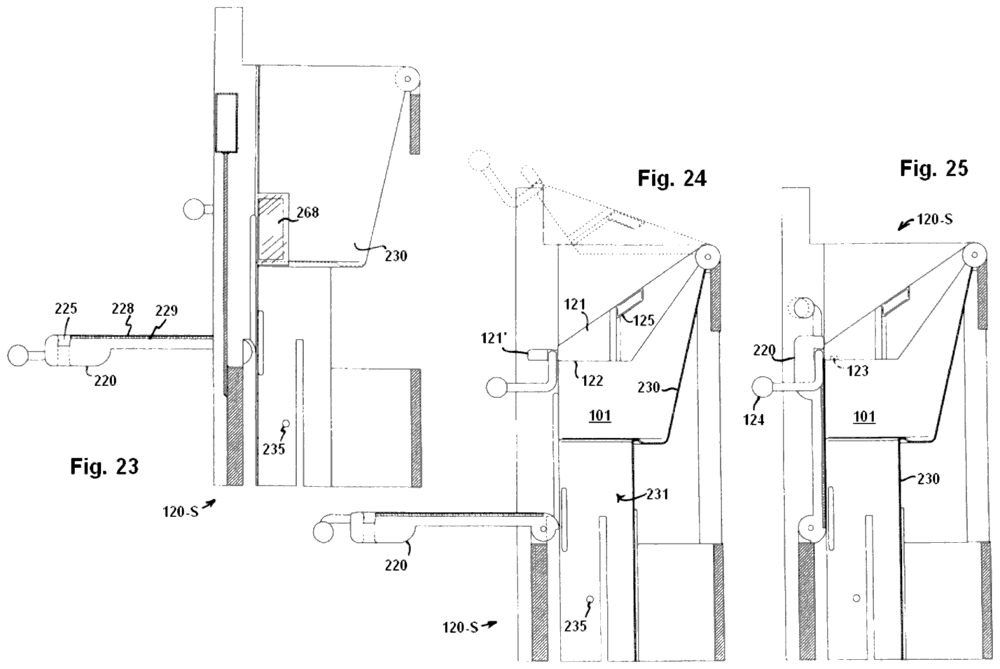

FIG. 23 is a side view with the outer parts of the moving frame partially cut away.

FIG. 24 is a side view with the outer parts and also the inner casing partially cut away to reveal the cavity in which the packages will be stacked.

FIG. 25 corresponds to FIG. 24 with the front plate raised and locked to the top plate.

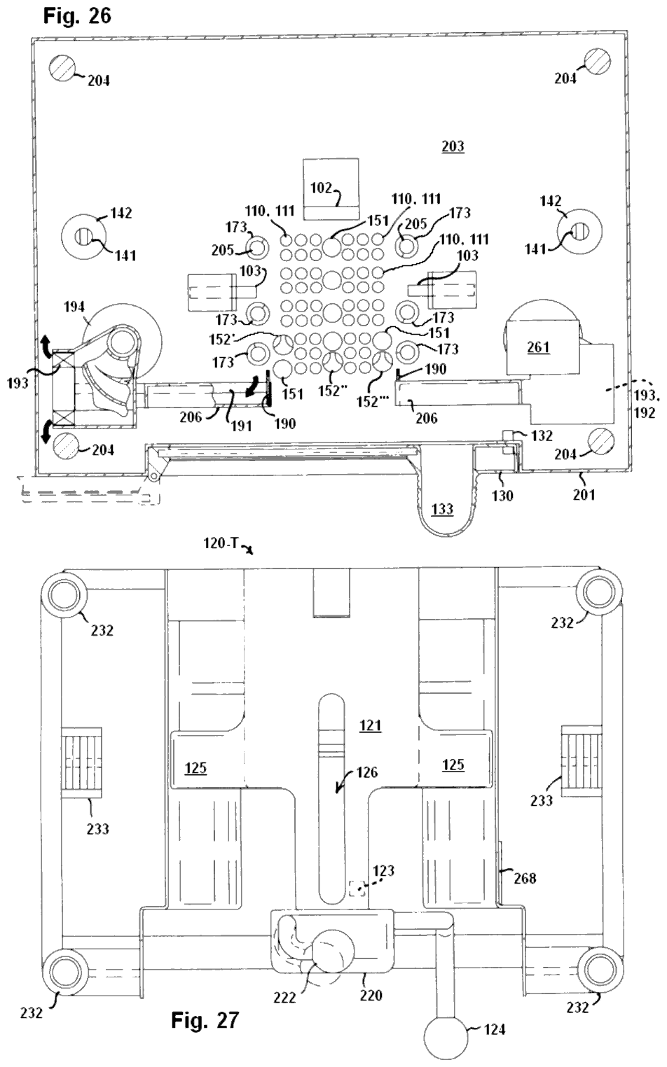

FIG. 26 is a plan view of the BODY of the first machine, partially cut away to show various internal components including the pushrod assembly, but NOT INCLUDING the PEDESTAL BLOCK or the MOVING FRAME ASSEMBLY.

FIG. 27 is a plan view of the MOVING FRAME ASSEMBLY showing the top and front plates in the locked position of FIG. 25.

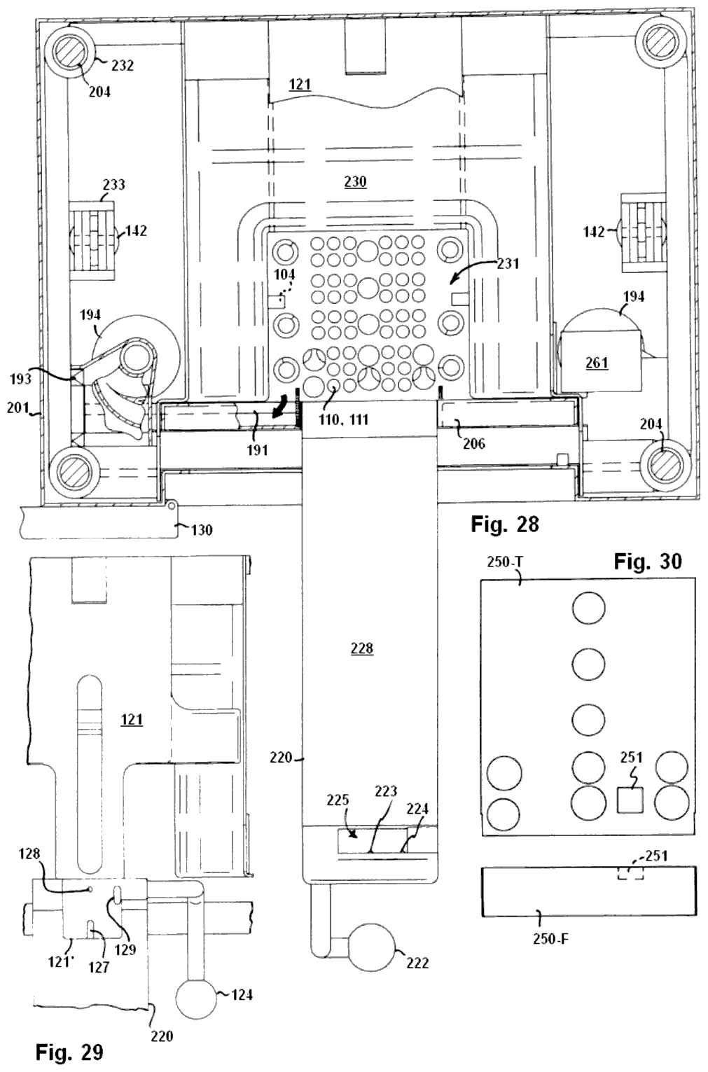

FIG. 28 is a plan view of the first machine, partially cut away, including the pushrod assembly and the moving frame assembly but NOT INCLUDING the PEDESTAL BLOCK, wherein the front and top plates are both in the lowered position and the top plate is partially cut away.

FIG. 29 is a plan view of part of the moving frame assembly with the front and top plates both in the lowered position, showing in particular the tab on the top plate which is received in the slot of the front plate, best seen in FIG. 28, when the front plate is raised and locked to the top plate in the position of FIG. 27.

FIG. 30 shows the PRESSURE PAD for use with the first machine, respectively in top and front view.

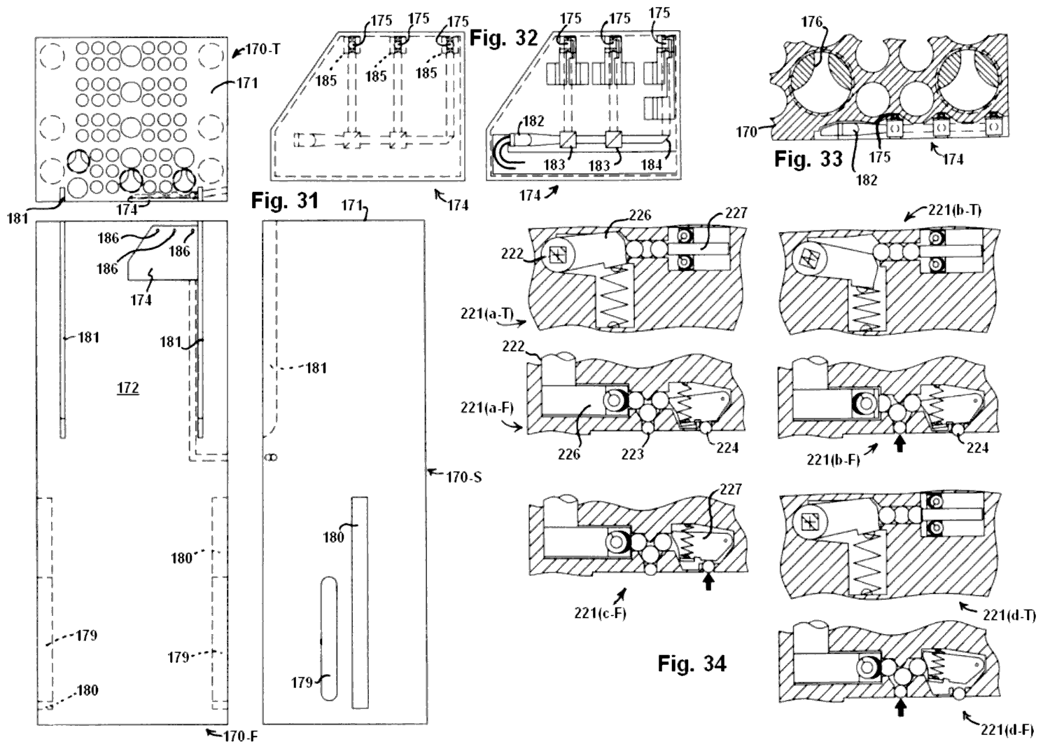

FIG. 31 shows the PEDESTAL BLOCK of the first machine, including the sensor unit and the profile rod sleeves, respectively in front, right side, and top view.

FIG. 32 shows two front views of the sensor unit forming part of the pedestal block as shown in FIG. 31, respectively with external details (left-hand view) and internal details (right-hand view).

FIG. 33 is a fragmentary section taken in a horizontal plane, parallel with and a few millimetres below the top surface of the pedestal block in its front right-hand corner, showing the profile rod sleeves and only internal features of the sensor unit.

FIG. 34 shows the locking mechanism of the front plate of the first machine, partially cut away in top plan view (suffix -T) and front view (suffix -F) in the raised position of the front plate, in four different positions indicated respectively by suffix a-, b-, c- and d-.

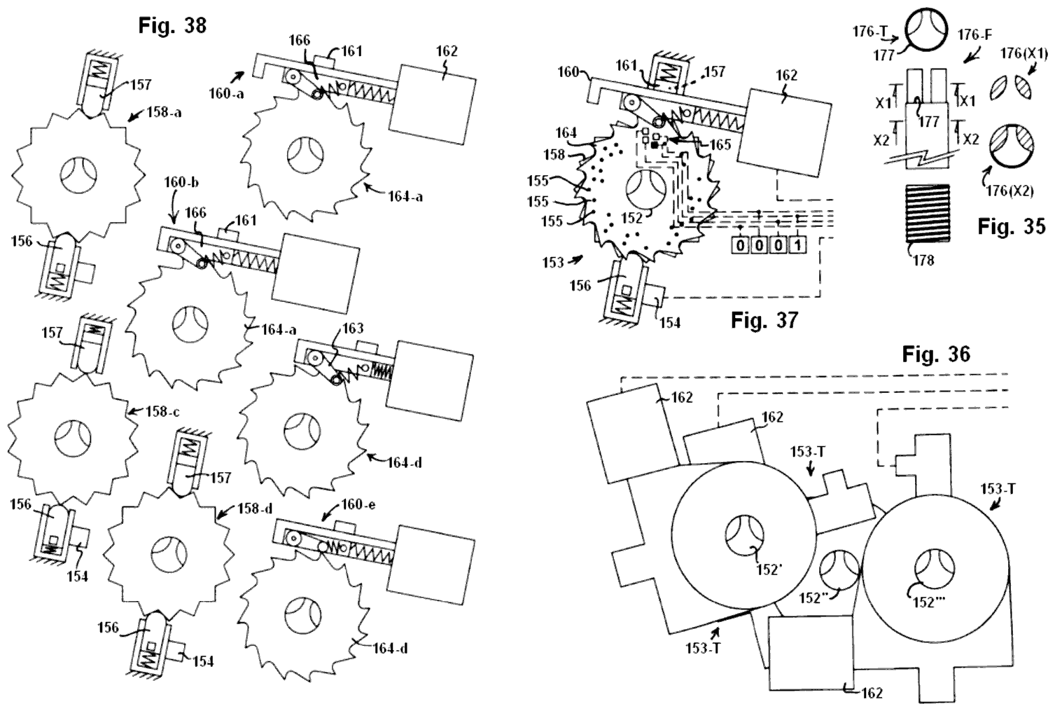

FIG. 35 shows various views of one profile rod sleeve of the pedestal block and its threaded retainer, with the sections suffixed (X1) and (X2) being taken respectively at section lines X1-X1 and X2-X2 of FIG. 35.

FIG. 36 shows the profile rod actuator assembly of the first machine.

FIG. 37 shows one of the three profile rod actuators as shown in FIG. 36.

FIG. 38 shows, separately, the ratchet plate and index wheel of the profile rod actuator of FIG. 37, illustrating five sequential steps during movement of the profile rod to the next index position (suffixes -a to -e).

FIG. 39 is a front view of the first machine with the door open, including the pushrod assembly but NOT INCLUDING the PEDESTAL BLOCK or the MOVING FRAME ASSEMBLY.

FIG. 40 is a front view corresponding to FIG. 39, but including the pedestal block.

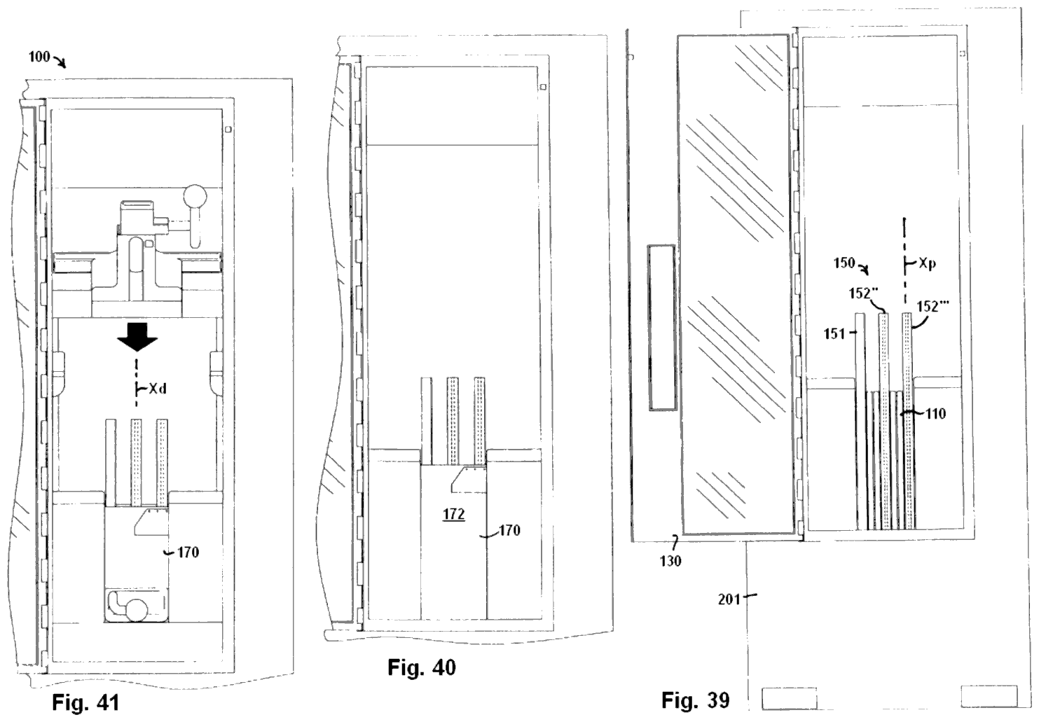

FIGS. 41-51 show the first machine in front view, in consecutive steps through the combination of the cap package, capsule body package and first and second drug packages to produce one end user package; wherein:—

FIG. 41 shows the machine including all its parts, with the door open and the moving frame assembly in an open position ready to receive the packages. The arrow indicates an initial adjustment of the moving frame assembly (which happened while the door was closed with the moving frame in a closed position, and responsive to a user command) to define the maximum height of the stack prior to opening the door to receive the packages.

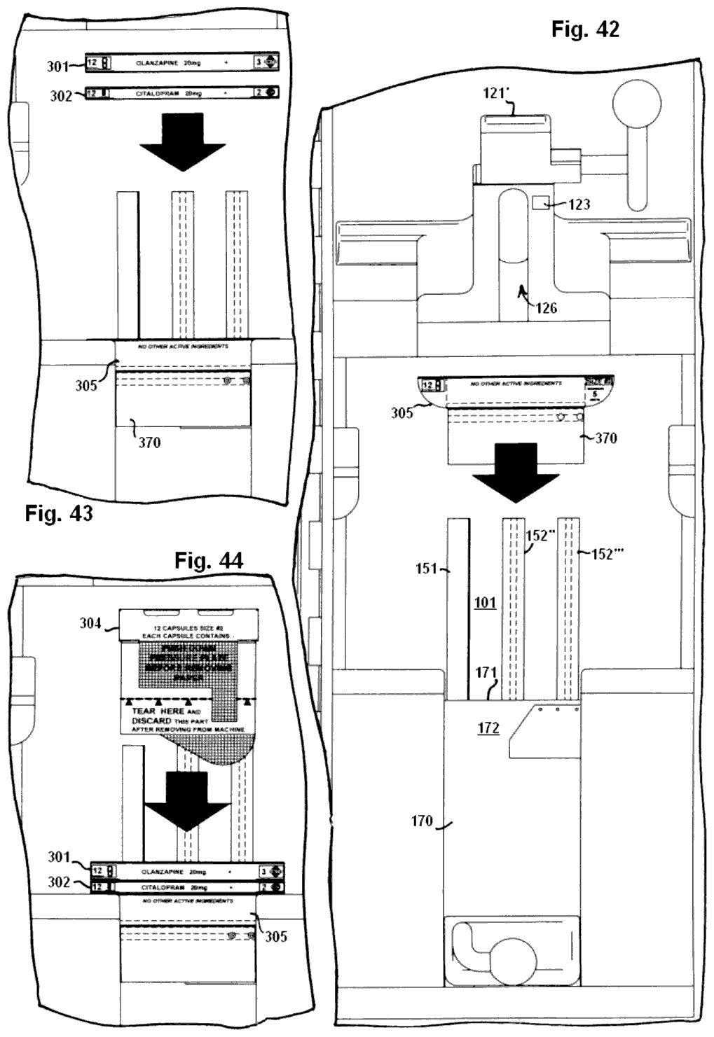

FIG. 42 shows the capsule body package with its flap open being placed over the pushrod assembly.

FIG. 43 shows the first and second drug packages being placed over the pushrod assembly and stacked onto the capsule body package, which rests on top of the pedestal block with its flap folded down over the front surface of the pedestal block.

FIG. 44 shows the cap package with its flap open being placed over the pushrod assembly.

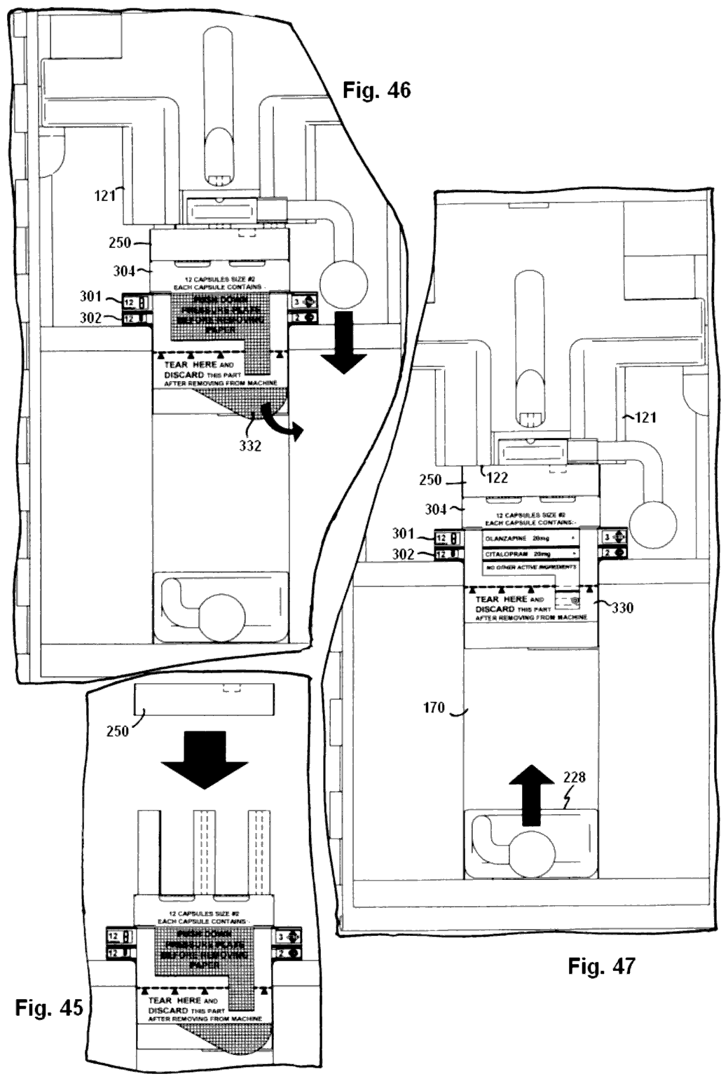

FIG. 45 shows the cap package stacked on top of the drug packages while the pressure pad is placed over the pushrod assembly.

FIG. 46 shows the top plate being pushed down to compress the stack before the release paper is peeled away from the rear, adhesive surface of the flap of the cap package.

FIG. 47 shows the flap of the cap package folded down to adhere to the front surfaces of the drug packages and the flap of the capsule body package, before the front plate is raised and locked to the top plate.

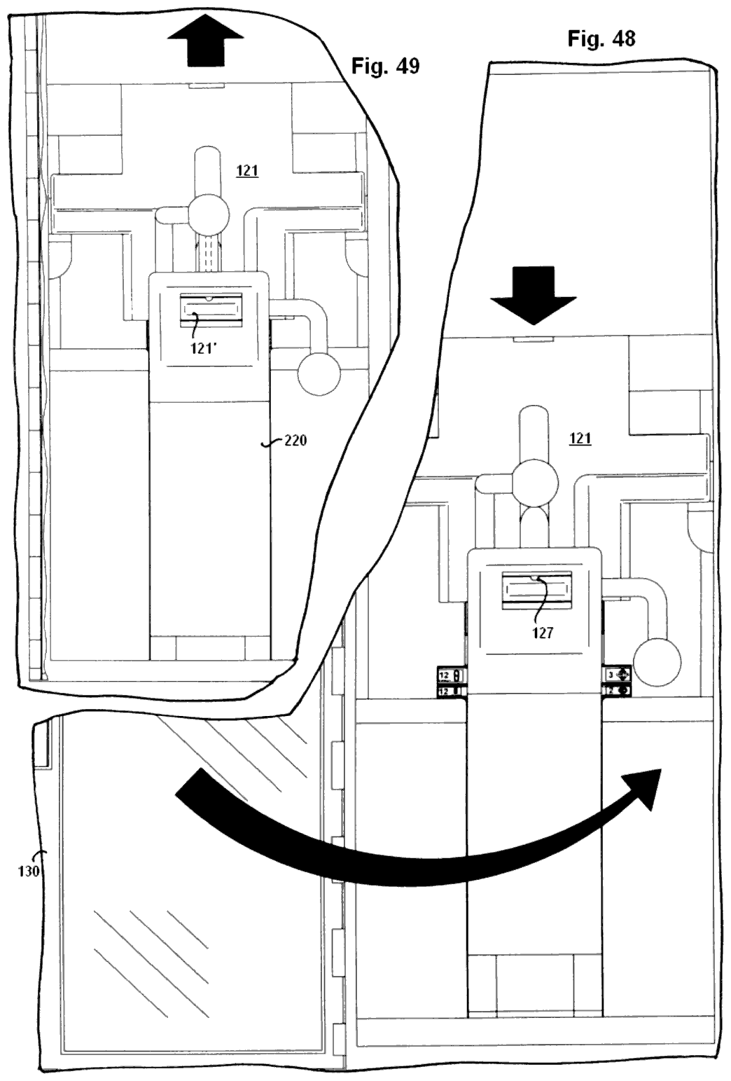

FIG. 48 shows the front plate locked to the top plate before closing the door, as indicated by the curved arrow, and then actuating the moving frame assembly, as indicated by the upper arrow, to urge the pushrods through the stack.

FIG. 49 shows the machine with the door (cut away) in the closed position and the moving frame assembly at its downward limit position for this operation, before the moving frame assembly reverses direction and moves upwards as indicated by the arrow to the package removal position.

FIG. 50 shows the machine with the door open and the moving frame assembly in the package removal position, after unlocking and opening the front and top plates, and before removing the stack from the pedestal block.

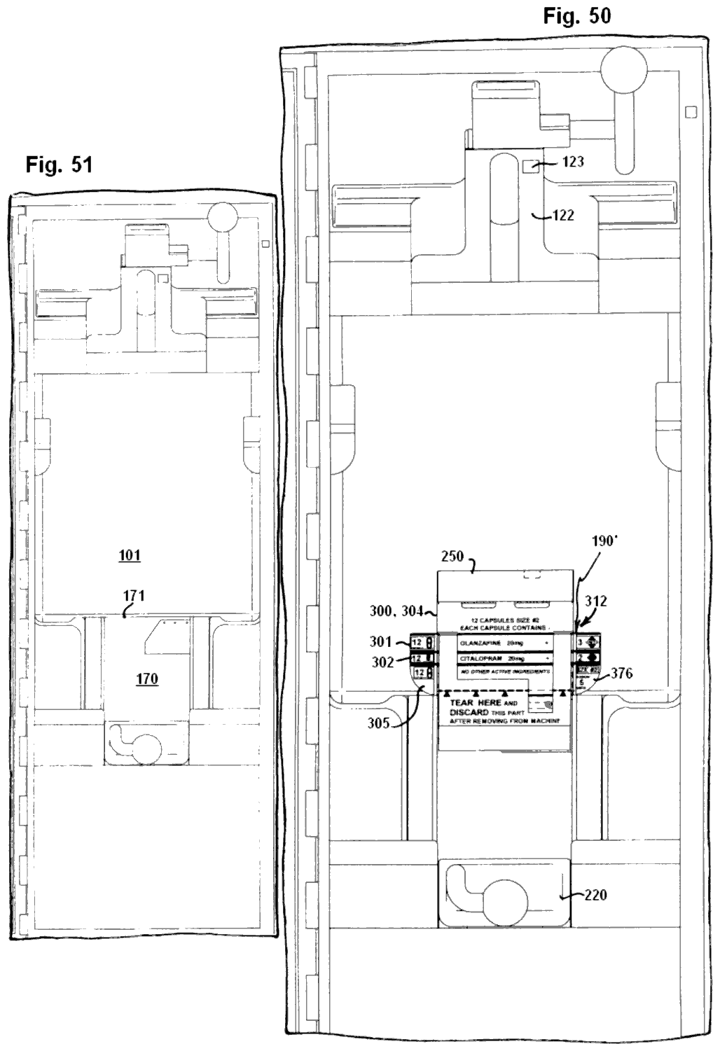

FIG. 51 shows the machine still in the position of FIG. 50, after removing the stack.

FIGS. 52-54A are sections taken in a vertical plane parallel with the front surface of the pedestal block, through the stack of packages, before and during the operating cycle of the first machine as shown in FIGS. 48-49, wherein:

FIG. 52 shows the components of the packages in their initial state as placed in the machine, with the upper end surfaces of the pushrods aligned with the package cells, just before actuation of the moving frame assembly;

FIG. 53 shows the pushrods extending part way into the stack at an intermediate point during the downward movement of the moving frame assembly;

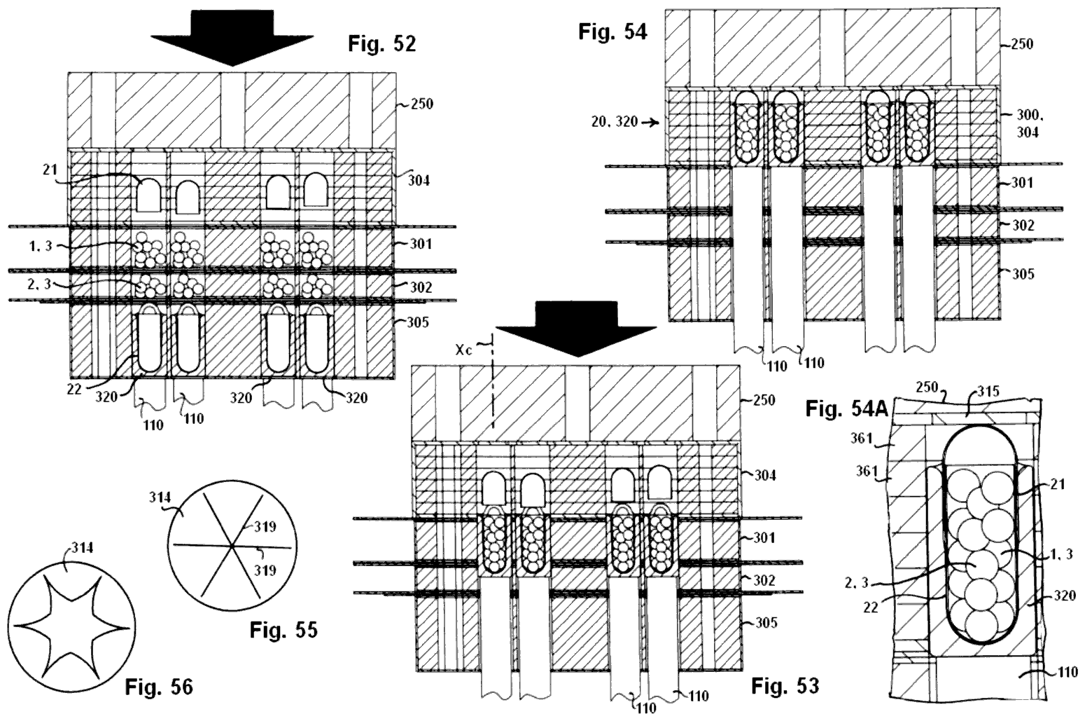

FIG. 54 shows the pushrods extending to their maximum distance into the stack in the downward limit position of the moving frame assembly as shown in FIG. 49; and

FIG. 54A is an enlarged view of part of FIG. 54.

FIG. 55 shows the frangible foil closing one end of one cell of a respective one of the packages, as seen in a direction along the cell axis, in an intact condition.

FIG. 56 shows the frangible foil of FIG. 55, partially broken.

FIG. 57 shows various views of the FIRST END USER PACKAGE 300 together with the empty drug and capsule body packages, after removal from the machine, with the adhesive flap forming the composite label assembly in an unfolded position before being folded against the rear surface of the end user package as indicated by the curved arrow.

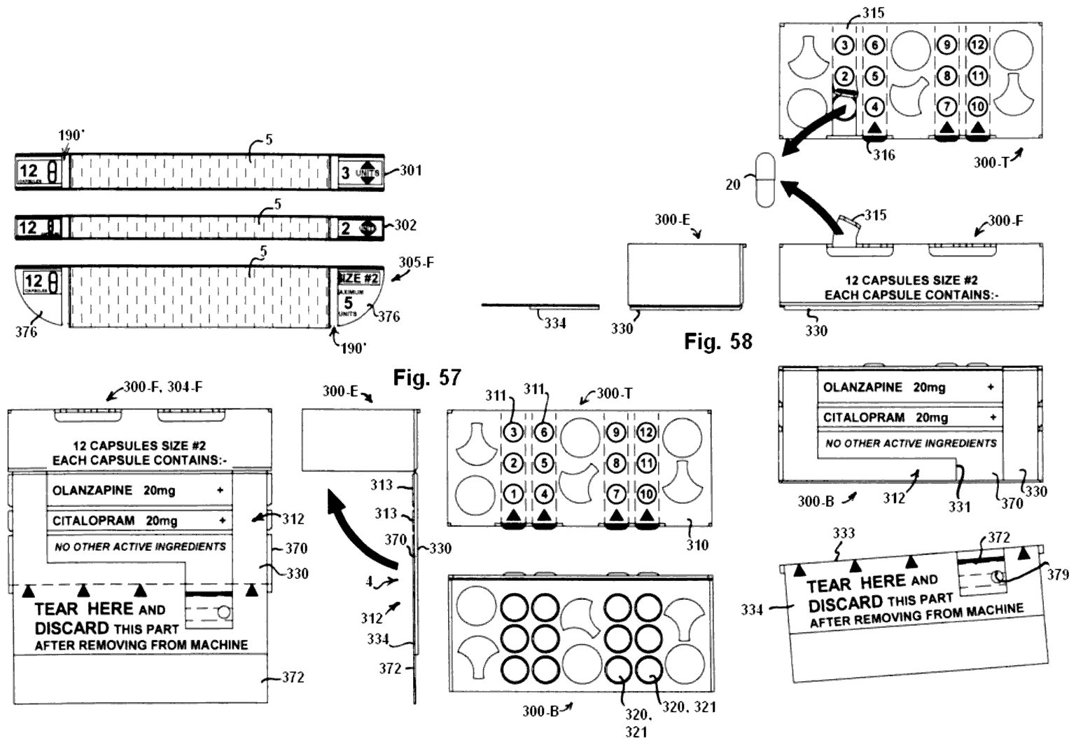

FIG. 58 shows various views of the finished FIRST END USER PACKAGE 300 together with the waste portion of the adhesive flap after detachment, and showing one FINISHED CAPSULE 20 removed from its cell.

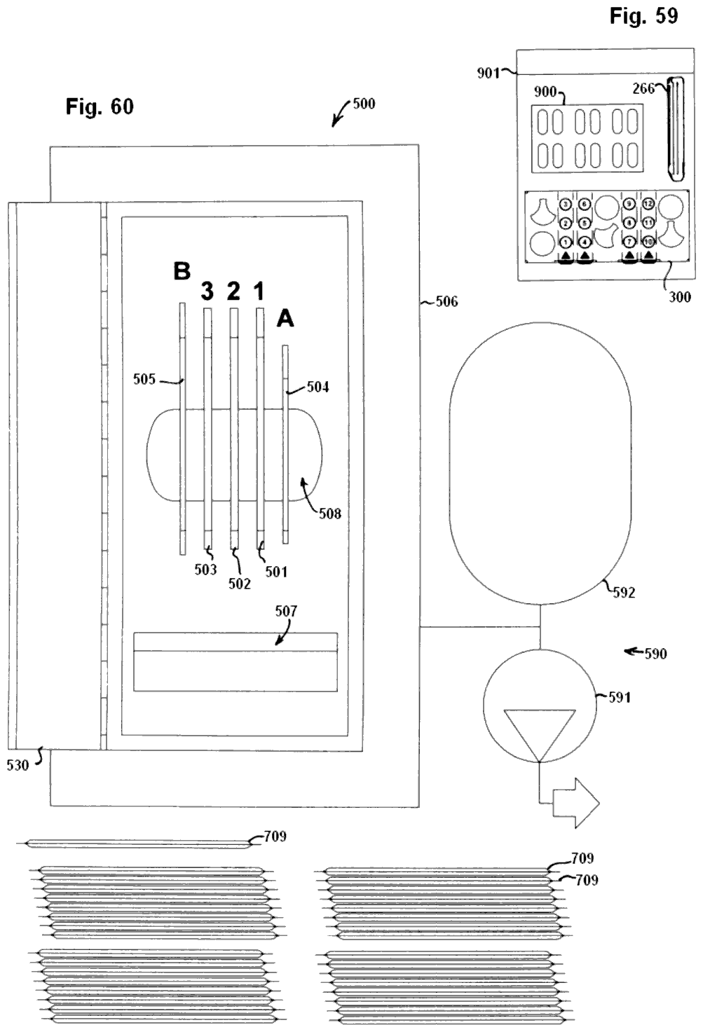

FIG. 59 shows a KIT OF MEDICAMENTS contained in a pharmacy bag and including the first end user package of FIG. 58 and a conventional drug package containing the first drug in fixed dosage units.

A SECOND EMBODIMENT and variants thereof are illustrated in FIGS. 60-101, wherein:—

FIG. 60 shows a second assembly apparatus, which hereafter will be referred to also as the SECOND MACHINE, in front view together with some wrapped packages for use therewith. Co-operating, internal and external system elements of the second machine are not shown in this figure, but are generally the same as those of the first machine, as shown in FIG. 1 and as described with reference thereto.

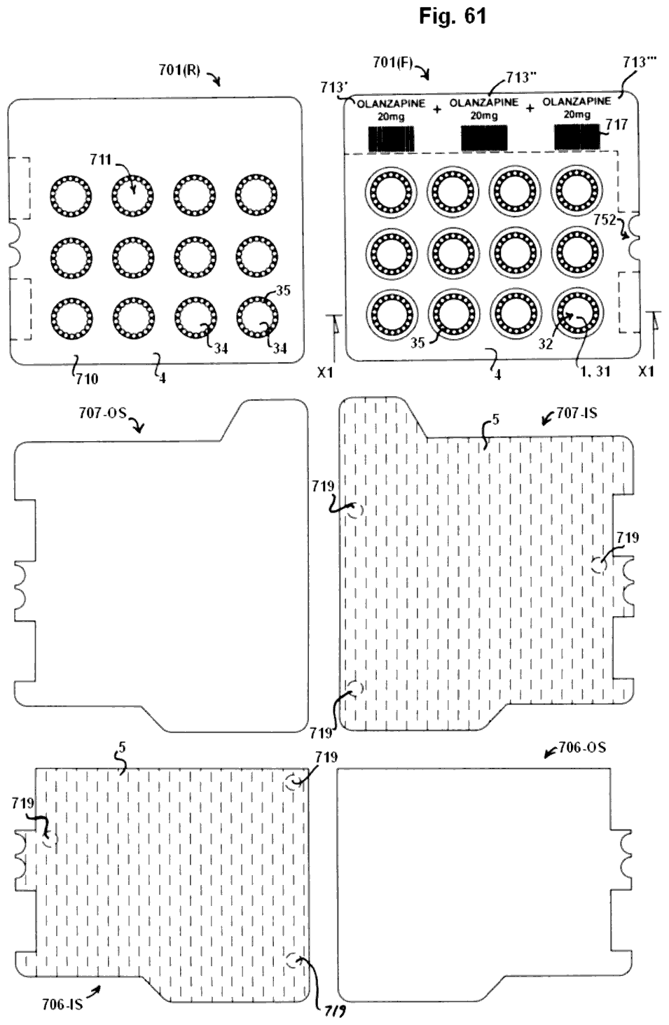

FIG. 61 shows, in front and rear views, the frame and front and rear covers of a FIRST DRUG PACKAGE including a first drug (olanzapine, 20 mg) for use with the second machine.

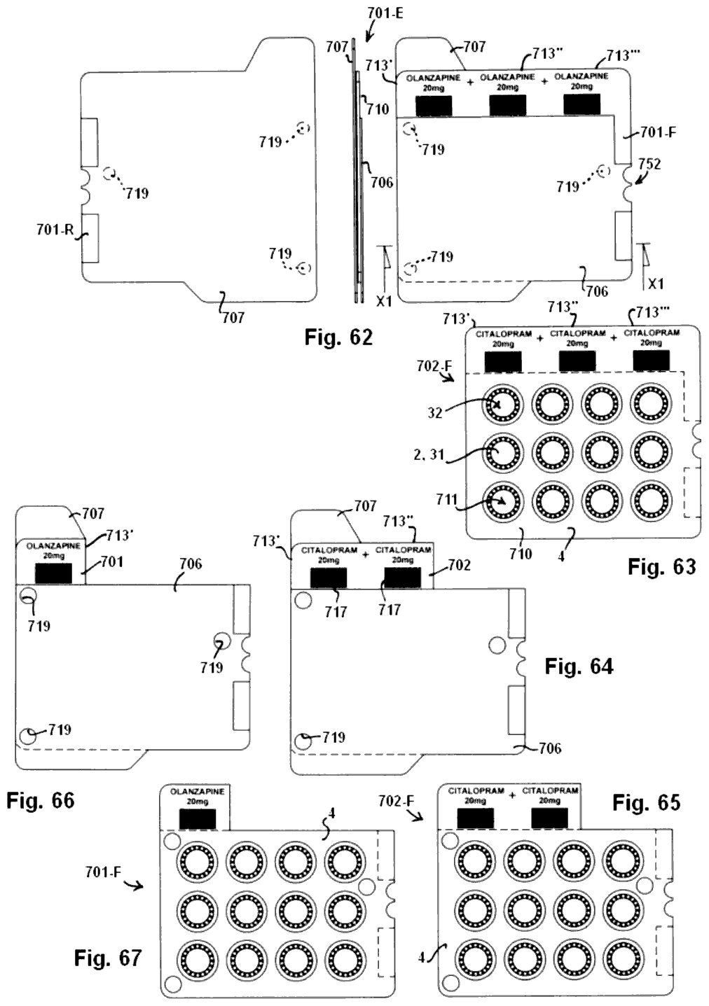

FIG. 62 shows, in front, rear and trailing end views, the FIRST DRUG PACKAGE comprising the frame enclosed between the front and rear covers as shown in FIG. 61.

FIG. 63 shows the frame of a SECOND DRUG PACKAGE similar to that of the first drug package of FIG. 61 except that it includes a different, second drug (citalopram, 20 mg).

FIG. 64 shows the second drug package including its frame and covers, as contained in slot no. 2 of the second machine following a first stage of operation in which its connector portions and one of its label portions have been punched out.

FIG. 65 shows the frame of the second drug package as contained in the second machine after it has been separated from its covers and displaced from slot no. 2 in a second stage of operation.

FIG. 66 shows the first drug package as contained in slot no. 1 of the second machine following the first stage of operation in which its connector portions and two of its label portions have been punched out.

FIG. 67 shows the frame of the first drug package as contained in the second machine after it has been separated from its covers and displaced from slot no. 1 in the second stage of operation.

FIG. 68 shows front, rear and trailing end views of a REAR COVER PACKAGE for use with the second machine (at the bottom of the drawing), together with front and rear views of its frame and cover as separate components.

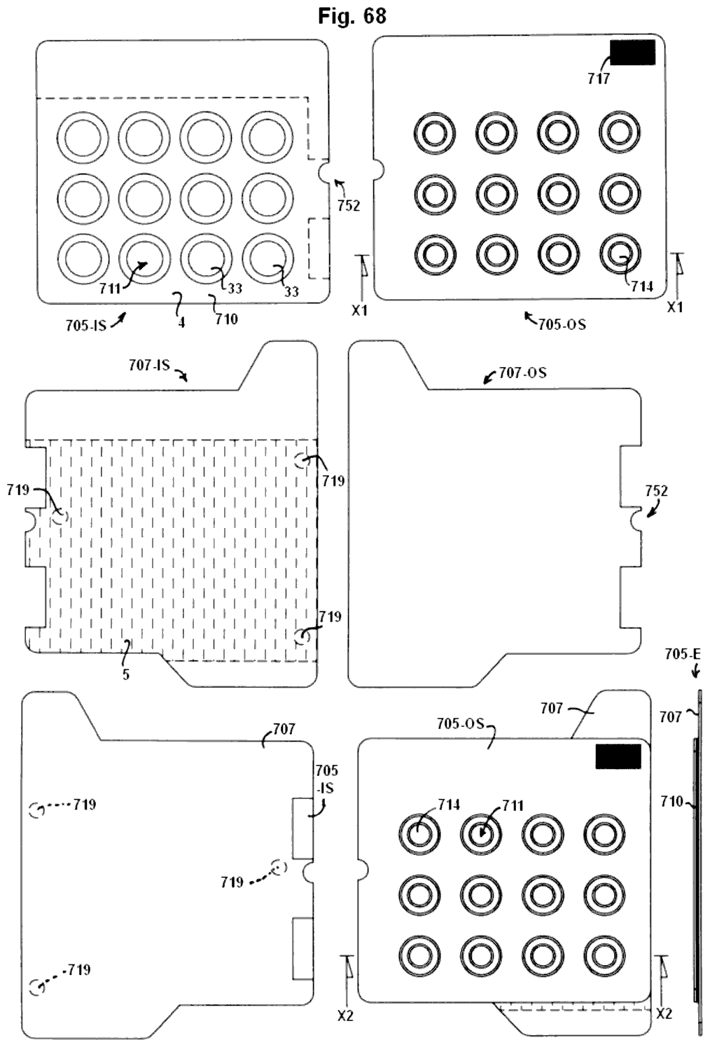

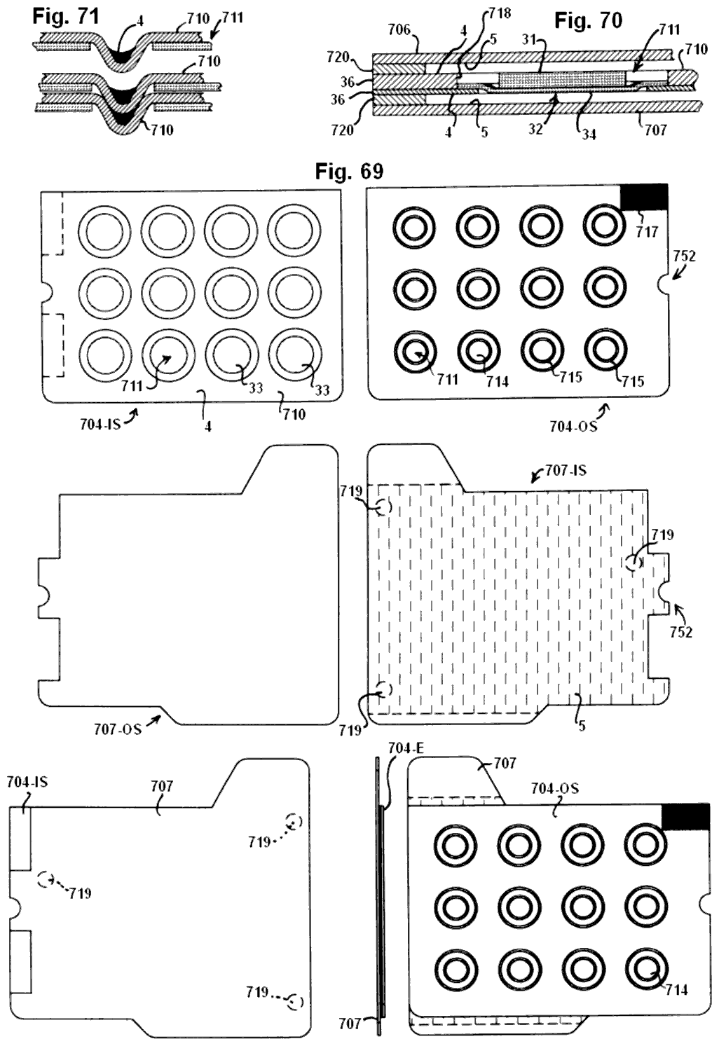

FIG. 69 shows front, rear and trailing end views of a FRONT COVER PACKAGE for use with the second machine (at the bottom of the drawing), together with front and rear views of its frame and cover as separate components.

FIG. 70 is a cross-section through one cell of the second drug package for use with the second machine, in an alternative construction.

FIG. 71 is a fragmentary cross-section through a part of the frame, between two adjacent cells, of each of three drug packages for use with the second machine, showing the three packages being stacked together, in another alternative construction.

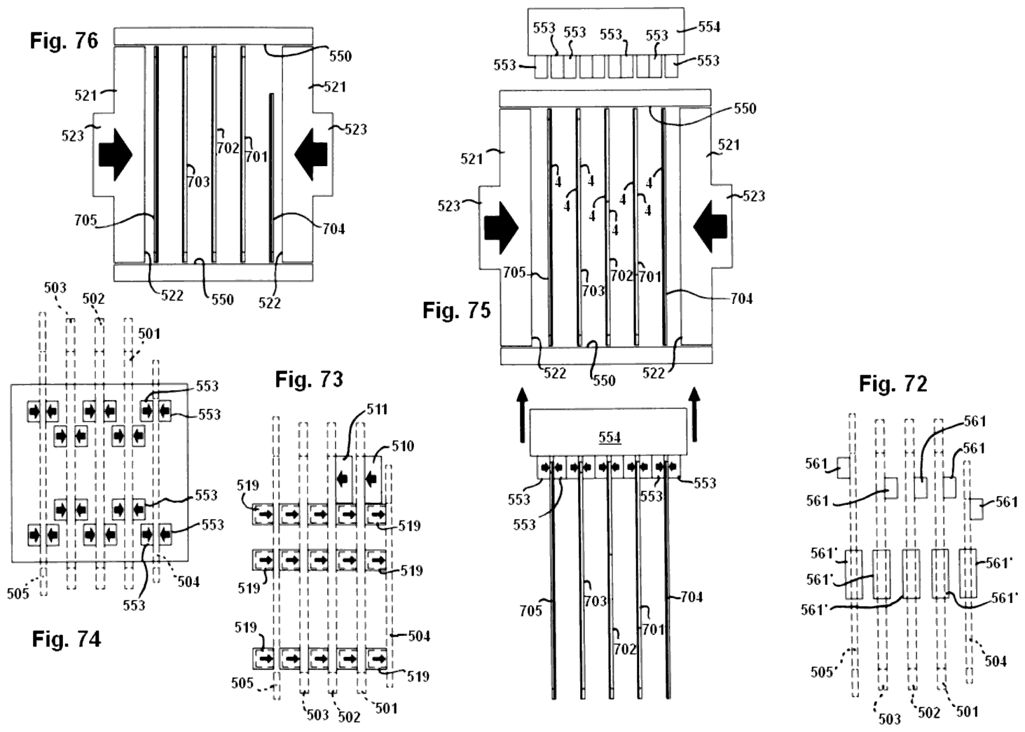

FIGS. 72-74 are schematic front views of the five package receiving slots of the second machine, showing different functional parts of the second machine; wherein:

FIG. 72 shows the package sensors;

FIG. 73 shows the package cutters; and

FIG. 74 shows the package transfer assembly.

FIG. 75 is a schematic plan view of the package transfer assembly and compression assembly of the second machine, illustrating the package transfer assembly with five packages, both before and after movement from the initial position to the assembly position.

FIG. 76 is a schematic front view of the compression assembly of the second machine, in the assembly position of the packages as shown in FIG. 75.

FIG. 77 is a schematic side view of the package receiving space defined by slot no. 2 of the second machine, looking from the right hand side of the machine as shown in FIG. 60.

FIG. 78 is a view corresponding to FIG. 77, showing the positions of the package sensors, package cutters, and package transfer assembly in slot no. 2.

FIG. 79 is a view corresponding to FIG. 78, illustrating the movement of the package transfer assembly with the second drug package, from the initial position of the second drug package in slot no. 2 to the assembly position of the second drug package, as shown in FIG. 75.

FIG. 80 shows the finished SECOND END USER PACKAGE in front, rear, top, and trailing end view. It should be noted that only two drug packages are shown in the front view, whereas three drug packages are shown in the top and trailing end views.

FIG. 81 shows an enlarged front view of one cell of the first drug package as shown in FIG. 61; and below the front view, four sections taken at the position of section line X1-X1 of FIG. 61 through the same cell and components thereof in different stages of assembly, showing from top to bottom:
the API film and carrier film assembly before assembly to the frame;
the frame before insertion of the API film and carrier film assembly;
the frame after insertion of the API film and carrier film assembly, i.e. all the components as shown in FIG. 61; and
the frame after insertion of the API film and carrier film assembly and enclosed between the front and rear covers to form the first drug package, as at section line X1-X1 of FIG. 62.

FIG. 82 shows the edible disc of one cell of the rear cover package as shown in FIG. 68, in front view and, immediately below the front view, from the side showing its thickness dimension; and below those two views, three sections taken at the position of section line X1-X1 of FIG. 68 through the same cell and components thereof in different stages of assembly, showing from top to bottom:
the frame and frangible foil before assembly of the edible disc;
the frame and frangible foil after assembly of the edible disc; and
the frame and frangible foil after assembly of the edible disc and with the cover attached, as at section line X2-X2 of FIG. 68.

FIGS. 83-85 show one cell of another drug package in three consecutive assembly steps in accordance with an alternative construction, in each case in front view and in section at section line X1-X1 of the same figure; wherein:

FIG. 83 shows the frame before introducing the carrier film;

FIG. 84 shows the frame with the carrier film ready to receive the API film; and

FIG. 85 shows the frame with the API film deposited on the carrier film.

FIG. 86 is a section taken at section line X1-X1 of FIG. 80 through one cell of the second end user package (as shown, including only two drug packages), showing how the frangible foils are urged together by ambient air pressure to form the pastille.

FIG. 87 is a section corresponding to FIG. 86 after removal of the pastille for consumption, wherein the pastille is shown in front view and from the side showing its thickness dimension.

FIGS. 88-101 show a VARIANT of the SECOND MACHINE and packages for use therewith, wherein:—

FIG. 88 shows a variant of the second machine (referred to hereafter as the variant second machine), at reduced scale compared with FIGS. 89 and 90.

FIG. 89 is a front view of a VARIANT SECOND END USER PACKAGE as produced by the variant second machine.

FIG. 90 is a trailing end view of the variant second end user package received in the package delivery window of the variant second machine.

FIG. 91 shows a variant, second drug package, including its frame and covers, as contained within slot no. 2 of the variant second machine following a first stage of operation in which its connector portions and one of its label portions have been punched out, wherein the locator rods of the package transfer assembly are seen in cross-section.

FIG. 92 is a front view of part of the package transfer assembly as shown in FIG. 91.

FIG. 93 shows how the package transfer assembly is operated to remove the frame of the variant second drug package from between its covers.

FIG. 94 shows the package transfer assembly in the next stage of operation, showing how the package alignment mechanism is operated to align the packages in parallel relation.

FIG. 95 is a front view showing more of the package transfer assembly following the alignment step of FIG. 94.

FIG. 96 is a front view of part of the package transfer assembly and the compression assembly, showing how the package alignment mechanism is operated in a further step to reduce the distance between the packages.

FIG. 97 is a front view corresponding to FIG. 96, showing how the package transfer assembly is displaced in a further step to position the stacked packages in the assembly position prior to operation of the compression assembly.

FIG. 98 is a front view corresponding to FIG. 97, showing the stacked packages in the assembly position following a further step in which the package alignment mechanism is withdrawn.

FIG. 99 shows the operation of the compression mechanism in a further step to form the variant second end user package.

FIG. 100 shows a further step in which the package transfer assembly is displaced to move the variant second end user package to the package delivery window; and

FIG. 101 shows how the locator rods are withdrawn in a further step, leaving the variant second end user package ready for collection from the package delivery window as shown in FIG. 90.

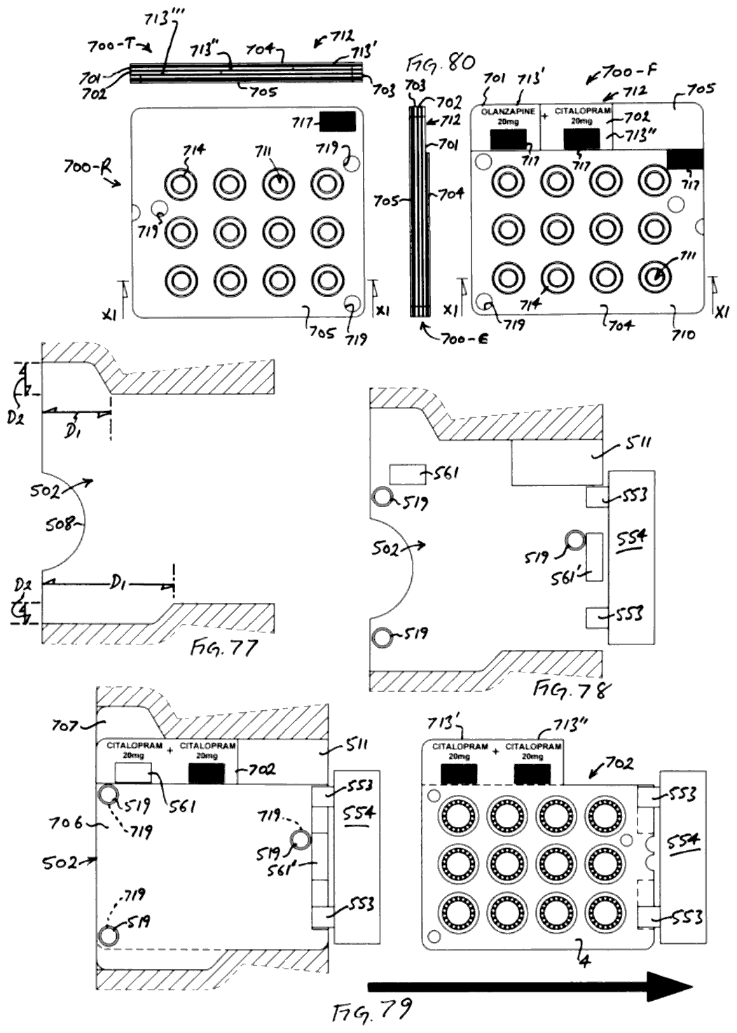

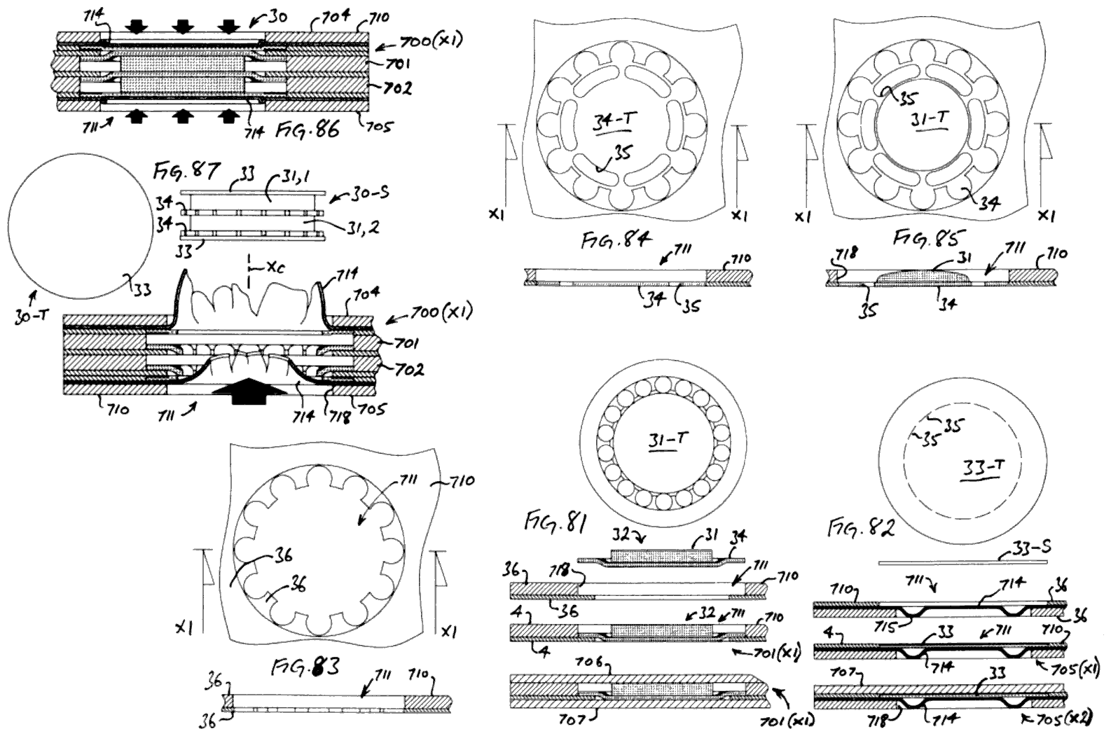

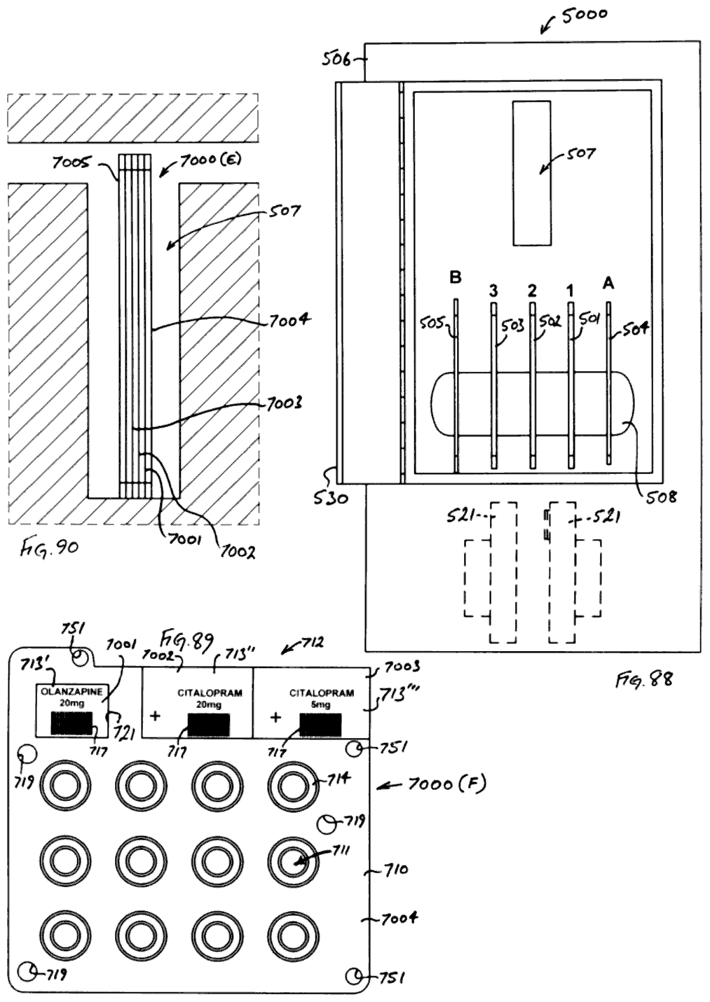

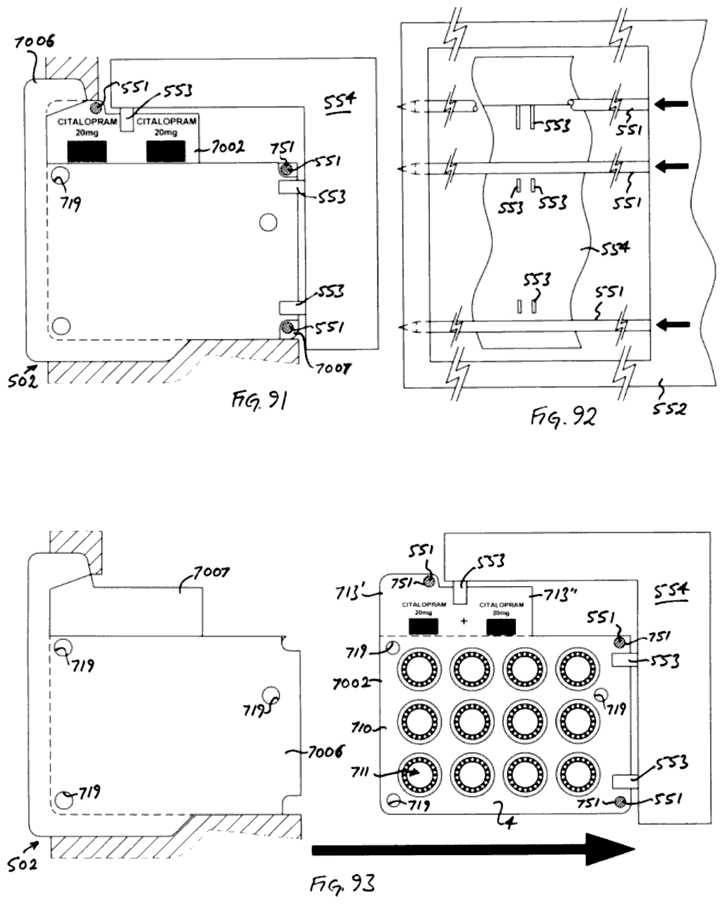

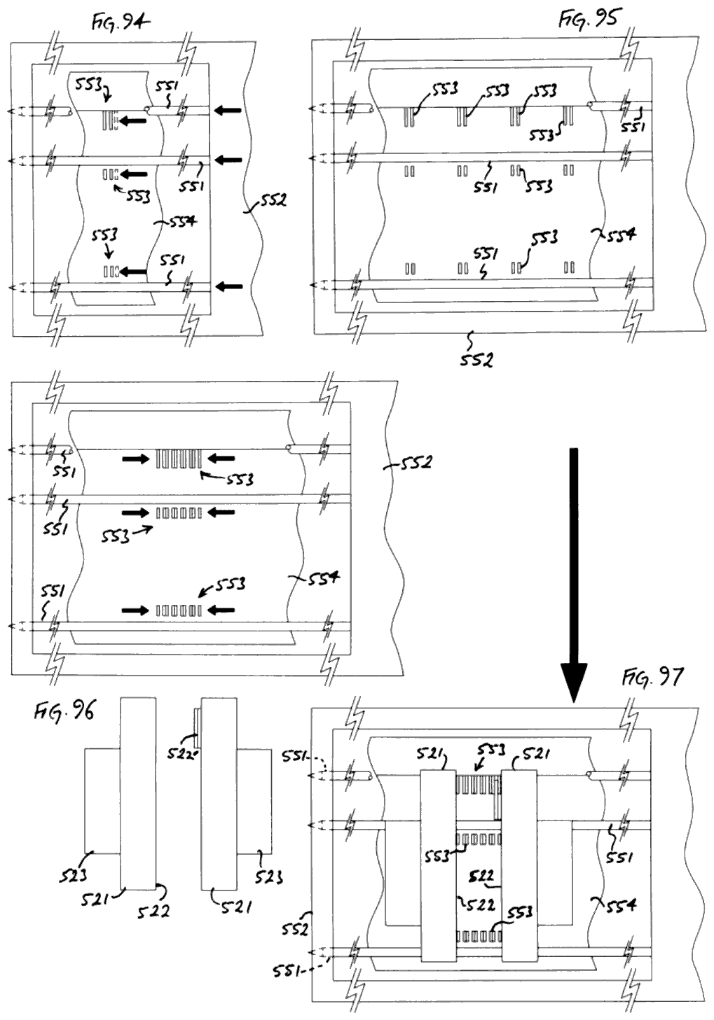

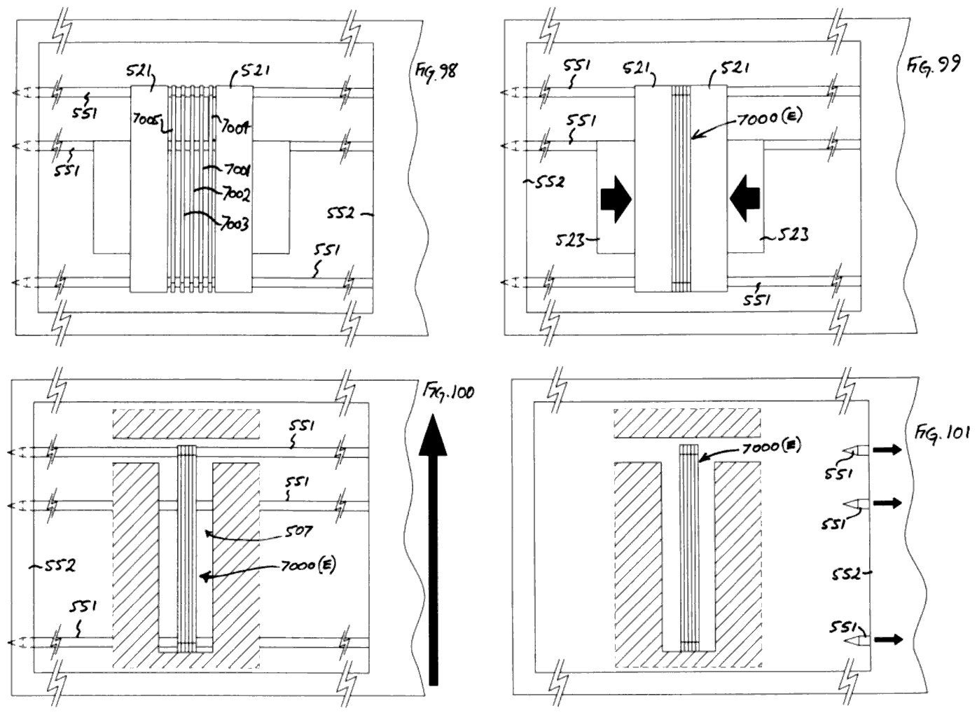

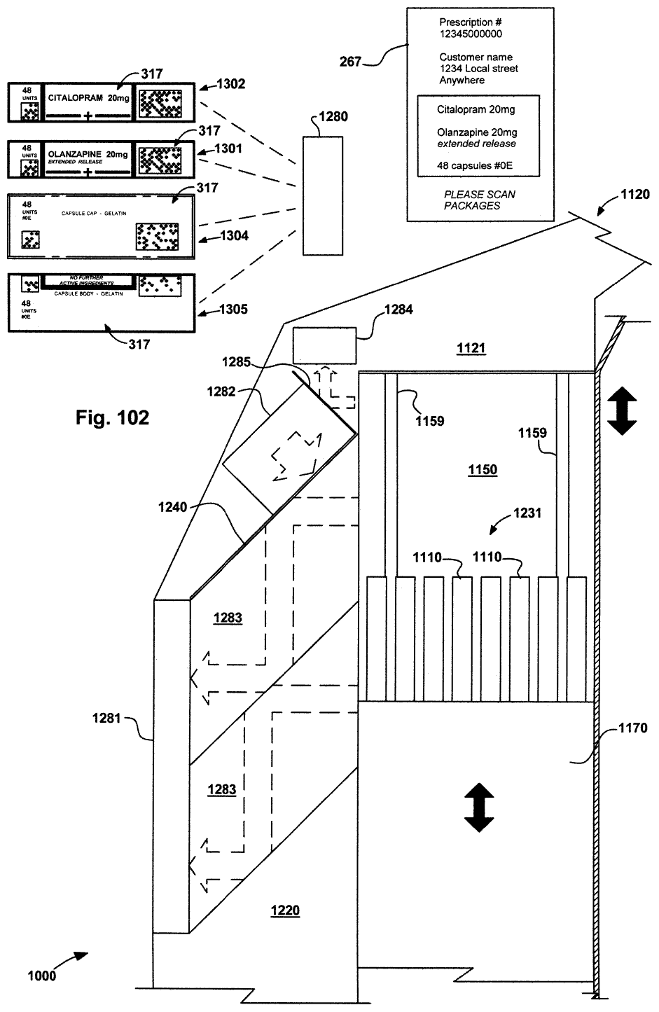

FIGS. 102-159 show a VARIANT of the FIRST MACHINE and packages and a carrier for use therewith, wherein:—

FIG. 102 is a schematic side view of the variant first machine showing how the packages are scanned before they are stacked in the machine.

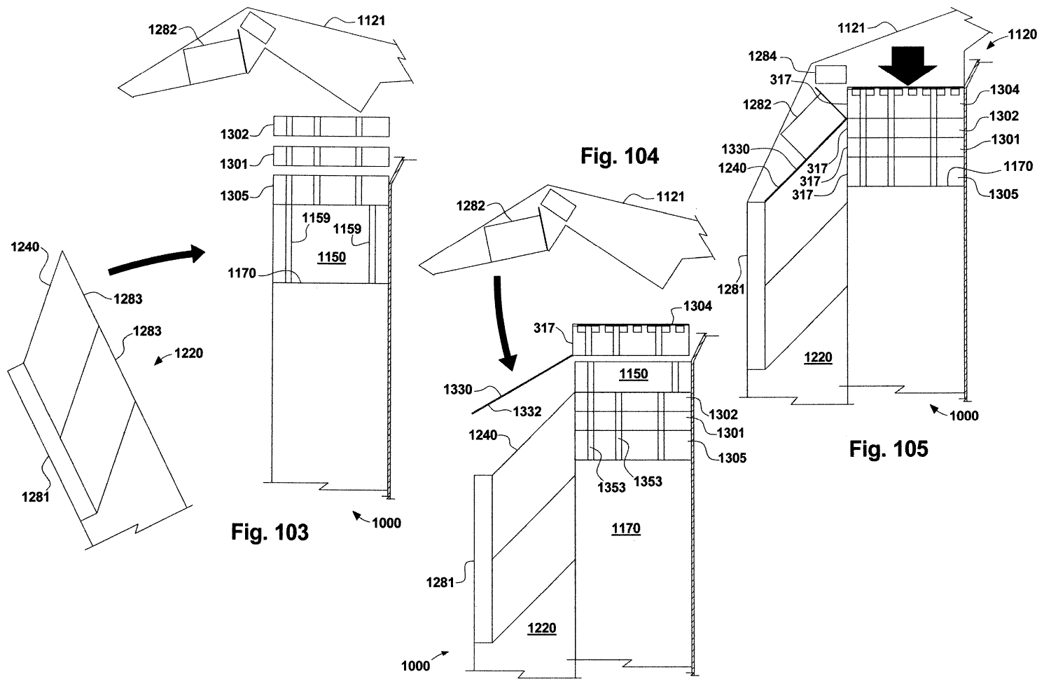

FIG. 103 shows how the capsule body package and drug packages are stacked in the machine.

FIG. 104 shows how the cap package is added to the stack with its flap opened out on the support surface.

FIG. 105 shows how the top plate is closed onto the stack ready for the assembly operation.

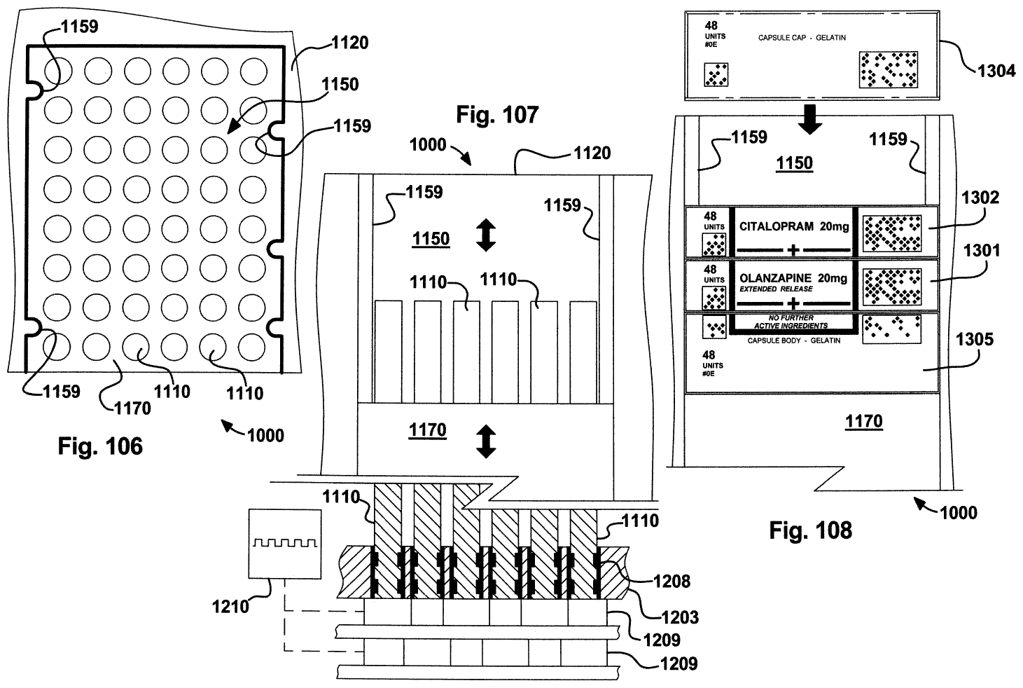

FIG. 106 is a top view of the pedestal block received in the profiled recess of the moving frame assembly.

FIG. 107 is a partial front view showing the pedestal block received in the profiled recess of the moving frame assembly, with part of the fixed pushrod assembly shown in vertical section.

FIG. 108 is a partial front view showing the packages stacked in the machine.

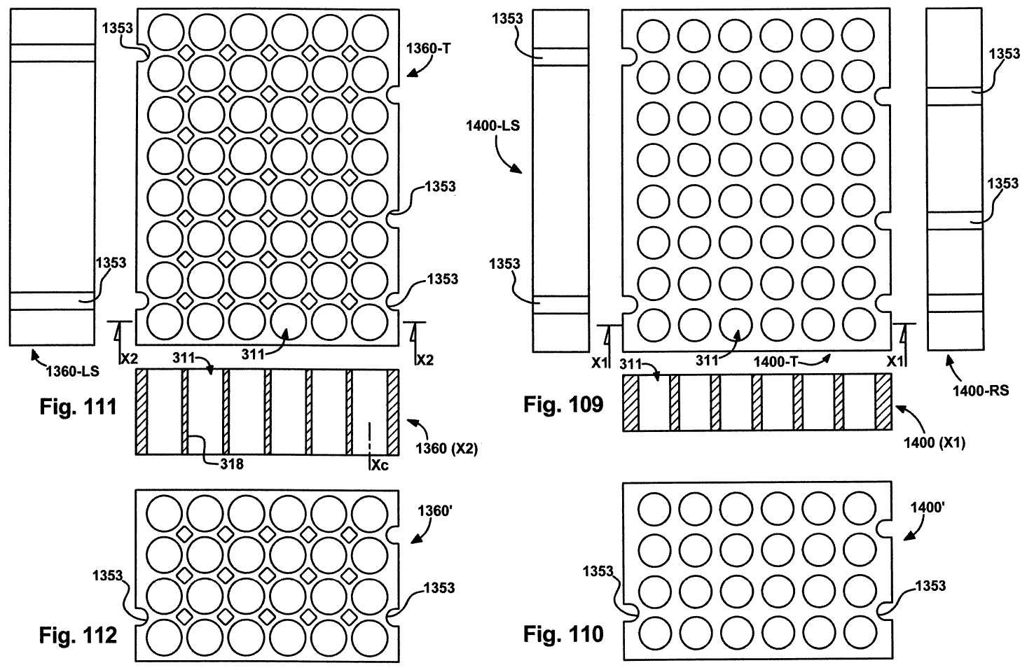

FIG. 109 shows various views of the block of a 48-cell drug package, including a section taken at X1-X1 of FIG. 109.

FIG. 110 is a top view of the block of a 24-cell drug package.

FIG. 111 shows various views of the block of a 48-cell cap package, including a section taken at X2-X2 of FIG. 111.

FIG. 112 is a top view of the block of a 24-cell cap package.

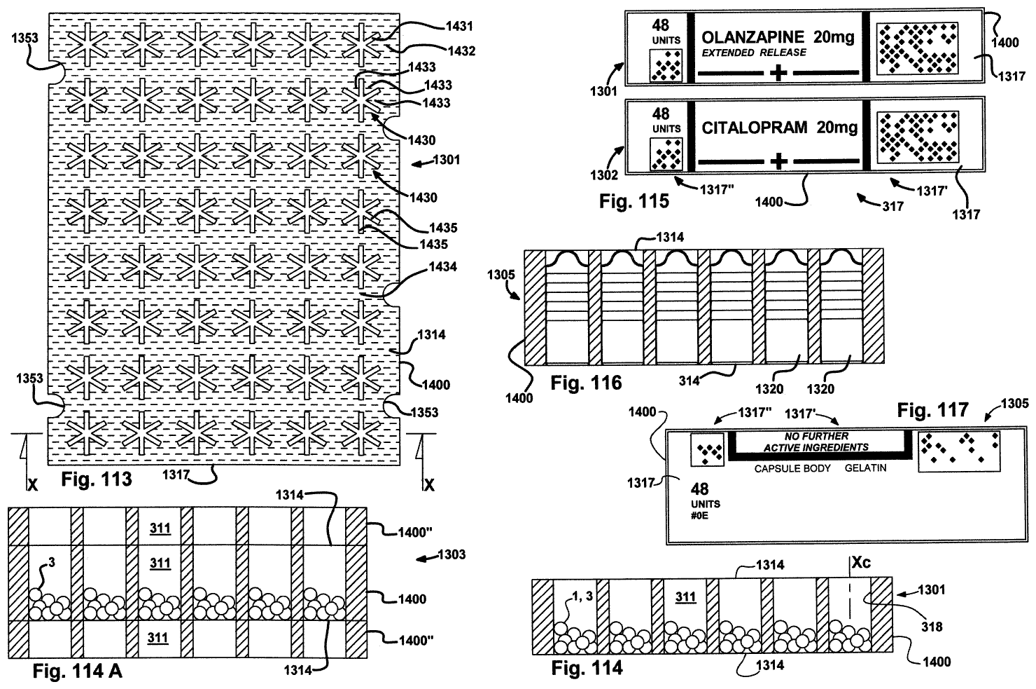

FIG. 113 is a top view of a 48-cell drug package closed by a patterned foil.

FIG. 114 is a section through the 48-cell drug package taken at X-X of FIG. 113.

FIG. 114 A is a section through an alternative, composite drug package, taken in the same position as FIG. 114.

FIG. 115 is a front view of two 48-cell drug packages.

FIG. 116 is a section through a capsule body package taken in the same position as X-X of FIG. 113.

FIG. 117 is a front view of the capsule body package.

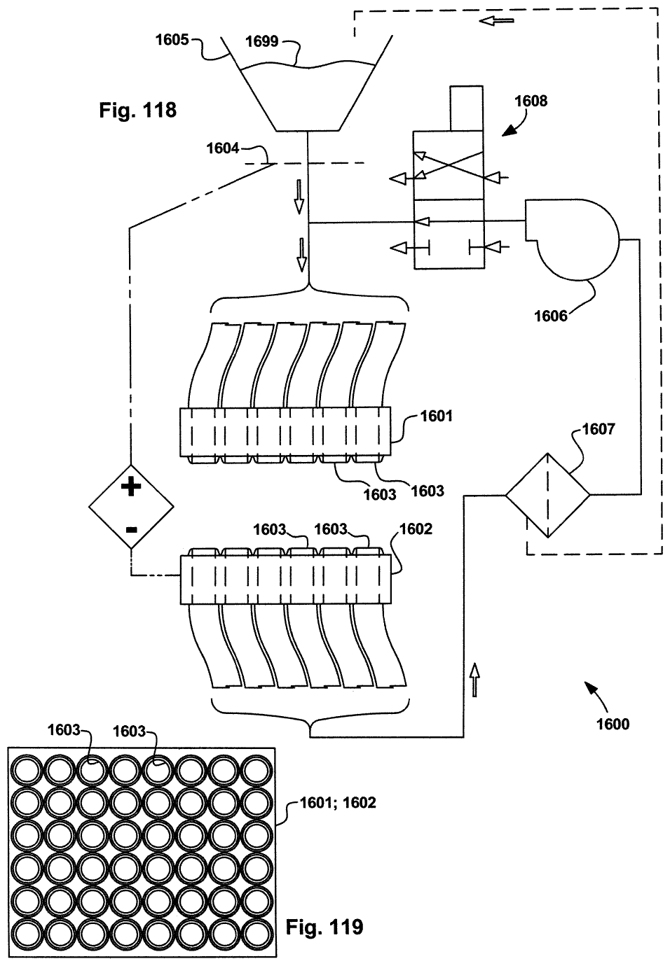

FIG. 118 shows an apparatus for flocking the cells of the block of the cap package as shown in FIG. 111.

FIG. 119 shows the face of the nozzle array of the flocking apparatus of FIG. 118.

FIGS. 120-135 show the CAP PACKAGE and parts thereof, wherein:—

FIG. 120 is a top view of the block of the 48-cell cap package, as shown in FIG. 111, after flocking the cells.

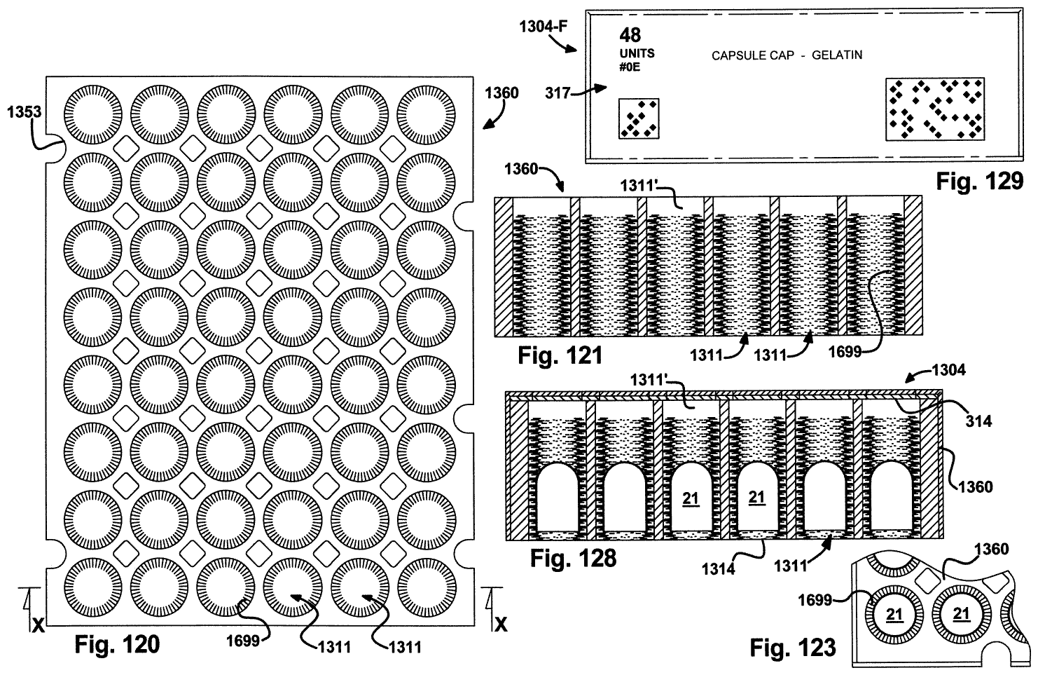

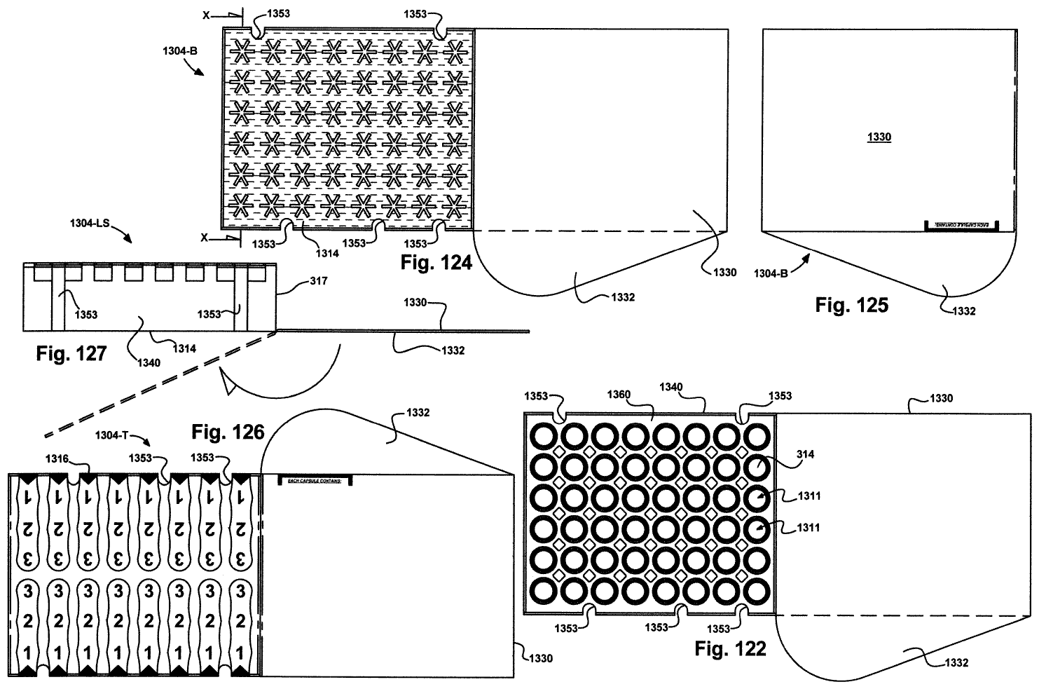

FIG. 121 is a section through the flocked cells taken at X-X of FIG. 120.

FIG. 122 is a bottom view of the cap package ready to be filled with the capsule caps.

FIG. 123 is a bottom view of part of the cap package as shown in FIG. 122, looking into the empty capsule caps which have been inserted into the flocked cells, and before the block is sealed with a patterned foil.

FIG. 124 is a bottom view of the finished cap package after filling the block with the capsule caps and sealing it with a patterned foil.

FIG. 125 is another bottom view of the cap package, showing the flap folded over for storage.

FIG. 126 is a top view of the finished cap package.

FIG. 127 is a left side view of the cap package.

FIG. 128 is a section through the filled cap package at X-X of FIG. 124, showing how the caps are located centrally in the flocked cells.

FIG. 129 is a front view of the cap package.

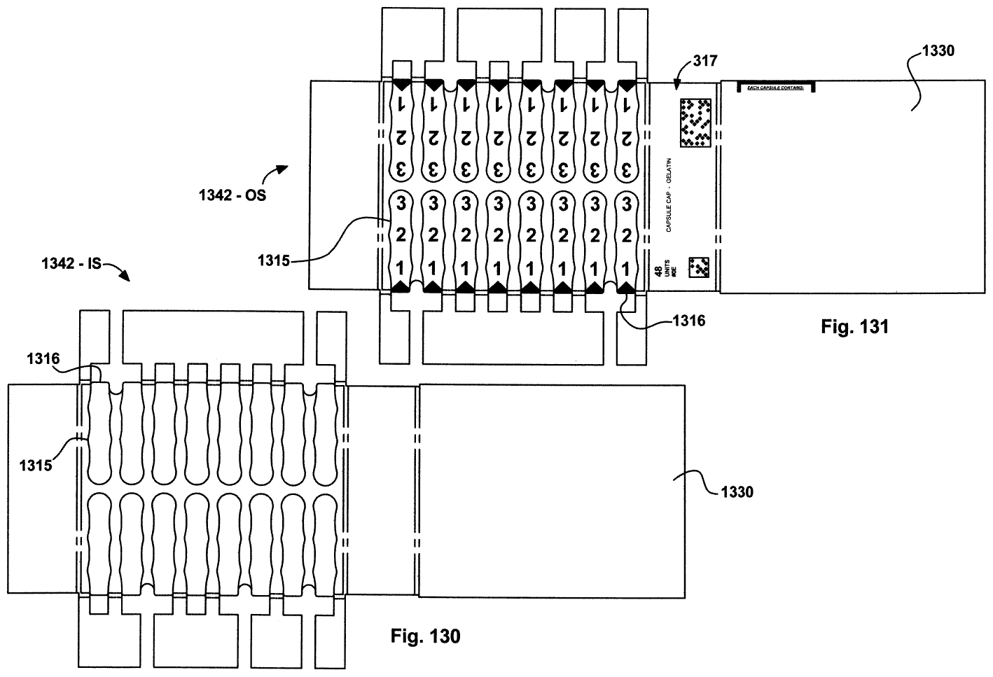

FIG. 130 is an inner side view of the outer casing sheet of the cap package, as formed from a flat sheet of card.

FIG. 131 is an outer side view of the outer casing sheet of FIG. 130.

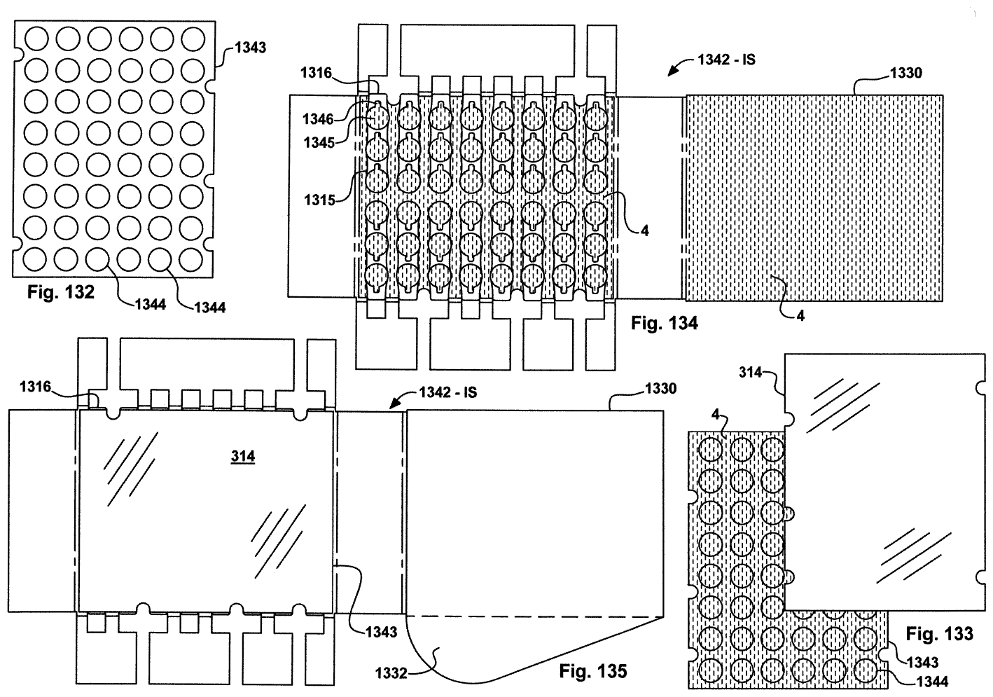

FIG. 132 shows the inner casing sheet of the cap package, forming an array of discs which have been punched but not removed from the sheet.

FIG. 133 shows how the inner casing sheet of FIG. 132 is coated on one side with adhesive and covered by a sheet of plain (unpatterned) foil which holds the punched discs in place.

FIG. 134 illustrates the outer casing sheet in the same position as FIG. 130, showing how adhesive is applied in a pattern to the inner side of the tear-off strips and to the inwardly facing side of the flap.

FIG. 135 shows the finished outer casing of the cap package, before folding it around the block of FIG. 120 to form the empty cap package as shown in FIG. 122. The outer casing sheet is shown in the same position as FIG. 134, after applying the reverse (non-foil covered) side of the inner casing sheet of FIG. 133 to the patterned adhesive of FIG. 134, and after applying a release paper to the adhesive side of the flap.

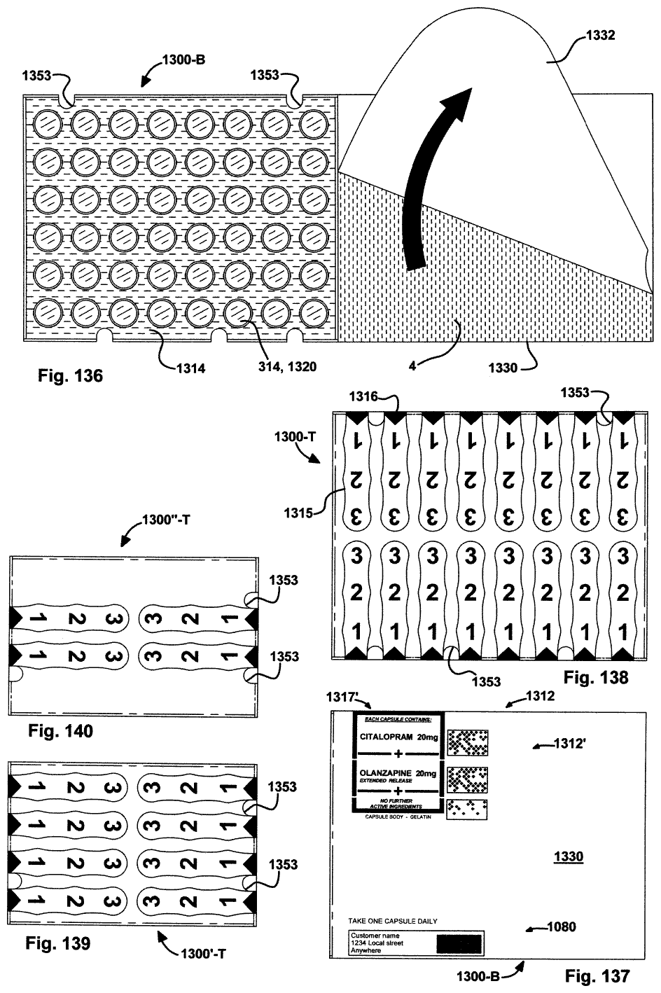

FIGS. 136-138 show the END USER PACKAGE incorporating the cap package of FIGS. 124-129, wherein:—

FIG. 136 shows the bottom side of the end user package with the carriers received in the cells, after it has been removed from the machine, and with the release paper partially peeled from the flap.

FIG. 137 is another bottom view of the end user package, showing the composite label printed on the flap which has been stuck in place to cover the ends of the carriers.

FIG. 138 is a top view of the end user package of FIG. 137.

FIG. 139 is a top view of another end user package, formed from the 24-cell block of FIG. 112 and containing 24 capsules.

FIG. 140 is a top view of another end user package, formed from the 24-cell block of FIG. 112 and containing 12 capsules.

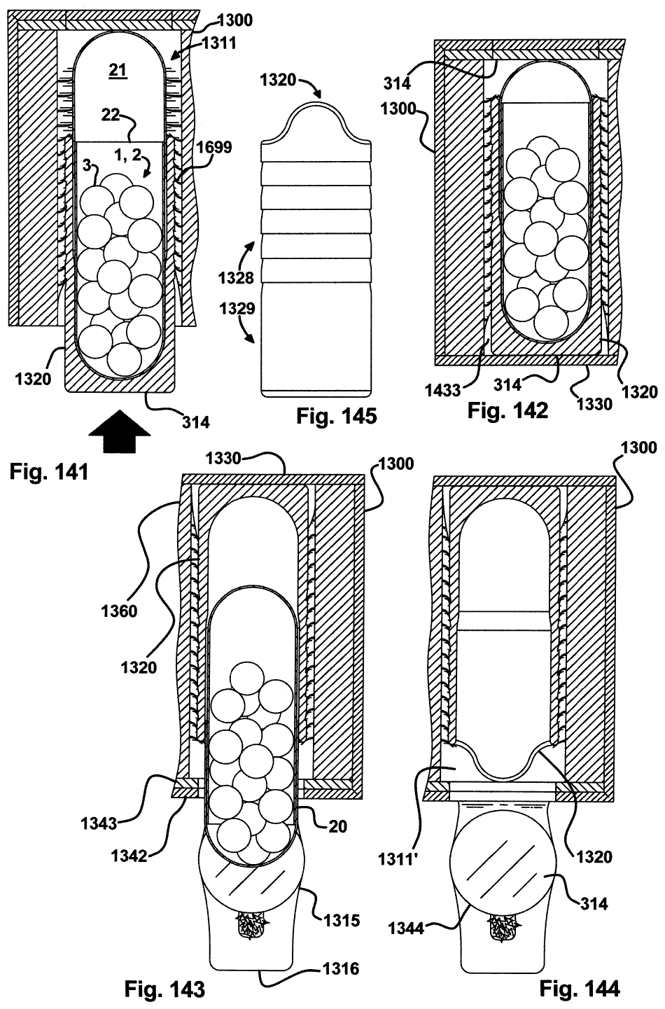

FIGS. 141-144 are SECTIONS THROUGH ONE CELL OF THE END USER PACKAGE of FIGS. 136-138, taken in the same plane as FIG. 128, wherein:—

FIG. 141 shows the carrier moving into the cell during assembly.

FIG. 142 shows the carrier inside the cell after assembly.

FIG. 143 shows how the cell is opened to remove the capsule.

FIG. 144 shows the empty carrier inside the cell, after removing the capsule.

FIG. 145 shows the carrier.

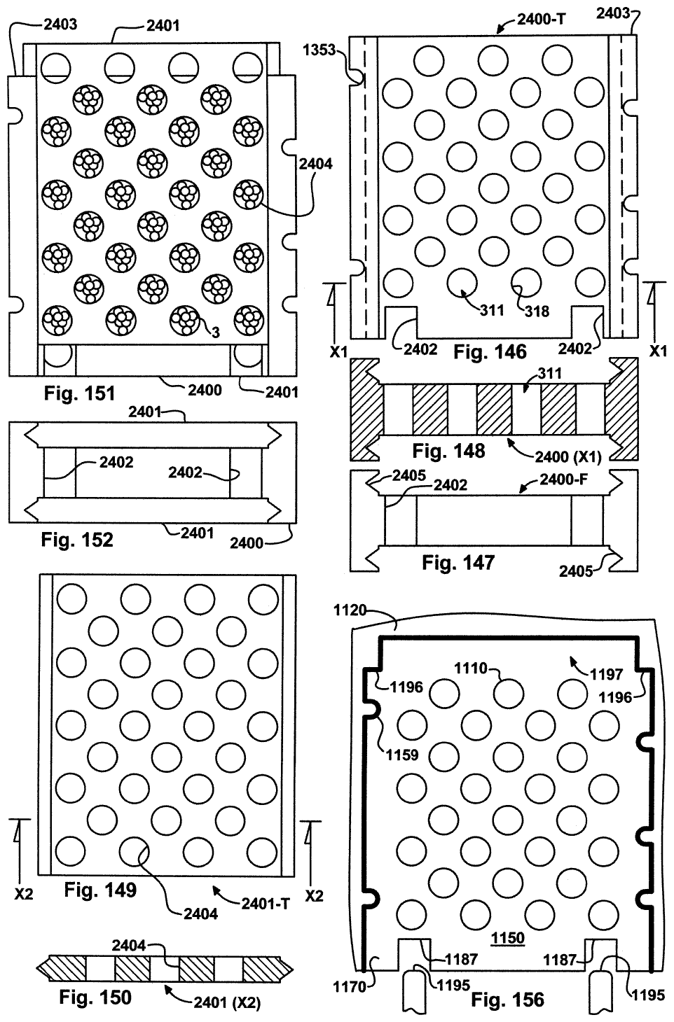

FIGS. 146-159 show an ALTERNATIVE DRUG PACKAGE with MOVABLE CLOSURE ELEMENTS, wherein:—

FIG. 146 is a top view of the empty block or frame.

FIGS. 147 and 148 show the block, respectively in end view and in section at X1-X1 of FIG. 146.

FIGS. 149 and 150 show a foraminous plate forming a movable closure element, respectively in top view, and in section at X2-X2 of FIG. 149.

FIGS. 151 and 152 show the partially assembled drug package with the cells filled and before closing the upper closure element, respectively in top view and end view.

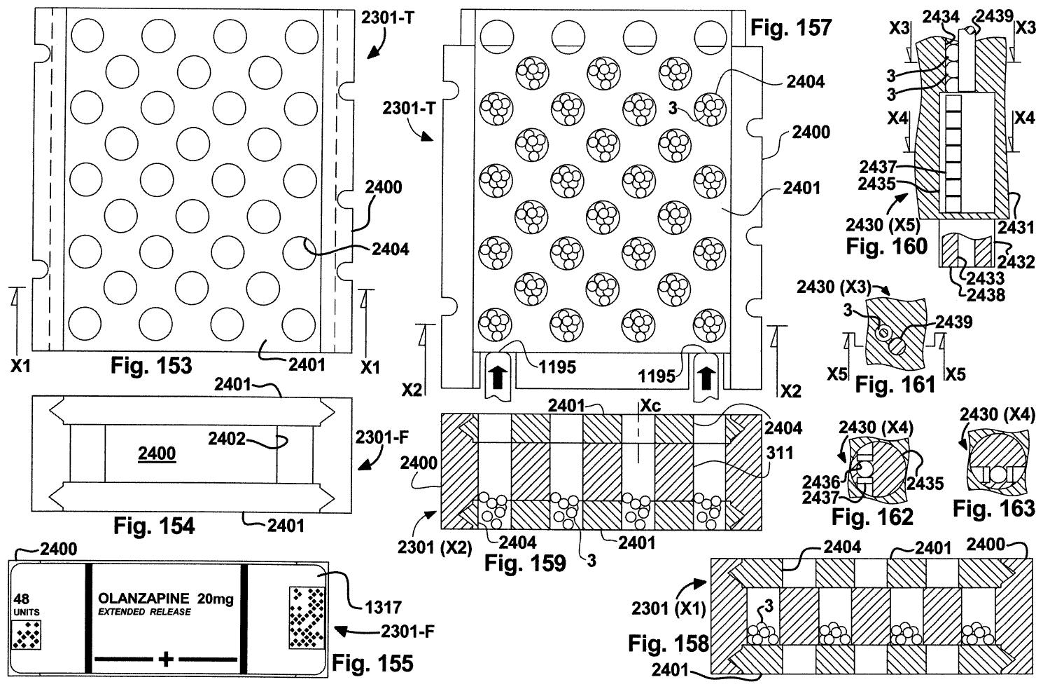

FIGS. 153, 154 and 155 show the drug package with both closure elements in the closed position, respectively in top view, in end view before applying the label, and in end view after applying the label.

FIG. 156 shows the profiled recess of the variant first machine as adapted to receive the alternative drug package.

FIG. 157 shows the alternative drug package received in the profiled recess (not shown, for clarity) with the movable closure elements displaced to the open position.

FIGS. 158 and 159 are sections through the alternative drug package, taken respectively at X1-X1 of FIG. 153 with the movable closure elements in the closed position, and at X2-X2 of FIG. 157 with the movable closure elements in the open position.

FIGS. 160-163 show a FILLING APPARATUS for use in filling the cells of the alternative drug package of FIGS. 146-159, wherein:—

FIG. 160 is a partial longitudinal section at X5-X5 of FIG. 161, showing the dosing body in the receiving position.

FIG. 161 is a cross-section at X3-X3 of FIG. 160.

FIG. 162 is a cross-section at X4-X4 of FIG. 160.

FIG. 163 is a cross-section taken in the same position as FIG. 162, showing the dosing body rotated to the discharge position.

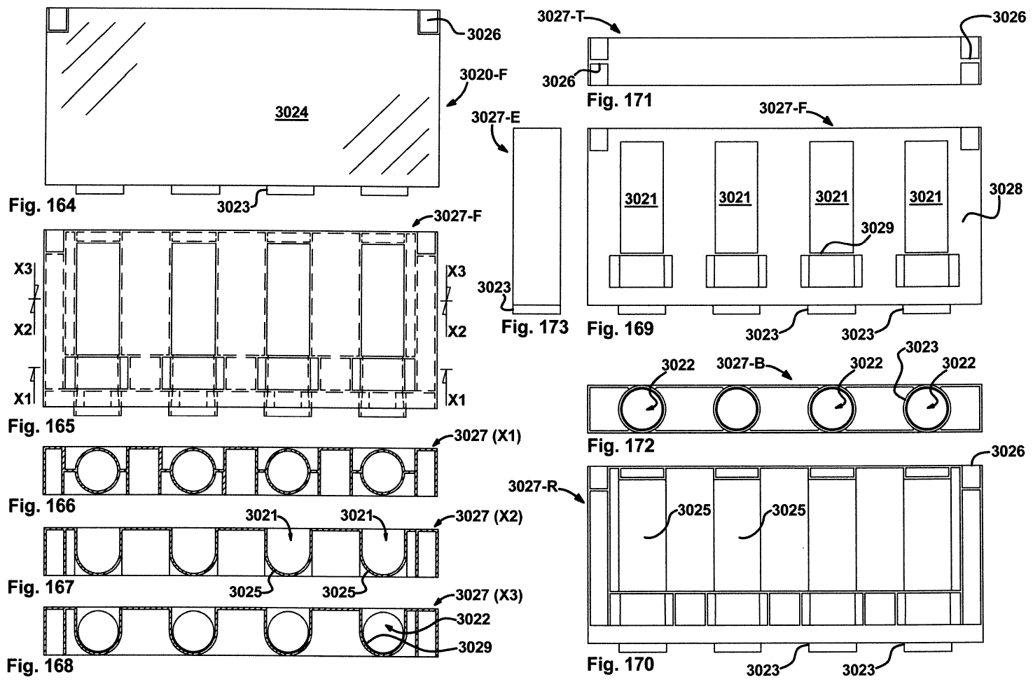

FIGS. 164-206 show a FURTHER VARIANT of the first embodiment comprising an END USER MULTIPACK in which the capsule is closed OUTSIDE the sub-package, wherein:—

FIG. 164 is a front view of one empty sub-package of the end user multipack, showing the foil wall.

FIG. 165 shows the body of the sub-package before attachment of the foil wall, with hidden parts shown in phantom lines.

FIGS. 166-168 are sections through the body of the sub-package taken respectively at X1-X1 (FIG. 166), X2-X2 (FIG. 167), and X3-X3 (FIG. 168) of FIG. 165.

FIGS. 169-173 show the empty body of the sub-package without its foil wall, respectively in front, rear, top, bottom, and end view.

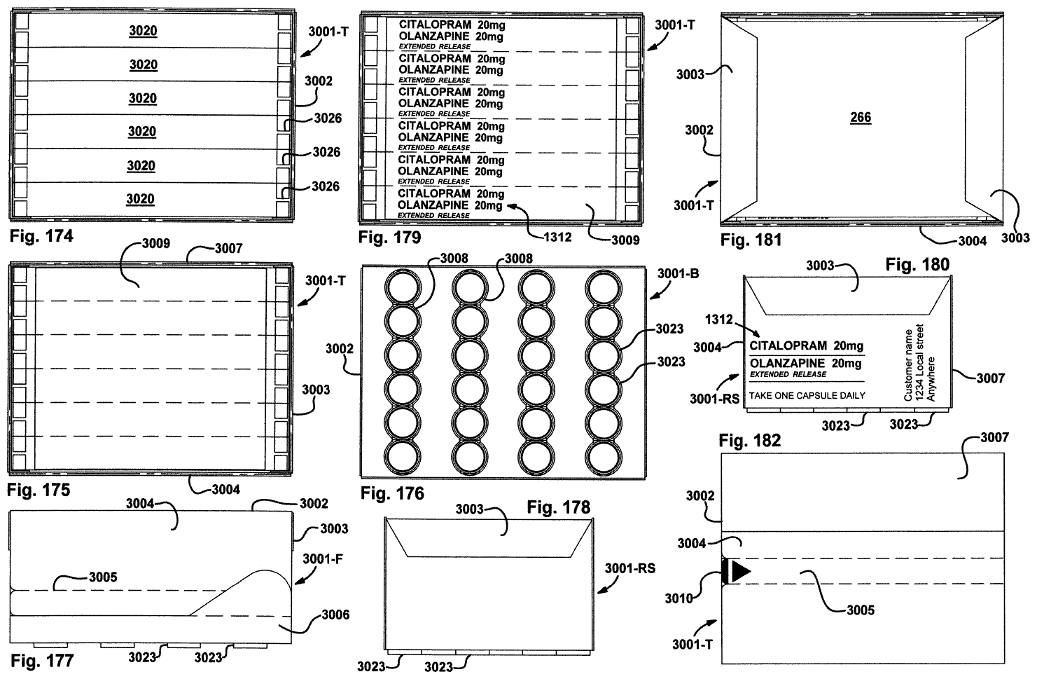

FIGS. 174-178 show the end user multipack comprising an assembly of seven empty sub-packages contained in a box, wherein:

FIG. 174 is a top view before attachment of the sub-package label;

FIG. 175 is a top view after attachment of the sub-package label;

FIG. 176 is a bottom view;

FIG. 177 is a side view showing the closure flap with a tear-off strip and release paper; and

FIG. 178 is an end view.

FIGS. 179 and 180 show the end user multipack containing the polypills after removal from the machine, respectively in top view (FIG. 179) and end view (FIG. 180).

FIG. 181 is a top view of the end user multipack with the patient information leaflet laid on top of the sub-package assembly and the end flaps folded over.

FIG. 182 is the same view as FIG. 181 after closing the side flaps, showing the end user multipack sealed and ready for delivery to the customer.

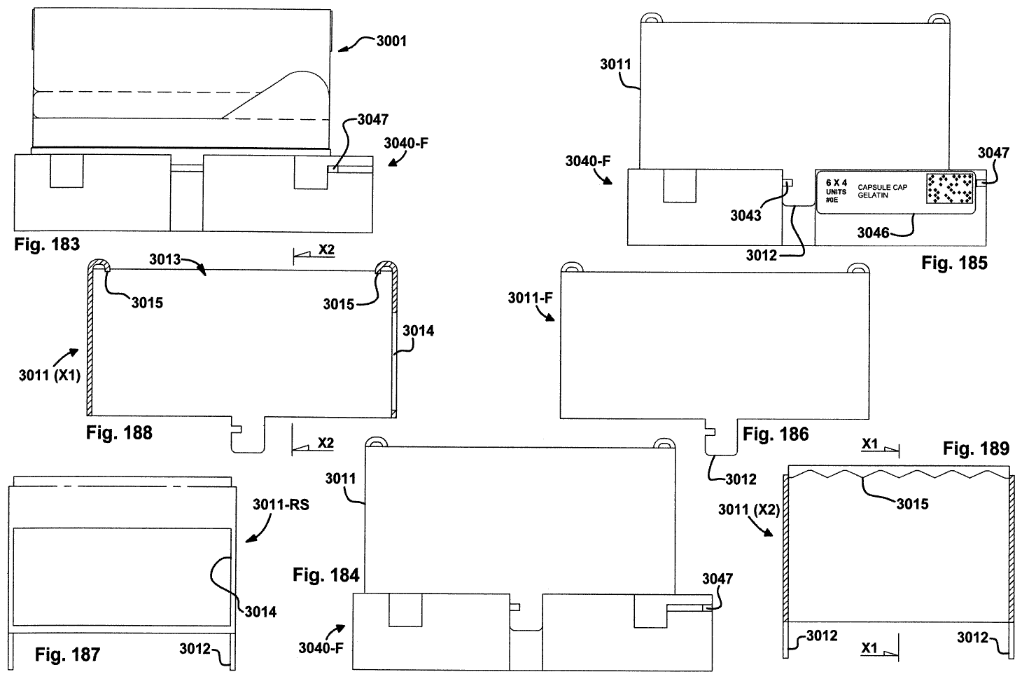

FIGS. 183-185 show the assembly of the empty end user multipack together with the cap package to form an end user multipack assembly, wherein:

FIG. 183 shows the empty end user multipack positioned on top of the cap package;

FIG. 184 shows the casing locked to the cap package to retain the end user multipack in position; and

FIG. 185 shows the anti-tamper label applied to the finished assembly.

FIGS. 186-189 show the casing of the multipack assembly, respectively: in front view (FIG. 186); in right end view (FIG. 187); in longitudinal section at X1-X1 of FIG. 189 (FIG. 188); and in cross-section at X2-X2 of FIG. 188 (FIG. 189).

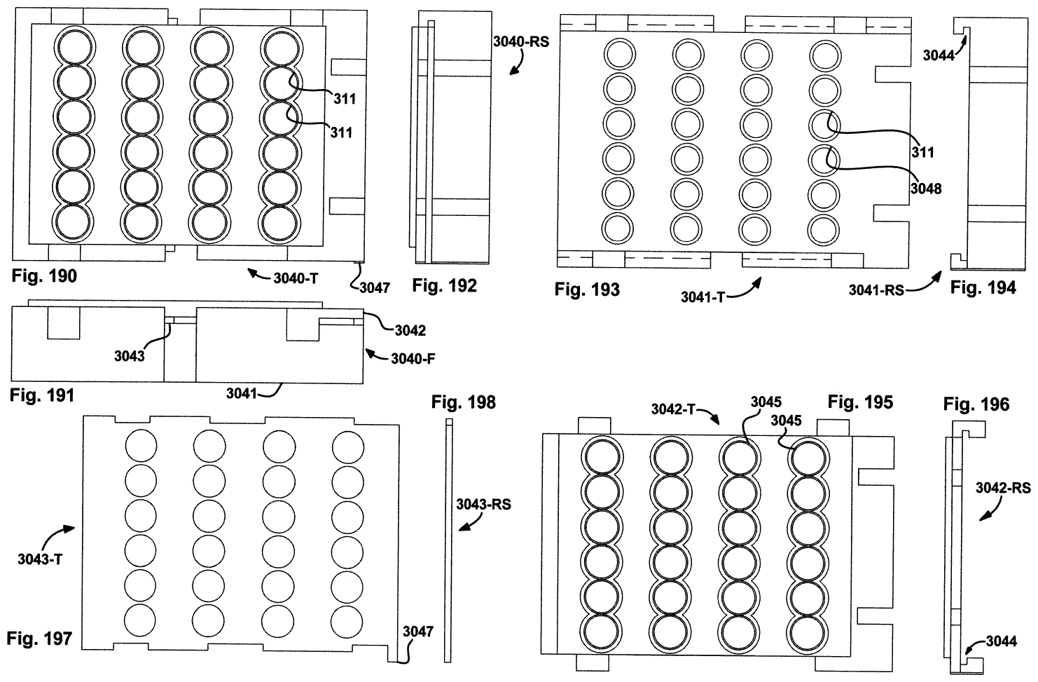

FIGS. 190-192 show the assembled cap package of the multipack assembly, respectively in top view (FIG. 190), front view (FIG. 191), and right end view (FIG. 192).

FIGS. 193-198 show the three body components of the cap package, wherein:

FIGS. 193 and 194 show the lower block, respectively in top and right end view;

FIGS. 195 and 196 show the upper block, respectively in top and right end view; and

FIGS. 197 and 198 show the movable closure element which locks the upper and lower blocks together, respectively in top and right end view.

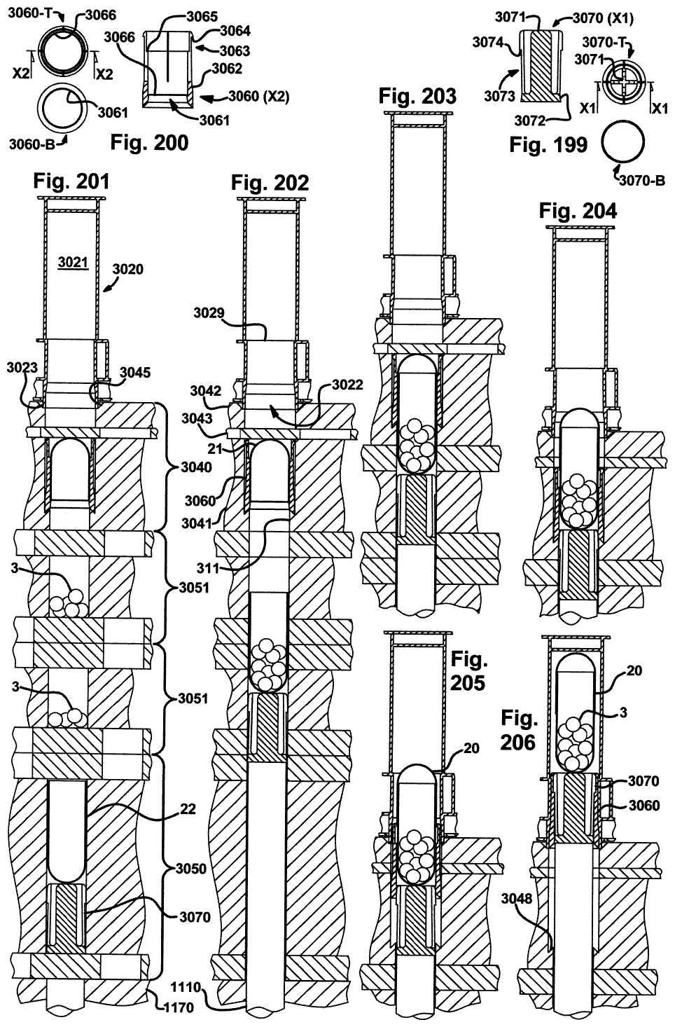

FIG. 199 shows the plug, respectively in top end view, in bottom end view, and in longitudinal section at X1-X1 of FIG. 199.

FIG. 200 shows the collar, respectively in top end view, in bottom end view, and in longitudinal section at X2-X2 of FIG. 200.

FIGS. 201-206 are longitudinal sections through one set of axially aligned cells of the stacked packages, showing consecutive stages in the operation of the assembly machine including filling and closing the capsule, inserting the capsule into an empty cell of the sub-package, and closing the cell with the plug and the collar.

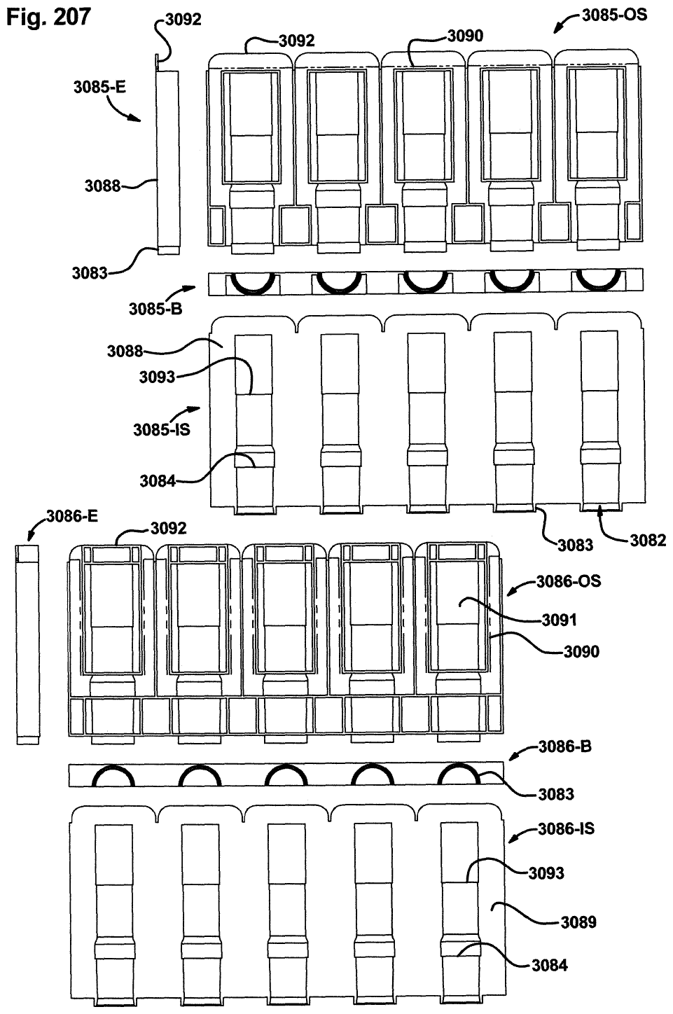

FIGS. 207-217 show a FURTHER VARIANT of the first embodiment comprising an END USER MULTIPACK in which the end user package forms a cap package so that the capsule is closed INSIDE the sub-package, wherein:—

FIG. 207 shows the front and rear mouldings of one clamshell sub-package, in each case in inner side (IS), outer side (OS), bottom (B) and end (E) view.

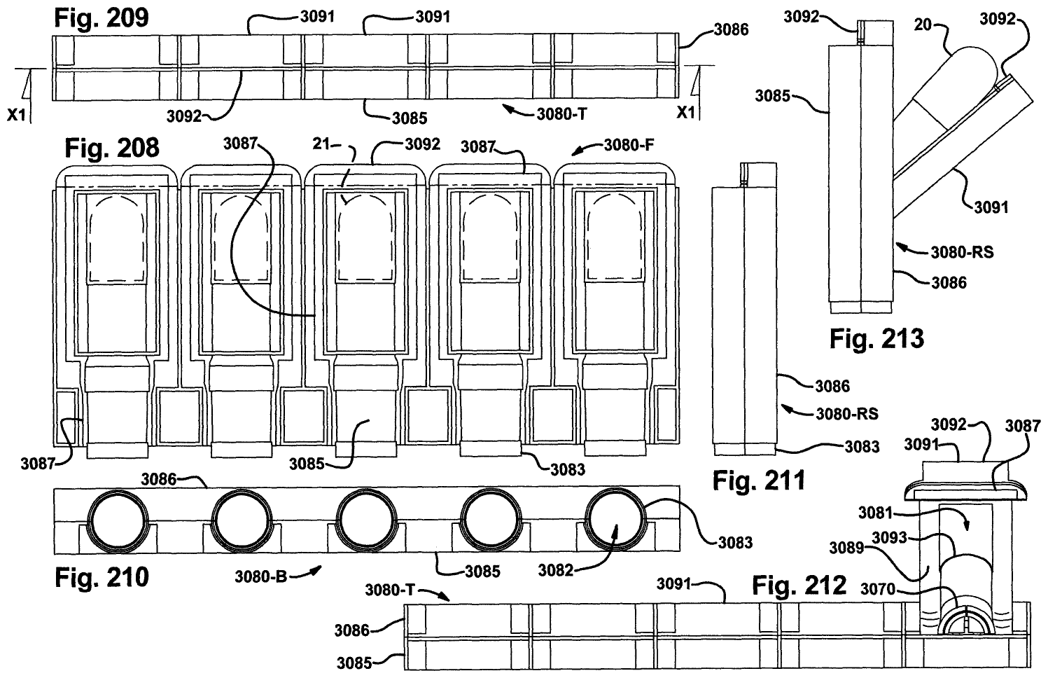

FIGS. 208-211 are respectively a front (F), top (T), bottom (B), and right end (RS) view of the assembled sub-package containing the capsule caps.

FIGS. 212 and 213 are respectively a top view and right end view of the sub-package containing the finished polypills, showing how the cell wall is opened to remove the capsule from the cell.

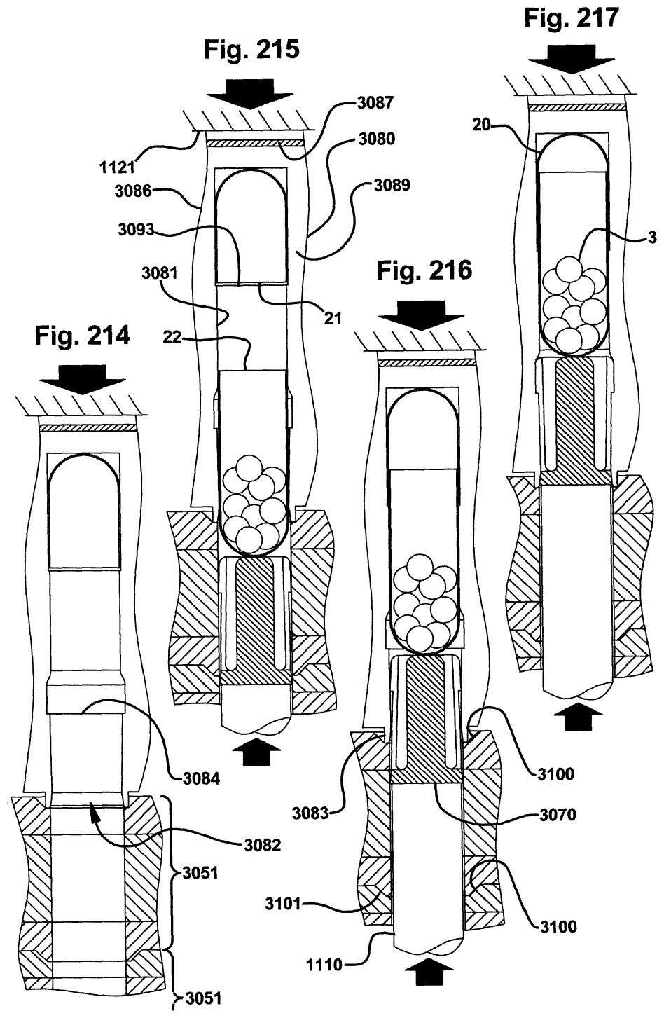

FIGS. 214-217 are longitudinal sections taken through the stacked packages showing consecutive stages of the assembly operation, the section plane being taken along the cell axis between the confronting surfaces of the front and rear mouldings of the sub-package at X1-X1 of FIG. 209.

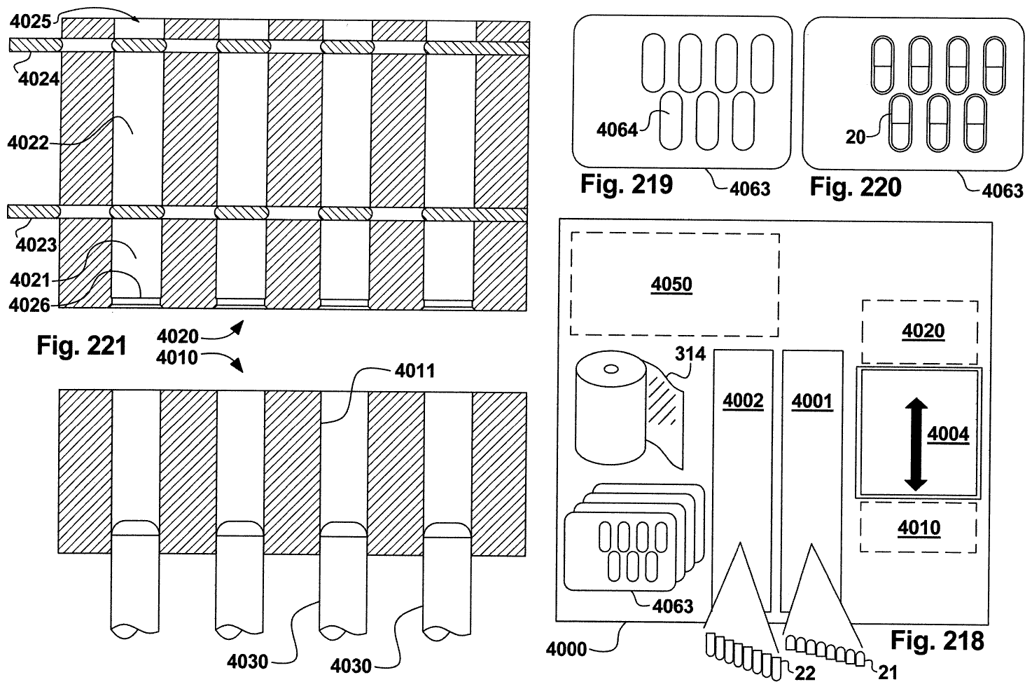

FIGS. 218-230 show FURTHER VARIANTS of the first embodiment in which the assembly machine is configured to receive bulk capsule components, wherein:—

FIG. 218 shows an assembly machine configured to receive bulk capsule packages and single dose drug packages.

FIGS. 219 and 220 show the tray of a blister pack forming one end user sub-package, respectively before and after placing the capsules 20 in the cells, and before sealing the tray with foil.

FIG. 221 shows the capsule assembly mechanism of the machine, comprising a capsule body sub-assembly and a capsule cap sub-assembly.

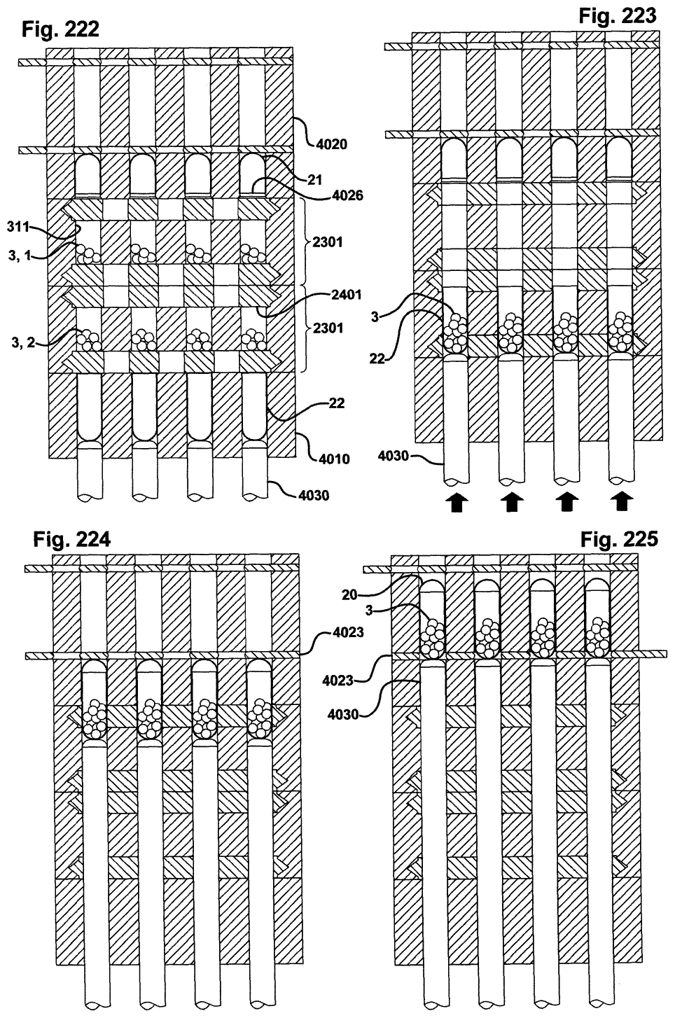

FIGS. 222-226 are longitudinal sections taken through one set of axially aligned cells of the capsule assembly mechanism, showing how the single dose drug packages are stacked between the capsule body sub-assembly and capsule cap sub-assembly, and illustrating respectively, five consecutive stages of the capsule assembly operation.

FIG. 227 shows how the capsule cap sub-assembly transfers the capsules to a blister pack tray in the packaging station.

FIG. 228 shows the finished blister pack forming one end user sub-package;

FIG. 229 shows how a set of blister packs are inserted into a box to form the end user package.

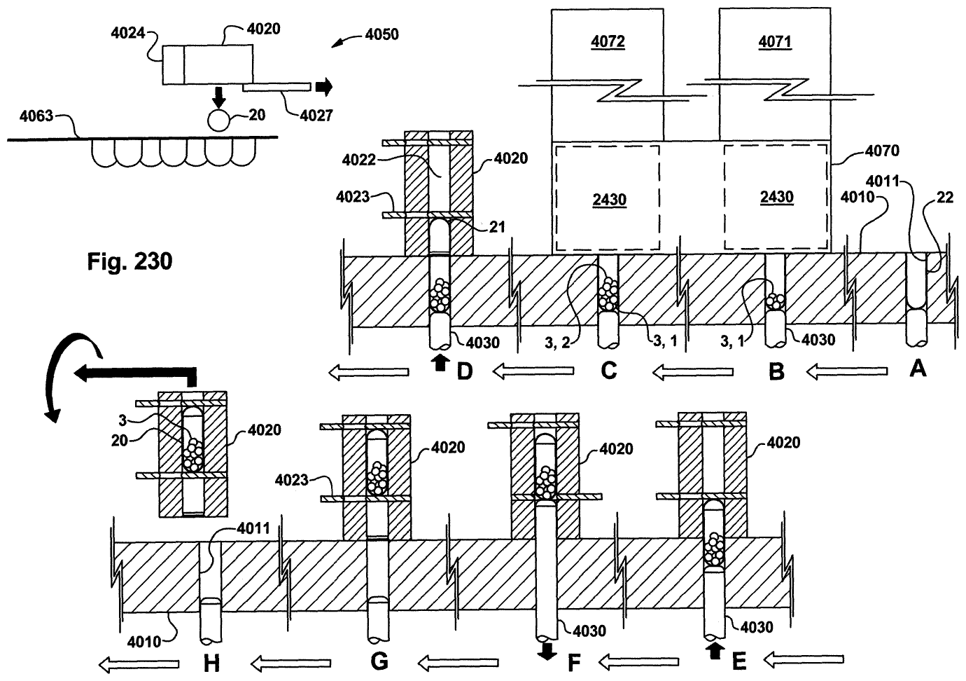

FIG. 230 shows consecutive steps in an alternative capsule assembly and packaging process wherein the capsule assembly mechanism is adapted for use with bulk drug packages.

It should be noted that the package transfer assembly is shown in FIGS. 92, 94, 95, 96 and 97 as if engaged with the packages, the packages being omitted from these figures purely for better illustration of the mechanism.

Where necessary, different views in the same figure are identified by the suffix to the respective reference numeral, wherein:
F indicates a front view
R indicates a rear view (opposite the front view)
T indicates a top or plan view
B indicates a bottom view (opposite the plan view)
E indicates an end view
S indicates a side view
IS indicates an inner side view
OS indicates an outer side view
LS indicates a left side (left end) view;
RS indicates a right side (right end) view.

In some figures a positional term such as “front view” identifies a functional use position relative to the user, e.g. the front of the machine. In other figures the term “front view” is used just as a convenient way of identifying one reference view in that particular figure, to help understand the orientation of the other views in the same figure, in which case “rear view”, “end view”, “side view”, and “top view” or “plan view” should be construed accordingly as relative terms.

“Trailing end view” refers to the direction of insertion of the package into the respective slot of the second machine or its variant.

“Inner side view” and “outer side view” identify whether the illustrated side of the respective element faces inwardly or outwardly in use, for example, relative to a package to which the respective element is attached.

Reference numerals and characters appearing in more than one of the figures indicate the same or corresponding features in each of them.

In the following description, section headings are provided for ease of reference and should not be construed as limiting the scope of the respective disclosure.

Reference numerals are also provided for ease of reference, but should not be construed as limiting the scope of the respective disclosure, but rather to indicate a particular example of the element being discussed, by reference to an illustrated embodiment. In this regard it should be recognised that the embodiments illustrate examples of various features of more general application. Thus, when features are referred to in conjunction with reference numerals, but are under discussion in a way that implies a degree of generalisation, or are susceptible of application in embodiments other than the specific one containing the reference numeral, the presence of the reference numeral does not mean that the feature under discussion is of less general application than would be implied by the description without the reference numeral, or is only susceptible of application in combination with the other features of that specific embodiment.

### DETAILED DESCRIPTION

### Overview of the First and Second Embodiments

The first and second embodiments and their variants illustrate alternative implementations of various features that are common to both embodiments, as will now be described. It will be understood of course that features essential to each aspect of the invention are those defined in combination in the respective independent claim. Other features, whether common to both embodiments or not, are optional.

Both embodiments provide an assembly system comprising an assembly apparatus and a plurality of packages for use with the assembly apparatus. In each embodiment the assembly apparatus is configured as a small machine 100, 500, 5000 that can be mounted, e.g. on a countertop in a small pharmacy or a larger, central or “hub” pharmacy, to receive the packages which are produced in a factory and supplied in bulk to the pharmacy. The packages can be provided in different sizes, e.g. 12, 24, 36 or 48 units per package, and include different drug packages 301, 302, 701, 702, 703, 7001, 7002, 7003 containing individual, fixed dosage units of different drugs, and other packages 304, 305, 704, 705, 7004, 7005 for use in the machine in combination with the drug packages to produce an end user package 300, 700, 7000, as further explained below. The fixed dosage units may be, for example, in the form of spheroids 3, or in a flattened form, e.g. as a soft body or film, referred to hereafter as an API film 31.

Advantageously, the pharmacist does not need to handle directly any of the individual, fixed dosage units, which are pre-packaged, serialized and labelled at the factory. As illustrated in FIG. 60, each package may be wrapped in a protective outer wrapper 709 which is removed before the package is inserted into the machine. The pharmacy may be supplied with multiple units of each type of package (i.e. multiple units of each type of drug package, along with multiple units of each type of package for use therewith). FIG. 60 shows four stacks containing multiple units of different types of packages for use with the second machine, each package contained in a wrapper 709. The wrapped packages can be boxed for delivery to the pharmacy and can be labelled externally, or the wrappers 709 could be transparent to allow the package labels 313, 713 to be read through the wrappers.

Respective ones of the drug packages are selected in the pharmacy in accordance with an individual prescription or prescriptions, and then introduced into the machine 100, 500, 5000 in combination with the other packages as required. The machine is operated to combine together the first and second drugs to form a plurality of single, orally ingestible bodies or polypills 20, 30, which may be individually packaged in the same operation to form the end user package 300, 700, 7000. The prescription could specify that the drugs are to be provided to the end user in the form of a polypill, or the end user could request that their prescription drugs be supplied as polypills, or the pharmacist could decide to combine together drugs that have been prescribed for the same individual, for example, to reduce the pill burden.

The end user package may be assembled from respective ones of the packages inserted into the machine, with the remaining, empty packages being discarded after the assembly operation. Alternatively, the frames or blocks of the empty packages of the first embodiment or its variant may be returned to the factory to be cleaned, re-filled, re-labelled and sealed again with new foil to form new packages. Each machine 100, 500, 5000 is configured to provide a quick and simple assembly procedure, allowing repeated operation to fulfill multiple different prescriptions.

In the first embodiment, the first machine 100 (FIG. 1) is arranged to produce an end user package 300 (FIG. 58) containing the polypills in the form of filled capsules 20, each capsule 20 being sealed in an individual cell 311 of the end user package 300 and containing the first and second drugs 1, 2, preferably in granular, e.g. spheroidal form, so that each fixed dosage unit comprises the respective drug in the form of one or more granules, which preferably are spheroids 3. The capsules 20 may be arranged as shown with their length axis extending in the thickness dimension of the package 300 which is shaped as a small block. The capsule 20 is removed by tearing back a strip 315 using a tab 316 to reveal the cell 311, and then tapping the block so that the capsule 20 drops out of the cell 311 into the palm of the hand.

In the second embodiment, the second machine 500, 5000 (FIG. 60, FIG. 88) is arranged to produce an end user package 700, 7000 (FIG. 80, FIGS. 89-90) which in comparison with the first embodiment may have a relatively flat, thin form factor, and which contains the polypills in the form of compressed, relatively soft bodies that will be referred to herein as pastilles 30. The pastilles 30 are arranged in individual cells 711 of the package 700, 7000 between opposed foil walls 714 so that they can be pressed out of the cell into the palm of the hand.

Each of the machines 100, 500, 5000 is configured to combine together the first and second drugs 1, 2 inside the cells 311, 711 of the packages inserted into the machine, so that the individual fixed dosage units do not come into contact with any internal part of the machine during assembly.

Each end user package 300, 700, 7000 may include a composite label or label assembly 312, 712, which combines indicia, which may be in the form of physical label portions 313, 713 that carry the indicia, from each of the drug packages inserted into the machine to ensure that the end user package is correctly labelled.

Each machine may be arranged to read and authenticate serialization data, e.g. in the form of a barcode 317, 717, on the drug packages during the assembly operation and upload the data to a central database, as further described below. Since each drug package is used only once, and the serialization data is read during the assembly procedure, the system provides a robust, non-repeatable, one-time authentication step that positively identifies each drug package at the point of use.

### Packages

As exemplified by each of the first and second systems, each drug package, and also each package for use therewith, may include a frame 310, 710 which defines a plurality of cells 311, 711, so that the cells 311, 711 are separated by the frame 310, 710 to form a spaced array.

The plurality of packages include at least first and second drug packages 301, 302, 701, 702, 7002, 7003. Each cell 311, 711 of the first drug package 301, 701, 7001 includes a single dose of a first drug 1, while each cell 311, 711 of the second drug package 302, 702, 7002 includes a single dose (i.e. a single dosage unit) of a second, different drug 2. The single doses of the first and second drugs 1, 2 may be configured (e.g. as granules, e.g. spheroids 3 of the same colour) so that when combined together, they are substantially identical in appearance.

In the illustrated examples, the first drug package 301, 701, 7001 includes 12 cells containing a first drug 1 (olanzapine, 20 mg), while the second drug package 302, 702, 7002 includes 12 cells containing a second drug 2 (citalopram, 20 mg). (This particular combination of drugs is shown, purely as an example of two different drugs.)

Alternatively, the first and second drug packages could contain different doses of the same drug. For example, if a drug is provided in a binary series of multiples of a basic dosage unit (e.g. 5 mg, 10 mg, 20 mg, 40 mg, 80 mg, etc.) then any desired multiple of the basic dosage unit (5 mg) could be produced in this way by combining together an economical number of the packages. For example, a prescription could be fulfilled by combining together one drug package 7001 containing a first drug 1, and two drug packages 7002, 7003 containing similar or different doses of a second drug 2, as exemplified by the variant second end user package of FIGS. 89-90.

Each package may be a single use package—which is to say, it is configured to be used only once during the assembly process, after which it may be disposed of, or returned to the factory for recycling, or given to the end user as part of an end user package which, in turn, will be disposed of after the polypills are all consumed.

For example, each drug package, or each package for use therewith, may include at least one part which is configured, in use, by operation of the assembly apparatus to combine together the first and second drugs, either (a) to be irreversibly removed, destroyed or ruptured, or (b) to be irreversibly united with the corresponding at least one part of another respective one of the first and second drug packages.

In the first system, the frangible foil 314 of the drug package 301, 302 is ruptured and destroyed during the assembly process, as is the frangible foil 314 on each of the cap and capsule body packages 304, 305, as the carrier 320 is removed from each cell 311 of the capsule body package 305 and introduced into, and irreversibly united with, the corresponding cell 311 of the capsule cap package 304 which forms the end user package 300. Additionally (or alternatively, e.g. when the cells are closed by movable closure elements rather than foil), the package may include a label which is ruptured or destroyed during assembly. This process is irreversible in the sense that it is not reversed in normal use of the system—rather, the empty drug and cap packages are discarded after removal from the machine, or are returned to the factory so the frame or block can be cleaned and re-used, while the finished end user package 300 is given to the end user who will then discard it in due course.

Thus, it should be understood that a single use drug or capsule body package may include a re-usable frame or block which can be returned to the factory to be cleaned, re-filled, and re-labelled and/or sealed with new foil, to form a component part of another single use package.

In the second system, the frame 710 of each package, and the API film 31 of each cell 311, is irreversibly united with the corresponding component of the adjacent package or packages during the assembly process to form an end user package 700, 7000, as further explained below.

Each cell 311, 711 of each drug package may define an enclosure within which the respective single dose (e.g. granules or spheroids 3, or API film 31) is enclosed, optionally sealingly enclosed. Each dose may be separately sealingly (e.g. hermetically sealingly) enclosed within its respective cell, or the cells may retain the doses without hermetically sealingly separating them, so that the outer wrapper provides a seal to preserve the drugs in storage. For example, each package may be arranged as a foraminous block with sliding cover plates, as illustrated by the alternative drug package of the variant first embodiment, and the entire package sealingly enclosed within an outer wrapper.

As exemplified by the drug packages 301, 302 of the first embodiment, each cell 311 may be closed by a frangible foil or film 314 which sealingly encloses the single dosage unit (spheroids 3) within the cell 311. The film 314 may form a hermetic seal for the individual cell 311, and may be broken when combining together the first and second drugs 1, 2.

In this specification, the terms “frangible foil” or “foil” and “frangible film” or “film” are used synonymously to mean a thin sheet material that is arranged to tear or break or separate responsive to an applied force in normal use. It could be for example a sheet of thin aluminium or plastics or composite, plastic coated aluminium or aluminium coated plastics material, for example of less than 100 microns, or less than 50 microns thickness. Preferably the film or foil is selected to provide a seal sufficient to preserve the respective drug in storage, as known in the art. The film or foil could also be an animal or vegetable based material, e.g. alginate, cellulose, calcium caseinate, carragheenan, chitosan, corn starch, paper or tissue with an impermeable wax or equivalent coating, etc.

If the drug package is contained in an outer protective package, e.g. an impermeable film, then the film or foil that closes the cells could alternatively be an uncoated paper or other permeable material, or even an edible material that can be separated from the package and incorporated into the capsule together with the granules or spheroids 3.

The cell wall 318 of each cell 311 of the drug or other package 301, 302, 304, 305 may be configured to withstand a compressive force applied in a direction of the cell axis Xc to the frangible foil 314 and sufficient to break the foil.

In particular, the cell wall 318 may withstand the compressive force when the drug package 301, 302 is arranged in a stacked configuration with other, like drug packages, the respective cells 311 of the drug packages being axially aligned, and the compressive force is applied to the foil 314 of each cell 311 of the plurality of cells by a respective one of a corresponding plurality of sliding bodies. Each sliding body may be configured to enter slidingly into the respective cell 311 after breaking the foil 314, the sliding body having a diameter normal to the cell axis Xc and selected to be a sliding fit in the cell 311, such that the sliding body is constrained to move slidingly in translation in a direction of the cell axis Xc.

The sliding body may be a carrier 320 for a capsule part 21, 22. Alternatively, if the capsule part is not arranged in a carrier but is configured to contact and break the foil 314 as it moves through the axially aligned cells 311 of the stacked packages, the sliding body may be a capsule part 21, 22.

If the sliding body does not form part of the assembly machine—e.g. if it is a carrier 320 or a capsule part 21, 22—then it may be slidingly guided and constrained by the cell wall 318 as it moves.

Alternatively, if the end surface 111 of the pushrod 110 (as further discussed below) is configured to break the foil 314 in advance of the respective capsule part (instead of being flat, as shown), the sliding body may be the distal end region of the pushrod, in which case the sliding body is guided by the cell wall 318 in the sense that the pushrod is positioned in axial alignment with the cell wall 318 so that there is a sliding interface between the cell wall 318 and the distal end region of the pushrod.

The frangible foil 314 may be relatively more frangible than the cell wall 318.

Alternatively, as exemplified by the second embodiment, each cell 711 may be closed at either end by a cover 706, 707, 7006, 7007 that is removed before or during the assembly procedure. The cover or covers may provide a seal (e.g. a hermetic seal) for all of the cells 711 together, or for each individual cell 711 (i.e. the cells are sealingly separated) as does the frangible film 314 of the first embodiment.

In the second embodiment, the temporary, front and rear covers 706, 707, 7006, 7007 of each drug package 701, 702, 703, 7001, 7002, 7003 are secured at spaced attachment points 719 and are removed before the package frame 710 (containing the API film 31 in each cell 711) is assembled with the other package frames 710. The other packages include front and rear cover packages 704, 705, 7004, 7005 which seal (e.g. hermetically seal) the cells 711 of the end user package 700, 7000, thus replacing the front and rear covers 706, 707, 7006, 7007 of each drug package, as further explained below.

In the second embodiment as illustrated, the temporary, front and rear covers 706, 707, 7006, 7007 are principally intended to protect the adhesive surfaces of the frames 710 and need not hermetically seal the package. In this case the entire drug package 701, 702, 703, 7001, 7002, 7003 containing the drugs 1, 2 in their individual cells 711, together with its temporary covers 706, 707, 7006, 7007, can be sealingly enclosed within an outer wrapper 709 which maintains the drugs 1, 2 in good condition (e.g. by a hermetic seal) until the package is inserted into the machine 500, 5000 as further explained below.

### Packages Configured to Produce Capsules

Each cell 311, 711 of each drug package 301, 302, 701, 702, 703, 7001, 7002, 7003 may include a cell wall 318, 718 which extends along a cell axis Xc between opposite, first and second ends of the cell at opposite, first and second sides of the frame 310, 710. In the drawings, the first and second sides are identified in different views by the suffix to the reference numeral for the respective package or package component, viz: F (front) or R (rear).

In such arrangements, each of the first and second ends of the cell may be closed by a frangible foil 314 to sealingly enclose the respective, single dose of the drug within the cell 311.

In this configuration the drug packages 301, 302 can be used to produce polypills in the form of capsules 20. To assist assembly of the capsule 20, each single dose may be in the form of one or more granules, preferably one or more spheroids 3 (which is to say, granules having a shape that approximates a sphere), which may have a diameter of at least 1.5 mm, preferably at least 2 mm.

Most preferably, the diameter of each spheroid is less than one half, and greater than one third, of the internal diameter of the capsule body. This ensures that the spheroids can pass one another to achieve an optimal packing density without jamming inside the capsule body.

The capsule parts 21, 22 may be contained in further packages 304, 305, each package defining an array of cells 311 separated by a frame 310, to be inserted into the machine 100 together with the selected drug packages 301, 302.

As exemplified by the capsule cap and body packages 304, 305 of the first embodiment, each cell 311 may include a cell wall 318 which extends along a cell axis Xc between opposite, first and second ends of the cell at opposite, first and second sides of the frame, to contain a part 21, 22 of an empty capsule, but without a complementary part 21, 22 required to complete the capsule 20. At least a first end of the cell may be closed by a frangible foil 314 or, as exemplified by the variants of the first embodiment, by a movable closure element.

The other end of the cell may be closed by a carrier 320, as in the capsule body package 305 of the first embodiment, and optionally also a frangible foil (or another frangible foil) that covers the end surfaces of the carriers. Alternatively, the other end of the cell may be closed by a plug which is sealingly engaged in a respective aperture of the end user package or sub-package during the assembly operation.

Where a respective capsule part 22 is arranged in a carrier 320 or accompanied by a plug, the carrier 320 or plug may be received in the cell 311 and slidable out of the cell 311 along the cell axis Xc, as further described below. The carrier 320 may be configured to rupture the foil 314 by sliding movement of the carrier 320 out of the cell 311 along the cell axis Xc during the assembly procedure.

The cell 311 may include a locating structure supporting the respective part in spaced relation to the cell wall 318. For example, a series of radially inwardly projecting annular fins 325 may support the capsule cap 21 radially centrally in the cell 311, as illustrated by the cap package 304 of the first embodiment. Alternatively, the locating structure may be a lining of flock within the cell, as further described below with reference to the variant first embodiment, or a collapsible insert. This helps to align the two capsule parts 21, 22 during assembly.

Where a first end of the cell 311 is closed by a frangible outer wall, e.g. of frangible foil 314, or by a movable closure element or other package component, the second end of the cell 311 may be closed by a removable outer wall, e.g. a strip 315, which is configured to be lifted and torn outwardly away from the frame 310, e.g. by means of a tab 316, by a user to open the second end of the cell 311 to permit the capsule 20 to be removed from the cell, when the package (e.g. cap package 304) is formed into an end user package 300 containing the filled capsules 20.

### Packages Configured to Produce Pastilles

Each cell 711 of each drug package 701, 702, 703, 7001, 7002, 7003 (and/or of another package 704, 705, 7004, 7005 for use therewith) may include at least one edible wall 32, 33 fixedly mounted to the frame 711. The at least one edible wall 32 may be configured to be detached from the frame 711 in which it is mounted and consumed in normal use by an end user.

If the package is a drug package 701, 702, 703, 7001, 7002, 7003, then the at least one edible wall 32 will include or enclose the respective single dose of the respective, first or second drug 1, 2, e.g. in the form of an API film 31.

Alternatively, if the package 704, 705, 7004, 7005 is not a drug package, then the edible wall 33 could be configured, e.g. as a disc or wafer of rice paper or other edible sheet material, to form an outer surface or covering of the polypill 30 after assembly, in which case it need not include a drug.

Each cell 711 may be closed on one side of the frame 710 by an outer wall 714 which is sealingly attached to the frame 710 and configured to be ruptured or torn away from the frame 710 in normal use by an end user to expose the at least one edible wall 33. This arrangement is exemplified by the front and rear cover packages 704, 705, 7004, 7005 of the second embodiment and its variant, which serve both to seal each pastille 30 in its respective cell 711 of the end user package 700, 7000, and to provide the outer surface of the pastille 30 that can be gripped by the user without sticking to the fingers when the pastille 30 is removed for use.

The end user package 700, 7000 may include the frame 710, and the at least one edible wall 32 of each of the cells 711, of each of the first and second drug packages 701, 702, 7001, 7002.

The second and variant second end user packages 700, 7000 exemplify one such arrangement, wherein each edible wall 32 is formed as a film 31 that incorporates the respective drug 1, 2; alternatively, each cell 711 of the drug package could include two edible walls, sealed to form a compartment between them which encloses the drug 1, 2. The sealed compartment could form a cachet or envelope, for example, similar to the well-known “flying saucer” confectionery that has a sherbert filling between two discs of rice paper.

The frames 710 of such packages may be configured to be stacked and bonded together in an assembled configuration with each of the plurality of cells 711 of each package in axial alignment with a corresponding one of the plurality of cells 711 of an adjacent one of the packages, so that the cell axes X2 of corresponding ones of the cells are collinear. In this arrangement, a respective edible wall 32 of each cell 711 is configured to adhere to a respective edible wall 32 of a corresponding one of the cells 711 of an adjacent one of the packages when the frames 710 are stacked and bonded together in the assembled configuration.

In such arrangements, the at least one edible wall 32 of each drug package 701, 702, 703, 7001, 7002, 7003 may be exposed at each of two oppositely facing sides of the frame 710. The frame 710 of the drug package 701, 702, 703, 7001, 7002, 7003 may be coated with a tacky adhesive 4 on one or both of the two oppositely facing sides of the frame 710. In order to protect the adhesive surfaces and API films 31 until the moment of assembly, the frame 710 may be enclosed between two temporary, removable covers 706, 707, 7006, 7007, each of which is arranged to protect a respective one of the two oppositely facing sides of the frame 710, as further explained below.

### Operation of the Assembly Apparatus

As explained above, the first and second drugs 1, 2 are packaged in separate, first and second drug packages 301, 302, 701, 702, 7001, 7002, so that each cell 311, 711 of each drug package includes only one, single dose of the respective, first or second drug 1, 2.

The first and second drug packages 301, 302, 701, 702, 7001, 7002 (and any additional packages 703, 704, 705, 7003, 7004, 7005 as required) are then introduced into the machine 100, 500, 5000. After receiving the packages, the machine 100, 500, 5000 is operated to combine together the respective, single doses of the first and second drugs 1, 2 contained in each pair of corresponding cells 311, 711 of the first and second drug packages 301, 302, 701, 702, 7001, 7002, within the respective cells 311, 711 of the plurality of packages, to form a plurality of single, orally ingestible bodies or polypills 20, 30. Each polypill 20, 30 includes the respective single doses of the corresponding pair of cells 311, 711 of the first and second drug packages 301, 302, 701, 702, 7001, 7002 (together with any further single doses if more than two drug packages (e.g. third drug packages 703, 7003) were inserted into the machine 100, 500, 5000.)

The machine 100, 500, 5000 may be configured to sealingly enclose the first and second drug packages 301, 302, 701, 702, 7001, 7002 in a vacuum (which is to say, a full or partial vacuum) or a modified protective atmosphere, and to combine together the first and second drugs 1, 2 of the first and second drug packages 301, 302, 701, 702, 7001, 7002 in that vacuum or modified protective atmosphere. For example, the machine 100, 500, 5000 could include a dehumidifier so that the packages are processed, and the polypills 20, 30 packaged, in dry, filtered ambient air. The machine 100, 500, 5000 could be partially evacuated before replacing the evacuated air with dry, filtered air. The evacuated air could be replaced by bottled nitrogen or other inert gas. In the illustrated embodiments, the second machine 500 is shown with a vacuum generating apparatus 590, which (although not shown) is also present in the variant second machine 5000, and which could also be provided for the first machine 100 and/or the variant first machine, to evacuate the machine before packaging the polypills, either in the vacuum or in the protective gas that replaces it.

Advantageously, in each of its first and second embodiments, the machine may combine together the first and second drugs while each drug and capsule part or other polypill component remains in contact only with the internal surfaces of the packages, hence without contact with any part of the machine. Thus, cleaning and maintenance is simplified and the possibility of contamination of the finished polypills, either from machine surfaces or from packages previously introduced into the machine, is minimised, even when the machine is not cleaned between uses.

### End User Package

Preferably, as exemplified by each of the first and second embodiments, the assembly apparatus 100, 500, 5000 is configured to sealingly enclose each of the polypills 20, 30 in a respective one of a plurality of cells 311, 711 of an end user package 300, 700, 7000, each cell 311, 711 defining an enclosure which is openable by an end user to remove the polypill 20, 30 for use.

The end user package 300, 700, 7000 may incorporate the frame 310, 710 of at least one, or of each of two or more, of the plurality of packages received in the assembly apparatus 100, 500, 5000.

In this case, each cell 311, 711 of the end user package 300, 700, 7000 may include a cell 311, 711 of the respective package or packages whose frame 310, 710 is incorporated in the end user package 300, 700, 7000. The end user package 300, 700, 7000 may further include at least a part (e.g. a carrier 320, a plug, a plug and a collar, a foil wall 714, etc.) of another one or ones of the plurality of packages (e.g. capsule body package 305 or front or rear cover package 704, 705, 7004, 7005), which part is applied by the assembly apparatus 100, 700, 7000 to close each cell 311, 711 of the end user package 300, 700, 7000.

For example, in the first embodiment, the end user package 300 includes the frame 310 and cells 311 of the cap package 304, each cell 311 being closed by a carrier 320 from the capsule body package 305. In variants, each cell 1311 of the end user package may be closed by a plug from the capsule body package, optionally in combination with a collar from the end user package assembly, as further described below.

In the second embodiment, the end user package 700, 7000 includes the frames 710 and cells 711 of all of the packages received in the second machine 500, 5000, with the cells 711 being closed at each end, respectively by the frangible foils 714 of the front and rear cover packages 704, 705, 7004, 7005. Alternatively, one of the front and rear cover packages 704, 705, 7004, 7005 could have, for each cell 711, a flexible but not frangible cover that closes the respective end of the cell, and can be pressingly deformed by the user to urge the pastille 30 out through the frangible film 714 of the other cover package at the opposite end of the cell 711.

Where each cell 311, 711 of the package whose frame 310, 710 is incorporated in the end user package 300, 700, 7000 includes a respective, orally ingestible component when received in the assembly apparatus, that orally ingestible component may be incorporated in a respective polypill 20, 30 of the end user package.

In such arrangements, the respective orally ingestible component may not include a drug. Alternatively, it may include a drug—which is to say, the end user package may incorporate the frame of one (or each) of the drug packages, as in the second system. The orally ingestible component may form an outer surface of the respective polypill, such as a cap part 21 of the capsule 20 of the first embodiment, or a wafer 33 that forms the outer covering of the pastille 30 in the second embodiment.

Where the first and second drugs 1, 2 are packaged in granular, e.g. spheroidal form and combined together in capsules, each polypill 20 contained in the end user package will include a capsule containing a plurality of the granules or spheroids 3, wherein a first one or first ones of the granules or spheroids 3 contain the first drug 1 but not the second drug 2, and a second one or second ones of the granules or spheroids 3 contain the second drug 2 but not the first drug 1.

Where an end user package 300 is provided containing polypills in the form of capsules 20, each capsule 20 may be arranged in a carrier 320 (which is to say, at least a part, preferably most or all of the length of a respective part of the capsule is received in the carrier), the carrier forming a plug which is fittingly received within the respective cell 311 of the end user package 300. The carrier 320 can have an open end through which the capsule 20 may be removed from the cell 311, and a closed end opposite the open end. The carrier 320 can help guide one part 22 of the capsule and break the frangible foils 314 (if present) as it travels through the aligned cells 311 of the packages, and then can seal the cell 311 containing the filled capsule 20 to become part of the end user package 300, as illustrated by the first embodiment. The carrier 320 may isolate the drugs and capsule components from contact with the pushrods.

Alternatively, the capsule body can be arranged together with a plug, not in the form of a carrier, which is slidingly displaced by the pushrod through the aligned cells 311, 1311 of the stacked packages, and then fittingly engaged in the open end of the respective cell of the end user package to close the cell, optionally in combination with a collar which guides the capsule body as it engages telescopically in the cap. In such arrangements, the plug may isolate the drugs and capsule components from contact with the pushrods.

Granules comprising the first and second drugs 1, 2 may be particles which are sufficiently large (e.g. at least 1 mm in mean diameter, preferably at least 1.5 mm, more preferably at least 2 mm in mean diameter) that each granule may be coated with a coating thick enough to be effective, e.g. to delay release of the drug after ingestion, or to mask taste during ingestion, or simply to provide a desired appearance, e.g. colour, wherein the volume of the coating is less than that of the granule on which the coating is applied. It will be understood that the term “granules” may refer also to coated granules, i.e. to include the coating. The drug packages may be labelled to indicate whether or not the granules have an enteric or extended release or other coating.

Preferably the granules are formed as spheroids, which helps them to enter into the capsule body during assembly. Preferably the first and second ones of the spheroids 3 have a mean diameter of at least 1.5 mm, more preferably at least 2 mm, e.g. about 2.8 mm, which further helps the spheroids 3 to enter into the capsule during assembly.

The mean (i.e. average) diameter of the spheroids may be greater than the wall thickness of the carrier 320, wherein the wall thickness is 0.5·(OD−ID), wherein OD is the carrier external diameter proximate its open end, and ID is the carrier internal diameter proximate its open end. When considered in longitudinal section, the open end of the carrier 320 may be bevelled or rounded, both externally and internally, to help the carrier move through the stacked packages and to help the granules or spheroids to enter into the carrier. A spheroid mean diameter greater than the carrier wall thickness means that where the carrier wall is hemispherical in longitudinal section at its open end, the equatorial plane of the spheroid will contact the inwardly sloping face of the carrier so that the spheroid is urged into the capsule body inside the carrier and will not become trapped between the carrier and the cell wall. If the cell is internally flocked then the flock may prevent the spheroids from leaving the capsule body while it enters into the cap.

The mean diameter of the spheroids may be less than half the internal diameter of the capsule body 22, to ensure that the spheroids 3 are able to mix inside the capsule rather than being stratified inside the capsule. Using a vibration source during assembly may help to mix the spheroids or other drug particles together. Alternatively or additionally, the first and second ones of the granules or spheroids 3 may be substantially identical in appearance, for example, having coatings of an identical colour. Making the spheroids 3 identical in appearance, and preferably also mixing them together, helps ensure that the end user cannot readily separate the two drugs 1, 2 by opening the capsule 20, which may be desirable in certain applications such as the treatment of psychosis as further discussed below.

Most preferably, the diameter of each spheroid is less than one half, and greater than one third, of the internal diameter of the capsule body. This ensures that the spheroids can pass one another to achieve an optimal packing density without jamming inside the capsule body.

### Operation of the Assembly Apparatus to Form an End User Package Containing Pastilles

The second embodiment exemplifies how an end user package 700, 7000 can be formed as an assembly including two or more drug packages 701, 702, 703, each comprising a frame 710 defining an array of cells 711, wherein each cell 711 includes at least one edible wall 32 fixedly mounted in the frame 710. As explained above, each drug package 700, 7000 includes only one, first or second drug 1, 2, of which one single dose is included in, or enclosed by, the edible wall 32 of each respective cell. As exemplified by the illustrated embodiment, the drug may be incorporated into a body (exemplified by the API film 31) which is attached to a carrier body (exemplified by the carrier film 34) by which the API film body is attached to the frame 710, so that these two bodies 31, 34 together form the edible wall 32.

The frames 710 are connected together so that each of the drug package cells 711 of the first drug package 701 is in axial alignment with a respective one of the drug package cells 711 of the second drug package 702, the conjoined cells 711 thus forming together a respective one of the cells 711 of the end user package 700, 7000.

The edible walls 32 of the respective drug package cells 711 forming each cell 711 of the end user package 700, 7000 are connected together to form together the respective single, orally ingestible body or polypill 30 contained within the respective cell 711 of the end user package 700, 7000, which in the illustrated embodiment is configured as a pastille 30.

The edible walls 32 can be arranged to adhere together when pressed together. This can be achieved, for example, by coating the edible walls 32 with an edible, tacky adhesive, or forming them as a film of a material that adheres to itself, as well known in the art.

Adhesion may depend on pressure and time, so a bond may be created in a short time at higher pressure or a longer time at lower pressure.

In order to provide a quick and simple assembly procedure, a good bond, and a good shelf life for the pastille 30, the end user package 700, 7000 may be configured to compress the edible walls 32 together by atmospheric pressure. This means that pressure can be applied over an extended time period of much longer duration than the assembly procedure, terminating only when the pastille 30 is removed from the end user package 700, 7000 for consumption. At the same time, evacuation of the enclosure 711 helps preserve the drugs 1, 2 in storage.

In this arrangement, each enclosure 711 of the end user package 700, 7000 includes a pair of outer walls 714, which in the illustrated embodiment are formed as frangible foil walls 714, spaced apart in opposed relation. The edible walls 32 of each single, orally ingestible body or polypill 30 are bonded together by adhesion, and are compressed together between the outer walls 714 of the respective enclosure 711 by external ambient atmospheric pressure acting on the outer walls 714 as indicated by the arrows in FIG. 86.

The assembly apparatus 700, 7000 is configured to create a vacuum (which is to say, a full or partial vacuum), and to sealingly enclose the edible walls 32 of each single, orally ingestible body 30, in said vacuum, between the outer walls 714 of the respective enclosure 711. The outer walls 714 of each enclosure 711 are configured to be moveable together by external ambient atmospheric pressure to compress the edible walls 32 of the respective single, orally ingestible body 30 between the respective pair of outer walls 714.

### Operation of the Assembly Apparatus to Form an End User Package Containing Capsules

As exemplified by the first embodiment, each polypill 20 may include a capsule formed from at least first and second capsule parts 21, 22, e.g. a conventional capsule body 22 and capsule cap 21. The capsule parts 21, 22 may be arranged in the cells 311 of an additional package or packages 304, 305 to be received in the machine 100 together with the drug packages 301, 302; alternatively, a quantity of the capsule parts 21, 22 could be introduced into the machine 100, e.g. in loose, bulk format or in a cassette, for use with the packages 301, 302.

As used herein, the term “capsule” may indicate a capsule shell or a filled capsule 20, i.e. a capsule shell together with the drugs 1, 2 contained therein, according to the context. In the second sense of the term, the polypill may consist of the capsule 20.

After receiving the capsule parts 21, 22, the machine 100 is arranged to collect together, between the capsule parts 21, 22 of each capsule, one single dose of each drug 1, 2 from one corresponding cell 311 of each of the drug packages 301, 302, and to close together the capsule parts 21, 22 to enclose the single doses together within the capsule to form the polypill 20.

The capsules 20 can be provided in alternative sizes, e.g. size #1, #2, and #0E. In order to ensure that the total volume of the combined drugs 1, 2 does not exceed the capacity of the selected capsule size, each of the drug packages 301, 302 may have a different thickness in a thickness dimension, the thickness of each package 301, 302 being proportionate to a volume of the respective single dose of the drug 1, 2 of each of the cells 311 of that package.

The machine 100 may be configured to receive the drug packages 301, 302 in a stacked configuration in which corresponding cells 311 of the drug packages 301, 302 are axially aligned in the thickness dimension, and to receive an indication corresponding to a maximum volume capacity of each of the capsules 20. The assembly apparatus 100 may then prevent operation to combine together the drugs 1, 2 if a combined thickness of the drug packages 301, 302 is greater than a maximum combined thickness corresponding to the maximum volume capacity of each of the capsules 20.

The indication may be received in the form of a user input via a user interface (e.g. a control panel displayed on a touchscreen 267) of the machine, e.g. by selecting the desired capsule size on the control panel before inserting the packages, or by turning a knob (not shown) to move a profile rod 152 to a desired position in a manual embodiment. The profile rod 152 or other shape profile feature of the machine 100 may then be set to selectively permit or obstruct a corresponding shape profile, e.g. a profile aperture 352 of the packages, to prevent insertion of packages 304, 305 containing capsule parts 21, 22 other than the selected size, before the packages are introduced into the machine 100.

Alternatively, the indication could be detected by a scanner (e.g. scanner 261) of the machine 100, directly from package indicia (eg. label or serialization indicia 313, 317 or other indicia) on the inserted packages. Operation of the machine 100 can be prevented by selectively varying the distance between the pressure surface 122 and the upper end surfaces 111, 171 of the pushrods 110 and pedestal block 170, further described below, to prevent the pressure plate 121 from being closed to the operational position if the stack is too high. In the first machine 100, this is accomplished by adjusting the starting position of the moving frame assembly 120. In an alternative, manual embodiment, it could be achieved by rotating a profile rod (e.g. profile rod 152) to define the thickness of the pressure pad 250 that can be fitted into the machine, as mentioned under “Alternative embodiments”, below.

Alternatively, the machine may determine the required capsule size based on the indicia on the packages which indicates the total volume of the drugs or the number of spheroids contained in each cell, or from a lookup table based on the dose and identity of the drug. The required capsule size may be displayed on a user display screen. The machine may then prevent the assembly operation if the selected drug packages are introduced into the machine together with capsule packages of an incorrect size.

The first part (e.g. the cap 21) of each capsule may be contained in a first capsule package 304, comprising (like the other packages) a frame 310 defining an array of cells 311, each cell 311 defining an enclosure containing the respective capsule part 21.

The first capsule package 304 may be introduced into the machine 100 together with the drug packages 301, 302 before operating the machine 100 to sealingly enclose each capsule 20 containing the respective, single doses from the drug packages 301, 302 in a respective one of the cells 311 of the first capsule package 304 to form an end user package 300. Each cell 311 of the first capsule package 304 forms a cell 311 of the end user package 300 which is openable by an end user to remove the capsule 20 containing the respective, single doses for use.

The second capsule part 22 may be contained in a second capsule package 305, also comprising a frame 310 defining an array of cells 311, each cell 311 forming an enclosure containing the second capsule part 22. The machine 100 may be configured to receive the first and second capsule packages 304, 305 and the drug packages 301, 302 in a stacked configuration in which the drug packages 301, 302 are arranged between the first and second capsule packages 304, 305, and corresponding cells 311 of the first and second capsule packages 304, 305 and each of the drug packages 301, 302 are axially aligned. The machine 100 is then operated to urge the second capsule part 22 of each cell 311 of the second capsule package 305, through the respective, axially aligned cells 311 of the first and second drug packages 301, 302 into the respective, axially aligned cell 311 of the first capsule package 304.

In this arrangement, each cell 311 of the second capsule package 305 may contain a carrier 320, wherein each second capsule part 22 is arranged in the respective carrier 320. The assembly apparatus 100 is arranged to urge the carrier 320 containing the second capsule part 22 through the respective, axially aligned cells 311 of the first and second drug packages 301, 302 into the respective, axially aligned cell 311 of the first capsule package 304, such that the carrier 320 forms a part of the end user package 300. The carrier may thus act as a plug which closes the open end of the cell of the end user package containing the polypill.

Where each cell 311 of each drug package is dosed by a frangible foil 314, the carrier 320 may be arranged to penetrate the foil 314 during the assembly process.

Alternatively, as described in the multipack variant, below, the cap 21 of each capsule may be contained in a cap package, optionally in a collar, wherein both the cap and the collar (if present) are slidingly displaced from the cap package during assembly. The cap package is introduced into the machine together with an end user package, optionally in the form of a multipack defining an assembly of multiple end user sub-packages. The cap package and end user package may be assembled together as an end user package assembly and introduced into the machine in that form. The end user package defines a plurality of cells which receive the polypills, and which may be closed during the assembly operation by a plug from another one of the stacked packages, which may be the capsule body package. The plug may fittingly (e.g. sealingly, or hermetically sealing) close the open end of the respective cell of the end user package after the capsule enters the cell, optionally in combination with the collar (if present).

### Composite Label

Where the first drug package includes first label indicia identifying the first drug, and the second drug package includes second label indicia identifying the second drug, the assembly apparatus may be arranged to combine together the first and second label indicia from the first and second drug packages as received in the assembly apparatus, to form a composite label of the end user package identifying each of the first and second drugs. The label indicia may be printed on a label element of the package, e.g. an adhesive label, or may be printed or otherwise formed directly on the frame or block of the package.

The first and second label indicia may be transferred from the first and second drug packages to the end user package (e.g. by transferring them to the capsule cap or body package which forms the end user package), or may remain a part of the first and second drug packages when they are combined together to form component parts of the end user package, as exemplified by the second embodiment.

Providing a composite label than combines together the first and second label indicia from the first and second drug packages as received in the assembly apparatus—which is to say, the composite label includes each of the first and second label indicia that were present on the first and second drug packages as introduced into the assembly apparatus—ensures that the end user package is always correctly labelled, because whatever appears on the drug package will form part of the composite label.

As exemplified by the first and variant first embodiments, the first capsule package may include a flap with an adhesive surface (which may be protected, for example, by a release paper), which is movable to position its adhesive surface to cover a side of the first capsule package (e.g. the cap package) through which the carriers are admitted during assembly. The composite label formed during assembly of the end user package may be arranged to form a front surface of the flap facing away from the adhesive surface. This makes it possible to use the large, flat side of the end user package for the label, while also covering the exposed ends of the carriers and sealing them in their cells.

The first machine illustrates one way to provide the composite label as a mechanical assembly in which the first and second label indicia are transferred to the end user package. In this approach, the composite label is a label assembly including first and second label elements, the first label element bearing the first label indicia, the second label element bearing the second label indicia, wherein the first label element forms part of the first drug package as received in the assembly apparatus, and the second label element forms part of the second drug package as received in the assembly apparatus.

Following this approach, the end user package 300, 700, 7000 may include a label assembly 312, 712 including first and second label elements 313, 713, wherein the first label element 313, 713 bears label indicia identifying the first drug 1, and the second label element 313, 713 bears label indicia identifying the second drug 2. The label elements 313, 713 are provided as part of the first and second drug packages 301, 302, 701, 702, 7001, 7002, respectively, as received in the assembly apparatus 100, 500, 5000, and are combined together to form the label assembly 312, 712 of the end user package 300, 700, 7000. Further label elements 313, 713 can be included if more than two drug packages are used.

For this purpose, each drug package 301, 302, 701, 702, 703, 7001, 7002, 7003 may include a label portion 313, 713 bearing label indicia identifying the respective drug 1, 2 contained therein. The label portions 313, 713 of the first and second (and further) drug packages may be juxtaposed to form a composite label 312,712 of the end user package 300, 700, 7000, as exemplified by each of the first and second embodiments.

The label portion 313 may have an adhesive surface 4 that is separate or separable from the frame 310 of the respective package 301, 302. This enables the label portion 313 to adhere to a surface of the end user package 300 and to be detached from the frame 310 of the package 301, 302 of which it formed a part.

This is achieved in the first embodiment by means of a release surface (e.g. a release coating) 5 on the frame 310 that faces the adhesive side 4 of the label portion 313.

Where the drug 1, 2 is included in, or enclosed between, an edible wall or walls 32 of each cell 711, as exemplified by the second embodiment, the frames 710 of the drug packages 701, 702, 703 may be configured to be stacked and bonded together in an assembled configuration with each respective one of the plurality of cells 711 of each package 701, 702, 703, 704, 705 in axial alignment with a corresponding, respective one of the plurality of cells 711 of an adjacent one of the packages 701, 702, 703, 704, 705. In this arrangement, the frame 710 of each drug package may include at least one label portion 713, each of the label portions 713 bearing label indicia identifying the respective drug 1, 2 contained in the respective drug package 701, 702, 703. The label portions 713 are configured to form a composite label 712 displaying a respective label portion 713 of each of the drug packages 701, 702, 703 when the frames 710 are stacked and bonded together in the assembled configuration.

One way to do this, for example, not shown in the drawings, would be to configure each drug package with an array of cells having rotational symmetry about a point, and one label portion that extends radially outwardly relative to the point, next to an opening or recess in the frame at the same radial distance from the point. By arranging each package at a different, incrementally rotated position about the point, so that the cells of the packages are all axially aligned, the labels could be juxtaposed with the label of each package exposed at the opening or recess of the other package or packages. The labels could adhere to a part of a rear cover of the end user package. In such an arrangement, the drug packages could be arranged as discs that are rotated about their central axis to arrange their respective label portions, positioned at the circumference of the discs, to extend as a juxtaposed array around a part of the circumference of the end user package.

Another way to do it, as further exemplified by the second embodiment, is by including a plurality of label portions 713 in the frame 710 of each drug package 701, 702, 703, all of which may bear the same indicia, e.g. “olanzapine 20 mg” as shown in the first drug package 701, or “citalopram 20 mg” as shown in the second drug package 702. The assembly apparatus 500, 5000 includes a cutting mechanism, exemplified in the illustrated embodiment by first and second cutters 510, 511, which are arranged to cut away different respective ones of the label portions 713 of the plurality of drug packages 701, 702, 703, to leave each of the plurality of drug packages 701, 702, 703, after said cutting away, with a different one or different ones of its respective label portions 713 when compared with each other one of the plurality of drug packages 701, 702, 703.

This approach is exemplified by the second embodiment, wherein the frame 710 of each drug package 701, 702, 703 has three label portions 713, comprising a left-hand label portion 713′, a centre label portion 713″, and a right-hand label portion 713′″. The three drug packages 701, 702, 703 are received respectively in first, second and third slots 501, 502, 503 of the second or variant second machine 500, 5000, these slots being indicated respectively by indicia [1], [2], and [3] as shown. The first cutter 510 is positioned at the first slot 501 to cut away the centre 713″ and right-hand 713′″ label portions of the first drug package 701. The second cutter 511 is positioned at the second slot 502 to cut away the right-hand label portion 713′″ only of the second package 702. There is no cutter at the third slot 503 and so none of the label portions 713 are cut away from the third drug package 703. The cutters may cut away part of the temporary covers together with the label portions.

In such an arrangement, the assembly apparatus 500, 5000 is further configured to assemble together the packages 701, 702, 703, 704, 705 in the stacked configuration to display a remaining one of the label portions 713 of each drug package of the plurality of drug packages 701, 702, 703, said remaining ones of the label portions 713 being displayed in juxtaposed relation to form together a composite label 712 of the end user package 700, 7000. For example, in the second embodiment and its variant, the stacked packages display the left-hand label portion 713′ of the first drug package 701 which is positioned on the top of the stack of drug packages (ie. at the right-hand end of the stacked drug packages as viewed from the front of the machine with the stack arranged on a horizontal axis as shown), next to the centre label portion 713″ of the second drug package 702 beneath it, and, if a third drug package 703 is present (which is not shown in the front and rear views of FIG. 80, but can be seen in FIG. 89), the right-hand label portion 713′″ of the third drug package 703.

The number of label portions 713 on each drug package 701, 702, 703 (and the number of slots 501, 502, 503 with cutting stations 510, 511 for receiving the drug packages) can be increased to allow for more than three drug packages to be included in the end user package 700, 7000. For example, if the maximum number of packages is five, then the number of label portions 713 to be cut away would be, from the first (top or front-most) package to the last (bottom or rear-most) package: four, then three, then two, then one, then none.

### Package Authentication

Each of the first and second drug packages 301, 302, 701, 702, 7001, 7002 preferably includes indicia that identifies the drug 1, 2 and dose contained therein, optionally also any excipients, modified release coatings or other therapeutic properties. This information is preferably printed so as to be legible by the user, and may form part of a composite label assembly 312, 712 of an end user package as described above. The same information may also be provided in the form of machine readable indicia such as a barcode, as shown in the label portions 313, 713. Each package 301, 302, 304, 305, 701, 702, 703, 704, 705, 7001, 7002, 7003, 7004, 7005 may further be provided with machine readable indicia 317, 717 comprising further information, e.g. serialization data to uniquely identify each package, as well known in the art. The data may include a code that uniquely identifies the package, and/or other data such as batch number, time and/or date of manufacture, location where manufactured, and other supply chain information such as the route by which the package was distributed or the territory into which it was supplied.

One problem in authenticating drugs or other products using serialization data is that, when each package is provided with a unique serial number, that number can be copied to produce counterfeit packages. One way to address that problem is to read the serial number at the time of authentication of the package, and upload the serial number to a central database which checks to see whether the number (i) is contained in the database record of genuine serial numbers, and (ii) has ever previously been read and uploaded. If it is a genuine serial number and was not previously uploaded, then the database controller can indicate that no copies have been detected and so the package can be accepted as authentic. If it is a genuine serial number but has previously been read and uploaded, then the database controller can indicate that it may be (but is not necessarily) a counterfeit.

One problem with this methodology is that if the same package serial number is authenticated more than once, the database controller may indicate on the second occasion that the package is a counterfeit. In a busy pharmacy, it may not be practical to scan each package on arrival and then keep a detailed log of the authentication information; thus, the authentication procedure is subject to a degree of uncertainty, depending on the probability that different members of staff may have attempted authentication on more than one occasion. To address this problem, multiple codes and peel-off labels can be provided, but this adds cost and complexity.

Since the packages of the present system are used only once during the assembly process, this problem can be solved by capturing the authentication data 317, 717 from the package during the assembly process.

Accordingly, the machine 100, 500, 5000 (specifically, one or more readers or scanners 261, 561 and a local controller 262 with local memory 263 and external data link 264) may be arranged, after introducing the packages into the machine and either before, during or after combining the drugs 1, 2 to form the polypills 20, 30, to read the machine readable indicia 317, 717 from the drug packages and optionally also the other packages used therewith.

The machine 100, 500, 5000 may also receive information from a database 90 (e.g. via a remote computer 91 controlling the database 90), and, based on the indicia 317, 717 and the received information, may identify and authenticate the respective drug packages 301, 302, 701, 702, 703, 7001, 7002, 7003 including in particular the drugs 1, 2 contained therein, and optionally also may identify and authenticate the other packages 304, 305, 704, 705, 7004, 7005 used therewith.

Preferably, the machine 100, 500, 5000 is further arranged to upload information based on the machine readable indicia 317, 717 to the database 90.

The machine 100, 500, 5000 could download data relating to the packages from the remote database 90 to a local memory 263 of the machine before reading the package indicia 317, 717, and then compare the package indicia 317, 717 with the data stored in the local memory 263 and perform the authentication based on that comparison. In this case the machine 100, 500, 5000 could also upload the package indicia 317, 717 (i.e. the data represented thereby), optionally also the authentication result, to the remote computer 91 and database 90.

Alternatively, the machine 100, 500, 5000 could upload the information from the indicia 317, 717 to a remote computer 91 controlling the remote database 90, and then receive an authentication message from the remote computer 91 after the uploaded information has been compared with the corresponding package data stored at the remote database 90.

Alternatively, a combination of both methods could be employed, so the machine 100, 500, 5000 receives regular downloads and makes regular uploads, whereby its local memory 263 is constantly updated with the current information in the remote database 90, perhaps just that information that pertains to the packages that have been logged, at the remote database 90, for use (or potential use) with that individual machine 100, 500, 5000. This enables fast operation without waiting for a real-time response from the remote computer 91, while maintaining data integrity.

For example, the machine 100, 500, 5000 could receive data for all packages destined for the territory in which the machine is operated, or all packages included in a bulk container whose ID was previously logged and uploaded to the database 90 on despatch from the local supplier or on arrival at the pharmacy.

The information based on the package indicia 317, 717 may be transmitted to the remote computer 91 that controls the database 90 together with a unique machine identifier which is used by the remote computer 91 to identify details associated with the machine 100, 500, 5000 as stored in the database 90, e.g. its location and its owner or operator. Thus, the remote computer 91 may receive a packet of data identifying one operation of the machine 100, 500, 5000, including the package serialisation data 317, 717 and the identity of the machine 100, 500, 5000.

The remote computer 91 may then check whether the package serialization data 317, 717 has previously been uploaded by another machine or the same machine. If yes, the remote computer 91 may log the details of which packages and which machines are involved, for further investigation. Since the package manufacture and dispatch details will be stored in the database 90 from the time of manufacture, and the point of eventual use is identified by the machine ID, the affected supply chain can be identified.

Data sent between the machine 100, 500, 5000 and the central database 90 may be encrypted.

The machine 100, 500, 5000 may also include a removable data storage device 92, e.g. a USB flash drive, for transferring data between the machine and the remote computer 91 and database 90.

The machine 100, 500, 5000 may be further arranged to output information from the database 90 to a printer 265 to print an information leaflet 266 identifying the drugs 1, 2 contained in the drug packages 301, 302, 701, 702, 703, 7001, 7002, 7003. The information could be retrieved from local memory 263 or directly from the remote database 90 responsive to a real-time authentication request.

The information may include information on any interaction between the identified drugs 1, 2. Thus, the remote database 90 may include special cautionary notices or other information for any relevant drug combinations, which can be displayed on the same patient information leaflet 266 along with (or instead of) the information relating separately to each of the drugs 1, 2.

If a particular combination is contra-indicated then, instead of combining the drugs 1, 2 and providing the information leaflet 266 with a warning, the machine 100, 500, 5000 could be configured to reject the combination so that, instead of operating normally, it displays a warning message (e.g. via touchscreen 267). This could be triggered by information in the local memory 263 or a command from the remote computer 91, responsive to reading the package indicia 317, 717 before commencing the operation to combine the drugs 1, 2 or, if already commenced, before finishing it.

In the illustrated embodiments, the scanners 561 of the second machine 500, 5000 are arranged to read the package indicia 717 before combining the drugs 1, 2 (optionally, before removing the temporary covers 706, 707 from the packages 701, 702, 703, 704, 705). The scanner 261 of the first machine 100 is configured to read the package indicia 317 during the assembly operation, as the stacked packages 301, 302, 304, 305 are moved down onto the pushrod assembly, as further explained below. However, the scanner 261 of the first machine 100 could alternatively be configured (e.g. with a longer scanning window 268) to scan the entire stack of packages before assembly commences, in which case, e.g. if the requested operation is not permissible, the operation could be terminated before the pushrods 110 enter the stack.

The machine 100, 500, 5000 may be configured (or commanded by the remote computer 91) to terminate or disallow an assembly operation, for example, if some or all of the packages 301, 302, 304, 305, 701, 702, 703, 704, 705, 7001, 7002, 7003, 7004, 7005, and particularly the drug packages are identified as counterfeit, and/or the drug combination 1, 2 is contra-indicated.

The printer 265 could alternatively or additionally print an adhesive label (not shown) for attachment to the end user package 300, 700, 7000, e.g. including patient information such as name and address and registration number. The adhesive label could be a peel-off part of the patient information leaflet 266, or could be printed by a second printer 265. The machine 100, 500, 5000 could be arranged to interface with a local or remote, external system (e.g. remote computer 91 and database 90, or a further system and database, e.g. of the pharmacy or local health authority, not shown) to receive patient data and to output the patient data on the patient information leaflet 266 and/or the sticky label and/or to upload it to the remote computer 91 along with the information based on the package indicia 317, 717—for example, for regulatory compliance where the drugs 1, 2 are controlled substances. This enables a drug 1, 2 to be tracked from manufacture to the point of delivery to an identified end user.

The machine could include an external scanner (e.g. as shown in the variant first machine) which is used to identify the packages before introducing them into the machine, where they are scanned again to confirm their identity, and may communicate with the central database on either or both occasions. The moving assembly of the machine could be set to the required start position responsive to the input at the first scanner which is confirmed by the internal scanner before the assembly operation commences. Counterfeit packages could be identified and indicated to the user responsive to scanning them at the external scanner before introducing them into the machine, whereas the exhaustion event of the unique package ID is only recorded in the central database responsive to scanning the package again during the irreversible, one-time assembly operation.

### Go/No-Go Features

Each package 301, 302, 304, 305, 701, 702, 703, 704, 705, 7001, 7002, 7003, 7004, 7005 may include one or more shape profiles. Each shape profile could be, for example: an external contour of the package 701, 702, 703, 704, 705, 7001, 7002, 7003, 7004, 7005 as shown in the second embodiment, or as illustrated in the variant first embodiment; or a pattern of apertures such as locator rod apertures 751 for use with the variant second machine 5000; or a non-circular aperture defined by its angular orientation about an axis of rotation relative to the frame 310 of the package, such as a profile aperture 352 as shown in the first embodiment; or an alignment structure including alignment apertures 351, or alignment and profile apertures 351, 352, as shown in the first embodiment.

The assembly machine 100, 1000, 500, 5000 may include a corresponding shape profile configured to selectively fit or obstruct the shape profile of the or each package. The machine 100, 1000, 500, 5000 may include more than one shape profile to engage with more than one shape profile on each package, and/or to engage with corresponding shape profiles on different ones of the packages. Depending on the function of each shape profile, different package types may be configured to be profile-selective or profile-agnostic—which is to say, a package may be configured to accept a particular shape profile of the machine in any position of adjustment of that shape profile. This principle is illustrated by the first embodiment, in which different ones of the packages have one or more profile apertures 352 in different angular positions, or one or more circular profile apertures 352 which do not select for different angular positions of the corresponding profile rod 152 of the first machine 100.

Different shape profiles of the machine can select for different ones of the packages to ensure that an incompatible package cannot be received in the machine, and/or to ensure that each package type is inserted in the correct position. For example, in the second and variant second machines 500, 5000, each slot 501, 502, 503, 504, 505 is provided with a shape profile that selects for package type (so, distinguishing between drug packages, front cover packages, and rear cover packages), and further selects for the orientation of the package, to prevent the insertion into the slot of the wrong type of package, and/or of the right type of package in the wrong orientation, as further explained below.

The shape profile of the machine may be adjustable by a profile adjustment mechanism, e.g. the index mechanism 153 that rotates the profile rod 152 of the first machine 100, to selectively fit or obstruct the shape profile of the respective package or packages, e.g. profile aperture 352, so as to selectively permit or prevent reception of each drug or other package in the machine.

This can be useful, for example, to prevent incompatible packages (e.g. with different numbers of cells) from being used together. It can also be used to select for capsule capacity to prevent packages 304, 305 with capsule parts 21, 22 in a certain capsule size from being combined with drug packages 301, 302, 303 containing a volume of drugs 1,2 too great for the capsules, as discussed above under the heading “Operation of the assembly apparatus to form an end user package containing capsules”.

The shape profile may be configured as an axially continuous, non-circular cross section of a profile rod, e.g. profile rod 152, and may be rotationally asymmetric so that it fits the corresponding shape profile 352 of the package in only one index position. The shape profile of the packages is similar, but its index position corresponds to the selected criterion. A profile adjustment mechanism, e.g. index mechanism 153, may be arranged to rotate the profile rod 152 about its length axis to selectively fit or obstruct the corresponding shape profile of each of the packages 301, 302, 304, 305.

The first machine 100 includes by way of example three such profile rods 152, indicated (from left to right) as a first profile rod 152′, a second profile rod 152″, and a third profile rod 152′″. The second profile rod 152″ selects for capsule capacity, while the third profile rod 152′″ selects for package size (number of cells 311). The first profile rod 152′ is redundant, and could be used, for example, to control for package origin in a future segmented market (so unauthorised imports from a market region identified by one profile cannot be combined with authorised imports identified by a different profile), or for different package standards if the system parameters should change.

The scanner (i.e. sensor or reader) 261, 561 of the machine 100, 1000, 500, 5000 can also be used, additionally or alternatively to shape profile features, to detect the inserted packages 301, 302, 304, 305, 701, 702, 703, 704, 705, 7001, 7002, 7003, 7004, 7005 and, via the controller 262, to selectively permit or prevent operation of the machine 100, 1000, 500, 5000 responsive to the detected characteristics of the packages or combination of packages.

### Machine for Producing Polypills in the Form of Capsules—Overview

Byway of example, the first embodiment shows an assembly apparatus for filling capsules with drugs, configured as a small machine 100 for use in a pharmacy.

The first machine 100 includes a plurality of pushrods 110 spaced apart in parallel relation, each pushrod having an end surface 111. A pressure plate 121 defines a pressure surface 122 arranged in opposed, spaced relation to the end surfaces 111 of the pushrods to define a receiving space 101 between the pressure plate 121 and the end surfaces 111 of the pushrods.

The first machine further includes an actuation mechanism. The actuation mechanism may be a powered mechanism (i.e. a mechanism driven by power from a non-human power source). In the illustrated embodiment, the actuation mechanism includes a hydraulic piston 141 received in a cylinder 142 and driven by fluid pressure from a motor driven hydraulic pump 143 powered by an external electric power supply 10. Other types of actuation mechanism are possible; for example, the actuation mechanism could be a mechanical linkage, e.g. a geared linkage, driven by an electric motor. Alternatively, the actuation mechanism could be manually operated, i.e. driven by human effort, for example, via a lever.

It should be understood therefore that the term “machine” is used herein as a convenient shorthand for the assembly apparatus, both in its illustrated embodiments and more generally to refer to both automatic and manual variants.

The actuation mechanism 141, 142 is configured to cause relative movement between the pressure plate 121 and the pushrods 110, in a compression stroke, along a displacement axis Xd parallel with the pushrods 110. In the first machine and variant first machine this is accomplished by moving the pressure plate 121 relative to the pushrods 110 which are fixed in the body of the machine, although it could alternatively be arranged for the pushrods to move relative to a fixed pressure plate.

The machine is configured to receive the packages in a stacked configuration in which corresponding cells of the stacked packages are axially aligned, as shown in FIG. 45, and further includes an alignment structure 150. The alignment structure 150 is configured to maintain the stack of packages in alignment with the pushrods 110 when, in use, during the compression stroke, the pushrods 110 are urged through the stack of packages positioned in the receiving space 101, as shown in section in the sequence of FIGS. 52-54.

The machine 100 may further include a controller 262 for controlling the actuation mechanism, and a scanner or reader 261 for reading machine readable indicia 371 in use from the stacked packages. The controller 262 may be arranged to retrieve information from a remote database 90 to identify and authenticate the stacked packages, based on the indicia 371 captured by the scanner 261, as discussed above.

As illustrated, the alignment structure may include a plurality of alignment rods 151 which extend in parallel with the pushrods 110, beyond the end surfaces 111 of the pushrods and into the receiving space 101.

The alignment structure 150, and/or (if present) any shape profile (go/no-go) features, may be configured to prevent the packages from being received in the stacked configuration in more than one possible orientation. That is to say, with respect to the corresponding features of the packages which fittingly engage the alignment structure 150 and (if present) the shape profile features of the machine, the packages may be rotationally asymmetric about one axis or about all three orthogonal axes (or all possible axes).

In the illustrated example, the alignment rods 151 are slidingly received in alignment apertures 351 of the packages, and shape profile features are also provided in the form of profile rods 152 which are fittingly received in profile apertures 352 of the packages, as further described below. Together (and individually) these features define only one possible orientation of the packages in the stacked configuration.

The machine may further include an assembly surface 171 which extends between the pushrods 110. The assembly surface 171 is positionable in a start position (FIGS. 41, 42) proximate the end surfaces 111 of the pushrods, and movable along the displacement axis Xd during the compression stroke. As illustrated, the assembly surface 171 may lie in a common plane with the end surfaces 111 of the pushrods in the start position, so as to receive the packages which are stacked onto the assembly surface before operating the machine. The flush assembly surface and pushrod ends together present a continuous surface which can easily be wiped clean.

The assembly surface 171 may be biased towards the pressure surface 122 of the pressure plate 121 to compress the frames of the stack of packages between the assembly surface 171 and the pressure surface 122, in use, during the compression stroke. This maintains the stack of packages in tightly abutting relation during operation of the machine.

The machine may further include a flat, front surface 172 which extends in a plane parallel with the displacement axis Xd and normal to the assembly surface 171. The front surface 172 and the assembly surface 171 are fixed together to move together during the compression stroke.

In the illustrated example, the assembly surface 171 is formed as an upper surface of a pedestal block 170 (FIG. 31) which is resiliently biased, e.g. by compression springs 173 (FIGS. 19, 26, 28), towards the pressure surface 122. The flat, front surface 172 is a front surface of the same pedestal block 170.

The machine may further include at least one cutter, the at least one cutter being arranged to cut through a portion of the stacked packages, in use, during the compression stroke. In particular, the cutter may detach a label element from each of the drug packages 301, 302.

In the illustrated embodiment there are two cutters 190, configured as miniature circular saw blades mounted on rotating shafts 191 driven by electric motors 192 which also drive impellers 193 of a vacuum extraction and separation system for removing the fragments cut from the packages, as further explained below. The cutters 190 detach the label element 313 from each of the drug packages 301, 302, which adheres to the flap 330 of the cap package 304 to form part of the composite label assembly 312 of the end user package 300, as shown in FIGS. 50, 57, and 58.

Referring to FIG. 1, the first machine 100 may include a casing 201, e.g. of sheet metal, with a door 130 and two debris collection trays 202 that are slidably received in apertures in the casing 201 and accessible from the front of the machine. The casing 201 may be mounted on a base (not shown) that is attachable to a support surface, e.g. a worktop in the pharmacy, the base having two upwardly projecting lugs that extend through holes in the bottom of the casing 201. After fixing the base to the support surface, the casing 201 is placed over the base and then the trays 202 are removed by sliding them out of their apertures in the casing 201 to expose the lugs which project into the space behind the trays 202. Screws are inserted via the apertures to secure the casing 201 to the lugs so that the machine 100 is firmly secured to the support surface.

FIG. 1 shows various features, all of which may be present in the variant first machine and the second and variant second machines 500, 5000. These include the local controller 262 and memory 263 with a user interface and a data transmitter/receiver 269 for sending and receiving data via data link 264, e.g. via the internet, and/or via a removable data storage device 92, to and from the remote database 90 and computer 91.

The user interface could be a touchscreen 267 as shown and/or lights, buttons or other indicators and controls. The user interface may be integrated into the casing 201 or may be external to the casing and adjustable to a comfortable position for each user. It could be integrated into the door 130, e.g. to form part of a window 131 through which the user can observe the operation of the machine. An additional, external scanner 1280 (not shown in FIG. 1) may also be included, e.g. as shown in the variant second machine.

Local controller 262 receives sensor signals 270 from all of the various sensors of the machine. In the illustrated embodiment of the first machine 100 these include a moving frame position sensor 102, a door position sensor 132, optical sensors 175 of the pedestal block sensor assembly 174, a pressure plate sensor 123 for sensing the presence of the pressure pad 250, the scanner 261 for reading package indicia 317, and sensors 154, 165 of each index mechanism 153.

Local controller 262 sends control signals 271 via a control interface 272 which may control any or all of the various functional elements of the machine.

In the first machine 100 these include a door lock (not shown) for locking the door 130, pedestal block latches 103, the LED 182 of the pedestal block sensor assembly 174, profile rod index mechanisms 153, motors 192 for the cutters and debris extraction system, printer 265, and the modified atmosphere system or vacuum generating apparatus 590 (FIG. 60), if provided.

In the second and variant second machines 500, 5000 the controller 262 may similarly control the vacuum generating apparatus 590, cutters 510, 511, punches 519, door lock (not shown), locator rods, grippers and other alignment and transfer assembly elements 551, 552, 553, 554, and all other functional elements of the machine.

The controller 262 also controls the actuation mechanism, which in the first machine 100 includes a hydraulic valve control assembly 144 for controlling the supply of hydraulic pressure from tank 145 via pump 143 to operate the actuators or pistons 141. In the second and variant second machines the actuation mechanism includes actuators 523 which may be e.g. hydraulic, pneumatic, or electrically powered, e.g. comprising solenoids or motors, and could be integrated with the package transfer and alignment mechanism.

### Further Features of the Capsule Cap Package

Byway of example, FIGS. 10, 11 and 12 illustrate capsule cap packages similar to the capsule cap package 304 which has 12 cells, but configured respectively with 24 cells (package 304′), 36 cells (package 304″), and 48 cells (package 304′″). In the first machine 100 there are 48 pushrods 110 with additional alignment rods 151 so that each size of package can be accommodated; each package is rotationally asymmetric so that it can only be positioned at the front of the machine in the position of the packages as shown. Drug packages and capsule body packages may similarly be provided with corresponding numbers of cells.

For particularly voluminous drugs, the user may need to take more than one polypill at the same time; thus, the dose of each drug may be divided between different polypills. Multiples of 12 cells may be selected so that the user can take 1, 2, 3 or 4 polypills at the same time and finish the package with none left over.

In the illustrated embodiment, to ensure that only compatible packages are used together, the right-hand profile rod 152′″ is configured to select for package size, so that the corresponding, right-hand profile aperture 352 of each of the cap packages 304, 304′, 304″, 304′″ is in a different angular position.

Referring to FIGS. 2-9, the frame 310 of the capsule cap package 304 may comprise a block 360 enclosed within a casing 340.

The casing 340 may be formed from one sheet of cardboard with fold lines dividing it into integral parts that can be folded around the block 360, these integral parts including the flap 330 which may be about the same width as the rest of the package, as shown. The sheet may be define apertures for the cells 311 and alignment and profile apertures 351, 352. The sheet 340 may further define a window 331 in the flap 330 so that the remaining part of the flap 330 defines a frame surrounding the window 331, and perforations that delineate the tear-off strips 315 and tabs 316. All of the apertures may be formed e.g. by punching.

The front surface of the sheet 340 may be printed with indicia 317 and, on one part that will form a narrow side of the end user package 300, a legend indicating the number and size of the capsules: “12 capsules size #2”, and below that, the words: “EACH CAPSULE CONTAINS:—”

This phrase will appear at the top of the composite label assembly 312, on the narrow side of the end user package 300, followed on its adjacent broad, rear surface by a list of APIs as printed on the label portions 313 of the drug packages 301, 302.

The tabs 316 forming the ends of the strips 315 may be defined within small apertures 341 in the sheet so that, when the sheet 340 is folded over the edge of the block 360, the tabs 316 are exposed within the apertures 341 to be easily engaged by the user's finger at the edge of the end user package 300.

A sheet or sheets of frangible, i.e. tearable foil 314 (e.g. plastics film or metal, e.g. aluminium foil, or a laminate or metallised composite) may be applied to the surface of the cardboard sheet 340, which is further coated with an adhesive 4 on the surfaces that are to be stuck to the block 360 and on the rear surface of the flap 330. One one side of the block, the foil 314 forms the frangible wall of each cell. On the other side of the block, the foil 314 may extend over the perforations defining the strips 315 so that each cell 311 containing the capsule cap 21 is sealed by the foil also at that end, the foil 314 being broken only when the strip 315 is removed from the end user pack 300 by the user. Adhesion between the foil 314 and the cardboard of the strip 315 may cause the foil 314 to tear away from the block 360 together with the strip 315 to open the cell 311 to remove the finished capsule 20 from the end user package 300.

The adhesive 4 that is to stick the cardboard sheet 340 (and foil 314) to the block 360 need not be permanently tacky, and may be different from the tacky adhesive 4 that is applied to the rear surface of the flap 330 and covered by a release paper 332. The function of the adhesive coating 4 on the flap 330 is to stick the flap 330 to the label portions 313 of the drug packages 301, 302 and to the flap 370 of the capsule body package 305 when the packages are stacked in the machine 100 to be formed into the end user package 300, as further explained below.

Illustrated in FIGS. 2-9 is the 12-capsule size of capsule cap package 304. Comparing this package 304 with the 24-, 36- and 48-capsule variants 304′, 304″, 304′″ shown in FIGS. 10-12, it will be noted that the flap 330 of the 12-capsule size package 304 has a perforated tear line 333 defining a waste portion 334, and a window 331 which extends through the tear line 333 into the waste portion, being of a different shape compared with the window of the 24-, 36- and 48-cell variants. The window 331 of this (smallest) pack size 304 is shaped to co-operate with the pedestal block sensor assembly 174 while the tear line 333 is positioned so that after assembly, the remaining part of the frame defined by the flap 330 will fit the broad, bottom side of the package 304. In the larger package sizes the rear surface of the package will accommodate a window large enough to co-operate with the pedestal block sensor assembly 174, and so there is no need for a tear line 333.

Various other indicia may be printed on the casing 340, including instructions for removing the waste portion 334 and indicia identifying the tabs 316 and cells 311, which optionally may be numbered as shown, or could just have markings so that the position of each cell is evident to the end user.

The block 360 may be assembled from a stack of sub-blocks 361 separated by sheets 362 of paper or foil. Each sheet 362 may define (e.g. by punching) small holes 363 which are of smaller diameter than the apertures 364 formed in the sub-blocks 361, which extend through the thickness of each sub-block 361 to define the cell walls 318. After assembly, the portion of the sheet 362 surrounding these small holes 363 projects inwardly into the cell 311 to form the annular fins 325 that hold the capsule cap 21 co-axially on the axis Xc of the cell 311. The holes 363 can be made with a larger diameter to accommodate a cap for a larger capsule size, or a smaller diameter for a smaller capsule size, without changing the diameter of the cells 311.

The alignment and profile apertures 351, 352 are also formed in the sub-blocks 361 and sheets 362. The sheets 362 and sub-blocks 361 are coated with adhesive on their facing surfaces, and the sheets 362 are interleaved between the sub-blocks 361 before all these components are pressed together with all the apertures in axial alignment to form the block 360.

Alternatively, the whole block 360 could be made as a monolithic unit.

Instead of the annular fins 325, a body of starch foam or other compressible material (not shown) could be arranged in the cell 311 as a locating structure to locate the cap 21 on the cell axis Xc; the body is compressed by the carrier 320 as the cap 21 enters the carrier 320 in use, and may remain captive between the upper (open) end of the carrier 320 and portions of the casing 340 when the capsule 20 is removed from the cell 311 of the end user package 300.

Alternatively, an insert (not shown) could be assembled into each cell of a monolithic block to act as a locating structure which holds the capsule component in spaced relation from the cell wall. The insert could be a tube, e.g. of paper or polymer material, with small tabs cut from its wall to extend radially inwardly. The wall of the tube could be received between the carrier and the cell wall, so that as the carrier slides into the tube, it presses the tabs radially outwardly back into the apertures in the tube wall from which they were cut.

The sub-blocks 361 or monolithic block 360 may be moulded. A simple, monolithic structure makes it possible to make the block 360 or sub-blocks 361 from a material such as a carbohydrate glass, e.g. made from sugar, isomalt, or boiled sugar and glucose syrup; perhaps with a filler such as corn starch, kraft-lignin or lignosulphonate, chalk, microcrystalline cellulose, calcium carbonate, kaolin, talc, wood flour, or other powdered material to add volume, toughness, and/or temperature stability. The cell walls 318 and/or other exposed surfaces of the block 360 can be coated with a non-sticky or lubricating coating, e.g. an oil, a water based lacquer, a resin or varnish, e.g. rosin, copal, shellac, collodion, or a wax or synthesized wax ester, e.g. paraffin wax, beeswax, lanolin, or carnauba wax. When the casing 340 is made from cardboard, this provides a compostable assembly made from sustainable materials. The cardboard casing could be for example a hard cardboard with a thickness of about 1 mm or less, perhaps about 0.5 mm; for example, a hard kraft paper or paperboard. The cardboard could be faced with a white sheet.

Alternatively, the block could be moulded from another material, e.g. glass such as recycled cullet. Alternatively it could be injection moulded from plastics materials. In this case, the annular fins 325 or other locating structure for locating the cap 21 in the cell 311 could be formed integrally with the block, e.g. as fingers arrayed both angularly around the cell axis Xc and along its length so that the block can be formed in a two-part mould. In this and other alternative embodiments, the block could be used with or without a casing 340.

Referring again to the illustrated embodiment, after forming the block 360, the capsule caps 21 (optionally also the locating structures such as starch foam bodies or paper tubes) are introduced into the cells 311 before the casing 340 is folded around the outer surface of the block 360 to seal the caps 21 in the cells 311. The flap 330 remains hinged to the rest of the assembly via one of the fold lines of the cardboard sheet, with its adhesive surface 4 covered by the release paper 332.

The release paper 332 may be a sheet of any material that is readily removable to expose the adhesive side 4 of the flap, e.g. a paper coated with wax, oil or silicone or a textured polymer, and may be printed with a legend visible through the window: “PUSH DOWN PRESSURE PLATE BEFORE REMOVING PAPER”. This reminds the user to make sure that the packages are all pressed together before the adhesive is exposed and the flap adheres to the front of the stack, as further explained below.

### Further Features of the Capsule Body Package

Referring to FIG. 13, capsule body package 305 may include a flap 370 hinged to the rest of the package 305.

When stacked with the other packages in the machine 100, the flap 370 fills that portion (if any) of the window 331 of the flap 330 of the cap package 304 that is not filled by the label portions 313 of the drug packages 301, 302. This ensures that the composite label assembly 312 of the end user package 300 is correctly formed, irrespective of the stack height, which varies with the aggregate volume of the drugs 1, 2 in the packages. The variable stack height is reflected by multiple perforation lines 371 at which a surplus portion 372 of the flap 370, adhered to the waste portion 334 of the flap 330, may be detached from the end user package 300 after assembly.

The flap 370 is coated on its reverse side with a tacky adhesive surface 4, to stick to the bottom surface of the cap package 304 which forms the body of the end user package 300, when the composite label assembly 312 including the flap 300 and the flap 370 is folded and stuck in its final position, as shown in FIG. 57, after removing the stack from the machine 100. The flap 370 may have a fold line 373 at which it is folded so that it wraps around the body of the package 305 until ready for use. The outer surface of the body of the package 305 may have a release coating 5 so that the adhesive, rear surface 4 of the flap 370 can be separated from the rest of the package 305 when positioning the package 305 in the machine 100.

The flap 370 may form part of a cardboard sheet, which may form an outer casing 374 of the package 305. The flap 370 may be connected to a body portion 375 of the outer casing sheet 374 by a pair of wings 376, defining portions of the flap 370 which extend outwardly beyond the ends of the block 380 that forms the cells 311 as further described below. Between the wings 376 the flap 370 may be separated from the body portion 375 of the outer casing 374 by a slit 377, which may be positioned as shown collinear with a fold line 378 between the wings 376 and the body portion 375. When the body portion 375 is positioned to form the upper surface of the package 305, the wings 376 extend out within the thickness dimension of the package to form its front surface when the package is arranged in the stack in the machine 100, and the rest of the flap is unfolded to extend downwardly in the same plane below the wings against the front surface 172 of the pedestal block 170, as shown in FIGS. 42-43. In use, the portion of the flap 370 between the wings 376 will be detached by the rotary cutters 190 to become part of the composite label assembly 312.

The detachable part of the flap 370 may bear a legend: “NO OTHER ACTIVE INGREDIENTS”. This legend appears at the end of the list of APIs forming the composite label assembly 312 of the end user package 300.

The wings (or another part of the package 305) may be printed to indicate the number of capsules and the size of each capsule, e.g. twelve capsules of size #2 as shown in the illustrated embodiment. The system may be configured to work with only one capsule size, but as shown, can accommodate different capsule sizes from a #2 up to a maximum #0E. It could be designed for larger capsules, but they are harder to swallow. Different capsule sizes can be useful to allow for swallowing difficulties in different patients. For example, a patient might prefer to receive a dose divided between several, smaller capsules, each containing a single dose of each of the combined drugs 1, 2 but in a fractional quantity of the total required dose which is obtained by taking two or more capsules together.

By way of example, FIG. 13A shows a conventional, empty capsule 20 assembled from a body 22 and a cap 21, the cap 21 being arranged to fit telescopically over the body 22. The cap 21 may be retained to the body 22 after assembly by friction, or by complementary snap-fit features (not shown) such as an annular recess and protuberance, and/or by a tacky, edible adhesive region arranged, e.g. inside the cap 21, such as inside an inwardly open, radially outwardly extending, annular recess (not shown) of the cap 21, to adhere to the open end or co-operating, radially outwardly extending, annular protuberance (not shown) of the body 22. In such arrangements the adhesive contained in a recess of the cap only comes into contact with the body when the outer annular protuberance of the body (near its open end) enters into the inwardly open, annular recess formed in the cylindrical wall of the cap, so that the snap-fit action of the cap on the body also engages the adhesive bond between the two parts. The capsule parts 21, 22 can be made from any conventional material, e.g. gelatin or vegetable materials such as hydroxypropyl methylcellulose (HPMC) or pullulan.

The size of the frame 310 of each package 301, 302, 304, 305, and of the pushrod assembly of the machine, may be selected to accommodate capsules up to a maximum capsule size, which in the illustrated embodiment is a size #E capsule. To accommodate smaller capsules such as a size #1 or size #2, the wall thickness of the carrier 320 may be increased to reduce its inner diameter without changing its outer diameter.

Two small sensor apertures 379 are formed in the flap 370 to co-operate with the pedestal block sensor assembly 174 in use, as further explained below.

A sheet of foil 314 may be attached to cover the inwardly facing surface of the body portion 375 of the outer casing 374 to form the frangible walls which seal the upper ends of the cells 311 containing the capsule bodies 22 in the carriers 320, and which are penetrated by the carriers 320 as they exit the cells 311 during assembly.

The body of the package 305 may be formed from a monolithic block 380, which defines the cells 311 and cell walls 318 as cylindrical apertures extending through the block 380 between its opposite broad, flat sides. The block 380 may be made the same way as the sub-blocks 361 of the cap package 304 as described above, e.g. by moulding from a glass, e.g. a carbohydrate glass, or alternatively from plastics material.

The block 380 may be encased in an inner casing 381, which may be another sheet of cardboard formed with fold lines at which it is folded around the block 380. In the illustrated embodiment, the inner casing 381 has a sheet of foil 314 on its inwardly facing surface which adheres to the block 380 to form a foil wall that seals the lower end of each of the cells 311 proximate the bottom surface of the respective carrier 320. This foil wall 314 is penetrated by the upper end 111 of the respective pushrod 110 as it enters the stack during assembly. The inwardly facing surface of the inner casing 381 (except for the parts defining the foil walls that face the carriers 320) is coated with adhesive 4 to stick to the block 380.

Alternatively, the adhesive could cover also the parts of the foil walls that face the ends of the carriers, in which case the foil that overlies the end of each carrier will be detached from the capsule body package together with the carrier to become a part of the end user package, eventually being covered by the adhesive flap.

Alternatively, the end surface of each pushrod, and/or the closed (bottom) end surface of each carrier 320, could be slightly convexly domed rather than flat, so as not to trap the fragments of foil covering the end of the carrier, which remain attached to the capsule body package.

Alternatively, the lower side of the capsule body package could be formed without foil and closed by the end surfaces of the carriers 320. The carriers may be assembled into the frame of the capsule body package, or alternatively could be formed integrally with the cells of the capsule body package with break lines at which each carrier will separate from the frame.

The outwardly facing surface of the inner casing sheet 381 may be printed with indicia 317, e.g. including a barcode as shown which appears on the end of the package 305 and is read by the scanner 261 during assembly.

The outwardly facing surface of the inner casing 381 may be provided with a release coating 5 to prevent adhesion of the sticky side of the flap 370 when it is folded around the package 305 in transit and storage. Alternatively the flap 370 could be provided with another release paper that can be removed by the user.

In order to guide the capsule body 22 through the aligned cells 311 of the stack and to help penetrate the foil walls 314 of the cells 311 during assembly of the end user package 300, the capsule body 22 may be received in a carrier 320 as also shown in FIG. 13A. The carrier 320 may be moulded, e.g. from a glass or plastics material, and may define a generally cylindrical wall extending from a closed end 321 to an open end 322. The open end 322 may define one or more salient portions 323 which extend axially forwardly to penetrate the foil walls 314 of the cells as the carrier 320 is urged by the pushrod 110 through the stack of packages.

The capsule body 22 is received in the internal cavity defined by the cylindrical wall of the carrier 320 with the open end of the capsule body 22 at the open end of the carrier 320, as shown. The internal diameter of the carrier 320 is selected so that the capsule body 22 is a sliding fit in the carrier 320, and its cavity may also have a rounded lower, inner end to fit the curvature of the closed end of the capsule body 22, as shown.

The outer diameter of the carrier 320 is selected to be a sliding fit in the aligned cells 311 of the stacked packages, making allowance for the thickness of the ruptured foil walls 314 which may extend between the carrier 320 and the cell wall 318. The cells 311 of the capsule cap package 304 may be of slightly smaller diameter so that the carrier 320 is a tighter or interference fit in the respective cell 311 of the cap package 304 to close the cell containing the finished capsule 20. The carrier 320 may be coated externally with a lubricant such as a wax or silicone to assist it to slide through the stack during assembly.

The carrier 320 may have a slightly enlarged internal diameter portion 324 proximate its open end 322 relative to its slightly smaller internal diameter proximate its closed end 321, as shown, to accommodate the capsule cap 21 as it slides into the enlarged portion 324 between the capsule body 22 and the cylindrical wall of the carrier 320 during assembly.

The radially outer surface of the carrier may be textured or barbed for part or all of its length, e.g. as shown in the variant carrier 1320, to help retain the carrier in the cell of the end user package when the end user package is removed from the machine after assembly. The surface texture or barbs may engage an internal spacing or support structure within the cell 311, e.g. a flocked coating, as illustrated in the variant first embodiment.

After inserting the carriers 320 containing the capsule bodies 22 into the cells 311 of the block 380, the inner casing 381 may be folded around the block, and then the outer casing 374 folded around the inner casing 381, or alternatively the inner and outer casings 381, 374 may be assembled together before folding them around the block 380, to seal the carriers 320 containing the capsule bodies 22 in the cells 311.

### Further Features of the Label Portions of the Drug Packages of the First Embodiment

Referring now to FIGS. 14-17, the first and second drug packages 301, 302 of the first embodiment may be formed in a similar way to the capsule cap and body packages described above, comprising a monolithic block 400 encased in a casing. The casing may comprise an inner casing 410 and an outer casing 420, each of which may comprise a flat sheet of cardboard as shown in FIG. 15, with fold lines at which the sheet is wrapped around the block.

The casing, e.g. the outer casing 420 as illustrated, or any other part of the drug package 301, 302 may define the label portion 313, which may have indicia on its outwardly facing surface. Its opposite, inwardly facing surface may have a tacky adhesive coating 4, which after assembly may face a release coating 5 on the narrow side of the block or, as illustrated, of the inner casing 410.

The label portion 13 may be separated from the rest of the cardboard sheet forming the outer casing 420 by slits 421, so that it is attached to the rest of the sheet only by outer wing portions 422 which extend outwardly beyond the ends of the block 400. The release coating 5 allows the label portion 313 to be removed from the rest of the drug package 301, 302 together with the flap 330 of the cap package 304 after the label portion 313 is separated from the wing portions 422 by the cutters 190 during assembly. In this position during assembly, the indicia on the front surface of the label portion 313 are exposed in the window 331 of the flap 330.

In the illustrated embodiment these indicia read “OLANZAPINE 20 mg” on the first drug package 301, and “CITALOPRAM 20 mg” on the second drug package 302. There is also a “+” symbol which, when the label portion 313 is displayed as part of the composite label assembly 312 on the end user package 300, indicates that the stated drug is present in combination with the next drug in the list.

In the illustrated embodiment, the number of capsules (“12 CAPSULES”) is indicated to the left of the label portion 313, and to the right appears the indication “3 UNITS”. This latter indication corresponds to the thickness dimension of the package. In the illustrated embodiment, the first drug package 301 has a thickness dimension T1 of three thickness units, while the second drug package 302 has a thickness dimension T2 of only two thickness units—so, (T1=1·5 T2). In the illustrated embodiment, each thickness unit is about 4 mm and corresponds to about 0.07 ml of internal volume within the capsule. Each thickness unit corresponds to the same volume of drug, which in the illustrated embodiment is about three spheroids 3 of about 2.8 mm diameter. Thus, it can be seen that each cell 311 of the first drug package 301 contains 1-5 times as many spheroids 3 as the corresponding cell 311 of the second drug package 302. The thickness T1 or T2 of each package in the stack height dimension (e.g. the vertical dimension of the machine 100, as shown) is preferably a multiple of (i.e. perfectly divisible by) the thickness increment, i.e. the thickness unit (e.g. 4 mm).

In use, the pharmacist selects the required drug packages to be formed into the end user package 300 (which could be one, two, three, four or more drug packages) and then notes the total number of units. For example, if the prescription requires one first drug package 301 and one second drug package 302 as shown, then the total number of units is 3+2=5 units. Referring momentarily to FIG. 13, it can be seen that the size #2 cap package indicates “SIZE #2/MAXIMUM 5 UNITS”. So, the pharmacist can see that a #2 capsule size is suitable for the required drugs. If more drugs were needed then a larger capsule size package would be selected, for example, a size #1 or a size #0E. (Capsule sizes are standardised as well known in the art.)

### Further Features of the Drug Packages of the First Embodiment

Referring again to FIGS. 14-17, the remainder of the outer casing 420 of each drug package 301, 302 may be cut, e.g. punched to define apertures for the cells 311 and alignment and profile apertures 351, 352, which after assembly align with the corresponding apertures of the inner casing 410 and the block 400, as shown.

The block 400 may be formed similarly to the block 380 and sub-blocks 361 as described above, e.g. by moulding from a glass, e.g. a carbohydrate glass, or alternatively from plastics material. The block 400 defines the cell walls 318 of the cells 311, each cell containing the respective single dose of the first or second drug 1, 2, preferably in granular, more preferably spheroidal form, as shown. Similarly to the blocks 380 and 361 described above, each cell 311 may define a cylindrical hole which extends along a cell axis Xc and opens at the opposite, broad sides of the block 400. The alignment apertures 351 and profile apertures 352 similarly extend along their respective axes through the thickness of the block 400 to open at its opposite, broad sides. If the second or central profile rod 152″ is assigned to select for capsule size then the corresponding, central profile aperture 352 may be circular, as shown, so that the drug package is capsule size agnostic—i.e. it can be used with capsules of any size.

The inner casing 410 may be printed with indicia 317 on its outwardly facing surface, which in the regions that will be superposed on the broad sides of the block 400 is also covered by sheets of foil 314 to form the frangible walls at both opposite ends of each cell 311. The portion of the outwardly facing surface of the inner casing 410 that will face the adhesive side of the label portion 313 of the outer casing 420 is coated with the release coating 5. The inwardly facing surface (except for the frangible cell end walls 314) may be coated with adhesive 4 to stick to the block 400.

During manufacture, the single dose of the respective drug 1 or 2 is introduced into each of the cells 311 of the block 400 before the inner casing 410 is applied to sealingly enclose the drug, e.g. granules or spheroids 3 within the cells 311. The inner and outer casings 410, 420 can be assembled together before applying them to the block 400, or the inner casing 410 can be applied first before applying the outer casing 420.

### Further Features of the First Machine and its Method of Operation

Cyclonic Separator, Cutters and Profile Rod Assembly

Referring to FIGS. 18 and 19 and FIG. 26, the first machine 100 includes a fixed assembly comprising forty-eight pushrods 110 (one for each cell 311 of the largest pack size 304′″, FIG. 12 which the machine is designed to accommodate), and six alignment rods 151. The pushrods and alignment rods are spaced apart in fixed, parallel relation with the upper ends 111 of the pushrods lying in a common horizontal plane (when the machine 100 is in an upright use position as shown), and the upper end surfaces of the alignment rods 151 all lying in another horizontal plane, above that of the end surfaces 111 of the pushrods.

The pushrods 110 and alignment rods 151 may be fixed in a base plate 203, made for example of cast iron or cast aluminium, to provide rigidity. The base plate 203 may also support fixed guide rods 204 which guide the moving frame assembly 120, and spring guide rods 205 for the compression springs 173.

Instead of rigidly fixing the pushrods 110 and alignment rods 151 in the base plate 203, e.g. by brazing, welding, an interference fit, or a rigid adhesive bond, the pushrods and optionally also the alignment rods may be resiliently mounted, e.g. by fixing a lower end of the rod in the base plate 203 or other support by means of an elastomeric insert or bonding material. The variant first machine illustrates one possible arrangement. The pedestal block then slidingly supports the rods 110 or 151 in parallel relation, while the resilient mounting allows each rod to move slightly to compensate for lack of parallelism due to manufacturing tolerances. A resilient mounting may also help to decouple the pushrods 110 from the base plate 203 so that the pushrods can better conduct vibrational energy to the carriers, drug granules and capsule components during assembly, as further discussed below.

The casing 201 may be made from stainless steel sheet and/or cast aluminium. The casing 201 may define shrouds 206 which enclose the cutters 190, which may be formed as miniature circular saw blades mounted to rotate on shafts 191 so that the blade is enclosed from the front and sides but projects by a few millimetres from the open, rear or inwardly facing side of the shroud 206, as best seen in FIG. 19A. The shafts 191 are driven by electric motors 192 which also drive impellers 193 to evacuate debris from the cutters 190 in an airflow which is drawn in through the rear openings of the shrouds 206 and flows as indicated by the direction of the arrows, through a cyclonic separator 194 which separates the debris from the airflow. The particles fall into the collection trays 202 while the airflow is drawn through the impellers 193 and then flows back through perforated wall 207 into the receiving space 101 and back into the shrouds 206.

The profile rods 152 are arranged to rotate about axes Xp that are fixed in parallel with the pushrods 110 and alignment rods 151, and may be arranged to pass through holes in the base plate 203 so that the mechanism 153 that rotates them can be arranged conveniently beneath the base plate 203, as shown.

Byway of example, the first machine provides three profile rods 152, although it could have one or two or more than three. From left to right as shown in FIG. 28, the first (left-hand and rear-most) profile rod 152′ is reserved for future selection criteria; the second profile rod 152″ selects for capsule capacity (e.g. between a size #2, #1, or #E capsule); and the third (right-hand) profile rod 152′″ selects for package size (12, 24, 36 or 48 capsules).

Each profile rod has an axially continuous, non-circular cross-section which defines its shape profile, and extends along a profile rod axis Xp in parallel with the pushrods 110 beyond the end surfaces 111 of the pushrods and into the package receiving space 101 to selectively permit or obstruct each package that selects for that shape profile from being received in the receiving space 101. Packages that do not select for that particular shape profile have a circular hole 352 in that position, i.e. they are profile-agnostic.

Referring now to FIGS. 36-38, each profile rod 152 may be mounted on a profile adjustment or index mechanism 153, which may form an actuator operable by the controller 262 and which rotates the profile rod 152 about its length axis Xp. The mechanism or actuator 153 may be driven for example by a solenoid 162 as shown, or could be an electric motor or any other powered actuation device; alternatively the profile adjustment mechanism 153 could be manually operable by the user.

In the illustrated example there are sixteen angular index positions, defined by the position of two spring biased index plungers 156, 157 on an index wheel 158. (One index plunger could combine both functions if preferred.) A sensor 154 detects by the position of the index plunger 156 when the index wheel 158 is in the index position. The opposite index plunger 157 is locked in position by an abutment 161 formed on a reciprocating arm 160 driven by the solenoid 162, so that when the arm 160 is in a rest position (FIG. 38, suffix -a) the index wheel 158 is locked by the index plunger 157 to prevent the profile rod 152 from rotating.

A pawl 163 is mounted on a pivot block 166 which in turn is slidably mounted on the arm 160. When the controller 262 commands the mechanism 153 to rotate the profile rod 152, the solenoid 162 retracts the arm 160 from the rest position (FIG. 38, suffix -a) to an unblocked position (FIG. 38, suffix -b), taking up lost motion between the arm 160 and the pivot block 166 while the abutment 161 moves to unblock the index plunger 157.

Further retraction of the arm 160 now engages and moves the pivot block 166 so that the pawl 163 urges the ratchet plate 164 in rotation to the new index position (FIG. 38, suffix -d). As the index wheel 158 moves past an intermediate position (FIG. 38, suffix -c) the index plungers 156, 157 are urged inwardly by their springs, urging the index wheel 158 into its new rest position in the next index position (suffix -d). The arm 160 then extends through an intermediate position (FIG. 38, suffix -e) back to its rest position (FIG. 38, suffix -a) while the springs of the index plungers 156, 157 prevent movement of the index wheel 158, leaving the profile rod 152, ratchet plate 164 and index wheel 158 in their new index position (FIG. 38, suffix -d).

Sensors 165 identify the unique pattern of indicators 155 that represents each of the sixteen index positions, shown in FIG. 37 as a four-bit string.

### Moving Frame Assembly

Referring now to FIGS. 20-29, the pressure plate 121 may be pivotably mounted on a moving frame assembly 120, which may further include a front plate 220 which is pivotable from a lowered position (FIG. 24) to a raised position (FIG. 25). The pressure plate 121 may be arranged as an upper plate of the assembly which is pivotable from a raised position to a lowered position, shown respectively in broken lines and in solid lines in FIG. 24. A mechanism, e.g. an over-centre mechanism, may be provided to retain the pressure plate 121 in the raised position. When the front plate 220 is pivoted to the lowered position and the upper, pressure plate 121 is pivoted to the raised position, the packages may be inserted into and removed from the receiving space 101. When the upper, pressure plate 121 is pivoted to the lowered position, the front plate 220 may be pivoted to the raised position and locked to the upper plate 121. In this position the moving frame assembly may be moved by the hydraulic pistons 141 or other actuation mechanism so that the pressure plate 121 urges the stacked packages down over the fixed pushrods 110. The moving frame assembly 120 including the upper plate 121 and front plate 220 moves as a single unit, with the pressure surface 122 of the upper plate 121 supported at the rear by its pivot axis and at the front of the machine by its connection to the front plate 220.

The inwardly facing surface 228 of the front plate 220 may be arranged in parallel, opposed relation to the front surface 172 of the pedestal block 170 when the front plate 220 is locked to the pressure plate 121. When the front plate is locked to the pressure plate 121 the composite label assembly 312 may be compressed between the inwardly facing surface 228 of the front plate and the front surface 172 of the pedestal block, as best seen in FIGS. 47-48 and further described below. To ensure that the applied pressure is always in a desired range, the surface 228 may be resiliently compressible towards the body of the front plate 220. For example, the surface 228 can be formed as a sheet, e.g. of stainless or chrome plated steel, separated from the body of the front plate 220 (which may be formed as a thicker metal plate) by a resilient interlayer 229 of resiliently compressible material, e.g. a layer of sponge foam a few millimetres thick. The surface 228 may be reflective, e.g. by polishing the sheet, so as to provide a target for the optical sensors of the pedestal block 170 as further described below.

The moving frame assembly 120 may further define an inner casing 230 which has a lower cavity 231 in which the pedestal block 170 is slidingly received, and an upper cavity that defines the receiving space 101. The lower cavity may have clearance slots 236 to accommodate the pedestal block latches 103 as further described below.

The moving frame assembly 120 may be guided to reciprocate along a vertical axis relative to the fixed body of the machine 100. By way of example, in the illustrated embodiment, this is achieved by means of guide sleeves 232 slidably mounted on the guide rods 204 and fixed to the inner casing 230. The guide sleeves 232 may be connected to the inner casing 230 as shown via an outer frame with brackets 233 at which the moving frame assembly 120 is connected to the hydraulic pistons 141.

A scanning window 268 may be arranged in the inner casing 230 so that the scanner 261 can read the indicia 317 on the stacked packages as the moving frame assembly 120 moves relative to the scanner 261; the scanner could alternatively be arranged to read the indicia 317 while the moving frame assembly 120 remains static.

The upper (pressure) plate 121 may include a handle 124, which may be configured as a knob as shown, to be pushed or grasped by the user when pivoting the pressure plate 121 up and down.

As a safety feature, the machine 100 may be arranged such that the pressure plate 121 cannot be operated to form the end user package unless the door 130 is closed. This can be accomplished, for example, by arranging the controller 262 to prevent operation unless a door position sensor 132 indicates that the door is closed.

Where, as in the illustrated embodiment, a front plate 220 is arranged to provide a mechanical support for the front end of the pressure plate 121, the machine 100 may be arranged such that the pressure plate 121 cannot be operated to form the end user package unless the pressure plate 121 is locked to the front plate 220.

This can be accomplished, for example, by means of three complementary functional features (a), (b) and (c) as follows.

In accordance with feature (a), a part of the pressure plate 121 (e.g. the handle 124, as illustrated) may project forwardly through the doorframe to prevent the user from closing the door 130 when the pressure plate 121 is pivoted away from its lowered position. In accordance with feature (b), a part of the front plate 220 may be arranged to project forwardly through the doorframe to prevent the user from closing the door 130 unless the front plate 220 is pivoted to a fully raised position. (In the illustrated embodiment, most of the front plate 220 extends through the doorframe when lowered.) In accordance with feature (c), the pressure plate 121 may be arranged, when pivoted to a lowered position, to prevent the front plate 220 from pivoting to a fully raised position unless the front plate 220 is locked to the pressure plate 121. In the illustrated embodiment, this is accomplished by arranging for the front plate 220 to lock to the pressure plate 121 simply by pivoting the front plate 220 to the fully raised position when the pressure plate 121 is pivoted to the lowered position.

Accordingly, features (a), (b) and (c) in combination define a simple and intuitive method of operation which requires that the user first pivots the pressure plate 121 to its lowered position, then pivots the front plate 220 to its raised position to lock it in one movement to the pressure plate 121, before the door 130 can be closed and the machine 100 operated.

When the pressure plate 121 is pivoted to a lowered position, the handle 124 may be received in an inwardly opening cavity 133 defined by the handle of the door 130, within which cavity it can reciprocate up and down with the rest of the moving frame assembly 120 while the door is closed.

The pressure plate 121 may have abutments 125 to rest on corresponding abutments 234 of the inner casing 230, as shown in FIG. 21, when the user pivots the pressure plate 121 to its lowered position and then pushes down on the handle 124 to move the moving plate assembly vertically downwardly through a very short distance (e.g. one or two millimetres) so as to apply pressure via the pressure surface 122 to compact the stacked packages against the assembly surface 171 of the pedestal block 170, before removing the release paper 332.

The hydraulic valve control assembly 144 may be arranged to allow this movement; for example, the controller 262 may control the valve assembly 144 to permit fluid to flow through a non-return valve or valves of the assembly 144 as the moving frame assembly 120 is pressed down by the user.

The pressure plate 121 may have a slot 126 to accommodate the central group of alignment rods 151 and the second profile rod 152″ which project into the receiving space 101, as shown.

FIG. 34 illustrates one possible mechanism 221 by which the front plate 220 can be locked to the pressure plate 121 simply by pushing forward on a handle 222, and then unlocked again simply by pulling back on the handle 222.

As best seen in FIGS. 28 and 29, pressure plate 121 includes a forwardly projecting tongue 121′ with an upper surface in which are formed a shallow recess 127 that opens at the forward end of the tongue, and shallow recesses 128 and 129 proximate its rear end.

Front plate 220 includes a recess 225 into which the tongue 121′ slidingly extends when the front plate 220 is pivoted upwards towards the lowered pressure plate 121, and a rotatable handle or knob 222 which operates via a lever 226 its internal mechanism 221 as shown in FIG. 34.

The mechanism 221 includes an assembly of steel balls including a locking ball 223 and a control ball 224. The control ball 224 is outwardly spring biased by a control lever 227 which in turn acts on the rest of the balls.

Suffix a—indicates a rest position of the locking mechanism 221 wherein the two plates 121, 220 are locked together around the stacked packages, ready for the machine 100 to press the stack to form the end user package. In this position the control ball 224 is received in recess 129 so that the control lever 227 is maintained in its rest position by its bias springs. In this position the control lever 227 maintains the rest of the balls in compression. The handle 222 is forwardly spring biased to a rest position in which the lever 226 also maintains the balls in compression. In this state the locking ball 223 is received in recess 128 and cannot retract, retaining the tongue 121′ in the recess 225 so that the two plates 121, 220 remain locked together.

Suffix b- indicates an initial, unlocked position of the mechanism 221 as the handle 222 of the front plate 220 is pulled backwardly to begin to move the front plate 220 away from the top or pressure plate 121. In this state, the lever 226 releases the balls so that the locking ball 223 can retract as it is urged by the tongue 121′ out of the recess 128 and back into its hole in the bulbous housing formed at the end of the front plate 220 as the tongue 121′ starts to move out of the recess 225.

Suffix c- illustrates an unlocked position of the mechanism 221 as the tongue 121′ is nearly withdrawn from the recess 225. The same, unlocked position is obtained when the tongue 121′ initially enters the recess 225 as the user pushes forward on the handle 222 to close the front plate 220 onto the top, pressure plate 121. Pushing forward on the handle 222 moves the lever 226 to its blocking position. However, as the tongue 121′ enters the recess 225, the locking ball 223 is able to move into recess 127 while the tongue 121′ forces the control ball 224 to retract, urging the control lever 227 to a release position in which the rest of the balls are able to move, which allows the locking ball 223 also to retract as it moves out of the recess 127.

Suffix d- illustrates an almost-locked position of the mechanism 221 as the handle 222 is pushed further forward to urge the front plate 220 through the final, short distance of travel into the locked, rest position as illustrated by suffix a- to lock the plates 121, 220 together. The control ball 224 has entered the recess 129, allowing the control lever 227 to move to its rest position; however, the control lever 227 is maintained in its retracted position by the pressure of the balls responsive to the locking ball 223, which is maintained in its retracted position because it is engaged with the upper surface of the tongue 121′, in-between recesses 127 and 128. Further movement allows the bias spring pressure of the control lever 227 to urge the locking ball 223 to enter recess 128, allowing the control lever 227 to move to its blocking, rest position to leave the mechanism in the locked, rest position of suffix a-.

The controller 262 may monitor the position of the moving frame assembly 120 by means of a moving frame position sensor 102 which senses the position of the moving frame assembly 120 and sends position data to the controller 262. In the illustrated embodiment, the sensor 102 senses the vertical position of the inner casing 230 relative to the fixed casing 201 and base plate 203.

The pressure plate 121 may include a pressure plate sensor 123 (FIG. 42) for sensing the presence of the pressure pad 250. Referring now to FIG. 30, a pressure pad 250 may be provided to help distribute the pressure from the pressure plate 121 over the whole plan area of the stacked packages. The pressure pad may be a solid block, e.g. of plastics material, with holes to accommodate the alignment rods 151 and profile rods 152 when it is placed on top of the stack, as shown in FIG. 45. The pressure pad may include an identifier 251 arranged to be sensed by the pressure plate sensor 123 which sends a signal to the controller 262 indicating that the identifier 251 has been sensed, which confirms that the correct pressure pad has been fitted in the correct position. The holes or other features of the pressure pad may be asymmetric so that the pressure pad can only be fitted in one position.

In the illustrated embodiment, the holes formed in the pressure pad 250 to accommodate the profile rods 152 are all round, so that the pressure pad does not select for any particular profile rod index position. As further discussed below under “Alternative embodiments”, in alternative, manual embodiments, multiple pressure pads with different thicknesses and profile rod apertures that select for different profile rod index positions could be provided as a simple, mechanical way to correlate stack height with capsule capacity without an electronic control system.

Vibration Source and Graduated Pressure

The assembly apparatus of the first or variant first embodiment may include at least one vibration source, which is configured to transfer vibrational energy to the capsule parts or the single doses during assembly.

For example, the pressure plate and/or pedestal block of the first machine could be adapted to include the vibration source, or multiple vibration sources to make the granules or spheroids 3 more mobile and so assist them to enter the capsule body. The vibrational energy may also introduce mechanical dither between the capsule components, assisting the cap to locate in the correct position on the capsule body.

One or more vibration sources could be arranged to induce vibrations in the pressure plate and/or the pushrods and/or the pedestal block or other machine components; the variant first machine, further discussed below, illustrates one possible arrangement in which a vibration source is located at the lower end of each pushrod which transfers vibrational energy through the stacked packages to the carrier. The frequency and/or amplitude (power) of the vibration may be constant or may vary between different stages of the operation.

The actuation mechanism may be operable to control the speed and pressure at which the pushrods 110 move through the stacked packages, and to reduce the speed and/or pressure at the moment that the capsule caps begin to move into the carriers 320 to engage the capsule bodies. One way to achieve this would be to provide a resilient buffer or lost motion mechanism in the power transmission, such as a gas spring, e.g. an accumulator in which an enclosed volume of air or other gas is separated from hydraulic fluid by a diaphragm or piston, which communicates via a valve with the hydraulic circuit to the actuator pistons 141 that drive the moving frame assembly 120 or other moving parts of the machine. The carriers 320 are advanced through the packages in a first, fast phase of movement, and then, with the accumulator in a de-energised condition, the valve is opened so that the hydraulic pressure is substantially decoupled from the moving parts and, instead, gradually energises the gas spring. The pressure applied at the point of contact between the capsule cap and body components now increases only very gradually in a slow phase of movement until the capsules are closed. The gradually increasing pressure provides enough time for the capsule components to become properly aligned—particularly where a vibration source is also used to provide dither—before the pressure increases to close them fully together and urge the carriers 320 into their final position. The gas spring or lost motion arrangement may be decoupled from the actuation mechanism (e.g. by closing the valve to the accumulator) in this final, third phase of movement. The accumulator is then de-pressurised before the next operation.

Pedestal Block

Referring to FIG. 31, the pedestal block 170 may be moulded from plastics material. The front surface 172 of the block (including the front surface of the sensor assembly 174) may have a low surface energy to prevent adhesion of the tacky adhesive coating of the flap 370 of the capsule body package 305 which is pressed against it in use. This can be achieved for example by making the pedestal block 170 from a low surface energy plastic, for example, a thermoplastic polyolefin, polytetrafluoroethylene, polyvinyl fluoride or polyvinylidene fluoride, or by providing the front surface 172 with a low surface energy treatment, for example, a texture or a low surface energy coating. A nanoglass coating could be used.

The pedestal block 170 defines through-holes to accommodate the pushrods 110, alignment rods 151 and profile rods 152, and may include further holes, opening downwardly but closed at their upper ends, to accommodate the compression springs 173 that apply an upwardly directed bias force to the pedestal block 170.

Referring to FIGS. 33 and 35, each profile rod 152 may extend within the block 170 through a respective profile rod sleeve 176, which is rotatably received in a respective one of the circular through-holes of the block 170 but retained in its axial direction in a fixed position relative to the block 170.

At its upper end the profile rod sleeve is configured to occupy the space between the profile rod 152 and the wall of the through-hole in which it is housed, so that the upper end surface of the profile rod sleeve 176 remains flush with the assembly surface 171.

The profile rod sleeve 176 may be retained in the axial direction between an abutment formed by a step in the diameter of the through-hole to abut against an upper shoulder 177 of the sleeve 176, and a lower retainer 178 which is screwed or otherwise engaged into the block 170.

The pedestal block 170 may be urged downwardly against the upward, restoring force of the compression springs 173 by the actuation assembly, e.g. hydraulic pistons 141, which act on the block 170 via the pressure plate 121 and stacked packages during the assembly procedure, or, when the packages are not present, via abutments 235 formed on the inner casing 230 of the moving frame assembly 120 to engage an abutment surface, which may be formed by the lower end of a slot 179, of the pedestal block 170.

The pedestal block is slidably received in the lower cavity 231 of the inner casing 230 with the rod assembly extending upwardly through it, and is vertically movable in this position through a range of movement between a maximally depressed position (not shown), which is obtained when the pressure plate is fully lowered with the highest possible stack of packages, through an initial, rest position (FIG. 41) in which its upper, assembly surface 171 is flush with the upper end surfaces 111 of the pushrods 110, to a maximally raised position (FIG. 51) in which its upper, assembly surface 171 is flush with the upper end surfaces of the alignment rods 151 and profile rods 152.

The compression springs 173 may provide a substantial spring force to urge the pedestal block towards the maximally raised position, which also provides a firm assembly surface against which the user can compact the stacked packages by gently pushing down on the pressure plate handle 124 when the pedestal block is in its initial, rest position (FIGS. 41 and 42).

Each abutment 235 of the moving frame assembly 120 may be arranged also to engage the upper end of the respective slot 179 as the moving frame assembly 120 is raised to its uppermost position, for removal of the end user package 300 at the end of the assembly operation, so as to assist in raising and retaining the pedestal block 170 in its maximally raised position while the packages are removed from the machine.

In alternative arrangements, instead of using return springs 173, the pedestal block 170 could be moved upwards by the actuation mechanism, perhaps using separate actuators, or movable abutments to selectively engage and disengage the pedestal block with the moving frame assembly.

In the initial, rest position (FIGS. 41 and 42) the alignment rods 151 and profile rods 152 project upwardly through the assembly surface so that the packages can be stacked onto those rods, before the door 130 is closed by the user and the pedestal block 170 is urged downwardly together with the stack of packages by the descending pressure plate 121 (FIG. 48).

Referring also to FIGS. 18, 19 and 28, the maximally raised position of the pedestal block 170 (FIGS. 50 and 51) may be defined by abutment of a fixed abutment surface or surfaces 104 of the machine body (which may be fixed to the base plate 203) against an abutment surface or surfaces of the pedestal block 170, which may be defined by the lower end of another slot 180 in the pedestal block 170 into which the abutment surfaces 104 project.

The initial, rest position (FIGS. 41 and 42) may be defined by abutment of the pedestal block latches 103 against an abutment surface of the block 170, which may be the same lower end of the slot 180 as shown.

After the pressure plate 121 has reached its downward limit, defined by the thickness of the capsule cap package 304 above the upper end surfaces 111 of the pushrods 110, the pistons 141 are actuated in the reverse direction (FIG. 49) to raise the moving frame assembly 120 to its maximally raised position. The controller 262 is arranged to retract the latches 103, which normally project into the slot 180, to allow the pedestal block 170 to move upwards past its initial, rest position to its maximally raised position at the end of the assembly operation. In the maximally raised position of the pedestal block (FIGS. 50 and 51) all of the rods 110, 151, 152 are withdrawn from the stacked packages, and the upper ends of the rods 151 and 152 may be flush with the assembly surface 171, so that the end user package 300 along with the other, empty packages below it can all be removed from the machine 100 simply by sliding them forward off the assembly surface 171.

When assembled in the body of the machine 100, the front surface 172 of the pedestal block 170 extends on either side for a short distance behind the shrouds 206. The portions of the cutters 190 that extend a few millimetres from the rear openings of the shrouds 206 are received in slots 181 formed in the front surface 172 of the pedestal block 170, very close to the ends of the shrouds 206.

Referring now to FIGS. 31, 32 and 33, the pedestal block 170 may include a sensor assembly 174 by means of which the controller 262 can sense and confirm the presence and correct configuration of the stacked packages before commencing the assembly operation.

Byway of example, the illustrated embodiment provides three optical sensors 175, each of which detects light emitted by a light source, which may be the same light source for all three sensors, e.g. an LED 182. Light from the LED may be transmitted via first beam splitters 183 and mirror 184 to second beam splitters 185, which direct the three beams of light through windows 186 in a direction normal to the front surface 172 of the pedestal block 170. If the beam is reflected from the reflective surface 228 of the front plate 220, then it is received via the respective, second beam splitter 185 by the sensor 175 which sends a signal to the controller 262.

The controller may turn on the LED 182 when the door position sensor 132 indicates that the door 130 is closed—which in turn means that the pressure plate 121 and front plate 220 must be locked together. The controller 262 then determines by the combination of signals from the three sensors 175 whether or not the packages are correctly stacked in the machine 100. The first beam splitter 183 closest to the LED 182 may be configured to transmit two thirds of the light and reflect one third; the remaining beam splitters may transmit half and reflect half.

As shown in FIG. 43, when the packages are correctly positioned in the machine 100, as seen from the front of the machine, the left-hand one of the windows 186 and sensors 175 is covered by the flap 370 of the capsule body package 305 while the two windows and sensors 186, 175 to the right are aligned with the sensor apertures 379 in the flap 370 of the capsule body package 305.

When the cap package 304 is correctly positioned as shown in FIG. 45, the window and sensor 186, 175 furthest to the right are now covered by the flap 330 of the cap package while the window and sensor 186, 175 in the middle of the array are still exposed through the window 331 in the flap 330 but are covered by the release paper 332.

After the user removes the release paper 332, the middle window and sensor 186, 175 are visible through the window 331 while the left-hand and right-hand windows and sensors 186, 175 are concealed.

The controller 262 may interpret a signal indicating “0-1- 0”, which is to say, the central sensor 175 detects a reflected beam while the other two do not, to indicate a correct package configuration. Other signals may be interpreted to generate an appropriate error message. If for example the signal is “0-0-0” then the controller may infer that the release paper 332 has not been removed, in which case an error message may be displayed on the screen 267 for the user to open the door and remove the release paper. If the same fault code occurs again then the controller 262 may prompt the user to check that the surface 228 is not obscured and then remove the packages and close the door, so that the controller 262 can illuminate the LED 182 to check that it is functional. Alternatively, an LED function check can be carried out automatically every time the machine is used, immediately before opening the door 130 to receive the packages.

Sequence of Operation

The operation of the machine will now be described with reference to FIGS. 41-51.

FIG. 41 shows the machine 100 with the door open and the pedestal block latches 103 engaged to retain the pedestal block 170 in its initial, rest position. The profile rods 152 are set to an index position that selects for a particular package size (number of cells per package) and capsule size (e.g. a #0E, #1, or #2 capsule). The position of the moving frame assembly 120 determines the height of the pressure plate 121 above the assembly surface 171 and so, the maximum height of the stack of packages that can be received in the machine before closing the pressure plate 121 and locking it to the front plate 220.

Referring momentarily to FIG. 24, it may be noted that the position of the pivot axis of the pressure plate 121 may be arranged, not only to provide easy access to the receiving space 101 and to ensure that the handle 124 extends through the doorframe when not pivoted to the lowered position, but also to provide a substantial, horizontal vector component of the direction of movement of the pressure surface 122 as the pressure plate is pivoted through the final few degrees into its lowered position. If the user tries to pivot the pressure plate 121 onto a stack that is too high, it causes a very obvious problem as the pressure plate 121 will not move back into position; instead, it sticks in a forward position on the stack, without generating a large mechanical advantage that could crush the stacked packages. This prompts the user to check whether the selected capsule size is large enough for the total number of units in the stack (indicated by the indicia on the forwardly facing sides of the drug packages).

If the capsule size is not large enough, then the user must remove the stacked packages, re-lock the plates 121, 220 together and close the door 130 before the controller 262 will allow the machine to be reconfigured to accept a larger package size. Similarly, if the user changes his mind and wishes to use a smaller capsule size, he may select a smaller capsule size after closing the door 130. The controller 262 rotates the respective profile rod 152 to select for a smaller capsule size before releasing the door.

FIG. 42 shows a new position of the moving frame assembly 120. Note that the pedestal block 170 remains in the same, initial position, but the pressure plate 121 and inner casing 230 are lower relative to the fixed machine body and shrouds 206. (Although not shown in the drawing, it should be understood that the respective profile rod 152 would also be rotated to the corresponding index position to select for the smaller capsule size before the controller 262 unlocks the door 130.)

The user selects the correct package size and capsule size to fit the new position of the profile rods 152, and starts by placing the capsule body package 305 onto the assembly surface, with its flap 370 unfolded against the front surface 172 of the pedestal block (FIG. 42). The rear surface of the flap 370 is adhesive but does not stick to the pedestal block 170 because of the anti-adhesion properties of its front surface 172.

Of course, if the package has a profile rod aperture that is indexed to select for a different profile rod index position from that set by the controller 262, then the package will not fit. This prevents the user from inadvertently selecting the wrong number of cells or the wrong size of capsule, or from using a package that is disallowed, e.g. for commercial or regulatory reasons related to the market area in which the machine is being operated.

If (as shown) the apertures formed in this and the other packages to receive the profile rods 152 and alignment rods 151 are covered by a foil 314, then the foil 314 is ruptured when the package is placed over the rods. In alternative arrangements, the foil 314 need not cover these apertures in any of the packages.

The user then stacks the required drug packages 301, 302 in any order onto the capsule body package 305 (FIG. 43). More than two drug packages can be used if desired. Similarly, if for any reason the user wants to make an end user package with only a single drug, then only a single drug package could be used. If the user requires a dose of any given drug that is not available in a single package, then two packages containing the same drug could be used.

Next, the user unfolds the flap 330 of the capsule cap package 304, and stacks the cap package 304 on top of the drug packages with the flap 330 hanging down in front of the rest of the packages in the stack (FIG. 44).

The legend: “PUSH DOWN PRESSURE PLATE BEFORE REMOVING PAPER” printed on the release paper 332 reminds the user to do exactly that. The user places the pressure pad 250 on top of the stacked packages (FIG. 45). The controller 262 verifies the presence of the pressure pad 250 by means of the pressure plate sensor 123 which senses the presence of the identifier 251; this can be done at this time and/or after the door is closed. The user then pivots the pressure plate 121 to the lowered position (FIG. 46). If the pressure plate fits then the user knows that the stack height is OK. The user then presses gently down on the handle 124 of the pressure plate 121 to urge the pressure plate down, ensuring that the packages are snugly stacked against the assembly surface 171. The controller 262 may command the valve assembly 144 to permit flow through a non-return valve so that pressure applied by the user is maintained by the pistons 141. Permitting flow through the non-return valve may be subject to verifying the presence of identifier 251.

Then, the user lifts the flap 330, peels away the release paper 332 to expose its adhesive, rear surface (FIG. 46), and then replaces the flap 330 against the forwardly facing surface of the stacked packages below it (FIG. 47). The flap 330 sticks to the forwardly facing surfaces of the label portions 313 of the drug packages 301, 301 and the flap 370 of the capsule body package 305. The indicia printed on the label portions 313 and the legend: “NO OTHER ACTIVE INGREDIENTS” printed on the flap 370 are visible in the window 331 of the flap 330, which also exposes the sensor aperture 379 for the middle window 186 of the pedestal block sensor assembly 174. Thus, the flap 330 forms a frame around the indicia that will become the composite label assembly 312 of the end user package 300.

Next, the user must pivot the front plate 220 to the raised position (FIGS. 47-48) and lock it to the pressure plate 121. When the front plate 220 is locked in its raised position (FIG. 48) the composite label assembly 312 is compressed between the surface 228 of the front plate 220 and the front face 172 of the pedestal block 170, which ensures a good adhesive bond between the flap 330 and the other components 313, 370 of the composite label assembly 312.

The user then closes the door 130 and the controller 262 (responsive to the door closing, or to a further user instruction via the user interface, e.g. touchscreen 267) operates the pistons 141 to cause the moving frame assembly 120 to descend until the pressure surface 122 is separated from the upper surfaces 111 of the pushrods 110 by a distance equal to the thickness of the capsule cap package 304. The front plate 220 acts in tension to maintain the pressure surface 122 in alignment with the horizontal surface of the stacked packages. The pedestal block 170 is urged downwards together with the moving frame assembly against the restoring force of the springs 173 by the pressure surface 122 acting on the frames 310 of the stacked packages which bear against the assembly surface 171.

As the moving frame assembly 120 descends past the scanner 261 (which is fixed to the body of the machine and base plate 203), the controller 262 commands the scanner 261 to read the package indicia 317 from the exposed ends of the packages through the scanning window 268, and, using this data, contacts the remote computer and database 91, 90 to authenticate the packages and download the data for the patient information leaflet 266. The controller 262 may command the printer 265 to print the patient information leaflet 266 while the machine 100 is operating. If patient identity information is required then that may be downloaded from a local database at the pharmacy or health authority and combined with the data transmitted to the remote database 90, and/or printed on the leaflet 266 and/or an adhesive label (not shown) to be applied to the end user package 300, e.g. stating the patient's name and the dosage regimen.

Although not shown, the machine 100 could further include a printer for printing patient information and/or contents information (drug name and dose) directly onto a surface of the end user package 300, either instead of or in addition to the composite label assembly 312.

If there is an authentication issue, or if the identified combination of drugs is contra-indicated, then the controller 262 may terminate the operation and/or issue a warning via the screen 267 and/or on the patient information leaflet 266, while the remote computer 91 updates the database 90 with the received information. As mentioned above, the packages may also be scanned via an additional, external scanner 1280 (FIG. 102) before introducing them into the machine, in which case any necessary warning may be issued without unlocking the door to receive the packages in the machine.

A unique identifier of the machine 100 may be stored in local memory 263 or in separate hardware, e.g. of the controller 262 so that it is difficult to remove, and transmitted along with the rest of the data by the controller 262. From this information and the corresponding record for the machine 100 in the database 90 the remote computer 91 can recognise where and by whom the machine 100 is being operated, and can update the database 90 to link this information to the package data while logging any authentication issue for investigation. Since the package indicia are read (for the first time, or again for a second time) during the assembly process which is a one-time event, there is a high degree of confidence in the received data. Alternatively or additionally, the scanner 261 could be configured to read the package indicia before commencing the assembly operation so as to identify counterfeits or contra-indicated drug combinations before the door 130 is closed.

Then, the movement is reversed (FIG. 49) to raise the moving frame assembly 120 to its maximally raised position. The latches 103 are also retracted so that the pedestal block 170 moves upwards together with the moving frame assembly to the maximally raised position of the pedestal block 170.

In this position the door 130 is released and opened (FIG. 50) so that the capsule cap package 304 which now forms the end user package 300 can be removed together with the now empty frames 310 of the drug packages 301 and 302 and the capsule body package 305, and the pressure pad 250, leaving the machine empty at the end of the assembly operation (FIG. 51).

Referring again to FIG. 50, after opening the door 130 it can be seen that the cutters 190 have cut a slit 190′ (best seen in FIG. 57) through the casings of the stacked packages, which in combination with the slits 377 and 421 separates the label portions 313 and the flap 370 from their respective packages, leaving them adhesively attached to the flap 330 to become part of the composite label assembly 312. The adhesive, rear surfaces of the label portions 313 separate from the release coating 5 on the facing surface of their respective packages 301, 302 while the adhesive, rear surface of the flap 370 separates from the anti-adhesion front face 172 of the pedestal block 170, so that the entire composite label assembly 312 presents a tacky, adhesive rear surface.

After the user removes the end user package 300 together with the remaining, empty packages from the machine 100 (FIG. 51), he locks the pressure plate 121 and front plate 220 together and closes the door 130. An appropriate instruction could be displayed on the screen 267. The controller 262 senses that the door has been closed and operates the pistons 141 to urge the moving frame assembly 120 to move downwards until the abutments 235 contact the lower end surfaces of slots 179, and then continues the movement so that the pedestal block 170 is urged downwards by the abutments 235, together with the moving frame assembly 120, from the maximally raised position of the pedestal block 170 (FIGS. 50 and 51) to its initial, rest position (FIG. 41) in which it is retained by the latches 103 which re-engage in the slots 180. The machine 100 is now ready to receive another stack of packages.

Referring now to FIGS. 52-54, the stacked packages are shown in section during the assembly operation just described.

FIG. 52 shows the pushrods 110 resting against the carriers 320 of the capsule body package 305, immediately before the pressure plate 121 starts to move.

As the pressure plate descends, the frames 310 of the packages depress the pedestal block 170, revealing the pushrods 110 which rupture the foil end walls 314 of the cells 311 containing the carriers 320 and urge the carriers through the aligned cells 311 of the packages. As they move through the packages, the salient portions 323 at the open ends of the carriers 320 rupture the foil end walls 314 of the cells 311 containing the first and second drugs 1, 2, which are collected inside the capsule body 22, and then pass into the cells 311 of the capsule cap package 304 which contain the caps 21 of the capsules. (FIG. 53.)

Further movement urges the carriers 320 containing the capsule bodies 22, now filled with the granules or spheroids 3 comprising the drugs 1, 2, up into the cells 311 containing the caps 21 which are held axially centrally by the annular fins 325. The fins 325 are crushed by the carrier 320 as each cap 21 passes into the enlarged inner diameter region of the upper part of the carrier 320, between the wall of the carrier and the outer surface of the open end of the capsule body 21. The force applied to the cap 21 is reacted against the strip 315 that closes the cell 311, which in turn is supported by the pressure pad 250, so that the capsule cap and body are closed together to form the filled capsule or polypill 20, as best seen in FIG. 54A. The filled capsule 20 is contained inside the carrier 320, so that the closed end 321 of the carrier closes the lower end of the cell 311; the open end 322 of the carrier faces the strip 315.

As shown, the packages may be stacked and the machine configured so that the capsule bodies approach vertically upwardly to the capsule caps; other orientations are possible.

Referring now to FIG. 57, after removing the stack from the machine the user discards the empty frames of the packages 301, 302, 305. The cells 311 of the cap package 304 containing the polypills 20 are closed by the carriers 320 whose closed ends 321 form part of the rear surface of the package. The rear surface of the composite label assembly 312 is entirely covered by the tacky adhesive coating 4, comprising the rear surfaces of label portions 313 and flap 370 which are connected together in juxtaposed relation by the flap 330 so that the indicia on their front surfaces are visible through the window 331 to form the front surface of the flap. The user now folds the flap 330 against the rear surface of the package 304 so that it adheres to the rear surface, covering the closed ends 321 of the carriers.

Referring to FIG. 58, after sticking the flap in its final position, the legend: “TEAR HERE AND DISCARD THIS PART AFTER REMOVING FROM MACHINE” reminds the user to tear off the waste portion 334 of the flap 330 together with the surplus portion 372 of the flap 370, leaving the composite label assembly 312 that now forms the rear surface of the end user package 330. It will be noted that this is only necessary in the smallest package size; larger sizes do not have any waste portion.

Starting from the narrow side of the package and then turning it to continue on the adjacent, broad side, the composite label assembly reads: “12 CAPSULES SIZE #2 EACH CAPSULE CONTAINS:—OLANZAPINE 20 mg+CITALOPRAM 20 mg+NO OTHER ACTIVE INGREDIENTS”. Since the composite label assembly comprises the label portions 313 of each of the packages of the stack, the user has a high degree of confidence that the package is correctly labelled.

The user (e.g. the pharmacist operating the machine) can then retrieve the accompanying information leaflet 266 and any adhesive label (not shown) from the printer 265. The leaflet 266 may include data on any specific interaction between the combination of drugs selected. If an adhesive label has been printed then it is applied to the end user package 300, conveniently to one narrow side opposite the printed indicia.

The end user package 300 and the accompanying patient information leaflet 266 are then handed to the end user, who can lift the tabs 316 to tear back the strips 315 to open each cell 311, and then remove the capsule 20 by turning the package over and tapping it against the palm of the hand. The carrier 320 remains in the cell 311 and will be discarded along with the frame 310 once all the capsules 20 are finished.

### Patterned Foil

Referring to FIGS. 55 and 56, the foil end wall 314 of each cell 311 may have a weakened region 319 (FIG. 55) that causes it to tear in a predefined pattern as the carrier 320 presses against it. The weakened region 319 may be a pattern of relatively thinner parts that cause the foil to break in a star shaped pattern as shown in FIG. 56, so that the fragments adhere to the frame 310 and are captured between the carrier 320 and the cell wall 318 as the carrier 320 passes through the cell. The weakened region 319 could be formed for example by pressing the foil with a patterned tool.

Alternatively, referring to FIG. 113, the foil 1314 may be patterned to define regions 1430, each region 1430 of the foil closing a respective one of the cells 311 and including a first layer 1431 and a second layer 1432. Within each region 1430, the first layer 1431 is undivided, i.e. continuous, while the second layer 1432 is divided into sub-regions 1433 which extend radially inwardly away from the cell wall 318 and towards the central axis Xc of the cell.

In FIGS. 113 and 124, the second layer 1432 is indicated by the small horizontal dash fill, while the first layer 1431 is visible through the lacunae 1435.

The second layer 1432 is selected to have sufficient toughness and tensile strength to resist rupture when force is applied to the foil 1314, e.g. by the carrier 320, 1320, so that break lines are defined between the sub-regions 1433. The foil 1314 ruptures at the break lines in-between the sub-regions 1433, which remain as fragments attached to the block or frame. The carrier 320, 1320 folds the fragments back against the cell wall 318, as shown in FIG. 142, and slides past them as it passes axially through the cell 311.

The first layer 1431 may be a metal, and the second layer 1432 may be a polymer.

The polymer could be dissolved in a solvent and applied to the first layer as a solution. Alternatively, the second layer could be produced as a cut or stamped sheet, e.g. of a solid polymer or nonwoven fabric, and laminated to the first layer.

The polymer could be, for example: ethylene-vinyl acetate, polyvinylpyrrolidone, cellulose acetate, collodion, polyester, polyethylene, or a thermoseal lacquer. It could be filled with fibres or particles.

The first layer 1431 may be a thin aluminium foil of a similar thickness to that used for wrapping chocolate confectionary, e.g. around 6-20 microns, e.g. about 8-16 microns, so that it breaks easily.

The metal first layer 1431 may be entirely coated on one or both sides with a thin coating material, e.g. a lacquer or thermoseal lacquer, to prevent oxidation and ensure a good bond to the frame, and to seal pinholes to improve its barrier properties.

The second layer may be thicker than the thin coating material, and may be the same material as the thin coating material, or a different material. The second layer may face away from the cell or towards the cell, and may include a bond region 1434 which surrounds the cell 311. The second layer may be bonded directly to the first layer, or to the thin coating material which may be interposed between the first and second layers.

The thin coating material or second layer may be bonded sealingly to the block or frame (or to a compatible coating thereon) in the bond region surrounding the open end of the cell 311, e.g. by welding or adhesive, to hermetically seal the cell. The bond may be formed by applying heat, pressure, and/or high frequency electromagnetic or ultrasonic energy, e.g. via a sonotrode or other tool.

The thin coating material may be a polymer such as a vinyl or polyester lacquer or thermoseal lacquer as known in the art. The first and second layers and/or any coating of the block may be selected to be removable from the re-usable block or frame by thermolysis or hydrolysis or chemical, e.g. acid washing.

The second layer may be applied to the first layer, e.g. by printing, before the foil is sealingly bonded to the block or frame.

The patterned foil may be applied by a tool that locates the regions 1430 of the pattern, either relative to the tool or axially centrally over the cells 311 of the block. This could be accomplished by a controller using a camera and pattern recognition software to detect the pattern or registration marks on the foil. A dye visible to the camera could be incorporated into the coating material for this purpose. The tool could have a plate that is the same shape as the block, with cutting edges to stamp out the patterned foil from a printed roll, and holes corresponding to the cells which apply a partial vacuum to grip the stamped portion of foil. The plate may then position and press the foil on the block and apply the welding energy in the bonding regions, or over the whole surface of the block surrounding the cells 311.

Alternatively the second layer could be applied after the foil is bonded to the block, e.g. by spraying through a mask.

As illustrated by the variant first embodiment, the patterned foil 1314 may be applied, for example, to both sides of the drug packages (as shown in FIG. 113), to the top side of the capsule body package through which the carriers will leave the cells, and/or to the bottom side of the cap package (as shown in FIG. 124) through which the carriers will enter the cells.

The patterned foil may be applied to one side of the drug package before filling the cells, optionally in a protective atmosphere (e.g. dry air or nitrogen), and then applying the foil to the other side.

As shown in FIG. 113 and FIG. 124, the second layer 1432 may be divided into sub-regions 1433 by lacunae 1435 in the form of elongate gaps, as shown, or alternatively in the form of lines of smaller perforations (not shown). In the illustrated example, the elongate lacunae 1435 are united at the cell axis Xc and extend continuously and radially outwardly from the cell axis Xc to the cell wall 318 to define the break lines between the sub-regions 1433.

The pattern is selected so that the first layer 1431 will rupture at the break lines when the desired amount of pressure is applied, e.g. by the carriers 1320. For example, the foil 1314 may be made stronger by increasing the separation distance between adjacent perforations, or by dividing longer lacunae 1435 into shorter portions separated by narrow bridges (not shown) which connect the sub-regions 1433 together.

### Spacer Block

As the carrier 320 enters the cap package, fragments of foil adhering to the uppermost drug package may be drawn, together with fragments of foil from the lower surface of the cap package, into the interstice between the carrier and the cell wall of the cap package.

In order to make it easier for the trapped fragments, still connected to the uppermost drug package, to slide out of the interstice when the user separates the finished end user package from the uppermost drug package, the radially outer surface of the carrier 320 in its lower end region may be smooth, so that any barbed or textured surface region (if present, and as illustrated in the variant first embodiment) is confined to the upper end region. The lower end region of the carrier may also have a slightly smaller diameter than its upper end region. Alternatively or additionally, each cell of the cap package may continuously taper, or reduce in diameter step-wise, from the lower end region to the upper end region of the cell, so that the upper end region of the carrier fits more tightly into the cell than its lower end region. This helps to retain the carrier in the cell while allowing the fragments of foil to slip out of the interstice when the packages are separated.

Alternatively or additionally, a spacer block (not shown), comprising a block with an array of empty cells, similar to the blocks of the drug packages of the variant first embodiment but without foil, may be inserted between the cap package and the uppermost drug package. The spacer block is of a thickness sufficient to accommodate in its empty cells the fragments of foil, so that when the finished end user package is withdrawn from the machine there are no fragments of foil connecting the end user package with the rest of the stack. The spacer block may be disposable or re-usable, like the blocks of the drug packages and the capsule body package, and one spacer block may be supplied together with each cap package to avoid cross-contamination. Of course, the same spacer block could be used for multiple operations if desired.

### Spheroids

Granules may be formed as spheroids 3 in various ways as known in the art, for example, by spheronization or by pressing to form micro-tablets or pellets, e.g. rounded tablets with belly bands.

The granules, spheroids or other particles containing a drug may include excipients as known in the art, including for example microcrystalline cellulose as a spheronization enhancer. The particles or spheroids may be coated as known in the art to control (e.g. delay or prolong) the release of the drug into the body. Different drug packages could contain similar or different drugs with different coatings, so that the system can be used to prepare combinations that separately control the release of the similar or different drugs following their simultaneous consumption in the same capsule.

The capsule components could also have an enteric or other coating which is indicated on the package indicia.

Spheronization may be performed by forming an appropriate composition including the respective drug and extruding the composition to form rods which are cut to length and then introduced into a spheronizer (typically a machine having a rotating bowl with a textured surface) as well known in the art.

The diameter of the spheroids may be controlled by selecting an appropriate diameter for the extruded rods and by cutting the extruded rods to an appropriate length. The ratio of length to diameter may determine whether each cut length forms a single spheroid or breaks into multiple spheroids. For example, if the length is approximately the same as the diameter then the rod may form a single spheroid, whereas if the length is twice that required to form a rod of given diameter into a single spheroid, then the rod may form into a characteristic waisted, “dumb-bell” shape as known in the art before separating into two portions which form two respective spheroids of equal diameter.

For drugs that are provided in liquid form, the liquid may be encapsulated in a spheroidal shell. One way to do this is to form the liquid into a solid or partially solidified composition at low temperature, and then spheronize the composition at low temperature, and then coat the resulting spheroids with a coating, still at low temperature. Once the coating has solidified, the temperature may be returned to ambient so that the coating forms a shell to contain the liquefied contents. The coating material can be selected for example from suitable film forming materials as well known in the art, some of which are discussed further herein. It could be applied hot to the spheroids, which could be further sub-cooled after spheronization to a temperature selected so that the coating solidifies on the frozen composition before the composition liquefies. Alternatively the coating could be applied as a powder, e.g. in a fluidized bed, before very briefly heating the outer surface of the coated spheroids (e.g. radiatively, or with a hot gas, or by conduction) to melt and fuse the powder into a solid coating which solidifies before the frozen composition liquefies. Heating by conduction could be accomplished by maintaining the coated spheroids in motion on a hot surface, e.g. the surface of the bowl of the spheronizer, or a rapidly vibrating plate.

### Variant First Embodiment

Referring to FIGS. 102-145, the variant first embodiment corresponds generally to the first embodiment already described, but illustrates various optional and alternative features which may also be applied to the first embodiment, including: a profiled recess 1150 for aligning the stacked packages; a printed composite label 1312; a resilient pushrod mounting and transducer assembly; re-usable blocks 1360, 1360′, 1400, 1400′; an alternative drug package with a re-usable block and movable closure elements, and a filling apparatus for use therewith; a cap package featuring flocked cells 1311; and patterned foil 1314.

Parts of the variant first machine 1000 are illustrated in FIGS. 102-108; the remaining parts are generally the same as those of the first machine 100, including its internal and external system elements as illustrated in FIG. 1, except as described below.

Instead of the profile rods and alignment rods of the first machine, the variant first machine 1000 illustrates how the shape profile or alignment structure 150 of the first machine may instead be provided as a profiled recess 1150, which as shown may extend to form the lower cavity 1231 of the moving frame assembly 1120 in which a pedestal block 1170 is slidingly received to slide over the pushrods 1110. The moving frame assembly 1120 includes a pivotable pressure plate 1121 and front plate 1220. The above mentioned parts of the variant first machine are similar in structure and function to the corresponding parts of the first machine, except as further explained below. However, since the variant first machine 1000 does not produce a mechanical composite label assembly, it does not include the cutters, cyclonic separators, and resiliently compressible surface 228 of the first machine 100.

Since there are no rods projecting above the upper ends of the pushrods 1110, the pressure plate 1121 need not be apertured, and so can bear directly on the whole surface area of the stacked packages; thus, the pressure pad 250 need not be used.

Re-Usable Block

The variant first embodiment illustrates how each single use package may incorporate a re-usable block, which may be a monolithic, foraminous block, and which may be closed by foil or alternatively by movable closure elements such a sliding, foraminous plates, which may also be re-usable.

Referring to FIG. 109 and FIGS. 113-117, each of the first and second drug packages 1301, 1302 and the capsule body package 1305 may be formed from a block 1400 which defines the frame of the package. After the empty package is removed from the machine 1000, the block can be returned to the factory where it was filled, where it is cleaned to remove the foil 1314 and the package label 1317 and any traces of the drugs 1, 2, and then re-used to form a fresh package with new foils and label. The cleaning process could include mechanical brushing and washing, e.g. in a solvent which is selected to dissolve the adhesive or other coating materials and possibly also the metal or polymer components of the foil 1314.

The re-usable, foraminous block 1400 may be monolithic with a simple shape selected to minimise its surface area, so that it is easy to clean. It may be made from a smooth, impermeable, durable plastics material or alternatively from a ceramic or glass (i.e. silica glass as opposed to carbohydrate glass). For example, it could be made as a monolithic block of thermoplastic or engineering thermoplastic so that it can be recycled as a raw material at the end of its life—for example, acrylonitrile butadiene styrene, polyoxymethylene, or rigid polyvinyl chloride. It could be moulded or cut from an extrusion. The cells 311 could be moulded or machined. The outer surfaces of the block 1400 may be etched or otherwise textured to improve adhesion. Before filling the cells and applying the foil, the block could be coated (partially or entirely) with a material that can facilitate bonding and can be removed in the cleaning process. Example coating materials may include those mentioned under Patterned Foil. above, for the second layer 1432 or the thin coating material of the metal first layer 1431.

The profiled recess 1150 of the machine forms an alignment structure which maintains the packages with their respective cells in axial alignment when stacked in the machine. The alignment structure may comprise any surfaces of the profiled recess, optionally but not necessarily including profile features such as asymmetric profile protuberances 1159, which may be provided to prevent incorrect orientation of the package. Alternatively, this could be detected by the machine when scanning the package indicia.

Corresponding profile features, such as profile recesses 1353, may be formed at the periphery, e.g. the right and left sides of the block 1400, to engage profile features, e.g. profile protuberances 1159, of the profiled recess 1150 of the machine 1000. The profile features 1353, 1159 may be asymmetric and arranged (e.g. as shown) so that the stacked packages will only fit in the profiled recess 1150 in the correct position (FIG. 106, FIG. 108).

FIG. 110 shows a similar block 1400′ with twenty-four cells, for use in the same machine 1000. The asymmetric profile features 1353, 1159 ensure that this smaller block also will fit into the profiled recess 1150 only in a single, correct position.

The block 1400 of the drug package 1301, 1302 may be sealed on one side with a sheet of patterned foil 1314 as described above under the heading Patterned foil, and then filled with the drug 1 or 2, e.g. granules or spheroids 3, before closing the other side with a second sheet of patterned foil 1314 as shown in FIG. 113. The block 1400 of the capsule body package 1305 may be sealed on its upper (exit) side with a patterned foil 1314, but on its lower side with a sheet of plain foil 314 which adheres to the block 1400 and also to the closed ends of the carriers 1320 containing the capsule body component 22. A disc of the plain foil 314 remains attached to each carrier 1320 to form part of the end user package 1300 as shown in FIG. 136.

An adhesive package label 1317 may be applied to the front side of the block 1400 to carry the package indicia 317, which may include both human readable and machine readable indicia, as shown in FIGS. 115 and 117. Alternatively, the package indicia 317 could be printed directly on the block or on a coating material of the block.

Thus, each drug package 1301, 1302 may consist substantially of the block, the foil, the package indicia or package label, and the drugs, while the capsule body package 1305 may consist substantially of the block, the foil, the package indicia or package label, the capsule body and the carrier.

The drug packages 1301, 1302 and capsule body package 1305 may be supplied in stacks with sheets of card in-between them to protect the foil 1314 from damage. The foil 1314 of the cap package 1304 may be protected in storage by folding the flap 1330 over the block 1360 as shown in FIG. 125.

FIG. 114 A shows an alternative arrangement wherein a block 1400 containing drug particles 3 is sealed on each face with a foil, e.g. a patterned foil 1314, in the same way as the drug package 1301. Two additional, empty blocks 1400″, which may be identical in form to the block 1400 or may be smaller in the thickness dimension as shown, are connected to the block 1400 so that the cells 311 are aligned. Each foil 1314 is sandwiched between the block 1400 and the respective empty block 1400″, whose cells 311 are open at their outer ends. The blocks 1400, 1400″ may be bonded together, e.g. by adhesive, or connected by mechanical interlocking features. Two blocks 1400, 1400″ may be assembled together with one of the panels of foil 1314 before filling the drug particles 3 into the cells 311 of the block 1400. Then the filled cells 311 may be sealed by applying the second panel of foil 1314 which may already be connected to the second empty block 1400″.

Together, the blocks 1400, 1400″ form a composite package 1303 in which each portion of foil 1314 that seals the drug particles 3 in a respective cell 311 of the central block 1400 is recessed within the respective open cell 311 of the outer, empty block 1400″, which protects it against accidental damage. The outer block 1400″ also accommodates the fragments of foil formed during assembly so that they are not trapped in the cells of the end user package 1300. The composite package 1303 could be labelled after assembly, or the label could be applied to one of the component blocks 1400, 1400″ before assembly. A composite capsule body package could be formed in a similar way. Like the packages previously described, each composite package 1303 may be supplied in a sealed outer bag or wrapping, which prevents contamination of the open cells in storage. The blocks 1400, 1400″ may be disassembled at the factory for cleaning and re-use, e.g. by soaking to dissolve the adhesive.

Alternative Package with Movable Closure Elements

Referring now to FIGS. 146-159, an alternative package is illustrated as a drug package 2301, although alternatively it could contain a capsule component (optionally, with a carrier).

As with the packages of the first embodiment, the alternative package 2301 includes a block 2400 which forms a frame defining a plurality of cells. The cells 311 are separated by the frame to form a spaced array, wherein each cell includes a cell wall 318, the cell wall extending along a cell axis Xc between opposite, first and second ends of the cell at opposite, first and second sides of the frame or block 2400. Each cell 311 of the package 2301 defines an enclosure, and the respective single dose of the drug, (or if a capsule part package (not shown), then the respective capsule part, optionally in a carrier) is enclosed within the enclosure, e.g. in the form of spheroids 3, as shown.

Instead of a frangible foil, as shown in the previously described packages, the package 2301 includes at least one movable closure element 2401 which closes at least one respective end of each cell and is movable by the machine 1000, relative to the frame or block 2400, to open the at least one respective end of each cell 311 while the packages are stacked in the machine. As illustrated, the at least one movable closure element 2401 may close and open both respective ends of each cell 311. By displacing the at least one movable closure element, relative to the block, is meant that the machine causes relative movement between those parts; this can be accomplished, for example, by moving the at least one movable closure element while the block remains static, or by moving the block while the at least one movable closure element remains static.

For example, also as illustrated, the package 2301 may include two separate movable closure elements 2401 arranged on opposite sides of the block 2400 to close, respectively, the first and second ends of the cells 311. Alternatively, one movable closure element could be arranged to close both ends of each cell; for example, it could include two parallel plates joined together in fixed relation, wherein the block 2400 is received between the plates. For example, the movable closure element could be configured as a casing, with the block slidingly received inside the casing. The block could be made from metal or plastics material or glass; the movable closure element could be made from metal or plastics material. For example, a plastics block could be received inside a metal casing forming the movable closure element.

Alternatively, the cells 311 could be closed on one or each side of the package by a frangible foil and a movable closure element—which is to say, both the frangible foil and the movable closure element are arranged on the same side of the package, and that arrangement could be provided on one or both sides. For example, the movable closure element could protect the frangible foil against accidental damage. Alternatively, the cells could be closed at one end by a frangible foil, and at the other end by a movable closure element.

As with the other described embodiments, each drug or capsule part package 2301 may be a single use package, which is to say, the package is only used one time. However, as shown, the block 2400, which forms a component part of the package 2301, may be a re-usable, monolithic, foraminous block. After using the package 2301 to form the plurality of single, orally ingestible bodies or capsules 20, the block 2400 is cleaned (e.g. to remove any traces of the drugs, and perhaps also the package label 1317 and/or any sealant) and re-used (e.g. with new drugs or a new capsule component, optionally with a new carrier, and perhaps also a new package label 1317 and/or sealant) to form part of another single use drug or capsule part package 2301.

The variant first machine 1000 is arranged to displace each movable closure element 2401, optionally slidingly, relative to the block 2400, from a closed position (FIGS. 153, 154, 155, 158) to an open position (FIGS. 157, 159), to open the cells 311 while the packages are stacked in the machine 1000, as one step in the process of combining together the first and second drugs to form the polypills 20. This places each cell 311 in coaxial communication with the corresponding cells 311 of the other packages stacked in the machine 1000, as previously described.

As illustrated, the machine 1000 may displace the movable closure elements 2401 simultaneously by means of an actuator (not shown) which moves a displacement surface or surfaces 1195 that engage the movable closure elements 2401. The or each displacement surface 1195 may be formed, for example, as a bar that extends along the height of the stack of packages, and which as shown may form a movable assembly of the front plate 1220 of the machine 1000, or alternatively could form a moving part of the moving frame assembly or other machine part. The pedestal block 1170, if present, may have corresponding recesses 1187 to accommodate the bars. The or each displacement surface 1195 may move into a respective recess 2402 in the block 2400. The block 2400 may be supported at abutment surfaces 2403 by shoulders 1196 at the rear of the adapted profiled recess 1150, which may include a recess 1197 (FIG. 156) to accommodate the closure elements 2401 as they move to the open position, which is shown (but for clarity, without the profiled recess 1150) in FIG. 157.

As illustrated, the or each movable closure element 2401 may define an array of through-holes 2404 which are juxtaposed in conterminous, coaxial relation with the cells 311 of the block 2400 in the open position (FIGS. 157, 159), while in the closed position (FIGS. 153, 158), the through-holes 2404 are misaligned with the cells 311 of the block 2400 so that the closure element 2401 closes the ends of the cells 311 on the respective side of the package 2301. By “conterminous relation” is meant that the walls of the through-holes are aligned with the walls 318 of the cells 311, so that in the open position, the through-holes 2404 and cells 311 have a cross-section that is substantially continuous (i.e. substantially unvarying) along the common axis Xc of the cells 311 and through-holes 2404. This enables the capsule part 22 to capture the drug particles 3 as the capsule part 22 or carrier 320, 1320 slides through the package 2301.

As is the case also in the first embodiment and the other packages of the variant first embodiment, each cell of the package (and, in this embodiment, also each through-hole of the package) is arranged to form a respective portion (i.e. a portion of the axial length) of a respective, continuous passageway, which is formed (wholly or mostly) by the corresponding cells of all of the packages when stacked together in the machine. For example, a stack of packages with 48 cells will form 48 continuous passageways when stacked together in the machine. The passageway preferably has a substantially constant cross-section along the axis of the passageway, which is defined by the collinear axes Xc of its respective cells 311; which is to say, its cross-section does not vary substantially along its length. The cross-section is defined by the axially aligned walls of the cells (and, in this embodiment, also of the through-holes).

The or each closure element 2401 may be slidingly retained by the block 2400 for movement between the closed and open position, for example, in slideways 2405 formed in the block 2400. Each movable closure element 2401 may be separable from the block 2400. Each movable closure element 2401 may be formed as a foraminous plate, which may be monolithic and re-usable, so that after use, the block 2400 and plates 2401 can be separated, cleaned, and re-assembled to form component parts of a new single use package 2301. Thus, illustrated, a foraminous block 2400 may be arranged between a pair of slidable, foraminous plates 2401.

The closure element 2401 and block 2400 may include co-operating retaining features (not shown), such as small protrusions that engage co-operating abutment surfaces or recesses. Such features may retain the closure elements 2401 in either or both of the closed and open positions relative to the block 2400. The retaining features may be overcome (e.g. by resilient deformation) by application of a threshold force to move the closure elements 2401 from the closed to the open position, and then again by application of a further threshold force to separate the parts 2400, 2401 of the assembly for cleaning after the opened package 2301 is returned to the factory.

In alternative arrangements, the outer plates of the package, described herein as movable closure elements, could remain static, and could be arranged to engage with alignment or profile features of the machine, while the inner plate, described herein as the block, is displaced by the actuator of the machine.

After filling and closing the package 2301 (FIGS. 153, 154), the adhesive package label 1317, where present, may be applied (FIG. 155) over the joint or sliding interface between the closure element(s) 2401 and the block 2400 to retain the parts 2401, 2400 in the closed position, and then irreversibly ruptured when the closure elements 2401 are moved by the actuator of the machine 1000 to the open position. The label 1317 may be provided with a tamper evident pattern or indicium (illustrated in the multipack embodiment, below) that covers the region to be ruptured, perhaps in combination with a mechanical tamper evident feature (e.g. a pattern of cuts, as used in a tamper evident price labels on retail items). The machine 1000 may be configured to scan the tamper evident feature so as to recognise, by the broken pattern or indicium, a package 2301 that has been opened prematurely.

The label 1317 could be arranged to be ruptured in a direction out of its plane (as illustrated), or alternatively, as illustrated in FIG. 185, could be arranged on a different surface to be ruptured in shear in its plane by sliding movement between the respective parts 2401, 2400, with the package 2301 being arranged to expose the sliding interface on that surface. For example, the package could comprise a pair of sliding plates slidably arranged on opposite sides of the block, wherein the plates define surfaces that lie in a common plane with a respective surface of the block, and the plates are slidable relative to the block in a direction parallel with the common plane. For example, the parts 2400, 2401 could be adapted to extend the foraminous plates 2401 to the edges of the foraminous block 2400 to define a surface for the package label 1317, and the image capture device 1281 (further explained below) arranged to read the package labels on one side of the stack rather than the front of the stack as illustrated. Or, the image capture device 1281 could be arranged to read the package labels on the front surface of the stack, whether as part of a front plate 1220 or otherwise, and the stack arranged in that orientation with the or each displacement surface 1195 arranged to push the moving parts of the packages (e.g. block 2400 or plates 2401) from right to left or left to right to open the package cells.

Each cell 311 may contain a single dose of a drug to form a drug package 2301, as illustrated. Alternatively, although not illustrated, each cell 311 may contain a part of an empty capsule, which may be arranged in a carrier 320, 1320, but without a complementary part required to complete the capsule 20. The carrier 320, 1320 may be received in the cell 311 and slidable out of the cell 311 along the cell axis Xc, as in the other embodiments. The capsule part may be a capsule body 22, forming a capsule body package, similar to the capsule body package of the other embodiments.

As with the other described embodiments, although not illustrated, each package may be enclosed in a protective outer wrapper.

Each dose may be separately enclosed in its respective cell 311, while the outer wrapper (e.g. a sealed film or foil bag) provides a seal (e.g. a hermetic seal) to preserve the drug in storage. For example, the outer wrapper could be sealed to retain the package in a vacuum or partial vacuum. The wrapper may be disposable and may be removed before placing the package 1301 in the machine 1000.

Alternatively, rather than a disposable wrapper, the whole package 1301 could be arranged in a re-usable outer casing, e.g. a box with a lid, that is sealingly closed for storage, and openable to remove the package for use.

Alternatively, the package components may be sealingly engaged together to provide a seal that preserves the drug; for example, a mechanical seal could be arranged between the components 2400, 2401 of the package, or the whole assembly (FIGS. 153, 154) could be coated with a sealant. As with the other embodiments, the drug or capsule component may be assembled into the package in a protective atmosphere.

Where the package contains the capsule cap, the capsule cap 21 may be supported in spaced relation to the cell wall 318 by flock 1699 or other locating structure as previously described. The machine 1000 may displace the foraminous plate or other movable closure element relative to the block or frame, e.g. slidingly, in a similar way to the closure elements of the drug and capsule body packages. The closure element may form a part of the end user package or may be discarded or returned to the factory for re-use.

Filling Apparatus for Use with the Alternative Drug Package

Referring now to FIGS. 160-163, an example filling apparatus 2430 is shown, which may be used to fill the spheroids 3 into the cells 311 of the alternative drug package 2301. The apparatus has a main body 2431 with an array of discharge nozzles 2432, only one of which is shown in the drawings. Each discharge nozzle 2432 has a discharge chute 2433, a filling chute 2434 which is filled with a column of equally sized spheroids 3, e.g. by gravity from a vibrating hopper (not shown), and a dosing body 2435 that moves (e.g. rotates, as shown) relative to the main body 2431 between a receiving position (FIGS. 160, 162) and a discharge position (FIG. 163). The dosing body has a dosing chute 2436 and optionally also a sensor or an array of sensors, e.g. an array of through-beam photoelectric sensors 2437, that detect the presence of spheroids 3 in the dosing chute 2436.

In use, the block 2400 of the partially assembled drug package 2301 may be arranged as shown in FIG. 151, with the lower movable closure element 2401 in the closed position and the upper movable closure element 2401 in the open position. The main body 2431 is engaged with the upper movable closure element 2401 so that each discharge nozzle 2432 completely fills one of the through-holes 2404, with the lower end 2438 of the discharge nozzle lying in the plane of the sliding interface between the movable closure element 2401 and the block 2400. The dosing body 2435 is moved to the receiving position (FIGS. 160, 162) in which the column of spheroids 3 can fall from the filling chute 2434 into the dosing chute 2436. The sensors 2437 may indicate whether or not the dosing chute 2436 is completely filled. Sensor 2437 output may be evaluated after moving the dosing body to an intermediate position (not shown) in which the dosing chute 2436 is isolated from both the filling chute 2434 and the discharge chute 2433. If sensor 2437 output indicates that the dosing chute 2436 is completely filled, the dosing body 2435 is moved to the discharge position (FIG. 163) in which the dosing chute 2436 no longer communicates with the filling chute 2434 but instead is axially aligned in communication with the discharge chute 2433 and with a plunger 2439, which is extended from the main body 2431 through the dosing chute 2436 and discharge chute 2433 until its lower end face is flush with the lower end 2438 of the discharge nozzle. This ensures that all of the spheroids 3 are ejected from the dosing chute 2436 into the cell 311 of the block 2400. The plunger 2439 remains in this extended position while the main body 2431 then translates from the open position to the closed position of the upper movable closure element 2401, closing the cells 311 of the package, which ensures that none of the spheroids 3 can escape from the cell 311 as the package is closed. The main body 2431 is then withdrawn and the plunger is retracted to the start position. The filled package (FIGS. 153, 154) may then be labelled (FIG. 155).

The length of the dosing chute 2436 is selected (or may be adjustable, not shown) to accommodate the desired number of spheroids to be filled into each cell 311, for example, seven spheroids 3 as illustrated. Alternatively, the operation could be repeated before closing the package, with only one or more than one spheroid 3 being ejected per operation.

Package Indicia and Sequence of Operation

The package indicia 317 may include first indicia 1317′ identifying the contents, which will be transferred from each of the drug packages 1301, 1302 and optionally also the capsule body package 1305 to the end user package 1300, and second indicia 1317″ providing data to facilitate operation of the machine 1000, which need not be transferred to the end user package 1300. The first indicia 1317′ may include the name and dose of the drug in words and numerals, and may further include a machine readable code that embodies the same information and/or serialization data. The serialization data could alternatively form part of the second indicia 1317″. The second indicia 1317″ may indicate, for example, the number of spheroids 3 or volume of the drug 1, 2 per cell, the vertical thickness of the package, the size of the block or total number of cells 311, and/or the number and/or location of the cells 311 that are filled (for example, all the cells or only some of the cells, providing different end user package sizes based on the same blocks 1360, 1360′, as shown in FIGS. 138, 139 and 140). The local controller 262 (FIG. 1) can use this information to set the position of the moving frame assembly 1120 or pressure plate 1121, pedestal block 1170, or other moving parts of the machine 1000 to match the expected stack height, and to ensure that all the packages are compatible and the capsule size is correct for the combined volume of the drugs.

To facilitate this process, and also to detect counterfeit drugs or incorrect package combinations or to check that the drugs match the prescription, the packages may be scanned at an external scanner 1280 before loading them into the machine 1000, where they may be scanned again by an internal scanner or scanners as further explained below.

If preferred, the machine 1000 could operate with only external scanner 1280, or only the internal scanner(s). Since the drug volume and other information may be verified by scanning the package indicia (either for the first or second time) when present inside the machine, the vertical (thickness) dimension of the drug packages need not vary with the volume of the drugs contained therein as previously described for the first embodiment.

Referring to FIG. 1 and FIG. 102, an example sequence for manual operation of machine 1000 might be as follows:

i) Controller 262 receives prescription data, e.g. via external data link 264, via user interface 267, or by scanning a barcode via external scanner 1280. Controller 262 may retrieve further information to identify or confirm the prescription, or to check for contra-indicated combinations, or to add patient-specific information, e.g. maximum capsule size.
ii) Controller 262 may indicate the correct capsule size and package size to match the combined drug volume and the required number of doses. The user may confirm the selection, or select a smaller capsule size (and larger package size) if it's preferred to divide the dose. The prescription information may be displayed on screen 267 as illustrated.
iii) The user selects the packages 1301, 1302, 1304, 1305 and scans them at external scanner 1280. Alternatively, the process could begin with this step.
iv) Controller 262 validates the scanned packages, e.g. via database 90. If the packages are OK, controller 262 may set the machine for the expected stack height, and then opens the door.
v) The user places the packages in the machine and closes the door.
vi) The machine 1000 scans the packages a second time to verify their identity, to create the composite label 1312, and to update the database 90 with the serialization data, and performs the assembly step to form the end user package 1300.
vii) The user removes the end user package 1300 and pairs it with the patient information leaflet 266. The empty packages 1301, 1302, 1305 are placed in a bin to be returned to the factory.

Printed Composite Label

As in the first embodiment, each drug package 1301, 1302 of the variant first embodiment may include human readable and/or machine readable label indicia (indicated collectively in this embodiment as 317) identifying the respective drug, and the machine 1000 is arranged to combine together the label indicia 317 from respective ones of the packages (particularly the drug packages) as received in the machine to form a composite label 1312 of the end user package 1300 which identifies each of the drugs 1, 2. However, instead of assembling together the mechanical parts of the drug packages that carry the indicia as described with reference to the first machine 100, the variant first machine 1000 is arranged to accomplish this by reproducing on the end user package 1300 a copy 1312′ of the indicia 317 that appear on the drug packages.

For this purpose the machine 1000 may include an image capture device 1281 for capturing an image of the label indicia 1317′ from the stacked packages, and an image reproduction device 1282 for reproducing (e.g. printing) the captured image 1312′, as a composite label 1312, on the end user package 1300. The image 1312′ may be reproduced at the same size as the original or enlarged or reduced as shown. The machine 1000 may be arranged to ensure that the packages are correctly stacked (e.g. with the pressure plate and front plate closed and locked together) before the image capture device 1281 captures the image. Since the image reproduction device 1282 simply reproduces exactly what the image capture device 1281 sees (i.e. the image that it captures) in the target region of the stacked packages, anything visible on the target portion of the package labels will appear on the end user package, so the end user package 1300 cannot be mis-labelled.

Together, the image capture device and image reproduction device may be considered as a photographic reproduction or photocopying apparatus, and the reproduced image 1312′ as a photocopy or photographic print of the package indicia.

Alternatively or additionally, the end user package could be labelled (or further labelled) with indicia generated from data received by the controller 262, e.g. by looking up the scanned package indicia in the database 90 to download the associated information. For example, the image reproduction device 1282 may also print, as part of the composite label 1312, customer information 1080 associated with the prescription (e.g. name, address, human or machine readable customer ID code, dosage information, etc.), which may be derived from the inputted prescription information or from a database. The same information may be reproduced on the patient information leaflet or associated adhesive label.

The image 1312′ may be reproduced on a front surface of an adhesive flap 1330 of the capsule component package that will form the end user package 1300, which in the illustrated example is the cap package 1304, as in the first embodiment. The flap 1330 has a mostly blank front surface facing away from an opposite rear surface, which may be coated with adhesive 4 and protected by a release paper 1332. The flap 1330 is hinged to the frame and movable to position its adhesive surface to cover the bottom side of the package 1304 through which the remaining capsule and drug components, and also the carriers if present, are admitted during assembly.

Where the machine includes a movable front plate and pressure plate, as illustrated, the drug and capsule body packages may be stacked in the machine 1000 before raising the front plate 1220 (FIG. 103). The front plate 1220 may include a support surface 1240 on which the unfolded flap 1330 (with its release paper 1332 in place) can rest when the cap package 1304 is then added to the stack (FIG. 104). The top (pressure) plate 1121 is then closed to position the image reproduction device 1282 against the front surface of the flap 1330 which is sandwiched between the window or print head or other active face of the image reproduction device 1282 and the support surface 1240 (FIG. 105). The package indicia 317 are then scanned, and if validated, the controller 262 then actuates the moving frame assembly 1120 to produce the end user package 1300 as previously described.

The image capture device 1281 may be arranged in the front plate 1220, which may include one or more reflectors or prisms 1283 to direct the scanning beam. A second internal scanner 1284 may be arranged to read the indicia 317 from the cap package 1304 via a reflector 1285.

The image capture device 1281 may be a scanner or camera, and the image reproduction device 1282 may be a printer or writer of any suitable type. The scanner may scan line by line and send the rasterized image to the printer which prints each line as it is received. The printer could be for example an ink based printer, e.g. an inkjet printer, or a thermal printer with a moving print head, or a laser printer that works on a thermally sensitive substrate or on a plain paper or card substrate, e.g. as taught by WO2014158019 to Tocano V.O.F. The front surface of the flap 1330 could be configured accordingly, e.g. to include a black layer behind a white layer (the layers being e.g. paper or polymer) to provide a durable image when the front layer is locally heated by the laser, or incorporating a pigment that is actuated by heat as known in the art.

The cap and body packages 1304, 1305 may include indicia 317 similar to that of the drug packages to indicate, e.g. the material or special properties (e.g. enteric coating) of the capsule components together with serialization data if required, as well as the capsule size, package size, number and position of the filled cells, and other information required by the machine 1000. These indicia may remain visible on the capsule component package 1304 that forms the end user package 1300, and may be captured and transferred from the other capsule component package 1305 along with the indicia from the drug packages to form the composite label 1312. As in the first embodiment, the captured indicia from the capsule component package 1305 may include a legend: “NO FURTHER ACTIVE INGREDIENTS” which will appear at the end of the list of drugs in the composite label 1312.

Cap Package: Flocked Cells

Where the cells of the block of the first capsule package include a locating structure for locating the capsule body or cap centrally within the cell in spaced relation to the cell wall, the locating structure may be a lining of flock within the cell, as will now be described with reference to the cap package 1304.

Referring to FIGS. 111 and 112, the cap package 1304 may be formed from a monolithic block 1360 which forms the frame defining a spaced array of cells. The block 1360 has the same overall shape as the block 1400 of the drug and capsule body packages, optionally including profile recesses 1353 or other asymmetric peripheral shape profile features that serve to prevent incorrect insertion into the profiled recess 1150 of the machine. A half size variant 1360′ corresponds to the block 1400′.

The blocks 1360, 1360′ may be moulded from a similar material to the block 360 of the first embodiment, for example, a biodegradable material, e.g. a carbohydrate glass, perhaps filled with lignin or wood flour or the like. The block may be coated, e.g. with a coating material similar to that of the block 1400.

As shown in FIGS. 120-121, the cell wall 318 of each cell 311 of the block 1360 may be lined with flock 1699 to form a flocked cell 1311. The flock 1699 comprises short fibres, e.g. of rayon or nylon, which may be for example around 1 mm-3 mm in length. The outer end of each fibre is bonded by adhesive to the cell wall 318, so that the fibre extends radially inwardly towards the cell axis Xc. The adhesive may be applied over only part of the cell wall 318, leaving a short, unflocked region 1311′ at the upper end of the cell through which the finished capsule 20 will be removed by the end user (FIGS. 143, 144).

The capsule component (in the illustrated embodiment, the cap 21) is inserted into the flocked cell 1311 of the package 1304 so that the fibres of the flock hold it centrally in the cell in spaced relation to the cell wall 318 (FIGS. 123, 128).

During assembly, as the carrier 1320 enters the flocked cell 1311 (FIG. 141), the carrier 1320 moves into the space occupied by the flock 1699 which is folded back against the cell wall 318 as the cap 21 enters the annular space between the capsule body 22 and the carrier.

As shown in FIG. 145, the carrier may be similar to the carrier 320 of the first embodiment but with a radially outwardly facing, textured, e.g. barbed or serrated surface portion 1328. The texture engages the fibres of the flock 1699 as the carrier enters the cell (FIG. 141) so that they retain the carrier 1320 in the cell as the end user package 1300 is removed from the machine.

A smooth surface portion 1329 may be left at the lower end of the carrier 1320 to help release fragments of foil 1314, forming part of the uppermost drug package, from in-between the carrier 1320 and the cell wall 318 when the end user package 1300 is separated from the stack. Alternatively, the carrier may be serrated or textured for its whole length, which may be preferred where the foil of the uppermost drug package is spaced apart from the cap package, or where the drug packages are closed instead by movable closure elements, e.g. sliding foraminous plates as described above.

The carrier 1320 could be made from a similar material to the carrier 320, e.g. a carbohydrate glass with a filler material for strength, or hard gelatin, HPMC, biodegradable cellulose acetate, or other plastics material. It could be partially coated with a wax. A filler material may include fine grained particles, whiskers, or a mixture thereof.

The flocked lining of the cell may accommodate and retain carriers of different diameters for different sized capsules.

Cap Package: Casing

FIGS. 130 and 131 show the opposite sides of the outer casing sheet 1342 of the cap package 1304, in the form of a flat sheet of card, before it is folded around the block 1360 to form part of the casing 1340. The sheet is printed on its outer surface with indicia 317 and, on the portion that will form the flap 1330, with the legend “EACH CAPSULE CONTAINS:”, which will appear above the printed copy of the indicia captured from the other packages in the stack. Strips 1315 terminating at tabs 1316, identified by printed triangles, are defined by cut lines that separate the strips from the sheet, but leaving them in position for assembly. Printed numerals on each strip identify the position of each of three cells 1311 that will be covered by the strip.

FIG. 132 shows the inner casing sheet 1343, also a flat sheet of card with discs 1344 defined by cut lines but left in place in the sheet. The inner casing sheet 1343 is coated on one side with adhesive, which is then covered by a sheet of plain foil 314 (FIG. 133). The opposite side of the inner casing sheet 1343, not shown, is not coated with adhesive.

Referring to FIG. 134, the inner surface of the outer casing sheet 1342 is coated selectively with adhesive. The adhesive covers entirely the inner surface of the part forming the flap 1330, which is subsequently covered by a release paper 1332 (FIG. 135).

On the inner surface of each strip 1315, the adhesive 4 is applied in a pattern which defines three circular patches 1345, each of which will be conterminous with a respective one of the discs 1344 when the inner casing sheet 1343 is superposed on the outer casing sheet 1342 as shown in FIG. 135. Additionally, a small dot 1346 of adhesive 4 extends from each circular patch 1345 towards the tab 1316 of the respective strip 1315. The rest of the strip 1315 is free from adhesive. The remaining parts of the inner surface of that portion of the outer casing sheet 1342 which defines the strips 1315—which is to say, the parts in-between the strips 1315—are also covered with adhesive 4, as shown.

FIG. 135 shows how the uncoated side of the inner casing sheet 1343 with its attached sheet of plain foil 314, as shown in FIG. 133, is then superposed on the patterned adhesive over the inner surface of the outer casing sheet 1342 defining the strips 1315, so that each disc 1344 is superposed on a respective patch 1345 of adhesive. In this position, the body of the inner casing sheet 1343 (i.e. the solid part in-between the discs 1344) adheres strongly to the body of the outer casing sheet 1342, i.e. the solid part in-between the strips 1315, so that both sheets 1343, 1342 are united into a single assembly.

Each disc 1344 is connected to the body of the inner casing sheet 1343 only by the sheet of plain foil 314. Each strip 1315 overlies three discs 1344, and is connected strongly to each of those discs 1344 by the respective patch of adhesive 1345. However, the strip 1315 is connected to the body of the inner casing sheet 1343, i.e. the solid part in-between the discs 1344, only by the three small dots of adhesive 1346.

Referring now to FIG. 122, the block 1360 with flocked cells 1311 is now coated with adhesive on one flat side and on its narrow sides, and then the adhesive side is placed onto the plain foil sheet 314, and the flaps of the outer casing sheet 1342 are folded around the narrow sides of the block 1360, to unite the assembly of inner and outer casing sheets 1343, 1342 with the block 1360. In the regions in-between the strips 1315, the foil sheet 314 adheres strongly to the surface of the block 1360 in-between the flocked cells 1311. The regions of the foil sheet which sealingly close the upper end of the cell 1311 are supported by the respective disc 1344, backed by the strip 1315, which as explained above is connected to the rest of the assembly only by the three small dots of adhesive 1346. However, the discs 1344 and strips 1315 are also held in place securely by the bodies of the sheets that surround them, and so resist any accidental force applied in the plane of the sheet, until the end user pulls upwards on the tab 1316 to lift them out of their assembled position.

The capsule caps 21 are then inserted into the flocked cells 1311, as described above and as shown in FIG. 123, before the open end of each cell 1311 is sealed by a sheet of patterned foil 1314 as previously described, as shown in FIG. 124. The flap 1330 with its attached release paper 1332 is then folded over the patterned foil 1314 to protect it in storage, as shown in FIGS. 125 and 127, before placing the finished cap package 1304 in a protective outer wrapper (not shown).

Referring to FIG. 136, in use, after removing the cap package 1304 containing the finished polypills 20 from the machine 1000 as previously described, each cell 1311 is closed by the closed end of the respective carrier 1320, to which adheres a disc of foil 314 that was detached from the capsule body package. Then, the release paper 1332 is peeled away before closing the flap 1330 over the remains of the patterned foil 1314 and the carriers that fill the open ends of the cells 1311 to seal the end user package 1300. The printed indicia 1317′ captured from the stacked packages are exposed on the outer surface of the flap 1330 as shown in FIG. 137. The strips 1315 are exposed on the opposite side of the end user package 1300 as shown in FIG. 138. A similar arrangement is provided in smaller variants of the end user package 1300′, 1300″ which may use the same block and an adapted casing so that only some of the cells are filled, as shown in FIGS. 139 and 140.

Referring to FIGS. 143 and 144, when the user lifts the tab 1316, the first small dot of adhesive 1346 tears away from the body of the inner casing sheet 1343 as the strip 1315 lifts out of the body of the outer casing sheet 1342, pulling the first disc 1344 out of its position in the inner casing sheet 1343 as the foil 314 ruptures around the edge of the respective cell 1311, so that a disc of foil 314 (adhering over its whole surface area to the cardboard disc 1344) is removed cleanly from the cell, exposing the open end of the cell through which the finished polypill 20 drops out when the end user package 1300 is inverted and tapped against a hard surface or the palm of the hand. The carrier 1320 is retained in the cell 1311 by the body of the assembled sheets 1342, 1343. Once the third cell 1311 is opened, the strip detaches from the package.

In order to assist in removing the polypill 20 from the end user package, in this and other embodiments where the polypill 20 is located inside a carrier 320, 1320, the carrier may include a small resilient body (not shown) which is compressed during assembly between the closed end of the capsule body 22 and the closed end of the carrier. The resilient body may remain in an uncompressed state in the carrier or plug package, so that it is compressed only during assembly of the capsule. The resilient body remains under slight compression when the end user package is sealed, and resiles when the cell is opened to urge the capsule 20 out of the cell so that it can be grasped by the user. To maintain the resilient body in compression, the holding force of the carrier in the cell of the end user package is preferably greater than the spring force applied to the capsule 20 by the resilient body. The resilient body could be a moulded, integral feature of the carrier, such as an elastically deformable internal projection, e.g. in a straight or spiral shape, or a plurality of such projections, or a co-moulded elastomeric element. Alternatively, it could be, for example, a solid piece of foamed material which could be assembled into the carrier. The resilient body could react the full axial force applied to the capsule during assembly, or it could be partly received in a recess in the carrier so that it is only partially compressed as the capsule body engages a seat in the carrier during assembly with the cap.

Flocking Apparatus

Referring to FIGS. 118 and 119, a flocking apparatus 1600 may be used to apply the flock 1699 to the cells of the block 1360.

In use, the cell walls of the block 1360 (not shown) are coated with an adhesive, and then the block 1360 is clamped between plates 1601, 1602, each plate having an array of nozzles 1603, so that the open ends of each cell 311 are engaged between a respective pair of nozzles 1603. One of the plates 1602 is at ground or negative potential, and applies that potential to the adhesive, relative to a positive electrode or grid 1604 which charges the flock 1699 which is dispensed from a hopper 1605. A blower 1606 circulates a gentle flow of gas, e.g. dry air, which carries the flock in suspension between the opposed nozzles 1603, through the cells of the block where the fibres stick to the adhesive. Surplus flock 1699 may be removed by a filter 1607 and returned to the hopper 1605. A valve 1608 may be operable to introduce clean air or gas to flow through the cells to clear loose fibres after the flocking operation. Although illustrated in spool valve notation, valve 1608 might be implemented e.g. as a baffle or flap.

Resilient Pushrod Mounting and Transducer Assembly

As discussed above under the heading “Vibration source and graduated pressure”, in embodiments where pushrods are urged through the aligned cells of the stacked packages, the pushrods (e.g. the pushrods 110 of the first machine) may be arranged to transmit vibrational energy to the drug particles 3 and/or capsule components 21, 22 to assist assembly.

Referring to FIG. 107, the variant first machine 1000 illustrates one way to achieve this, wherein each pushrod 1110 is connected to a rigid baseplate 1203 (similar to base plate 203 of the first machine 100) via a resilient connector, e.g. an elastomeric body 1208, which decouples vibration of the pushrod from the baseplate. The pushrods may be slidingly received in through-holes in the pedestal block 1170, similarly to the first embodiment, which helps to maintain them in parallel relation.

Each pushrod 1110 is provided with a respective transducer 1209, which may be mounted beneath the baseplate 1203 as shown. The transducer 1209 is driven by a signal source 1210 to generate vibration energy in the pushrod 1110, which transmits the vibration axially along its length to its end surface which contacts the carrier 1320, which transmits the energy to the capsule components and drug particles.

### Further Variants of the First Embodiment, Having a Plug not Formed as a Carrier: End User Multipacks

The first embodiment and variants thereof exemplify a method of assembling a capsule by receiving in the assembly apparatus at least first and second capsule parts of a plurality of capsules, collecting together between the at least first and second capsule parts of each capsule the single dose of the first drug of a respective one of the cells of the first drug package, and the single dose of the second drug of a respective one of the cells of the second drug package, and closing together the at least first and second capsule parts to enclose within each capsule the respective single dose of the first drug and the respective single dose of the second drug.

As illustrated by each of the above described first embodiment and variants thereof, and further illustrated by two further variant embodiments which will now be described, the packages may include an end user package and a plug package, wherein each cell of the plug package defines an enclosure containing a plug. In such arrangements, the assembly machine 1000 is configured to receive the end user package together with the plug package and the first and second drug packages, and to slidingly displace each plug through respective axially aligned cells of the plurality of packages, to close an open end of a respective one of the cells of the end user package. In this way each capsule containing the single doses of the first and second drugs is sealing enclosed in the respective cell of the end user package. Each cell of the end user package is openable by an end user to remove the respective capsule 20 containing the single doses of the first and second drugs for use.

The method of operation may include receiving an end user package together with the plug package and the first and second drug packages in the assembly machine, and slidingly displacing each plug through respective axially aligned cells of the plurality of packages, to close an open end of a respective one of the cells of the end user package, to sealingly enclose a respective capsule containing the single doses of the first and second drugs in said respective one of the cells of the end user package. Each cell of the end user package is openable by an end user to remove the respective capsule containing said single doses of the first and second drugs for use. Optionally in such arrangements, the plug may form a carrier that contains the capsule body.

Conveniently, as illustrated, each cell of the plug package may define an enclosure containing the plug together with a respective capsule body 22; alternatively (not shown), the capsule body 22 could be contained in a different package from the plug. Each cell of the end user package, as received in the assembly machine, may contain a respective capsule cap 21, and optionally also a locating structure which supports the capsule cap 21 in spaced relation to the cell wall 318.

As illustrated by the first embodiment and above described variants thereof, the plug may be formed as a carrier 320, 1320, wherein the capsule body 22 is arranged in the carrier. The carrier may constrain the cap and body in axial alignment as the cap slides into the annular gap between the outer wall of the carrier and the capsule body.

In such arrangements, it is convenient to remove the finished polypill 20 from the axial end of the cell of the end user package opposite the open end through which it enters.

Alternatively, as will now be described with reference to further variants of the first embodiment, the plug need not be formed as a carrier. The end user package may have a slim form factor with length, width and thickness dimensions, wherein the thickness dimension is less than the length and width dimensions. The thickness dimension may be only slightly greater than the diameter of the capsules and less than the capsule length, so that the length axis of the capsule extends in the width or length dimension of the end user package, conveniently in the width dimension as will now be described.

The capsules may be removed from an axial end of the cell via a narrow side of the end user package extending in its thickness dimension, as in the above described embodiments. Alternatively, the capsules may be removed via a broad side of the end user package that extends in its length and width dimensions, as will now be described.

In the latter case, the broad side of the package may be defined by a foil wall which is ruptured when the capsule is removed, optionally by pressing against an opposite wall of the cell to force the capsule through the foil wall. In this case, since the foil wall may not be configured to maintain the capsule cap and body in axial alignment, the cap may be arranged in a cap package separate from the end user package, so that the capsule is closed before it enters the cell of the end user package, as illustrated by the first of these further variants, which also illustrates how the plug may be combined with a sliding collar to close the open end of the cell containing the finished capsule 20.

If assembly occurs outside the end user package, then a restraining element may be arranged to restrain the cap 21 while it engages with the body 22, and then to move to allow the finished capsule 20 to enter into the end user package. The restraining element could be an elastically or plastically deformable or frangible element which is moved aside or broken by the finished capsule, e.g. a membrane that tears at a force greater than the force required to close the cap and body together, or it could be a movable closure element which is displaced by an actuator of the machine 100, 1000 after the capsule assembly step, as illustrated by the first of these further variants, described below.

Alternatively, as illustrated by the second of these further variants, also described below, a part of the cell wall at the broad side of the package may be detachable by the end user to open the cell to remove the capsule 20 via the broad side of the package, wherein the cell wall is configured to maintain the capsule cap and body in axial alignment as the capsule is closed. In this way, the cap may be located inside the end user package which then forms the cap package, so that the capsule is closed inside the cap package.

During manufacture of the cap package, the cap 21 may be introduced into the cell before closing together the two parts of a clamshell package, as shown. Alternatively (not shown), the cap 21 may be introduced axially through an open end of the cell, and then retained by closing the open end of the cell or by a cylindrical sleeve which is inserted after the cap and which then forms a guide for the capsule body during assembly.

As further illustrated by each of these two further variants, the end user package having a slim form factor may be a sub-package forming part of a multipack comprising a plurality of such end user sub-packages; thus, the term “end user package” may refer to the end user multipack or to the individual end user sub-package. Each sub-package includes a row of cells which can be opened by the end user through one or both of the broad sides of the sub-package. The sub-packages could be held together by frangible or releasable connections or by a film or wrapper or (as illustrated) a box, to form an assembly so that they can be separated by the end user as required. Conveniently, the box can be arranged to display the package indicia and to contain the patient information leaflet, if provided.

Thus, as illustrated by each of the further variants which will now be described, the end user package may include an assembly of sub-packages, each sub-package including a plurality of cells, wherein the sub-packages are separable for use by the end user. The first of these variants illustrates how the sub-package may be separate from the cap package, wherein the capsule is assembled inside the cap package and outside the end user sub-package. The second illustrates how the sub-package may form the cap package, so that the capsule is assembled inside the sub-package.

Capsule Assembly Outside the End User Package; Plug and Collar

Referring now to FIGS. 164-206, the stacked packages may include a cap package 3040 which defines a spaced array of cells 311, each of which includes capsule cap 21 and a collar 3060. The collar 3060 defines an aperture 3061 and is configured to guide the respective capsule body 22 to enter telescopically into the respective capsule cap 21 via the aperture 3061. The cap 21 may be arranged in the aperture 3061, as further described below.

The assembly machine 1000 is arranged and operated generally as described above with reference to the variant first embodiment, but with some adaptations as will be evident from the following discussion. For example, the image reproduction device (not shown) may be arranged in a different position; there may be more than one image reproduction device; and the machine may have an additional actuator (not shown) which is configured to actuate an additional movable closure element 3043 of the cap package 3040, as further explained below.

The assembly machine is configured to displace the collar 3060 slidingly into the open end 3022 of the respective cell 3021 of the end user package or sub-package 3020, and to slidingly displace the plug 3070, from the respective cell of the plug package 3050 into the aperture 3061 of the collar 3060, so that the plug 3070 and the collar 3060 together close the open end 3022 of the respective cell 3021 of the end user package or sub-package 3020 containing the capsule 20.

As illustrated, the cap package 3040 and the end user package 3001 may be connected together to form an end user package assembly, wherein the machine 1000 is configured to receive the end user package assembly together with the drug packages 3051 and plug package 3050.

The drug packages 3051 and plug package 3050 may have re-usable monolithic foraminous blocks and movable closure elements, as illustrated, thus corresponding generally to the alternative package 2301 previously described. Alternatively, the drug packages and/or plug package could be closed by foil, corresponding to the drug packages 1301, 1302 or 1303 previously described. Each cell of the plug package 3050 may contain a plug 3070 and a capsule body 22, with the plug being arranged in use between the capsule body 22 and the pushrod 1110, as shown in FIG. 201.

Referring to FIGS. 165-173, each sub-package 3020 may include a body 3027 formed as a unitary plastics moulding defining a single row of cells 3021, each cell opening through the broad, front side of the body 3027, which defines a flat surface 3028, and via an aperture 3022 formed in the narrow, bottom side of the body 3027. The aperture 3022 may have a projecting rim 3023. A pair of location tabs 3026 may be moulded at the opposite narrow, top side of the body.

The foil wall 3024 is applied (e.g. ultrasonically welded) to the flat surface 3028 to sealingly close the front side of each cell 3021 as shown in FIG. 164.

Referring to FIGS. 174-178, a number of sub-packages (e.g. six sub-packages 3020, as illustrated) are then assembled together, e.g. in a box 3002 as shown, to form the empty end user package 3001. The box may have apertures 3008 formed in its base, through which the rims 3023 of the sub-packages project.

The top of the box may be left open to expose the narrow, top sides of the sub-packages, which may have a surface suitable (e.g. having a suitable coating) for indicia to be printed directly onto said surface, or (as illustrated) may be covered by an adhesive label or labels 3009 to facilitate printing the package indicia captured from the stacked drug packages on each sub-package, optionally with additional package indicia and/or customer details, as earlier described. The label may be perforated between the sub-packages so that it holds them together but makes it easy to separate them by tearing along the perforation; alternatively, each sub-package could have a separate label. The box 3002 may have closure elements, e.g. end flaps 3003 and side flaps 3004, 3007 which are folded down against its outer surface. The distal edge region of the front side flap 3004 may have a band of adhesive covered by a release paper 3006, and may be connected to the rest of the side flap via a tear-off strip 3005 with a tab 3010.

Referring to FIGS. 190-198, the cap package 3040 may be an assembly of re-usable, separable parts. For example, as illustrated, it may include upper and lower foraminous blocks 3042, 3041 having marginal projections defining slideways 3044 which are aligned when the blocks are superposed. The blocks are connected together by a movable closure element 3043 formed as a foraminous plate which, when in the closed position (wherein its holes are misaligned with the holes in the blocks) engages in both slideways. When the plate 3043 is moved by the machine actuator (not shown) to the open position, its holes are axially aligned with the corresponding holes or cells in each block, and recesses in its edges align with the marginal projections in the upper block 3042 so that the upper block 3042 can be lifted away from the lower block 3041 after the packages are removed from the machine. The plate 3043 can then be removed from the lower block 3041 so all three components can be cleaned and re-assembled at the factory to form a new package.

As shown in FIG. 201, the upper part of each cell 311 of the lower block 3041 is enlarged, relative to its lower part, to define an annular seat 3048 which axially retains the collar 3060 which is received in the enlarged upper part of the cell, with the cap 21 being arranged in the aperture 3061 of the collar. The upper end of each cell of the upper block 3042 may define a chamfered recess 3045 which receives a respective one of the rims 3023 of a respective sub-package 3020, locating the cell 3021 of the sub-package in axial alignment with the corresponding cell of the cap package 3040.

Referring to FIGS. 183-189, the end user package 3001 is placed on top of the cap package 3040 so that the rims 3023 locate in the recesses 3045, and may be retained by a casing 3011 which is locked to the cap package, e.g. by marginal portions of the plate 3043 which engage in hooks 3012 of the casing; when the plate 3043 is moved to its open position, the hooks 3012 are disengaged so that the casing 3011 can be lifted away from the cap package 3040 after the packages are removed from the machine 1000.

Pressure bars 3015 may be provided at the upper end of the casing to engage the location tabs 3026 of the sub-packages 3020 so as to maintain the upper ends of the sub-packages 3020 in the correct position. During assembly, the stacked packages are maintained in compression between the pressure plate 1121 of the machine 1000, which bears against the top surface of the casing 3011, and the pedestal block 1170 on which the packages are stacked as previously described.

It should be understood that although the pedestal block and pressure plate have been described as features of the first machine and each of its variants, they are merely convenient ways to maintain the stacked packages in compression; alternative arrangements are possible.

Windows 3013, 3014 may be formed in the top and side of the casing 3011, through which the image reproduction devices (not shown) can write the captured package indicia onto one side of the box 3002, as shown in FIG. 180, and/or onto the labels 3009 of the sub-packages 3020, as shown in FIG. 179.

After the casing 3011 is locked to the cap package 3040, a tamper evident label 3046 may be applied to the finished assembly as shown in FIG. 185. When the plate 3043 is moved by the machine actuator (not shown) to its open position, a projection 3047 of the plate tears the label 3046 so that the label indicia are disrupted. The damaged indicia can then be detected by a scanner and processor 262 of the machine 1000, optionally also by validating the label indicia via its data link with the remote computer 91 and database 90 (as shown in FIG. 1 and FIG. 102, mutatis mutandis).

Referring to FIG. 200, the collar 3060 may include a radially outwardly facing, annular sealing surface 3062 which is configured to seal against a corresponding, inwardly facing surface of the aperture 3022 of the sub-package, and a set of resilient, radially inwardly compressible prongs 3063 which define latching surfaces 3064. An internal annular seat 3066 may be arranged to retain the cap 21 in the aperture 3061.

Referring to FIG. 199, the plug 3070 may include, similarly, a radially outwardly facing, annular sealing surface 3072, which is configured to seal against a corresponding, inwardly facing surface of the aperture 3061 of the collar 3060, and a set of resilient, radially inwardly compressible prongs 3073 which define latching surfaces 3074. A central post 3071 may project axially between the prongs 3073 to bear against the closed end of the capsule body 22 in use.

The sealing surfaces 3062, 3072 may be arranged not to contact the cell walls of the packages during assembly. Thus, either or both of the sealing surfaces 3062, 3072 may include a sealing material, e.g. an adhesive or a co-moulded elastomer.

The re-usable package components may be moulded or machined from plastics material, e.g. engineering plastics. The collar and plug may be moulded in plastics material, e.g. biodegradable cellulose acetate.

Referring now to FIGS. 201-206, the movable closure elements (if present) of the plug package 3050 and the drug packages 3051 are moved to the open position by the machine actuator and displacement surface (1195, FIG. 156, mutatis mutandis), as previously described, and then the pushrods 1110 are urged to push the plug 3070 through the axially aligned cells of the stacked packages. The plug 3070 in turn pushes the capsule body 22 towards the cap package, collecting the drug particles (e.g. spheroids) 3 inside it (FIG. 202). The capsule body 22 passes through the aperture 3061 of the collar into the cap 21, which is restrained by the plate 3043 which remains in the closed position until the capsule is closed (FIG. 203). The plate 3043 is then opened by the additional actuator and displacement surface of the machine (1195, FIG. 156, mutatis mutandis), and further movement of the pushrod 1110 urges the filled capsule 20 through the aperture 3022 of the end user sub-package 3020 into the respective cell 3021 (FIG. 204). At the same time, the plug 3070 enters the aperture 3061 of the collar, while the collar 3060 is also slidingly displaced to enter the aperture 3022 of the sub-package 3020 (FIG. 205). Finally, the prongs 3073 of the plug 3070 move outwardly so that the latching surfaces 3074 engage a confronting, axial abutment surface 3065 of the collar, while the prongs 3063 of the collar move outwardly so that the latching surfaces 3064 engage a confronting, axial abutment surface 3029 of the aperture 3022 of the sub-package, locking the plug and collar in position to close the cell 3021 as permanent parts of the sub-package 3020 (FIG. 206).

Referring to FIGS. 179-182, the end user package 3001 containing the polypills 20 sealed in the individual cells 3021 of the sub-packages 3020 is then removed from the machine 1000 and separated from the cap package 3040 and casing 3011 which are returned to the factory to be cleaned and re-assembled to form a new package. The box 3002 and sub-package labels 3009 may display the composite label 1312 which may be a reproduced image comprising the package indicia, which may be captured from the stacked packages, uploaded and validated as previously described, and patient indicia downloaded from the server and database 91, 90 (FIG. 1) as previously described; as illustrated, the sub-package labels may bear a subset of the indicia to fit the available space.

The end flaps 3003 are then folded over to retain the patient information leaflet 266, which is printed from data downloaded from the remote server 91 (FIG. 1) as previously described, folded and placed on top of the sub-package assembly. Then, after removing the release paper 3006, the front flap 3004 is folded over the rear flap 3007 so that the adhesive strip seals the flaps together. The end user package 3001 is now ready for delivery to the customer. In use, the customer opens the box by pulling the tab 3010 to remove the tear-off strip 3005, and then separates the sub-packages 3020 as required for use. Each polypill 20 can be removed by pressing against the rear wall 3025 of the cell to push the capsule out through the opposite foil wall 3024.

Capsule Assembly Inside the End User Sub-Package

Referring now to FIGS. 207-217, this variant is generally the same as the last described variant, except that the end user sub-package is formed as a cap package 3080 having a plurality of cells 3081 containing the capsule caps 21. Thus, the separate cap package and collar, and the additional actuator of the machine as shown in the last described variant are not required.

Each cell 3081 of the cap package 3080 has an open end forming an aperture 3082, which may have a projecting rim 3083 similar to the rim 3023 of the last described embodiment. In this variant, and in the first embodiment and other variants thereof, the packages may be arranged to mutually interlock with their cells in axial alignment. For example, the drug packages 3051 and plug package 3050 may be adapted to include a recess or chamfer 3100 at the upper end of each cell, and a corresponding projecting collar 3101 to engage in the corresponding recess 3100 of the package immediately below. The recess 3100 of the uppermost drug package 3051 in the stack receives the rim 3083 of the cap package 3080 (which may project through the bottom of the box 3002 as previously described) to locate the cell 3081 in axial alignment with the cells 311 of the rest of the packages. Alternatively, the recess 3100 and collar 3101 could be arranged respectively on the lower and upper sides of each package rather than, respectively, the upper and lower sides, and the open end of the cell 3081 of the cap package 3080 could define a recess to receive the upwardly projecting collar of the uppermost drug package in the stack. Alternatively, other mutually interlocking alignment features could be provided.

In such arrangements, the recess 3100 and collar 3101 may be formed on the previously described movable closure elements 2401, but the packages adapted so that the previously described block 2400 of the package is displaced by the machine 1000 while the movable closure elements 2401 remain static.

A plurality of sub-packages 3080 are assembled together, e.g. with releasable connectors, in a wrapper, or in a box as shown in the last described variant, and then stacked in the machine 1000 (FIG. 1, FIG. 102, mutatis mutandis) together with the drug packages 3051 and the plug package 3050, which may contain the capsule body 22 as well as the plug 3070 (FIG. 201).

As previously described and shown in FIGS. 214-217, during operation of the machine 1000, the pushrod 1110 urges the capsule body 22 followed by the plug 3070 axially slidingly through the aligned cells 311 to collect the drug particles 3. The capsule body 22 enters the cell 3081 via aperture 3082 before sliding telescopically into the capsule cap 21 to close the capsule, followed by the plug 3070 which is received fittingly, optionally sealingly or even hermetically sealingly in the aperture 3082 to close the open end of the cell 3081 containing the finished capsule 20. In the illustrated example, the prongs 3073 move resiliently outwardly to engage latching surfaces 3074 with the axial abutment surface 3084 of the aperture 3082, retaining the plug 3070 in the aperture 3082 to form a permanent part of the end user sub-package 3080.

Referring to FIGS. 207-211, the cap package 3080 may be formed as a clamshell style assembly of front and rear mouldings 3085, 3086 which are connected together, e.g. by an ultrasonic weld line 3087 that extends around each cell to join together the confronting, flat surfaces 3088, 3089 of the two parts.

During assembly of the cap package 3080, each capsule cap 21 may be arranged over the rounded end of a respective cylindrical rod (not shown), equal in diameter to the capsule body 22. The rods are laid in the open half cells 3081 of the rear moulding 3086 with the caps positioned in the slightly larger diameter, upper end portion of the cell, in which they are retained by the axial abutment surface 3093 located between the upper and lower portions of the cell 3081. The rods (not shown) remain in place while the two mouldings 3085, 3086 are welded together, and are then withdrawn axially through the cell apertures 3082 leaving the caps in place inside the cell.

Weakened break lines 3090 may be defined in the package mouldings 3085, 3086 to define a detachable portion 3091 of the cell wall. A tab 3092 may be formed as part of the detachable portion 3091 so that in use, the end user may press against the tab 3092 to break away the detachable portion 3091 of the cell wall to open the cell 3081 to remove the finished capsule 20, as shown in FIGS. 212 and 213. The detachable portion 3091 may detach entirely or, as shown, partially to form a hinged flap.

### Automated Assembly

In each of its embodiments, instead of a human operator, the machine may be configured as an integral part of a fully automated system, so that all its functional features are machine controlled. In this case the machine need not include features configured for human use, e.g. handles, a user interface or an outer casing or door, except as required, e.g. to control and monitor the system or to retain a modified atmosphere or negative pressure. The pressure plate and/or front plate or equivalent features, where present, may be operated by actuators.

The machine may not include a front plate. The machine may not include a moving frame assembly. The machine may not include a pedestal block. The pressure plate, if present, could move in translation, rather than in rotation, to accommodate loading of the packages as well as to accomplish the assembly stroke.

The steps of reading the package labels and printing the end user package label could be accomplished during, before, and/or after the assembly stroke.

Where the cap package includes a flap that will become the end user package label, the flap could be printed before or after sticking it to cover the respective side of the finished end user package. The release paper could be a flexible sheet or a stiffer sheet of card. The machine could grip and rotate the tab of the release paper to open the flap for the assembly operation, and then rotate and lift the tab to remove the release paper before sticking down the flap. Alternatively, the adhesive could be applied to the flap during assembly.

Alternatively, the end user package label could be formed as a separate part that is applied to the end user package after the assembly stroke, rather than as a surface of the box of an end user multipack or as a flap that forms a part of the cap package. For example, it could be a self adhesive label mounted on a release paper, perhaps formed on a roll. The label could be peeled from the release paper as the release paper passes around a roller. The end user package label could be applied to the end user package either before or after it is printed.

Robotic handling machines may be arranged to select and retrieve the packages from a storage location and deliver them to the machine. Similar handling machines may remove the finished end user package from the machine and prepare it for delivery to the end user, e.g. via a local pharmacy, or by post to the user's home address. The customer name, address and other details may be printed by the apparatus on the end user package, the patient information leaflet and/or a separate adhesive label when preparing the end user package. The empty packages may be removed by the handling machine and placed in a bin to be returned to the manufacturing location for recycling as previously described.

### Common Features of the First Embodiment and Variants Thereof as Described Above

It will be understood that each of the first embodiment and its variants as described above defines, by way of example, an assembly system and method for filling drugs into capsules to produce customised polypills in the form of capsules 20.

The assembly system includes the following common features:—
An assembly apparatus (e.g. the first or variant first machine 100, 1000).
A plurality of packages, including at least first and second drug packages, each package of the plurality of packages including a plurality of cells.
Each cell of the first drug package includes a single dose of a first drug, while each cell of the second drug package includes a single dose of a second drug.
Each cell of each drug package includes only one single dose of the respective drug.
The assembly apparatus is configured to receive the plurality of packages including at least the first and second drug packages, to receive a plurality of capsule caps 21 and capsule bodies 22, and to combine together the first and second drugs of the first and second drug packages to form a plurality of capsules 20.
For each capsule 20 of the plurality of capsules, the single dose of the first drug of a respective one of the cells of the first drug package is encapsulated, together with the single dose of the second drug of a respective one of the cells of the second drug package, between a respective said capsule cap 21 and capsule body 22.

The method includes the following features:—
Providing first and second drugs, and a plurality of packages, the plurality of packages including at least first and second drug packages, each package of the plurality of packages including a plurality of cells.
Packaging the first drug in the first drug package so that each cell of the first drug package includes a single dose of the first drug, and packaging the second drug in the second drug package so that each cell of the second drug package includes a single dose of the second drug, wherein only one said single dose is packaged in each respective cell of each respective drug package.
Receiving in an assembly apparatus (e.g. the first or variant first machine 100, 1000) the plurality of packages including at least the first and second drug packages together with a plurality of capsule caps 21 and capsule bodies 22, and operating the assembly apparatus to combine together the first and second drugs of the first and second drug packages to form a plurality of capsules 20.
For each capsule 20 of the plurality of capsules, the single dose of the first drug of a respective one of the cells of the first drug package is encapsulated, together with the single dose of the second drug of a respective one of the cells of the second drug package, between a respective said capsule cap 21 and capsule body 22.

Preferably, in each of the first embodiment and its variants as described above, each single dose comprises one or more particles, wherein the particles of all said single doses are of equal size and shape, and each of the cells of the first drug package includes an equal number of particles, and each of the cells of the second drug package includes an equal number of particles.

By equal shape is meant, of substantially the same shape—for example, all of the particles may have a spheroidal shape. A spheroid may be, for example, a sphere, or a sphere with a raised belly band.

By equal size is meant, of substantially equal size, or within a relatively narrow target size range. For example, each particle could have a maximum dimension within +/−5%, preferably +/−3%, more preferably +/−2%, yet more preferably +/−1%, of a mean maximum dimension.

Further preferably, in each of the first embodiment and its variants as described above, each particle has a dimension of at least 1.5 mm.

Further preferably, in each of the first embodiment and its variants as described above, each particle is a spheroid 3 with a diameter of at least 1.5 mm.

Preferably the dimension of each particle, or the diameter of each spheroid, is at least 2 mm, more preferably at least 2.5 mm, for example, about 2.8 mm.

The dimension is taken in a straight line through the geometric centre of the particle.

Most preferably, the diameter of each spheroid is less than one half, and greater than one third, of the internal diameter of the capsule body. This ensures that the spheroids can pass one another to achieve an optimal packing density without jamming inside the capsule body.

### Further Variant Embodiments for Producing Polypills as Capsules, Wherein Each Single Dose is Packaged in a Respective Cell of a Respective Drug Package

Further variant embodiments which include the common features of the first embodiment and variants thereof, as listed above, will now be described, including a variant described below under the heading “Single dose drug packages and bulk capsule components”.

In each of these further variant embodiments, each single dose is packaged in a respective cell of a respective drug package, and each single dose comprises one or more particles 3, wherein the particles of all said single doses are of equal size and shape, and each of the cells of the first drug package includes an equal number of particles, and each of the cells of the second drug package includes an equal number of particles. That is to say, all of the cells of the first drug package include the same number n1 of first particles, and all of the cells of the second drug package include the same number n2 of second drug particles, wherein n1 and n2 are whole numbers equal to or greater than 1. Each particle has a dimension of at least 1.5 mm.

By equal shape is meant, of substantially the same shape—for example, all of the particles may have a spheroidal shape. A spheroid may be, for example, a sphere, or a sphere with a raised belly band.

By equal size is meant, of substantially equal size, or within a relatively narrow target size range. For example, each particle could have a maximum dimension within +/−5%, preferably +/−3%, more preferably +/−2%, yet more preferably +/−1%, of a mean maximum dimension.

Preferably, each particle is a spheroid 3 with a diameter of at least 1.5 mm.

Preferably the dimension of each particle, or the diameter of each spheroid 3, is at least 2 mm, more preferably at least 2.5 mm, for example, about 2.8 mm.

The dimension is taken in a straight line through the geometric centre of the particle.

Most preferably, the diameter of each spheroid is less than one half, and greater than one third, of the internal diameter of the capsule body. This ensures that the spheroids can pass one another to achieve an optimal packing density without jamming inside the capsule body.

As with the previously described embodiments, if the volume of the combined drugs 1,2 is too great to be enclosed in a single capsule 20, then the dose may be divided between two of more capsules 20. Thus, the single dose contained in each capsule 20 may represent a total therapeutic amount constituting a single dose of the respective drug 1, 2, or a fraction of a total therapeutic amount, wherein two or more identical polypills 20 are to be taken together, said fractions when combined together constituting the total therapeutic amount of the respective drug 1, 2 in a single dose.

The term “particle” is synonymous with the term “granule” as referred to above. The first and second particles may be configured and manufactured as described for the earlier embodiments, preferably as spheroids 3. Advantageously, by packaging and subsequently encapsulating each single dose in the form of one or more relatively large, discrete particles, preferably spheroids, the method avoids contaminating the assembly apparatus with a finely divided powder, as is commonly employed when encapsulating conventional, fixed dose combinations. (This is the case even where the machine is optionally arranged to come into direct contact with the particles during assembly, as further described below in another variant embodiment under the heading “Bulk drug packages and bulk capsule components”.) This makes it possible to form multiple batches of customised polypills without stripping down and radically cleaning the machine between batches. The machine may incorporate a cleaning system (e.g. cleaning rods, not shown, that reciprocate in its internal passageways, e.g. in the chutes of the filling apparatus 2430 when configured as a dosing apparatus 4070, further discussed below under the heading “Bulk drug packages and bulk capsule components”) that can be used to remove any broken or stuck drug particles between operations.

Thus, each single dose of the first particles 3 may be defined by a first target number of the first particles 3, and each single dose of the second particles 3 defined by a second target number of the second particles 3. That is to say, the single dose of each drug 1, 2 may be defined by the number of particles 3, rather than in terms of mass or volume as is conventional in powder filling techniques.

This may be facilitated particularly by arranging for each particle 3 to contain a consistent quantity of the respective drug 1, 2, e.g. by forming the particles 3 by known tabletting techniques, or perhaps by spheronization where the diameter of each spheroid is closely controlled, e.g. including a grading step after spheronizing to remove, optionally also reprocess any spheroids outside a narrow, target diameter range. Alternatively or additionally, spheronized particles could be further processed to obtain a constant diameter (e.g. by abrading or rolling between opposed surfaces) and then perhaps also graded by mass.

Preferably, the particles 3 are of equal mass, which is to say, of substantially equal mass, wherein the mass of each particle lies within a relatively narrow range. For example, the mass of each particle could lie within +/−10%, preferably +/−5%, more preferably +/−3% of a mean mass. The particles could be graded by mass to remove out-of-specification particles, for example, by centrifugal separation, or by a winnowing process in which the particles fall through a moving current of dry gas, with lighter particles being deflected by the gas to a reject bin, over-weight particles being deflected relatively little and falling nearly directly into another reject bin, and particles in the target range being deflected by an intermediate degree to fall into a target bin.

As in the previous embodiments, the assembly apparatus may be further configured to package the plurality of polypills 20 in individual cells of an end user package, each cell sealingly enclosing a respective one of the polypills 20 and being openable by an end user to remove the polypill from the cell, as further discussed below. This may be preferred because it extends shelf life after delivery to the customer, and obviates the possibility of misdentification which could occur for example after a bottle containing a quantity of capsules is first opened, after which it is no longer possible to verify its contents. Alternatively however, the polypills 20 could be packaged together in a single container.

The capsule caps 21 and bodies 22 may be packaged singly in individual cells 311 of capsule cap and body packages, e.g. as illustrated in the first and variant first embodiments, or may be provided as bulk capsule components as further described below.

### End User Package Containing Polypills in the Form of Capsules

It will be appreciated that each of the first embodiment and its variants as described above provides an end user package including a plurality of capsules, wherein each capsule includes a single dose of a first drug, and a single dose of a different, second drug. Each capsule contains a plurality of particles, a first one or first ones of the particles containing the first drug but not the second drug, a second one or second ones of the particles containing the second drug but not the first drug.

In accordance with another aspect of the invention, in such an end user package, the first and second ones of the particles are spheroids 3 having a mean diameter of at least 1.5 mm, and all of the capsules contain an equal number of said first one or first ones of the particles, and all of the capsules contain an equal number of said second one or second ones of the particles. That is to say, all of the capsules contain the same number n1 of the first particles, and the same number n2 of the second particles, wherein n1 and n2 are whole numbers equal to or greater than 1.

By mean diameter is meant the average diameter of all of the particles. By diameter is meant the maximum diameter of the particle. For example, if the particle is a spheroid in the form of a sphere with a belly band, i.e. a slightly raised equatorial region (commonly formed when tablets are formed by pressing), then the diameter is taken across the belly band (which is to say, between oppositely facing surfaces of the belly band, in a diametric plane passing through the belly band).

In this aspect of the invention, preferably the spheroids have a mean diameter of at least 2 mm, more preferably at least 2.5 mm, for example, about 2.8 mm.

In this aspect of the invention, preferably all the spheroids are of equal diameter. By equal diameter is meant, of substantially equal diameter, or within a relatively narrow target diameter range. For example, each particle could have a diameter within +/−5%, preferably +/−3%, more preferably +/−2%, yet more preferably +/−1%, of the mean diameter.

Most preferably, the diameter of each spheroid is less than one half, and greater than one third, of the internal diameter of the capsule body. This ensures that the spheroids can pass one another to achieve an optimal packing density without jamming inside the capsule body.

End user packages that may be produced in accordance with this aspect of the invention (e.g. end user packages 300, 1300, 3001, 1380) are described above, and further such packages (e.g. blister packages 4062) that may be produced in accordance with this aspect of the invention are described below. In yet further variants, the end user package need not include individual cells in which the polypills are packaged individually, as exemplified by each of the illustrated embodiments; it could be a container, for example a bottle or a packet, containing a quantity of the capsules.

Further Variant Embodiments with Bulk Capsule Components

The further variants of the first embodiment discussed below with reference to FIGS. 218-230 illustrate how the capsule caps 21 and bodies 22 may be introduced in bulk form into the assembly machine, instead of packaging them in individual cells of cap and body packages as in the first embodiment and its variants as previously described.

Referring to FIG. 218, the assembly machine 4000 may be adapted to receive the capsule components in bulk form, e.g. in a bulk capsule cap package 4001 and a bulk capsule body package 4002 as illustrated. The packages 4002, 4002 contain, respectively, in a single compartment or individual compartments, multiple caps 21 and multiple bodies 22, which are dispensed from the packages into a capsule assembly mechanism of the machine 4000. Alternatively, a single bulk package could contain, in one compartment or individual compartments, multiple empty capsules which the machine is configured (in a manner known in the art) to separate into their caps 21 and bodies 22 prior to assembly of the polypill 20.

The capsule assembly mechanism can take any conventional form as known in the art, but in the illustrated examples includes a capsule body handling mechanism 4010, which defines one or more capsule body chambers 4011 for receiving the capsule bodies 22, and a capsule cap handling mechanism 4020 which includes one or more capsule cap chambers 4021 for receiving the capsule caps. A closing mechanism is also provided, which as illustrated may include one or more pushrods 4030 which extend axially through the body chambers to urge the capsule components 21, 22 together.

In such arrangements, as illustrated by the variant embodiment described below under the heading “Single dose drug packages and bulk capsule components”, each of the first and second drug packages may include a plurality of cells 311, wherein each cell 311 contains only one said single dose of the respective, first or second drug. Each drug package may be as earlier described with reference to the first and embodiment and variants thereof, e.g. drug packages 301 or 1301 which are closed by foil or, as illustrated, drug packages 2301 which are closed by movable closure elements.

Alternatively, as illustrated by the further variant embodiment described below under the heading “Bulk drug packages and bulk capsule components”, each of the first and second drug packages may be a respective, bulk drug package 4071, 4072, including more than one single dose of the respective, first or second drug 1, 2 in the form of a quantity of relatively large particles 3 of equal size and shape contained in a single compartment. In this case, a dosing apparatus 4070 may be arranged to dispense a quantity of the particles 3 corresponding to a single dose of the first or second drug 1, 2 which are encapsulated between the cap and body components 21, 22 as further discussed below.

One way to do this is by defining a first target number of the first particles 3 and a second target number of the second particles 3 required to form said respective single dose, as mentioned above; and, for each of the plurality of polypills 20, removing from the respective first and second bulk drug packages 4071, 4072 and encapsulating between the respective capsule cap 21 and capsule body 22 the first target number of the first particles 3 and the second target number of the second particles 3 to form the respective polypill 20.

For example, as shown in FIG. 230 and further described below, the dosing apparatus 4070 may be arranged to transfer an exact number of particles 3 into a dosing chamber or capsule body in a single operation. In the illustrated example, the dosing apparatus 4070 comprises two filling apparatuses 2430, each of which is generally as previously described and illustrated in FIGS. 160-163, but adapted mutatis mutandis to fill a single capsule body 22 seated in the capsule body chamber 4011, instead of a respective cell of the drug package as previously described.

In the first embodiment and variants thereof as discussed above, the assembly apparatus may be configured to form the polypills 20 without direct contact between the assembly apparatus and any of the capsule cap 21, the capsule body 22, and the first and second drug particles 3. This may be achieved by packaging the capsule components 21, 22 and drug 1, 2 doses singly, combining the doses together within the cells of the packages, and arranging the carrier 320, 1320 or plug 3070 in-between the pushrods 110, 1110 and the capsule components. In this way, even if the machine 100, 1000 should become contaminated, e.g. by making a set of polypills from contaminated packages, or by a worker with dirty hands, a subsequent set of polypills made in the same machine, even without cleaning the machine, will not be contaminated by contact with the machine components. This helps to make it possible to operate the machine with high productivity, even in an environment such as a pick-and-place warehouse or logistics centre where less stringent cleaning routines are used in place of traditional pharmacy procedures.

Alternatively however, the assembly machine may be configured to manipulate the capsule components 21, 22 by direct contact with contact surfaces of the machine.

In such arrangements, as exemplified by the variant embodiment further discussed below under the heading “Single dose drug packages and bulk capsule components”, the machine need not come into contact with the drug particles 3, which by virtue of their consistent, relatively large size and shape, tend to roll in a predictable manner in contact with the surfaces of the package cells and capsule components during assembly. A spheroidal shape is particularly preferred because it ensures a rolling motion of the particles in any orientation. This minimises the risk of contamination of the machine by drug fragments. Thus, where sterile capsule components are loaded into the assembly machine in bulk packages 4001, 4002, the machine may still be operated substantially without the risk of contamination or cross-contamination between successive batches of polypills containing different drugs for different customers.

In yet further such arrangements, as exemplified by the further variant embodiment discussed below under the heading “Bulk drug packages and bulk capsule components”, the machine may contact both the capsule components and the drug particles 3. In such arrangements, although there is direct contact with machine components, the consistent shape and relatively large size of the preferably spheroidal particles 3 again minimises the risk of entrapment of particles 3 or damage to the particles 3 leading to cross-contamination.

Most preferably in each case, the particles 3 are spheroids with a diameter less than one half, and greater than one third, of the internal diameter of the capsule body 22. This ensures that the spheroids 3 can pass one another to achieve an optimal packing density without jamming inside the capsule body 22, which further ensures reliable and repeatable assembly without contamination of the machine.

In each of the below described variant embodiments, as in the previously described first embodiment and variants thereof, a package label may be produced by the machine 4000 to list the first and second drugs 1, 2 and optionally also related information, e.g. customer information 1080 which may be downloaded from the server as previously described. The label could be printed or stuck directly on the blister pack 4062 or other end user package and/or on an outer wrapper, e.g. a box 4061 or a bag in which the end user package is delivered to the end user, as shown in FIGS. 228 and 229. The label may be produced from indicia or data captured from the (bulk or cellular) drug packages 2301, 4071, 4072, either in visible or non-visible form, e.g. as a barcode or an electronic ID tag. In each case, the label may include a composite label 1312 which reproduces the visible indicia captured from the drug packages as a reproduced image as earlier described.

The (bulk or cellular) first and second drug packages 2301, 4071, 4072 may be arranged with other drug packages in the apparatus, e.g. in a carousel (not illustrated) or in a defined location in a pick-and-place mechanism, and the assembly machine 4000 arranged to select the required drug packages for each set of polypills according to the prescription data which are received by the machine; alternatively, the drug packages 2301, 4071, 4072 could be inserted and removed manually for each operation.

As with the other variants of the first embodiment, the assembly machine 4000 may include data upload/download and drug (optionally also capsule component) serialization data validation and anti-counterfeiting functions similar to those of the first and variant first machines 100, 1000 as described with reference to FIG. 1.

Single Dose Drug Packages and Bulk Capsule Components

Referring first to the example of FIGS. 218-229, as in the first embodiment and variants thereof as previously described, each of the first and second drug packages 2301 includes a plurality of cells 311, wherein each cell 311 contains only one said single dose of the respective, first or second drug 1, 2 as a defined number of relatively large particles 3 of equal size and shape. Each cell 311 contains the same number of particles 3. The assembly apparatus comprising assembly machine 4000 is configured, for each of the polypills 20, to urge at least one of the respective capsule cap 21 and capsule body 22 (as illustrated, the capsule body 22) through corresponding, axially aligned cells 311 of the first and second drug packages 2301 to encapsulate the respective first and second particles 3 between the capsule cap 21 and the capsule body 22.

In this way the machine is isolated from the drug particles 3 and contacts only the cap 21 and body 22 of the capsules, reducing the possibility of cross-contamination between successive operations, particularly when the particles 3 are formed as spheroids to minimise damaging interactions with the cell walls of the drug packages during assembly.

The relatively large dimension of the drug particles 3, which preferably is the diameter of the spheroids 3, ensures that the drug particles or spheroids 3 do not become trapped between the pushrods 4030 of the machine and the other machine parts or the cell walls of the drug packages 2301, and so helps avoid cross-contamination between different batches of polypills 20.

As shown in FIG. 218, the drug packages 2301 may be introduced into the machine 4000 via window 4004 and stacked with their corresponding cells 311 in axial alignment as in the earlier described embodiments. The cap 21 and body 22 of the capsule may be positioned in axial alignment with the cells 311 (hence, on the cell axis Xc.) This can be achieved as shown by dispensing the capsule components 21, 22 from their bulk packages 4001, 4002 into the capsule cap and body chambers 4021, 4011, and arranging the stacked drug packages 2301 in-between the capsule body handling mechanism 4010 and the capsule cap handling mechanism 4020 so that the capsule body and cap chambers 4011, 4021 containing the respective capsule body 22 and cap 21 are aligned with the cells 311 of the drug packages 2301, as shown in FIG. 222. As indicated by the arrow in FIG. 218, relative axial movement may be provided between the capsule body handling mechanism 4010 and the capsule cap handling mechanism 4020 to accommodate the stacked drug packages 2301 and maintain the stack in compression during the capsule assembly operation. The first and second drug particles 3 are then combined together within the axially aligned cells 311 of the drug packages 2301.

The drug packages may be formed as described in the earlier, first and variant first embodiments; for example, as drug packages closed by foil, e.g. drug packages 301 or 1301, or with movable closure elements such as drug package 2301 as illustrated. In the illustrated example, first and second drug packages 2301 are formed in the same way as drug package 2301 described earlier, and the machine 4000 is configured to move the movable closure elements or foraminous plates 2401 to the open position before each capsule body 22 is advanced by a respective pushrod 4030 through the aligned cells 311, collecting within it the drug particles 3 from each of the drug packages 2301 (FIG. 223).

As best seen in FIG. 221, the capsule cap handling mechanism 4020 may include a cap chamber 4021 separated by a shutter 4023 from a capsule transfer chamber 4022. The cap chamber 4021 may have a reduced diameter aperture defined by a radially inwardly extending retaining collar 4026 which retains the cap 21 in the cap chamber 4021 during assembly of the capsule.

Each cap 21 may be dispensed from the bulk cap package 4001 into the cap chamber 4021, for example, via a filling aperture 4025 closed by another shutter 4024. This could be achieved, for example, by opening the shutters 4023, 4024 and passing an insertion rod (not shown) via the collar 4026, through the cap and transfer chambers 4021, 4022, then positioning the cap 21 on the rounded distal end of the insertion rod (not shown) and retaining it there by another, confronting, collinear, smaller diameter retaining rod (not shown). The rods and cap are moved axially simultaneously to position the cap in the cap chamber 4021. Then the retaining rod is withdrawn via the filling aperture 4025 and the shutter 4024 is closed before withdrawing the insertion rod through the collar 4026. Alternatively, the insertion rod could retain the cap 21 by suction so that no retaining rod is needed. Those skilled in the art will appreciate that there are many alternative ways to dispense the capsule components from the bulk packages 4001, 4002 and position them in alignment with the cells 311 of the drug packages 2301, and the capsule assembly mechanism can be adapted accordingly. It is possible also that the capsule could be closed in a position not aligned axially with the cells 311 of the drug packages 2301, in which case the cap 21 need not be aligned on the cell axis Xc as shown.

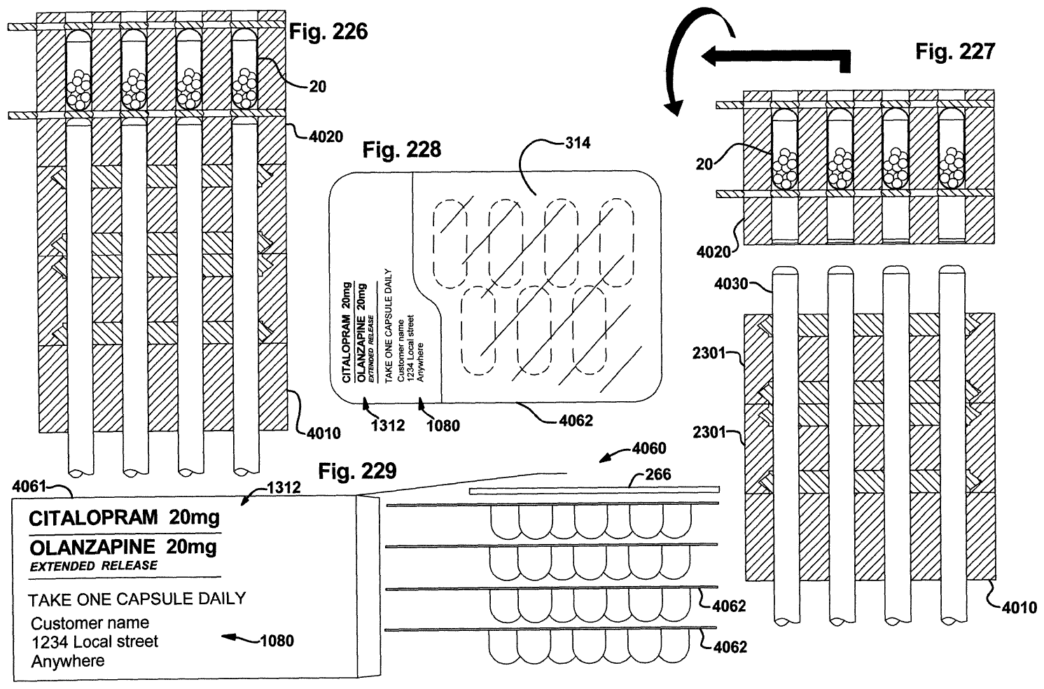

Returning to the assembly steps as illustrated, FIG. 224 shows how the capsule body 22 may enter telescopically into the cap 21 via the collar 4026. The cap may be restrained as shown by the closed shutter 4023 while the capsule is closed. Then, as shown in FIG. 225, the shutter 4023 may be opened, and the pushrod 4030 advanced to move the filled capsule 20 into the capsule transfer chamber 4022. The edges of the apertures in the shutter 4023 may be chamfered or rounded, as may be the distal end of the pushrod 4030, so that the shutter 4023 can then be closed between the capsule 20 and the pushrod 4030 without damaging the capsule, to urge the capsule 20 to enter fully into the transfer chamber 4022 and then retain it therein, as shown in FIG. 226.

The assembly machine 4000 may then be operated to package the plurality of polypills 20 in individual cells 4064 of an end user package, which as illustrated may be a blister pack 4062. Each cell 4064 sealingly encloses a respective one of the polypills 20 and is openable by an end user to remove the polypill 20 from the cell. Several such blister packs 4062 may be filled with the capsules produced from one set of drug packages 2301, thus forming end user sub-packages which can be assembled together, e.g. in a box 4061 to form the end user package 4060. A patient information leaflet 266 may be printed as previously described and included in the box 4061.

As shown in FIG. 227, the finished capsules 20 may then be moved to a packaging station 4050 of the machine 4000. This can be achieved, for example, by detaching the capsule cap handling mechanism 4020 from the capsule body handling mechanism 4010 and then moving it, e.g. in rotation and translation as shown, to the packaging station 4050. The packaging station 4050 may be configured to fill and seal blister packs 4062, and may be supplied in bulk with pre-formed, generally conventional blister pack trays 4063 (FIG. 219) and foil 314 (e.g. a plastics film, metal foil, metallised film or the like) for sealing the trays.

As shown in FIG. 230, each of the capsule transfer chambers 4022 may be arranged to communicate with an individual cell 4064 of a blister pack tray 4063. A capsule release shutter 4027 may then be opened to release each capsule from the transfer chamber 4022 into the respective cell 4064 (FIG. 220), optionally by means of a capsule ejection mechanism (not shown), before closing the cells with the foil 314, e.g. by heat, pressure, ultrasonic welding, adhesive or other techniques as well known in the art. Each cell 4064 is openable by the end user to remove the single polypill 20 contained therein by pressing the polypill 20 through the foil 314, as well known in the art. The blister pack 4062 may present a surface on which the package label can be printed, as shown in FIG. 228.

As with the previously described embodiment, the machine may include an array of pushrods 4030 corresponding to the array of cells in the drug package (e.g. 48 pushrods, or 48 pairs of pushrods, for a 48 cell package). The capsule assembly mechanism may be configured with separately movable machine parts (e.g. separately movable capsule cap handling mechanisms 4020) for each row of cells, so that each row of capsules 20 can be formed by axial movement through the cells 311 of the drug packages 2301, and then transferred laterally out of the respective machine part (e.g. transfer chamber 4022) into the respective cell 4064 of the blister pack 4062 or other end user package. For simplicity, only one such separately movable machine part 4020 is illustrated.

In alternative arrangements, not shown, the machine may include a pair of opposed pushrods for each capsule 20, wherein one or each pushrod defines a respective cavity to receive a part or the whole of the respective capsule body 22 or cap 21. The cavity for the capsule body 22 may be shaped similarly to that of the carrier 320 or 1320, defining an annular space which receives the cap 21 during assembly. The cap 21 and body 22 may thus be closed together within the aligned cells 311 of the drug packages. The closed capsule or polypill 20 containing the combined particles 3 forming a single dose of each of the first and second drugs 1, 2 may then be transferred to a packaging station 4050. The transferring step may include moving the capsule 20 out of the aligned cells 311 of the packages by the movement of one of the pushrods, or the simultaneous movement of both of the pushrods, in the axial direction of the cells 311, optionally followed by another movement away from the cell axis, e.g. in rotation and translation as in the illustrated embodiment.

Bulk Drug Packages and Bulk Capsule Components

FIG. 230 illustrates a further alternative variant in which the drug particles 3 are loaded into the assembly machine 4000 (FIG. 218) in first and second bulk drug packages 4071, 4072, each of which contains a large quantity of the relatively large, preferably spheroidal drug particles 3 of equal size and shape. The particles 3 in the first bulk drug package 4071 contain the first drug 1 but not the second drug 2, whereas the particles in the second bulk drug package 4072 contain the second drug 2 but not the first drug 1.

Of course, the machine 4000 may be arranged to receive more than two bulk drug packages, or even just one bulk drug package, for any given set of polypills 20. (As previously discussed, using the system to make capsules containing just a single drug may be helpful to obtain a customised dosage of the drug, where different drug packages contain particles with different dosages, or to reduce stockholding by supplying less commonly prescribed, single drugs in the same packaging as the polypills.)

In the illustrated example, the capsule cap handling mechanism 4020 and packaging station 4050 are arranged as described in the previous example to close the capsules and package them in blister packages 4062, but the machine 4000 does not have a window 4004 for receiving cellular drug packages 2301. Instead, the capsule body handling mechanism 4010 is adapted to cooperate with a dosing apparatus 4070, which may comprise two filling apparatuses 2430 as previously described and shown in FIGS. 160-163. Other dosing or filling mechanisms could be used as known in the art.

As illustrated, the capsule body handling mechanism 4010 may be configured as a carousel (e.g. a rotating disc) which moves in turn through a series of stations at which consecutive operations A-H are performed. Of course, many alternative arrangements are possible as well known in the art of filling capsules.

At stage A, each capsule body 22 may be dispensed from the capsule body package 4002 into the capsule body chamber 4011 to receive the drug particles 3, as shown. In an alternative arrangement (not shown) the drug particles 3 may be dispensed into the capsule body chamber 11 which acts as a dosing chamber, and afterwards transferred to the capsule body 22, e.g. by inserting the capsule body 22 into the dosing chamber to collect the drug particles 3 present therein.

At stage B, the first filling apparatus 2430 fills a single dose consisting of five particles 3 of the first drug 1 from the first bulk drug container 4071, then the capsule body chamber 4011 moves to the next filling position C in which the next filling apparatus 2430 fills it with another six particles of the second drug 2 from the second bulk drug container 4072, all of said particles 3 being of equal size and shape. Alternatively, as also described above with reference to the filling apparatus 2430, the dosing apparatus 4070 may be operated for a defined number of operations to introduce one or more particles 3 on each operation, until the defined number of particles 3 is received in the dosing chamber 4011 or capsule body 22.

The capsule is then closed as previously described in stages D-G, and at stage H moves to the packaging station 4050 where it is packaged in the blister package 4062.

Spheroidal particles 3 are particularly preferred since their packing density, and their mechanical interaction with the assembly apparatus, is not affected by their spatial orientation. Moreover, a rounded surface allows each particle to move rollingly through the assembly apparatus, for example, when transferring from the filling chute 2434 into the dosing chute 2436 of the above described filling apparatus 2430 as shown in FIGS. 160-163. The rounded surface avoids damage by entrapment between machine parts. For example, it allows the solid upper face of the dosing body to lift the column of spheroids 3 remaining in the filling chute 2434 through a small distance of less than half a particle diameter, representing the tolerance in the length of the dosing chute 2436 for variations in particle diameter, as the dosing body 2435 moves towards the dosing position. This is evident from the position of the lowest particle 3 visible in FIG. 160, which extends slightly below the upper end of the dosing body 2435. A rounded lower edge (not shown) of the filing chute 2434 can gently engage the lowest particle to assist this slight upward movement of the column.

### Machine for Producing Polypills in the Form of Pastilles—Overview

Byway of example, the second embodiment provides an assembly apparatus in the form of a second machine 500, 5000 for use in a pharmacy to assemble together a plurality of second packages 701, 702, 703, 704, 705, 7001, 7002, 7003, 7004, 7005. Each of the second packages includes a frame 710 defining a plurality of cells 711, which are separated by the frame to form a spaced array.

The second and variant second machines 500, 5000 include an alignment structure for guiding the plurality of packages in a stacked configuration with each of the cells 711 of each package in axial alignment with a corresponding one of the cells 711 of each other package of the plurality of packages 701, 702, 703, 704, 705, 7001, 7002, 7003, 7004, 7005.

In this specification, a stacked configuration includes a stack in any orientation, irrespective of whether formed along a vertical or horizontal or inclined axis, and irrespective of whether the packages of the stack are spaced apart in superposed, parallel relation, or pressed together in superposed, parallel, abutting relation.

The second and variant second machines 500, 5000 further include a compression mechanism for compressing together the frames 710 of the plurality of packages in the stacked configuration to form an assembly defining an end user package 700, 7000 (FIG. 80).

In the illustrated embodiments, the compression mechanism includes a pair of opposed pressure plates 521 which are urged together by actuators 523 to compress the stacked packages between their opposed compression surfaces 522. Although the opposed compression surfaces 522 of the pressure plates 521 are illustrated as flat in the first machine of 500, they may be contoured, e.g. as shown in the variant second machine 5000, to correspond to the different thicknesses of different portions of the end user package 700, 7000, e.g. including protruding portions 522′ to engage the respective parts of the composite label assembly 712 as shown, and/or to apply pressure to the frames 710 of the stacked packages without applying pressure (or while applying relatively less pressure) to the cells 711.

The alignment structure may include alignment surfaces 550 that engage an outer profile of the packages in an assembly position. As exemplified by the second machine 500, such alignment surfaces may include upper and lower alignment surfaces 550 (FIG. 76) and front and rear alignment surfaces 550 (FIG. 75) which constrain the packages in an aligned, horizontally stacked configuration in the assembly position, as shown in FIGS. 75 and 76, before and during operation of the compression mechanism to produce the end user package 700.

Alternatively or additionally, the alignment structure may include alignment elements that engage the packages to maintain the packages in parallel relation. In use, the machine may be configured to slide the packages together along the alignment elements, optionally by operation of the compression mechanism. The alignment elements may form part of a package transfer assembly that is operable to move the packages from an initial, receiving position to an assembly position in which the packages are compressed together by the compression mechanism. The package transfer may be further operable to move the end user package, formed by compressing the packages together, from the assembly position to a delivery position, e.g. a delivery window 507 as illustrated in FIGS. 88 and 90.

The alignment elements could engage correspondingly profiled portions of the packages, e.g. at the edges of the packages or in apertures formed in the packages.

Such alignment elements could engage, for example, recesses or other profile features at an outer edge of the packages, so that they can engage and release the packages by moving inwardly or outwardly in a direction of the plane of the major, front and rear surfaces of the packages.

Alternatively, such alignment elements could engage the packages by gripping the packages, e.g. by means of opposed gripping portions that act on opposite parts of the major, front and rear surfaces of the packages, e.g. in a similar way to the grippers 553 of the alignment mechanism of the variant second machine 5000 as further described below.

Alternatively, such alignment elements could engage in apertures formed in the packages, so that they can engage and release the packages by moving axially through the packages in a direction normal to the plane of the major, front and rear surfaces of the packages.

This latter arrangement is exemplified by the variant second machine 5000 which provides a plurality of (e.g. three, as shown) alignment elements in the form of package locator rods 551 which are axially movable to engage in a pattern of locator rod apertures 751 in each of the packages 7001, 7002, 7003, 7004, 7005.

The package locator rods 551 may be mounted as shown to a package transfer actuator body 552 to form a package transfer assembly, which is movable from the position of FIG. 96 to the position of FIG. 97 to transfer the packages 7001, 7002, 7003, 7004, 7005 from the receiving slots 501, 502, 503, 504, 505 to the assembly position in which the stacked packages are located in-between the compression surfaces 522. The packages can slide along the locator rods 551 as they are compressed together to form the end user package 7000.

In another possible arrangement (not shown), where the packages are arranged to slide along the package locator rods or other alignment elements, the package locator rods or other alignment elements could be arranged to move together with the compression mechanism when removing the packages from the receiving slots. For example, the package locator rods could be advanced through holes in the pressure plates to engage in the locator rod apertures 751 in the stacked packages, before moving the pressure plates and locator rods away from the receiving position, to extract the packages from their temporary covers which remain in the receiving slots of the machine, as shown. Once the packages and locator rods are in the assembly position, clear of the receiving slots, the compression mechanism may be activated to compress the stacked packages to form the end user package. Then, the locator rods and pressure plates may be moved again to locate the end user package at a delivery window 507 of the machine, before retracting the locator rods and separating the pressure plates to allow removal of the end user package.

The locator rods may be arranged as shown to maintain the packages in parallel relation. In the variant second machine as illustrated there are three locator rods 551 which are spaced apart in two orthogonal dimensions (the height and width dimensions of the packages). Since the locator rods 551 are a sliding fit in the locator rod apertures 751, the packages are constrained by the locator rods to remain in parallel relation with their height and width dimensions normal to the locator rods.

Optionally as shown, in order to further constrain the packages in normal relation to the locator rods, an additional alignment mechanism can be employed, e.g. with grippers 553 mounted on an alignment body 554, to move the packages closer together along the alignment elements before compression begins; alternatively, the alignment mechanism 553, 554 need not be provided, and the packages could remain at their initial spacing as received in the machine until they are urged together by the compression plates 521.

The assembly apparatus 500, 5000 may include a plurality of elongate slots 501, 502, 503, 504, 505, each slot being configured to receive a respective one of the packages 701, 702, 704, 704, 705, 7001, 7002, 7003, 7004, 7005. Each slot may include a shape profile, e.g. as shown in FIG. 77, configured to selectively fit or obstruct a corresponding shape profile of a respective one of the packages introduced into the slot. Different ones of the slots may have different shape profiles.

The abutment surfaces defined by the slots or other package receiving portions of the machine may be configured, e.g. as shown, to engage and retain the temporary covers 706, 707 of each package when the frame 710 of the package is detached from the temporary covers and moved to the assembly position. The temporary covers 706, 707 remaining in the slots can then be removed by the user for disposal before the next assembly operation.

In the illustrated embodiment, the slots 501, 502, 503, 504, 505 are identified by indicia, with slots 504, 505 marked [A] and [B] respectively being configured to receive the front and rear cover packages 704, 705 respectively, and slots 501, 502, 503 marked [1], [2] and [3] respectively being configured to receive drug packages, wherein the first drug package 701 is inserted into slot [1] (501), the second drug package 702 (if present) is inserted into slot [2] (502), and the third drug package 703 (if present) is inserted into slot [3] (503).

It should be understood that any of the drug packages 701, 702, 703 can be introduced into any of the three drug package slots 501, 502, 503; the point is that if there is only one drug package then it should be in slot 501, and if there are only two drug packages then they should be in slots 501 and 502. The order of priority of the slots ensures that the composite label assembly 712 is correctly configured (because different slots have differently positioned label cutters), while the different shape profiles of the slots are arranged to prevent each package type from being introduced into the wrong slot, as further explained below.

In order to ensure that the user does not introduce packages into the third, or second and third slots 502, 503 while leaving the first, or first and second slots 501, 502 empty, the machine 500, 5000 may be configured to obstruct access to the second and third slots until the first slot is filled, and to obstruct access to the third slot until the second slot is filled. This could be done for example by advancing the respective punches 519 to an obstructing position. Alternatively or additionally, the scanners 561 or other sensors could be used to sense the presence of the packages and send a signal to the controller 262 to prevent operation of the machine if the slots are not correctly occupied. Lights could be used to indicate which slot should be filled next.

The alignment structure can be any convenient arrangement for maintaining the packages in alignment while the frames are pressed together. For example, the packages could be aligned using rods or other structures that engage corresponding shaped portions of the packages while the packages slide towards each other along the alignment structures, e.g. by operation of the compression mechanism, as exemplified by the variant second machine 5000.

Instead of moving all the packages simultaneously from an initial position into the assembly position as shown, the machine could be arranged to move the packages one by one into a stacked configuration, perhaps urging them together to adhere to one another as each package is added to the stack. Alternatively, the packages could be placed in a stacked configuration by the user, instead of moving them automatically from an initial receiving position as shown. The packages could be stacked vertically (i.e. one on top of another) instead of horizontally (i.e. in a row next to one another) as shown, either by the user (one by one) or by operation of the machine (simultaneously or one by one). For example, the package receiving portion of the machine could be arranged at 90° to the illustrated configuration so that the slots extend horizontally and are spaced apart to form a vertically rather than horizontally stacked configuration, one above another. The packages could then be moved, simultaneously or one by one, by the package transfer assembly so that each package rests on top of the one below in a vertical stack, either during or before operation of the compression mechanism to press them together.

The machine 100, 500, 5000 may include a vacuum generating apparatus which is arranged to create a vacuum, (which is to say, a full or partial vacuum), wherein the machine is arranged to compress together the frames 310, 710 of the plurality of packages within the vacuum.

In the illustrated embodiment, each of the second and variant second machines 500, 5000 includes a vacuum generating apparatus 590 (shown only in FIG. 60) including a vacuum pump 591 which evacuates a reservoir 592. A valve (not shown) is operated by a controller 262 of the machine, after closing the door 530 to seal the interior space within the machine, to connect the interior space within the machine to the reservoir 592. The evacuated volume of the reservoir 592 rapidly depressurises the machine 500, 5000. This speeds up the assembly procedure and may also help in detaching the temporary covers 706, 707 as described below, while the vacuum pump 591 starts up again to evacuate the reservoir 592 ready for the next operation.

The machine 500, 5000 may be arranged, before compressing together the frames 710 of the plurality of packages, to detach and separate the frame 710 of each of two or more of the packages from a cover or covers 706, 707 removably connected to the frame 710. For ease of reference these covers 706, 707 are also referred to herein as temporary covers to distinguish them from the front and rear cover packages 704, 705, which form the front and rear covers of the end user package and which may also include temporary covers that are removed before assembly.

As exemplified by the illustrated embodiment, the frame 710 of each package may be coated with a tacky adhesive 4. Each of the front and rear cover packages 704, 705 may be coated only on the inwardly facing side of its frame 710, while each of the drug packages 701, 702, 703 may be coated on both of the two oppositely facing sides of its frame 710, as shown.

As previously mentioned, in order to protect the adhesive surfaces 4 and API films 31 until the moment of assembly, the frame 710 of each drug package 701, 702, 703 may be enclosed between two removable, temporary covers 706, 707, each of which is arranged to protect a respective one of the two oppositely facing sides of the frame 710. The sticky, inwardly facing side of the frame 710 of the front cover package 704 is protected by a removable cover 707 (FIG. 69), while the sticky, inwardly facing side of the frame 710 of the rear cover package 705 is protected by a removable cover 707 (FIG. 68).

The removable covers 706, 707 are separated from the frame 710 of the respective package after closing the door 530 of the machine 500, 5000 by operation of a punch mechanism 519, assisted by the rapid depressurisation of the machine 500, 5000, as further explained below.

### Further Features of the Second Machine

Referring to FIG. 60, the second machine 500 has a casing 506 with a door 530 to form a sealable enclosure that can be evacuated by the vacuum generating apparatus 590. Within the enclosure are defined the package receiving slots and a package delivery window 507 from which the user can collect the finished end user package 700, 7000. A finger recess 508 may be provided to expose a part of each package to be gripped by the user's fingers while the package is inserted into, or the temporary covers are removed from, the respective slot.

The second machine may be configured to prevent each package type from being introduced into the wrong slot.

Byway of example, this may be achieved by arranging for the shape profiles of different ones of the slots to vary in two, mutually orthogonal dimensions D1, D2 of the slot, which limit corresponding dimensions of the packages by abutment between internal surfaces of the slot and the external surfaces of the package. A sequence may be arbitrarily defined for the slots fitting different package types—e.g. front cover package slot 504 may be first in the sequence, then drug package slots 501, 502, 503 may be second in the sequence, and rear cover package slot 505 may be third in the sequence. Dimension D1 is then arranged to increase progressively for each slot type in the sequence, while dimension D2 decreases progressively for each slot type in the same sequence. This ensures that one of the two dimensions acts as a no-go feature if the wrong package type is introduced into any of the slots.

This arrangement is illustrated in FIG. 77 with reference to the second machine 500, wherein the go/no-go abutment surfaces of each slot are arranged to engage the varying profiles of the different front and/or rear, temporary covers 706, 707 of each package. Alternatively or additionally, the frame 710 of the package could define the go/no-go abutment surfaces.

Alternatively or additionally, the controller 262 could be arranged to read the package indicia 717 during assembly, via the reader 561 of each slot, and to interrupt the assembly procedure and/or generate an error message (e.g. via screen 267) if the wrong package type is detected in any of the slots, or if less than a full complement of drug packages are present but the wrong slots are occupied.

In use, the user selects the required combination of packages and inserts each package into the correct slot. The front and rear cover packages 704, 705 or 7004, 7005 are inserted respectively into slot 504 (marked [A]) and slot 505 (marked [B]), while the drug packages are inserted into the drug package slots in their numerical order, i.e. slot 501 first, then slot 502, then slot 503, and then the next if further drug package slots (not shown) are provided. Then, if the screen 267 indicates that the machine 500, 5000 is ready, the user closes the door 530 which is locked by controller 262 before starting the assembly operation, commencing with evacuation of the casing 506. The door 530 could be configured to press against the exposed ends of the packages to ensure they are fully inserted into the slots.

The package authentication procedure via the remote computer and database 91, 90 may be carried out generally as described with reference to the first machine 100, except that the package data are read by controller 262 of the second or variant second machine 500, 5000 individually, optionally simultaneously, from each package via the respective reader 561, 561′ associated with the slot in which that package is received (FIG. 72). As with the first machine 100 and the variant first machine, the package data may be read both before and after the door is closed; reading the package data before the door is closed allows the user to identify and correct any error, while reading the package data after the door is closed allows a high level of confidence to be placed in that data when used for the authentication procedure, since the data is collected during a one-time operation after which each package is irreversibly changed and cannot be used again. Although not illustrated, each machine 100, 500, 5000 may be configured to identify and reject a previously used package, e.g. by sensing the altered feature, such as a punched-out region of the package.

The controller 262 may then activate punches 519 and label cutters 510, 511 to separate each package from its removable covers and to remove the respective label portions so that the cut regions will reveal the corresponding label portions of the rearwardly positioned packages in the assembled stack (FIG. 73).

Each machine may include a package transfer assembly, having an actuator controlled by the controller 262, for moving the packages from the receiving position (e.g. slots, as shown) to the assembly position in which they will be pressed together by the pressure plates 521. Removing the packages from the receiving position may also accomplish the step of removing the packages from their temporary covers, which may be left behind in the receiving position to be removed by the user after opening the door to collect the end user package 700, 7000 at the end of the assembly process.

Where the packages are received respectively in slots 501, 502, 503, 504, 505 or other positioning features at which an initial operation is performed (e.g. reading indicia from the packages, cutting parts of the packages, and/or removing temporary covers from the packages), the slots or other positioning features may be spaced apart by a sufficient distance to accommodate the cutters 510, 511, punches 519, scanners 561, grippers or other functional parts of the machine associated with the slot, which conveniently may be positioned in-between the slots.

The packages may then be moved to the assembly position in which they are urged together by the compression plates 521.

FIGS. 74 and 78 show the package transfer arrangement of the second machine 500, comprising grippers 553 which are mounted on an alignment mechanism body 554.

The grippers 553 engage the package frames 710 before the alignment mechanism body 554 is moved from the receiving position to the assembly position (FIGS. 75 and 79). The grippers are then released and removed with the body 554 leaving the packages between alignment surfaces 550 (FIGS. 75 and 76) before it is dropped or otherwise urged into the package delivery window 507.

The variant second machine 5000 illustrates an alternative arrangement in which package locator rods 551 are used, both to move the packages to the assembly position, and also as an alignment structure to maintain the packages in parallel relation while they are pressed together from their initial, spaced-apart arrangement by the compression plates 521.

As best seen in FIG. 91, the package locator rods 551 can be arranged to engage the package frame 710 without engaging the temporary covers 706, 707 during movement of the package locator rods 551 away from the receiving position into the assembly position.

FIG. 92 shows how the package locator rods 551 (shown in cross-section in FIG. 91) are mounted on an actuator body 552 which is operable by the controller 262 to slide the rods 551 axially to insert them through all the packages in their initial position in the slots of the machine 5000. After inserting the rods 551 through the apertures 751 of the packages, the controller 262 commands the actuator body 552 to move to the assembly position, withdrawing all the package frames 710 from between their temporary covers 7006, 7007 which remain in the slots, as shown in FIG. 92. For clarity, only one package is shown in FIG. 92, but it will be understood that the rods 551 pass through all of the packages which are held in parallel relation and moved simultaneously in this way.

As shown in FIG. 97 (in which, for clarity, the packages are present but not shown), the actuator body 552 moves the packages between the compression plates 521 which are then actuated to compress the stacked package frames 710 together as shown in FIGS. 98-99. The adhesive facing surfaces of the packages stick together so that the frames are combined to form the end user package 7000.

It will be understood that after closing the door 530 and evacuating the casing 506, all of the foregoing steps may be performed in a partial vacuum, so that the cells 711 of the end user package 7000 may be hermetically sealed by the mutual adhesion of the frames 710 with their internal space at a pressure below ambient. If gas is present then it may be dried or otherwise modified, e.g. to be mostly inert to better preserve the drugs 1, 2.

The controller 262 may then command the actuator body 552 to move to deliver the finished end user package 7000 to the delivery window 507 (FIG. 100) before withdrawing the rods 551 axially from the end user package (FIG. 101). The controller 262 then opens a valve (not shown) to admit air (or dried air or inert gas) into the casing 506 before releasing the door lock (not shown) so that the user can remove the end user package 7000 from the delivery window.

If desired, a similar arrangement to the grippers 553 and alignment mechanism body 554 could be used to move the packages closer together along the package locator rods 551 before operation of the compression mechanism of the variant second machine 5000. Although optional, this additional arrangement is illustrated in FIGS. 91-97, which show how the grippers 553 are actuated (FIGS. 94-95) by the alignment mechanism body 554 to grip each package before the actuator body 552 withdraws the package from the slot (FIG. 93). Then, the grippers 553 are moved together to reduce the spacing between the packages (FIG. 96) while maintaining the packages in parallel relation before the actuator body 552 moves to position the packages between the pressure plates 521 (FIG. 97).

Although only one package size is illustrated, the second machine 500, 5000 may be configured to receive different sized packages (e.g. 12, 24, 36 or 48 cells). For example, the slots could be adjustable (e.g. by moving a machine component forming their lower boundary) to accommodate different sized packages. The controller 262 could be configured to permit operation only subject to the scanners 561 detecting package indicia 713, 717 indicating the correct package size for the selected slot size, and/or the different sized packages could be provided with different shape profiles that interact with a corresponding shape profile of the slot (which may be an adjustable shape profile) to prevent insertion of a package of an incorrect size.

Byway of example, the illustrated second machine 500 includes additional scanners and adjustable shape profile blocks 561′ at the rear of the slot, operable respectively to sense, and to selectively permit or obstruct, corresponding, additional shape profile features 752 of the packages.

### Further Features of the Second Packages

As exemplified by the second embodiment, each drug package may comprise a frame 710 made from a sheet or two or more sheets laminated together, wherein apertures are formed in the sheet or sheets to define the spaced array of cells 711. The or each sheet may be made from cardboard, although of course plastics or other materials could alternatively be used.

As illustrated for example in FIG. 61, the carrier film 34 or other structure of the edible wall 32 of each cell 711 is exposed at the aperture forming the cell 711 and connected to the frame 710. The carrier film 34 or other structure forming the edible wall 32 of the cell 711 of the drug package may be provided with perforations 35 or otherwise weakened, to allow its central region to be more easily detached from the frame 710 to remove the finished polypill 30 from the end user package 700, 7000.

FIG. 81 shows one possible structure for the frame 710 of each drug package, which may be formed from two sheets 36 of cardboard laminated together by adhesive. Apertures are formed in both sheets but of slightly different diameters, so that the lower sheet forms a supporting structure upon which the carrier film 34 is attached, for example, by adhesive. The respective drug 1 or 2 is incorporated into the API film 31 which is applied to the carrier film 34, either before or after attaching the carrier film 34 to the frame 710. The API film 31 could be extruded and/or rolled to a predefined thickness and then punched to form discs, or extruded as a round bar and cut into slices, before the discs or slices are attached to the carrier film 34. Alternatively for example, the edible wall 32 could comprise two sheets of rice paper or other edible material which are attached to the frame 710 to enclose the drug in-between them, e.g. in particulate or pulverulent form. Or, the drug 1 or 2 could be impregnated into the edible wall 32 formed from rice paper or other suitable carrier material. In each case the or each edible component may include an edible adhesive on both sides.

After the package comprising the frame 710 and the edible wall 32 of each cell 711 has been formed, the tacky adhesive surfaces 4 of the frame 710 (and the adhesive surfaces of the edible walls 32) are protected by the temporary covers 706, 707, which may be sheets of cardboard with a release coating 5 on their inwardly facing surfaces, except in regions that form the attachment points 719. The release coating is absent from these regions so that the tacky adhesive sticks the temporary covers 706, 707 to the frame 710, which is released during assembly by the punches 519 which punch out these regions of the temporary covers 706, 707 and frame 710.

Other arrangements could be used to attach the temporary covers to the frame; for example, the temporary covers could be attached in regions proximate the edges of the frame 710 which are cut away during assembly.

FIG. 70 shows another possible arrangement, in which the temporary covers 706, 707 are separated from the frame 710 of the drug package by separators 720, which may be formed as strips that extend around the margins of the frame 710. The separators could be cut away during assembly, or removed as part of the temporary covers 706, 707, which are separated from the frame 710 by cutting away respective attachment portions connecting together the temporary covers 706, 707 and the frame 710, or just by pulling the frame 710 out of an enclosure formed by the covers 706, 707. The air- (or inert gas-) filled space surrounding the frame may assist in forcing the covers and separators (coated with release coating 5) away from the frame 710 as the air or other gas expands responsive to depressurisation of the machine.

FIG. 71 illustrates an alternative arrangement whereby the frame 710 of each package is formed, rather than in the form of a flat sheet as in the other illustrated embodiments, to define channels 721, which may be generally V-shaped or U-shaped as shown, and which may surround all of the cells and/or each of the cells 711. Such frames could be moulded from plastics material (e.g. polylactic acid) or could be pressed, e.g. from cardboard. The channels nest together to facilitate alignment of the frames 710 of the packages as they are pressed together during assembly. An adhesive 4 may be arranged in each channel 721 so that it is protected from accidental contact until assembly, when the base of the corresponding channel 721 of the adjacent frame 710 contacts the adhesive 4 to bond the frames of the packages together into the end user package.

FIG. 82 shows one cell of a cover package in cross-section, which may be formed similarly to the drug packages from one or two flat sheets 36 of cardboard or other material which are punched or otherwise formed to define apertures. The apertures are aligned but may have different diameters as shown, to form an attachment surface to which the outer margin of the edible wall 33 is attached, e.g. by adhesive. Perforations 35 or other weakening features may be arranged as shown to facilitate detachment of its central region together with the rest of the polypill of which it will form a part.

The foil wall 714 of the cover package 704, 705, 7004, 7005 may form part of a single sheet of foil 714 which is laminated to the cardboard or other sheets of the frame 710, or could be applied as individual pieces of foil 714 to each cell. Each foil wall 714 may be shaped to facilitate its movement along the cell axis Xc while remaining intact and sealingly attached to the frame 710; the shape may be accommodated in the thickness of the frame 710 to protect the foil wall 714 from damage in storage and may comprise a region in which the foil wall 714 extends in the direction of the cell axis Xc, for example, in the form of an annular fold 715, as shown.

Each temporary cover 706, 707, 7006, 7007 may extend upwardly and downwardly as shown beyond the upper and lower edges of the frame 710 of the respective drug or cover package, so as to engage abutment surfaces of the slot to retain the temporary cover in the slot when the frame 710 is withdrawn during assembly. These upwardly and downwardly extending portions may define go/no-go features to ensure that the packages cannot be inserted into the wrong slots, as further discussed herein.

The frame 710 of the front cover package 7004 could incorporate a window 721 (FIG. 89) through which is revealed the label portion 713′ of the uppermost drug package in the assembly. The window 721 allows a more robust construction of the frame 710 of the front cover package when its trailing end extends upwards to provide a suitable location for a locator rod aperture 751, which is located proximate its trailing edge and above the upper edges of the drug packages. In this way the package locator rods 551 can be spaced apart both vertically and horizontally to better maintain the packages in parallel relation, without being obstructed by the temporary covers. At the same time, the temporary cover of the front cover package 7004 can extend upwardly at its trailing edge, similarly to the temporary cover 7006 of the drug package 7002 as shown in FIG. 91, to engage an abutment surface of the slot above the upper edge of the frame 710 of the respective package, so that the frame 710 can be withdrawn by the rods 551 while the temporary cover is retained in the slot.

FIGS. 83-85 show an alternative arrangement in which the cardboard or other sheets 36 forming the frame 710 of each drug package are shaped to form a cell 711 (FIG. 83) to which a carrier film 34 may be applied, e.g. as a liquid that fills the recesses formed by the cell wall at the margin of the cell, or by pressing it into the recesses, after which the carrier film may be punched to form perforations 35 (FIG. 84). The API film 31 can then be applied in a liquid state as a drop of defined volume to the surface of the carrier film 34 (FIG. 85).

In a further alternative arrangement (not shown), the carrier film 34 of each drug package could be formed as a unitary sheet which extends over a sheet of the frame 710, or between two sheets of the frame (wherein the or each sheet of the frame may be for example a cardboard sheet 36) to form a laminated assembly, wherein a respective portion of the unitary sheet of carrier film is exposed at each of the apertures or cells 711. For example, the carrier sheet can be laminated in-between two cardboard sheets 36 that define apertures forming the cells 711. This provides a simple assembly procedure since the apertures can be formed in the cardboard sheet or sheets of the frame before laminating it or them together with the carrier sheet, such that a portion of the carrier sheet remains a permanent part of the frame after the respective portions of the carrier sheet are removed together with the polypills of which they form a part. As in the illustrated arrangement, the carrier sheet may be perforated, thinned or otherwise locally weakened proximate a margin of each of the cells to assist in detaching the finished polypill. After forming the frame 710 including the carrier film 34 as a unitary sheet laminated to the frame, the API film 31 could be applied locally to each cell 711, e.g. as a disc as shown in FIG. 81, or as a drop of liquid as shown in FIG. 85.

A similar arrangement (not shown) could be adopted for the cover packages, by forming each disc or wafer as a respective portion of a unitary sheet of edible material (e.g. rice paper) which is laminated to the frame 710, e.g. between two sheets of cardboard 36, so that the portion forming each edible wall 33 is exposed at the aperture or apertures forming the respective one of the cells 711, while the remainder of the edible sheet forms a permanent layer of the frame 710. Again, perforations 35 or other weakening features may be arranged around the portion forming each edible wall 33 to facilitate removal of the polypill.

As shown in FIGS. 61 and 81, the edible wall 32 comprising the API film 31 and carrier film 34 may extend across the cell 711 of each drug package so that, after removal of the temporary covers 706, 707, it is exposed on either side of the frame 710. As shown in FIGS. 68 and 69 and FIG. 82, the edible wall 33 (e.g. rice paper disc or wafer) of each cell 711 of the front or rear cover package 704, 705, 7004, 7005 may be exposed on one side of the frame 710 while on the other side of the frame it is covered by the foil wall 714 of the cell 711. The surfaces of the package frames 710 that will be pressed together are coated with a tacky adhesive 4 so that they adhere together to form the composite frame 710 of the end user package 700, 7000. The edible walls 32 are also configured to adhere together, and to the edible walls 33 that will form the opposite sides of the pastille 30. For this purpose the edible walls 33 may be adhesive on that side that faces away from the foil wall 714 of the cell, but not adhesive on the side that faces the foil wall 714, so that the pastille 30 can be removed and handled easily.

The tacky adhesive coating 4 of the frames 710 can be any suitable tacky adhesive as known in the art. It could be for example a pressure sensitive adhesive with an extended open time or permanent tack, as well known in the art and as used in glue traps for vermin and various industrial and construction applications.

In order to adhere readily to the edible wall 32 of the drug package, the edible wall 33 of the cover package may be coated on one side with an adhesive layer, e.g. the same material that forms the API film 31 or carrier film 34, or a component thereof.

The API film 31 could be used on its own to form the edible wall 32 of the drug package, but may be applied to a carrier film which incorporates a perforated, thinned or otherwise weakened region allowing the entire quantity of API film 31 to be detached with the polypill 30 to ensure an accurate dose without wastage of the drug.

The drug or API may incorporated into the API film 31 in solution or in particulate form. It could be in granular form, in which case the granules may be coated with a coating to modify the release rate or other bioavailability characteristic of the drug, as may the spheroids 3 or other granules of the first embodiment. If the drug is homogeneously distributed in the API film 31 then the dose can be established by controlling the volume of the API film that is applied to each cell 711 of the drug package. For example, the API film could be formulated with a known concentration of the drug 1 or 2 and then rolled to a predefined thickness and stamped into discs of predefined diameter, or could be deposited onto the carrier film 34 as a drop of predefined volume.

The carrier film 34 could be the same material as the API film 31 or could be a different material, e.g. a film forming polymer as further discussed below.

The finished end user package 700, 7000 is shown in FIGS. 80, 89 and 90. It can be seen that the label portion 713′ of the uppermost drug package is adhered to the front surface of the underlying label portion 713′ of the second drug package, which (if three drug packages are provided) adheres in turn to the underlying label portion 713′ of the third drug package. The second label portion 713″ of the first drug package is absent, revealing the second label portion 713″ of the second drug package which adheres to the front surface of the second label portion 713″ of the third drug package. The third label portion 713′″ of the first and second drug packages is absent, revealing the third label portion 713′″ of the third drug package which, together with its concealed, first and second label portions 713′, 713″, adheres to the rear cover package 7005 (if three packages are included). If only two packages are included, as illustrated in the front view in FIG. 80, then the position of the third label portion 713′″ is occupied by the blank, non-adhesive, forwardly facing surface of the rear cover package 705. The juxtaposed label portions thus form a stepped configuration in which the overall thickness of the end user package reduces step-wise from left to right, which is why the pressure plates 521 may include a similar, stepped configuration 522′.

FIG. 86 shows a cross-section through one of the cells 711, while FIG. 87 shows the same section after removal of the pastille or polypill 30, which in the illustrations has only two API films 31 including two respective drugs 1, 2; if more than two drug packages are used then more than two edible walls 32, e.g. API films 31, will be present.

In the illustrated embodiment, each pastille 30 comprises the API films 31 which are compressed together with the carrier films 34 between the discs or wafers 33 of the front and rear cover packages, which provide a convenient, non-sticky surface for the user to handle. Ambient air pressure acting on the foil walls 714 may apply pressure over an extended time period, as long as the respective cell 711 of the end user package remains unopened, sufficient to cause full adhesion between the walls 32 and 33, e.g. film walls 31 and 34 and wafers 33 as shown.

In use, the end user simply presses against one of the foil walls 714 to push the pastille 30 through the opposite wall 714 out of its cell 711; the carrier films 34 and walls 33 rupture at the perforations 35 to release the pastille 30 from the frame 710.

### Edible Walls

The film of the at least one edible wall 32, e.g. the API film 31 and/or the carrier film 34, may be based on a film forming polymer as well known in the art, e.g. a water soluble polymer such as hydroxypropyl methylcellulose, carboxymethyl cellulose, e.g. sodium carboxymethyl cellulose, hydroxypropyl cellulose, or hydroxyethylcellulose.

The edible wall 33 forming the outer surface of the polypill 30 could be, for example, an edible paper, e.g. edible rice paper (made from dried starch, e.g. a mixture of rice and tapioca flour); a non-tacky film based on a film former as used for the edible wall 32; or an edible paper made from edible (i.e. non-toxic) fibres made from cellulose, e.g. cotton, vegetable protein, collagen or other fibre forming material and bound together, e.g. with a film former, optionally including one or more disintegrants, fillers, or other components as known in the art. The edible wall 33 may include a tacky layer facing away from the foil wall 714 of the cell to bond with the adjacent edible wall 32, and a non-tacky layer facing the foil wall 714 of the cell which allows the polypill 30 to be removed from the cell 711 without sticking to the foil 714.

Where the edible wall 32 of the drug package includes an API film 31 and/or a carrier film 34, either or both of the API film 31 and the carrier film 34 may be made in one layer or as a laminate of more than one layer with different compositions. Alternatively, the at least one edible wall 32 may be made from a similar material to the edible wall 33, but sticky on both sides so as to bond to the at least one edible wall 32 of the adjacent package during assembly.

For example, the at least one edible wall 32 could be made in two layers to enclose the drug 1, 2 in-between the layers, or in one or more layers with the drug 1, 2 included in the or each layer as an homogeneously distributed component of the film or wall, e.g. by mixing or impregnating it into the material of the film or wall before or after forming the film or edible wall 32, or printing onto the formed film or wall, e.g. in particulate, e.g. pulverulent or granular form, or as a solution or a dried suspension. The drug 1, 2 may be incorporated into the film or applied to the at least one edible wall 32 in micro- or nano-particulate form, e.g. as taught by US2016022599 A1. The at least one edible wall 32 could be coated on one or both sides, e.g. with a film former or edible tackifier to preserve the drug and/or to modify its release characteristics and/or to promote adhesion. Modified release may also be obtained by applying a modified release coating to the drug in granular form as contained in the film or between the layers of the at least one edible wall 32, and/or by incorporating the drug into multiple layers of the film with different compositions, e.g. different solubility.

The film forming polymer may be a combination of a first carboxymethyl cellulose of relatively lower molecular weight and a second carboxymethyl cellulose of relatively higher molecular weight, as taught by US2009035426 A1.

The film may include a tackifier to improve its adhesion to the films of the other packages. Films with tackifiers are taught for example in US2009035426 A1.

The film may include further ingredients such as one or more plasticizers, surfactants, disintegrants, etc. as known in the art.

Plasticizers may also assist adhesion of the films and may include, for example: glycerin, polyethylene glycol, propylene glycol, monoacetin, triacetin, triethyl citrate, sorbitol, 1,3-butanediol, and D-glucono-1,5-lactone.

Surfactants may include, for example: sodium dodecyl sulphate (i.e. sodium dodecyl sulfate or SDS), sunflower lecithin, polyoxyethylene sorbitan fatty acid esters (e.g., polysorbate), polyoxyethylene alkyl ethers, and polyoxyethylene castor oil derivatives.

Other possible film formers, tackifiers, disintegrants and other components of the film may include, for example: polyvinyl pyrrolidone, polyvinyl alcohol, polyethylene glycol, polyacrylic acid, methylmethacrylate copolymer, carboxyvinyl polymer, pullulan, sodium alginate, amylase, levan, elsinan, collagen, gelatin, zein, gluten, soy protein isolate, whey protein isolate, casein, modified food starches, e.g. dextrins, maltodextrins, high amylase starch, hydroxypropylated high amylase starch, or pregelatinized wheat starch; pectin, acacia gum, xanthan gum, guar gum, gum arabic, algin, methylated products of cellulose or starch derivatives, their carboxyalkylated products and hydroxyalkylated products; polyacrylates and polymaleates; other proteins and polysaccharides, and mixtures thereof as known in the art.

In order to promote adhesion to the edible wall 32 or 33 of the adjacent package, the edible wall 32 of the drug package may be an adhesive composition based on carboxypolymethylene as taught by U.S. Pat. No. 5,851,512, wherein “carboxypolymethylene” includes in particular copolymers of acrylic acid and polyallyl sucrose. Carboxypolymethylene may be mixed with glycerine to form a sticky gel. Suitable carboxypolymethylene compositions are commercially available as CARBOMER® CARBOPOL®, e.g. from The Lubrizol Corporation of Cleveland, Ohio, USA.

A particularly tacky film may be obtained by the addition of Gelucire® 44/14. Gelucire® 44/14, with HLB value of 14, is a mixture of monoesters, diesters and triesters of glycerol, and monoesters and diesters of polyethylene glycols, belonging to the lauryl polyoxylglycerides (macroglycerides) family, and available from Gattefossd SAS of Saint-Priest, France.

A film obtained by hot melt extrusion from a mixture of Eudragit RS PO powder with Gelucire® 44/14 is reported by Esra'a Albarahmieha, Sheng Qia, and Duncan Q. M. Craig—“Hot Melt Extruded Transdermal Films based on Amorphous Solid Dispersions in Eudragit® RS PO: The Inclusion of Hydrophilic Additives to Develop Moisture-Activated Release Systems”—International Journal of Pharmaceutics, 514 (1). 270-281. ISSN 0378-5173—available online at https://ueaeprints.uea.ac.uk/

Eudragit® RS powder (PO) grade is a glassy copolymer synthesized from acrylic acid and methacrylic acid esters with 5% of functional quaternary ammonium groups, and available from Evonik Nutrition & Care GmbH of Essen, Germany.

### Alternative Embodiments

In alternative embodiments, the capsule part (e.g. the capsule body 22) of the capsule body package could be arranged in the cell without a carrier, in which case it could be urged through the aligned cells of the packages by the respective pushrod (like the pushrods of the first machine or variant first machine) and could rupture the foil of each cell by direct contact.

In further alternative embodiments, the end surfaces of the pushrods of the first machine or variant first machine could have a recess to receive the respective capsule part (e.g. the capsule body) which is supported in the recess during assembly. In this case the leading end of the pushrod could contact and rupture the film of each cell during assembly. An inner pushrod could be arranged to expel the finished capsule as the main pushrod retracts from the cell. The cell could then be closed by the flap of the end user package forming the composite label or label assembly, without the need for an insert or carrier. The cells of the end user package could be configured to restrain the finished capsule to prevent it leaving the cell in the final stage of assembly. For example, the cell could include an element or elements located at or proximate the end through which the capsule enters, perhaps similar to the inwardly projecting tabs of the alternative locating structures as described with reference to the cap package of the first embodiment, which are deflected by the capsule or pushrod and then return to a rest position to retain it in the cell.

In alternative embodiments it is conceivable that a structural part of the end user package, and/or a part of each capsule or other polypill, could be formed from a part that is introduced into the machine without being part of a package. For example, the machine could be loaded with a cassette or a hopper containing multiple capsule parts which are introduced into the cells of the packages of the first embodiment or variant first embodiment to receive the combined drugs, or could include a roll or sheets of a frangible film that is applied within the machine to dose the cells of the end user package of the second embodiment or variant second embodiment to seal the polypills in the cells.

It is desirable to package the polypills individually in individual cells of an end user package, both because it better preserves them in storage, and because it obviates the risk of mixing them up with other medicaments. It is conceivable however, although less preferred, that the polypills could be delivered instead in a bulk end user package (such as a pill bottle).

In yet further possible arrangements, the first machine or variant first machine could be configured to urge the first and second drugs out of the aligned cells of the stacked drug packages using a first set of pushrods arranged generally as shown, and then to use another set of pushrods arranged orthogonally to the first set to encapsulate the drugs within the cells of the end user package which define cavities containing the capsule parts, which are spaced apart facing each other in the cavity with their length axis orthogonal to the length direction of the first pushrods.

In this arrangement the carriers 320 could be replaced by solid plugs or plungers, with each plunger being received (in the direction of movement of the first pushrods, orthogonal to the second pushrods) in a hole that intersects the capsule cavity to urge the drugs in front of the solid plunger, into the cavity between the opposed capsule parts 21, 22.

The second pushrod could then close the capsule at a position in the cavity beyond the plunger. The plunger could then be pushed deeper into the hole, by the first pushrod, after the second pushrod is withdrawn, to become a part of the end user package which seals both the hole that receives the plunger, and the hole that admits the second pushrod. This again provides encapsulation entirely within the cells of the introduced packages without contact between the drugs and any parts of the machine, and delivers an end user package with a somewhat slimmer form factor at the cost of a more complex machine.

In yet further embodiments, rather than having a powered actuator, the machine could be manually operated (e.g. by a lever or the like) so that the mechanism is driven only by the user.

For example, the first machine 100 could be adapted for the user to depress the pressure plate by means of a lever. A set of pressure pads (similar to pressure pad 250) could be provided in different thicknesses with a profile aperture defining a different shape profile index position for each pressure pad, so that each pad can be received in the machine in a different index position of a respective profile rod. A manual knob could be provided for rotating that respective profile rod to the desired position, to set the capsule size, which in turn defines the thickness of the pressure pad that will fit that profile rod, which in turn defines the height of the receiving space between the pressure plate and the upper surfaces of the pedestal block and pushrod ends. Thus, the stack height is controlled for capsule capacity in a similar way to the first machine, but in a simple, manual mechanism that does not need a powered actuator or position sensor to set the position of the pressure plate. The pressure pads could be colour coded for convenience.

The second machine or variant second machine could include a heat source or ultrasonic or other welding means for welding the package frames together.

Many further adaptations are possible within the scope of the claims.

### SUMMARY

In summary, in embodiments, customised polypills may be produced in the pharmacy from two or more drug packages using an assembly machine. Each drug package may comprise an array of cells containing individual doses of a single drug. The individual doses may be combined within the cells of the packages to produce an end user package with an array of cells containing the combined drugs in the form of individual polypills, which may be formed as capsules or pastilles. Label indicia of the drug packages may be combined together to form a composite label of the end user package. Serialization data may be read from the packages during assembly and sent to a remote server for authentication and supply chain management.

In another aspect, in embodiments, patients who have a history of poor compliance with a prescribed oral antipsychotic while periodically seeking symptomatic relief from another prescribed psychoactive, are provided with both medicaments in the form of polypills to be taken in place of the solo antipsychotic when the patient is in crisis. Providing the preferred psychoactive as a combination motivates the patient to resume their antipsychotic therapy when symptoms return, which may result in better compliance and more effective management of psychosis in the community.

### Treating Psychosis

The discussion turns now to the second broad aspect of the invention as directed to the treatment of psychosis.

Psychosis is characterised by symptoms including hallucinations (typically, hearing voices) and/or delusions (i.e. delusional thoughts or beliefs, e.g. paranoia). Psychosis is a defining aspect of chronic mental illness such as schizophrenia and so, in this specification, the treatment of psychosis is synonymous with the treatment of the underlying mental illness.

Individuals suffering from chronic mental illness resulting in psychosis are often treated in the community with a maintenance regimen of oral antipsychotics for self-administration (i.e. relying on the individual to take the pills regularly). One common problem is poor compliance with the therapeutic regimen, resulting in periodic episodes of acute psychosis with repeat hospital admissions.

As an alternative, a depot injection can be given periodically to release the antipsychotic over a prolonged period. However, some individuals are unwilling to receive a depot injection, or may respond better to antipsychotics which are unsuitable for delivery in that form.

Psychotic symptoms may worsen responsive to stressors in the patient's home or work environment. Typically it is impractical for the clinician to monitor the individual in the community closely enough to adjust their medication to respond to such events. On the contrary, as the patient's condition worsens it may become less rather than more likely that the patient will continue to comply with a regular maintenance regimen of oral antipsychotics.

The invention identifies, and is directed particularly to, a group of individuals who:

i) are diagnosed with a chronic mental illness, e.g. schizophrenia, characterised by psychosis;
ii) are prescribed an oral antipsychotic for self-administration as an ongoing maintenance therapy to treat the underlying mental illness to prevent the re-emergence of hallucinations and/or delusions as acute psychotic symptoms;
iii) are also prescribed, in acute phases of their illness, another psychoactive drug for use over a more limited time period to alleviate one or more affective symptoms associated with the re-emergence of the psychosis;
iv) have a history of poor compliance with the oral antipsychotic regimen; and
v) in contrast, demonstrate a positive inclination to take the psychoactive.

As a sub-group within this patient group, some individuals may further be diagnosed with a personality disorder. A personality disorder will affect thinking and behaviour and may be a persistent contributory factor in non-compliance, even when antipsychotic therapy is effective in managing the underlying mental illness. Thus, the invention may be applied particularly to improve compliance in this sub-group.

In this patient group, the antipsychotic is prescribed as an ongoing maintenance therapy for self-administration by the individual living in the community. The additional, psychoactive medication is prescribed for the same individual from time to time, only for a more limited period, to alleviate affective symptoms during an acute psychotic episode. Prescribing for a limited period may avoid the development of tolerance, dependence or addiction that could result from longer term use. An acute psychotic episode may result in hospitalization or, if symptoms are less severe, could be managed in the community.

The invention recognises that the causes of noncompliance with a maintenance regimen of oral antipsychotic therapy may be partly psychological. Objectively rational fears of adverse side-effects may be exaggerated while the behavioural consequences of the illness are denied. For such individuals, denial or lack of insight could be regarded rather as a coping strategy, which may have supported the individual for many years before diagnosis and treatment. The coping strategy may lead the individual to eschew treatment for their psychosis—which is to say, for the specific symptoms (hallucinations and/or delusions) that are targeted by their oral antipsychotic therapy.

A delusional belief system may include, for example, attributing negative intentions or hallucinatory voices to real or imagined people, and so may give rise to problematic behaviour which imposes a burden on friends, neighbours, work colleagues, health and criminal justice services and others in the affected community. Problematic behaviour results in adverse social experiences which in turn may reinforce the paranoid or other delusional beliefs that drive the behaviour. Thus, adherence to a maintenance regimen of antipsychotic therapy may be key to successful management of the illness in the community.

However, an individual who denies that they are ill and rationalizes imagined voices or other hallucinations as real sensory experiences may reject compliance with a regimen of antipsychotic medication, prescribed to treat the hallucinations and delusions, as unnecessary and even as an act of self-betrayal, insofar as compliance implies acceptance of a rejected diagnosis of mental illness.

The invention recognises that although such patients may be highly resistant to taking their oral antipsychotic when they believe they are well, for the reasons discussed above, the aversion may be to the act of compliance with the therapeutic regimen as much as to any real or exaggerated fear of adverse side-effects. This is reinforced by the relatively gradual and cumulative action of antipsychotics which may make it more difficult for the individual to be convinced that their medication is effective. Thus, noncompliance may result from denial in combination with lack of perceived benefit of the medication, as much as any perceived disbenefit of the medication.

The invention further recognises that an individual who has developed a coping strategy to tolerate the hallucinations and delusions that characterise their psychosis, and who eschews compliance with the antipsychotic regimen, may nevertheless actively seek to take another psychoactive drug, prescribed during acute phases of their illness, to alleviate distressing affective symptoms that may be associated in that individual with the re-emergence of their psychosis.

In contrast with the rejection of compliance with the antipsychotic regimen as an act of self-betrayal, compliance with a regimen of psychoactive medication which is perceived to alleviate an affective symptom may be regarded as an act of self-affirmation (taking back control).

This motivation may be stronger if the psychoactive is selected to exert a relatively more rapid therapeutic effect—for example, relatively more rapid than that of the antipsychotic—and so gives rise to a relatively stronger association with the expected benefit in the mind of the individual.

The invention further recognises that by adapting the range of choices available to the individual, this motivation can be harnessed to improve compliance with a therapeutic maintenance regimen of oral antipsychotics.

This can be achieved by means of a kit of medicaments; the use of the kit of medicaments for the treatment of psychosis; a method of treating psychosis; and/or a method for manufacturing medicaments for treating psychosis, as will now be discussed.

### Aspects of the Invention as Directed to the Treatment of Psychosis

In one aspect, the invention provides a kit of medicaments for use in the treatment of psychosis in an individual with a history of non-compliance with oral medication. The kit includes a plurality of crisis oral dosage units, and a plurality of maintenance oral dosage units. Each of the crisis oral dosage units includes a single dose of a first drug and a single dose of a different, second drug, said single doses being combined together as a single, orally ingestible body. Each of the maintenance oral dosage units includes a single dose of the first drug without the second drug. The first drug is an antipsychotic, and the second drug is a psychoactive selected to alleviate an affective symptom associated with the individual's psychosis.

In another aspect, the invention relates to the use of a kit of medicaments as defined above, for the treatment of psychosis in an individual with a history of non-compliance with oral medication.

The treatment of psychosis may comprise the treatment of schizophrenia.

Both the crisis oral dosage units and the maintenance oral dosage units may be provided to the individual for alternative self-administration at the discretion of the individual, wherein the maintenance oral dosage units are provided for self-administration when the individual feels well, and the crisis oral dosage units are provided for self-administration when the individual feels unwell.

In another aspect, the invention provides a method for treating psychosis in an individual with a history of non-compliance with oral medication. The method comprises providing different, first and second drugs, wherein the first drug is an antipsychotic, and the second drug is a psychoactive selected to alleviate an affective symptom associated with the psychosis. The method further comprises combining together the first and second drugs to form a plurality of crisis oral dosage units, each of the crisis oral dosage units including a single dose of the first drug and a single dose of the second drug, said single doses being combined together as a single, orally ingestible body. The method further comprises forming a plurality of maintenance oral dosage units, each of the maintenance oral dosage units including a single dose of the first drug without the second drug; and providing both the crisis oral dosage units and the maintenance oral dosage units to the individual for alternative self-administration at the discretion of the individual, wherein the maintenance oral dosage units are provided for self-administration when the individual feels well, and the crisis oral dosage units are provided for self-administration when the individual feels unwell.

The method may be applied for the treatment of schizophrenia.

In another aspect, the invention provides a method for manufacturing medicaments for treating psychosis in an individual with a history of non-compliance with oral medication. The method comprises identifying the individual, and identifying different, first and second drugs prescribed for the identified individual, wherein the first drug is an antipsychotic, and the second drug is a psychoactive selected to alleviate an affective symptom associated with the psychosis in the identified individual. The method further comprises combining together the first and second drugs to form a plurality of crisis oral dosage units customised for the identified individual, each of the crisis oral dosage units including a single dose of the first drug and a single dose of the second drug, said single doses being combined together as a single, orally ingestible body. The method further comprises forming a plurality of maintenance oral dosage units, each of the maintenance oral dosage units including a single dose of the first drug without the second drug; and packaging the plurality of crisis oral dosage units and the plurality of maintenance oral dosage units, respectively as two separate components in a kit of medicaments.

The elements of the kit may be supplied simultaneously or sequentially, but in either case are supplied for alternative self-administration by the individual at the discretion of the individual. The individual is instructed to take the maintenance oral dosage units but not the crisis oral dosage units when they are feeling well, and to take the crisis oral dosage units but not the maintenance oral dosage units for additional symptomatic relief when they are feeling stressed or otherwise unwell.

Byway of example, FIG. 59 shows a kit of medicaments, comprising first and second components 300, 900 packaged together in a pharmacy bag 901 for delivery to the user. The first component is the end user package 300 as described above, containing a plurality of crisis oral dosage units in the form of capsules 20. Each capsule contains in combination a single dose of the first drug 1, which is an antipsychotic (olanzapine, 20 mg), and a single dose of the second drug 2 (citalopram, 20 mg), which is a psychoactive selected to alleviate an affective symptom associated with the psychosis. This particular combination is shown purely by way of example. The second component 900 is a conventional blister pack containing a plurality of maintenance oral dosage units in the form of tablets or capsules, each containing a single dose of the antipsychotic (olanzapine, 20 mg). In alternative embodiments, both packages may be of similar appearance—for example, the second component could be another end user package 300 made with only one drug instead of two. The packages could be colour coded for easy recognition by the user. Included in the pharmacy bag may be a patient leaflet 266, instructing the user to take the maintenance oral dosage units when they feel well, and to take the crisis oral dosage units instead of the maintenance oral dosage units when they feel unwell.

In this context, “well” and “unwell” are defined by the presence or absence or relative severity or lack of severity of the affective symptom. For example, on a scale rating the affective symptom from not present, through mild to moderate to severe, “well” might be interpreted as not present or mild, and “unwell” might be interpreted as moderate or severe.

The psychoactive should be a drug that the individual is motivated to take in order to alleviate one or more affective symptoms associated with a recurrence of their psychosis. The psychoactive may be selected to alleviate, in particular, transient affective symptoms that increase and decrease in severity together with the psychosis.

In the context of this invention, the therapeutic action of the psychoactive is significant principally for its motivational influence on the individual. What matters is that the affective symptom makes the individual feel unwell, and the individual associates the psychoactive with its therapeutic effect in alleviating the affective symptom. The act of taking the combined medicament may be perceived on balance as an act of self-affirmation in which the presence of the antipsychotic is of relatively minor significance. Thus, the individual is motivated by the presence of the affective symptom to take the psychoactive in order to feel better.

For this reason it is important that the psychoactive is selected for the individual patient. Since there are numerous possible combinations and dosages, the crisis oral dosage units may be prepared as customised, single oral dosage units, which are preferably polypills but could alternatively be in liquid form and defined by a liquid measure at the point of consumption. Conveniently, such polypills may be prepared using the novel methods and apparatus as discussed above.

The psychoactive may be considered a reward substance, in the sense that the individual associates it with an emotional reward (e.g. elevated mood, reduced anxiety.) Importantly however, the psychoactive alleviates an affective symptom associated with the individual's psychosis, which will be different for different individuals—and so, the individual is motivated to take the second medicament, particularly by the onset of the affective symptom which occurs together with the recurrence of their psychosis.

A drug that provides more rapid relief from the affective symptom may be more strongly associated by the individual with its therapeutic effect, and so the individual may be more strongly motivated to take it at the onset of the affective symptom.

Accordingly, when taken at a prescribed frequency: the first drug (i.e. the antipsychotic) may be selected to alleviate a first symptom of the psychosis within a first time period from initial administration, the first symptom being one of hallucinations and delusions, and the second drug (i.e. the psychoactive) may be selected to alleviate the affective symptom within a second time period from initial administration; the second time period being shorter than the first time period.

That is to say, the psychoactive may be selected to relieve the target affective symptom, when taken by the individual at the prescribed frequency, in a relatively shorter time period from first administration than the simultaneously ingested antipsychotic relieves the first symptom (hallucinations or delusions) in that individual.

Alternatively or additionally, the psychoactive may be selected to relieve the target affective symptom, when taken by the individual at the prescribed frequency, in a period of less than 24 hours, preferably less than 12 hours, more preferably less than 6 hours, yet more preferably less than 3 hours from first administration.

The antipsychotic may be provided at the same dose in both the crisis and maintenance oral dosage units, or may be provided at a different dose (e.g. a higher dose) in the crisis oral dosage units relative to the maintenance oral dosage units.

The maintenance oral dosage units could be packaged as conventional fixed dosage units, e.g. capsules or tablets, or could be packaged using the novel assembly machine in packaging similar to that of the crisis oral dosage units. In either case, colour coding or other suitable indicia could be used to distinguish the maintenance oral dosage units (e.g. in a blue pack) from the crisis oral dosage units (e.g. in a red pack).

Affective symptoms associated with the psychosis may include one or more positive affective symptoms such as anxiety, agitation, and irritability. Such positive affective symptoms may particularly motivate the individual to turn to the psychoactive for relief.

Some individuals may also be motivated by depressive affective symptoms associated with the psychosis to turn to the psychoactive for relief from those depressive affective symptoms. Depressive affective symptoms may include sadness, anhedonia, feelings of guilt or low self-worth, or apathy.

Affective symptoms are symptoms expressive of mood or emotion. It will be understood of course that hallucinations and delusions are not classed as affective symptoms.

The psychoactive may alleviate the affective symptom associated with the psychosis by removing the affective symptom, or reducing the severity of the affective symptom.

Of course, the antipsychotic should be selected and prescribed in accordance with good clinical practice to minimise unwanted side-effects, in order that the presence of the antipsychotic should not deter the patient from taking the combined dosage unit. The invention is not intended to overcome noncompliance that represents a proportionate avoidance of severe side-effects.

It may also be noted that noncompliant patients may have relatively difficult or chaotic life circumstances with limited choices, and so the ease of availability of the psychoactive in the combination dosage unit on prescription from the pharmacy may be a relatively significant factor influencing the patient's behaviour. The balance of convenience may induce such patients to take the combination medicament rather that obtaining the desired psychoactive on its own via illicit sources.

The invention provides a further benefit in that the compliant behaviour encouraged by the novel combination is self-regulating. The patient is a sensitive barometer of their own mental state and will turn to the psychoactive medicament of choice for relief when their affective symptoms return. Through managing patient choice by combining the antipsychotic with the preferred, psychoactive medicament, the previously non-compliant patient may be encouraged to voluntarily resume their antipsychotic therapy with the first symptoms of their returning psychosis.

In contrast, conventional approaches rely on visiting clinicians to respond to the patient's condition, which may not happen until the patient's symptoms have worsened to such an extent that the clinician is called by concerned friends and neighbours, perhaps resulting in an emergency hospital admission.

The invention may thus provide cheaper, more effective and more responsive management of psychosis in the community.

For chronically ill and noncompliant patients who do not take the conventional antipsychotic or who only take it sporadically, the invention may be expected to result in a cycle of illness and wellness wherein the periods of illness are less severe than would be the case with the conventional approach of management in the community.

### Antipsychotics and Psychoactives

The psychoactive may be an antidepressant.

The psychoactive may be an anxiolytic or a sedative or hypnotic.

An anxiolytic or sedative or hypnotic may be a benzodiazepine or a “Z” drug. A “Z” drug means one of zaleplon, zolpidem, and zopiclone.

A benzodiazepine may be selected for effect principally as an anxiolytic; for example, it could be one of alprazolam, chlordiazepoxide, diazepam, lorazepam, and oxazepam.

Alternatively, a benzodiazepine may be selected for effect both as a sedative and as an anxiolytic; for example, it could be one of flurazepam, nitrazepam, loprazolam, lormetazepam, and temazepam.

The antipsychotic may be selected to alleviate a first symptom of the psychosis, the first symptom being one of hallucinations and delusions, while the psychoactive is selected to alleviate the affective symptom without alleviating the first symptom. Thus, the psychoactive need not be an antipsychotic.

Affective symptoms may include negative affective symptoms of psychosis. The psychoactive may be selected to alleviate at least one negative affective symptom of the psychosis without alleviating hallucinations, delusions, or other positive symptoms. In this case the psychoactive may also be an antipsychotic, for example tiapride, which is effective to alleviate negative symptoms but not positive symptoms of psychosis.

Alternatively, the psychoactive may also be an antipsychotic that helps to alleviate also hallucinations or delusions as principal symptoms of the psychosis. For example, the psychoactive could be an atypical antipsychotic effective also as an anxiolytic, such as carpipramine; or an atypical antipsychotic effective also as an antidepressant, such as amoxapine.

As referred to herein, an antipsychotic or a particular class of psychoactive may be a drug listed with a corresponding therapeutic indication (whether or not also with other therapeutic indications) in the pharmacopoeia for the jurisdiction where the respective prescription is issued—for example, the European Pharmacopoeia, the Japanese Pharmacopoeia, or the United States Pharmacopeia.

The antipsychotic may be an atypical antipsychotic. An atypical antipsychotic could be, for example: amisulpride; aripiprazole; asenapine; bifeprunox; blonanserin; brexpiprazole; brilaroxazine; cariprazine; carpipramine; clocapramine; clotiapine; clozapine; gevotroline; iloperidone; levosulpiride; lurasidone; melperone; mosapramine; nemonapride; olanzapine; paliperidone; perospirone; pimavanserin; piquindone; quetiapine; risperidone; sertindole; sulpiride; sultopride; ziprasidone; or zotepine.

For example, the antipsychotic could be one of: amisulpride; aripiprazole; asenapine; clozapine; lurasidone; olanzapine; paliperidone; quetiapine; and risperidone.

Alternatively, the antipsychotic could be a first generation or typical antipsychotic. A first generation or typical antipsychotic could be, for example: chlorpromazine; chlorprothixene; fluphenazine; haloperidol; loxapine; molindone; perphenazine; pimozide; promazine; thiothixene; timiperone; trifluoperazine; or triflupromazine.

An antidepressant could be, for example: amitriptyline, bupropion, citalopram, clomipramine, desipramine, desvenlafaxine, doxepin, escitalopram, fluvoxamine, fluoxetine, imipramine, isocarboxazid, maprotiline, mirtazapine, nefazodone, nortriptyline, paroxetine, phenelzine, protriptyline, sertraline, tranylcypromine, trazodone, trimipramine, or venlafaxine.

An antidepressant may be an SSRI (selective serotonin reuptake inhibitor); for example, it may be one of citalopram, escitalopram, fluvoxamine, fluoxetine, paroxetine, and sertraline.

Where the psychoactive is selected as an anxiolytic, sedative or hypnotic, it could be, for example: alprazolam, chlordiazepoxide, clonazepam, clorazepate, diazepam, estazolam, flurazepam, halazepam, loprazolam, lorazepam, lormetazepam, midazolam, nitrazepam, oxazepam, prazepam, temazepam, zaleplon, zolpidem, zopiclone, or a prodrug of desmethyldiazepam (which is to say, a drug which is metabolized to form desmethyldiazepam as a metabolite).

In this specification, drugs may be taken to include, where therapeutically equivalent, their respective prodrugs, pharmaceutically acceptable salts and structural analogues.

Of course, if a specific psychoactive is known to have an adverse interaction with a specific antipsychotic then that combination is not selected.

The crisis oral dosage units may include only one, or more than one antipsychotic in addition to the psychoactive, and/or only one, or more than one psychoactive in addition to the antipsychotic.

The antipsychotic and/or the psychoactive could be in solid or liquid form. For example, cannabidiol (CBD) may be included as an antipsychotic or as an adjunct to an antipsychotic, and may be in a liquid form, e.g. as a tincture or an oil.

### Efficacy

The efficacy of the novel approach to the treatment of psychosis can readily be assessed by means of a clinical trial which may be designed as follows.

A group of individuals are identified who satisfy the five criteria (i)-(v) as set out above.

Consent is obtained from each individual to comply with a regular assessment of their mental health, conducted in the conventional way, e.g. using the PANSS scale or equivalent, for the duration of the trial. The assessment may include a questionnaire on compliance; however, this is of limited use since it relies on the truthfulness of the patient.

Consent may be obtained from each individual to deliver their existing prescribed drugs in new packaging, and in combination. Preferably however, since efficacy turns on managing choice, if it is determined that consent is not required, then consent is not obtained.

For each individual, their existing prescribed antipsychotic and psychoactive are provided in three different end user packages. All three packages are of generally identical appearance except for their labels which correctly indicate the respective drugs contained therein. Each package contains a plurality of solid, single oral dosage units, e.g. tablets or capsules, which should also be generally similar in appearance, and for the three different packages comprise respectively: Package a) the antipsychotic alone; Package b) the psychoactive alone; and Package c) the antipsychotic and psychoactive in combination.

The patients are divided into two groups. For a first time period, the first group receives packages a) and b), while the second group receives packages a) and c). For a second, consecutive time period, the first group receives packages a) and c), while the second group receives packages a) and b).

Individuals receiving packages a) and c) are instructed to take the maintenance oral dosage units but not the crisis oral dosage units when they are feeling well, and to take the crisis oral dosage units but not the maintenance oral dosage units for additional symptomatic relief when they are feeling stressed or otherwise unwell. Individuals receiving packages a) and b) are instructed to take the antipsychotic when they are feeling well, and to take the psychoactive in addition to the antipsychotic for additional symptomatic relief when they are feeling stressed or otherwise unwell.

The patients are assessed regularly for the duration of the trial as mentioned above.

The visual similarity of the oral dosage units and the packaging controls for the placebo effect of new packaging and new routines, while the staged time periods control for global external factors like weather and socioeconomic environment. The objective is for the only change to be whether the psychoactive is available with or without the antipsychotic in combination.

Since cycles of illness and wellness can last a long time, the time periods should be as long as possible, e.g. one or two years or more, and the group should be as large as possible.

The groups are randomised. Of course, the trial is open label for the patients, but preferably is blinded for the psychiatrist or other clinician involved in the assessment—which is to say, they know what medications the patient is receiving but they don't know whether they are in the combined form or the separate form. This controls for bias in the assessment. This may have to change during the trial if a particular individual is hospitalised or their medication is changed.

After the trial, the assessment outcomes are collated and analysed for statistical significance.

### LIST OF REFERENCE NUMERALS

The following list of reference numerals is presented for ease of reference.
1 First drug (olanzapine, 20 mg)
2 Second drug (citalopram, 20 mg)
3 Spheroid
4 Adhesive, adhesive surface, adhesive side (also shown by small vertical dash fill in the variant first embodiment)
5 Release surface, release coating (also shown by vertical dash fill)
10 External electric power supply
20 Polypill (capsule)
21 Capsule cap
22 Capsule body
30 Polypill (pastille)
31 API film
32 Edible wall
33 Edible wall (disc or wafer)
34 Carrier film
35 Perforations
36 Cardboard sheet
90 Database
91 Remote computer
92 Removable data storage device
100 First machine
101 Receiving space
102 Moving frame position sensor
103 Pedestal block latches
104 Fixed abutment surfaces
110 Pushrod
111 Upper end surface of pushrod
120 Moving frame assembly
121 Pressure plate (compression plate)
121′ Tongue
122 Pressure surface (compression surface)
123 Pressure plate sensor
124 Handle
125 Abutment
126 Slot
127 Recess
128 Recess
129 Recess
130 door
131 window
132 door position sensor
133 Cavity
141 Hydraulic piston
142 Cylinder
143 Motor driven hydraulic pump
144 Hydraulic valve control assembly
145 Tank
150 Alignment structure
151 Alignment rods
152 Profile rod—umbrella term for:
   152′ First (left-hand) profile rod
   152″ Second (centre) profile rod
   152′″ Third (right-hand) profile rod
153 Index mechanism
154 Sensor
155 Indicators
156 Index plunger
157 Index plunger
158 Index wheel
160 Arm
161 Abutment
162 Solenoid
163 Pawl
164 Ratchet plate
165 Sensor
166 Pivot block
170 Pedestal block
171 Assembly surface (upper end surface of pedestal block)
172 Flat, front surface
173 Compression springs
174 Pedestal block sensor assembly
175 Optical sensors
176 Profile rod sleeve
177 Upper shoulder of profile rod sleeve
178 Lower retainer
179 Slot for abutment on moving frame assembly
180 Slot for latches and fixed abutments
181 Slot for cutter
182 LED
183 First beam splitter
184 Mirror
185 Second beam splitter
186 Sensor window
190 Cutter
190′ Slit
191 Rotating shaft
192 Electric motor
193 Impellers
194 Cyclonic separator
201 Casing
202 Debris collection tray
203 Base plate
204 Guide rods
205 Spring guide rods
206 Shrouds
207 Perforated wall
220 Front plate
221 Locking mechanism
222 Handle
223 Locking ball
224 Control ball
225 Recess
226 Lever
227 Control lever
228 Reflective, inwardly facing surface of front plate
229 Resilient interlayer
230 Inner casing
231 Lower cavity
232 Guide sleeves
233 Brackets
234 Abutment for pressure plate
235 Abutment for pedestal block
236 Clearance slot for latches
250 Pressure pad
251 Pressure pad identifier
261 Reader, scanner, sensor
262 Local controller
263 Local memory
264 External data link
265 Printer
266 Information leaflet
267 Touchscreen
268 Scanning window
269 Data transmitter/receiver
270 Sensor signals
271 Control signals
272 Control interface
300 End user package (first machine)
301 First drug package
302 Second drug package
304 Capsule cap package (first capsule package)
304′ Capsule cap package (24 cells)
304″ Capsule cap package (36 cells)
304′″ Capsule cap package (48 cells)
305 Capsule body package (second capsule package)
310 Frame
311 Cell, enclosure
312 Composite label assembly
313 Package label (label portion, label element), label indicia
314 Frangible foil, foil outer wall (also shown by horizontal dash fill)
315 Strip
316 Tab
317 Indicia/Barcode (serialization data)
318 Cell wall
319 Weakened region of foil end wall
320 Carrier
321 Closed end
322 Open end
323 Salient portions
324 Enlarged internal diameter portion
325 Annular fin (locating structure)
330 Flap of cap package
331 Window
332 Release paper
333 Perforated tear line
334 Waste portion
340 Casing (cap package)
341 Aperture for tab
351 Alignment aperture
352 Profile aperture
360 Block (cap package)
361 Sub-block
362 Sheet
363 Hole in sheet
364 Aperture in sub-block
370 Flap of capsule body package
371 Perforation lines
372 Surplus portion of flap
373 Fold line
374 Outer casing
375 Body portion of outer casing
376 Wings of outer casing
377 Slit
378 Fold line
379 Sensor apertures
380 Block (capsule body package)
381 Inner casing
400 Block (drug package)
410 Inner casing
420 Outer casing
421 Slits
422 Outer wing portions
500 Second machine
501 First drug package receiving slot (slot [1])
502 Second drug package receiving slot (slot [2])
503 Third drug package receiving slot (slot [3])
504 Front cover package receiving slot (slot [A])
505 Rear cover package receiving slot (slot [B])
506 Casing
507 Package delivery window
508 Finger recess
510 First cutter/cutting station
511 Second cutter/cutting station
519 Punch
521 Pressure plate (compression plate)
522 Pressure surface (compression surface)
522′ Protruding or stepped portion of pressure plate
523 Actuator
530 Door
550 Alignment surface
551 Package locator rod
552 Package transfer actuator body
553 Gripper
554 Alignment body
561 Reader, scanner, sensor
561′ Additional reader, scanner, sensor and adjustable shape profile block
590 Vacuum generating apparatus
591 Vacuum pump
592 Reservoir
700 End user package (second machine)
701 First drug package
702 Second drug package
703 Third drug package
704 Front cover package
705 Rear cover package
706 Removable, temporary cover (for front of package)
707 Removable, temporary cover (for rear or inwardly facing side of package)
709 Outer wrapper
710 Frame
711 Cell, enclosure
712 Composite label assembly
713 Package label (label portion, label element); label indicia—umbrella term for:
   713′ Left-hand label portion
   713″ Centre label portion
   713′″ Right-hand label portion
714 Frangible foil, foil outer wall
715 Annular fold
717 Indicia/Barcode (serialization data)
718 Cell wall
719 Attachment point (between package and temporary cover)
720 Separators
721 Window
721 Channels
751 Locator rod aperture
752 Additional shape profile feature
900 Conventional blister pack (maintenance oral dosage units)
901 Pharmacy bag

### Variant First Embodiment

1000 Variant first machine
1080 Customer information
1110 Pushrods
1120 Moving frame assembly
1121 Pressure plate
1150 Profiled recess
1159 Profile protuberances
1170 Pedestal block
1187 Recess of pedestal block
1195 Displacement surface
1196 Shoulders
1197 Central recess
1203 Baseplate
1208 Elastomeric body
1209 Transducer
1210 Signal source
1220 Front plate
1231 Lower cavity
1240 Support surface
1280 External scanner
1281 Image capture device
1282 Image reproduction device
1283 Prism
1284 Second internal scanner
1285 Reflector
1300 End user package (48 capsules)
1300′ End user package (24 capsules)
1300″ End user package (12 capsules)
1301 First drug package
1302 Second drug package
1303 Composite drug package
1304 Capsule cap package (first capsule package)
1305 Capsule body package (second capsule package)
1311 Flocked cell
1311′ Unflocked region
1312 Composite label
1312′ Reproduced image
1314 Patterned frangible foil
1315 Strip
1316 Tab
1317 Package label
1317′ First indicia
1317″ Second indica
1320 Carrier
1328 Textured surface portion
1329 Smooth surface portion
1330 Flap of cap package
1332 Release paper
1340 Casing of cap package
1342 Outer casing sheet
1343 Inner casing sheet
1344 Discs
1345 Circular patch of adhesive
1346 Small dot of adhesive
1353 Profile recess (fits profile protuberance 1159 of profiled recess 1150)
1360 Block—cap package (48 cells)
1360′ Block—cap package (24 cells)
1400 Block—drug package/capsule body package (48 cells)
1400′ Block—drug package/capsule body package (24 cells)
1400″ Empty block
1430 Region of foil
1431 First layer of foil
1432 Second layer of foil (also shown by small horizontal dash fill)
1433 Sub-region of second layer of foil
1434 Bond region of second layer of foil
1435 Lacuna in second layer of foil
1600 Flocking apparatus
1601 Plate
1602 Plate
1603 Nozzle
1604 Positive electrode
1605 Hopper
1606 Blower
1607 Filter
1608 Valve
1699 Flock
2301 Alternative drug package
2400 Block (alternative package)/foraminous block
2401 Movable closure element/foraminous plate
2402 Recess of block
2403 Abutment surface of block
2404 Through-hole of movable closure element
2405 Slideway of block
2430 Filling apparatus
2431 Main body
2432 Discharge nozzle
2433 Discharge chute
2434 Filling chute
2435 Dosing body
2436 Dosing chute
2437 Photoelectric sensor
2438 Lower end
2439 Plunger

### Further Variant: End User Multipack, External Capsule Assembly

3001 End user package (multipack)
3002 Box
3003 End flap
3004 Front side flap
3005 Tear-off strip
3006 Release paper
3007 Rear side flap
3008 Aperture in base of box
3009 End user package label
3010 Tab
3011 Casing
3012 Hook
3013 Top window
3014 Side window
3015 Pressure bars
3020 End user sub-package
3021 Cell of end user sub-package
3022 Aperture (open end) of cell
3023 Rim of aperture
3024 Foil wall
3025 Rear wall of cell
3026 Location tab
3027 Body of sub-package
3028 Flat surface
3029 Axial abutment surface
3040 Capsule cap package (multipack embodiment)
3041 Lower foraminous block of cap package
3042 Upper foraminous block of cap package
3043 Movable closure element/foraminous plate of cap package
3044 Slideway
3045 Chamfered recess
3046 Tamper evident label
3047 Projection of movable closure element
3048 Annular seat
3050 Plug package
3051 Drug package
3060 Collar
3061 Aperture
3062 Annular sealing surface
3063 Prongs
3064 Latching surface
3065 Axial abutment surface
3066 Annular seat
3070 Plug
3071 Central post
3072 Annular sealing surface
3073 Prongs
3074 Latching surface

### Further Variant: End User Multipack, Internal Capsule Assembly

3080 End user sub-package, cap package
3081 Cell of end user sub-package
3082 Aperture (open end) of cell
3083 Rim of aperture
3084 Axial abutment surface
3085 Front moulding
3086 Rear moulding
3087 Ultrasonic weld line
3088 Flat surface
3089 Flat surface
3090 Break line
3091 Detachable portion
3092 Tab
3093 Axial abutment surface
3100 Recess
3101 Projecting collar

### Further Variant Embodiments Using Bulk Capsule Components

4000 Assembly machine
4001 Bulk capsule cap package
4002 Bulk capsule body package
4004 Window
4010 Capsule body handling mechanism
4011 Capsule body chamber
4020 Capsule cap handling mechanism
4021 Capsule cap chamber
4022 Capsule transfer chamber
4023 Shutter
4024 Shutter
4025 Filling aperture
4026 Retaining collar
4027 Capsule release shutter
4030 Pushrod
4050 Packaging station
4060 End user package comprising blister packs
4061 Box
4062 Blister pack (end user sub-package)
4063 Tray (blister pack)
4064 Cell (blister pack)
4070 Dosing apparatus
4071 First bulk drug package
4072 Second bulk drug package
5000 Second machine (variant)
7000 End user package (second machine—variant)
7001 First drug package
7002 Second drug package
7003 Third drug package
7004 Front cover package
7005 Rear cover package
7006 Removable, temporary cover (for front of package)
7007 Removable, temporary cover (for rear or inwardly facing side of package)
D1, D2 Dimensions of slot shape profile
T1, T2 Thickness dimension
Xc Cell axis
Xd Displacement axis
Xp Profile rod axis
X1-X1, X2-X2, X3-X3
Section lines (refer to the brief description of the drawings)

### SUFFIXES

F or (F) Front view
R or (R) Rear view (opposite the front view)
Tor (T) Top or plan view
B or (B) Bottom view (opposite the plan view)
E or (E) End view
S or (S) Side view
IS or (IS) Inner side view
OS or (OS) Outer side view
LS indicates a left side (left end) view
RS indicates a right side (right end) view
(X1), (X2), (X3) or -X1, -X2, -X3
Sections identified by their respective section lines (refer to the brief description of the drawings)
(FIG. 34) a-, b-, c-, d-
(FIG. 38)-a, -b, -c, -d, -e
Different positions (refer to the brief description of the drawings)

Generally in the drawings, a horizontal dash fill pattern indicates foil 314 or 714, while a vertical dash fill pattern indicates a release surface or release coating 5.

### Non-Exhaustive List of Selected Feature Combinations

The following is a non-exhaustive, numbered list of selected feature combinations in accordance with various aspects of the invention as described above, wherein FC means “feature combination”, and FCn indicates feature combination number n as defined in this list. The feature combination numbers in this list refer only to this list.

FC1. An assembly system comprising:

an assembly apparatus, and

a plurality of packages, the plurality of packages including at least first and second drug packages; each package of the plurality of packages including a plurality of cells; each cell of the first drug package including a single dose of a first drug, each cell of the second drug package including a single dose of a second drug; the assembly apparatus being configured:

to receive the plurality of packages including at least the first and second drug packages, and

to combine together the first and second drugs of the first and second drug packages to form a plurality of single, orally ingestible bodies, wherein each single, orally ingestible body of the plurality of single, orally ingestible bodies includes the single dose of the first drug of a respective one of the cells of the first drug package, and the single dose of the second drug of a respective one of the cells of the second drug package;

characterised in that each cell of each drug package includes only one single dose of the respective drug, and the assembly apparatus is configured to combine together the first and second drugs within the cells of the packages received in the assembly apparatus.
FC2. An assembly system according to FC1, wherein each drug package is a single use drug package which includes at least one part which is configured, in use, by operation of the assembly apparatus to combine together the first and second drugs, either

(a) to be irreversibly removed, destroyed or ruptured, or

(b) to be irreversibly united with the corresponding at least one part of another respective one of the first and second drug packages.

FC3. An assembly system according to FC1, wherein each drug package includes a frame, the respective plurality of cells being separated by the frame to form a spaced array.
FC4. An assembly system according to FC3, wherein each cell includes at least one edible wall fixedly mounted to the frame, the at least one edible wall including or enclosing the respective single dose of the respective, first or second drug.
FC5. An assembly system according to FC1, wherein, when combined together, the first and second drugs are substantially identical in appearance.
FC6. An assembly system according to FC1, wherein the assembly apparatus is configured to sealingly enclose the first and second drug packages in a vacuum or a modified protective atmosphere, and to combine together the first and second drugs of the first and second drug packages in said vacuum or modified protective atmosphere.
FC7. An assembly system according to FC1, wherein the assembly apparatus is configured to sealingly enclose each of the plurality of single, orally ingestible bodies in a respective one of a plurality of cells of an end user package, each cell of the end user package defining an enclosure which is openable by an end user to remove the respective single, orally ingestible body for use.
FC8. An assembly system according to FC7, wherein each package of the plurality of packages includes a frame, the respective plurality of cells being separated by the frame to form a spaced array; and the end user package incorporates the frame of at least one of the plurality of packages received in the assembly apparatus.
FC9. An assembly system according to FC8, wherein each cell of the end user package includes a cell of said at least one of the plurality of packages received in the assembly apparatus; and the end user package includes at least a part of another respective one of the plurality of packages received in the assembly apparatus, wherein the assembly apparatus is arranged to close each cell of the end user package with said at least a part.
FC10. An assembly system according to FC8, wherein the end user package incorporates the frame of each of two or more of the plurality of packages received in the assembly apparatus.
FC11. An assembly system according to FC8, wherein each cell of said at least one of the plurality of packages whose frame is incorporated in the end user package includes a respective, orally ingestible component when received in the assembly apparatus; and each orally ingestible component is incorporated in a respective single, orally ingestible body of the end user package.
FC12. An assembly system according to FC11, wherein said orally ingestible component does not include a drug.
FC13. An assembly system according to FC11, wherein said orally ingestible component forms an outer surface of the respective single, orally ingestible body.
FC14. An assembly system according to FC9, wherein each drug package includes a frame, the respective plurality of cells being separated by the frame to form a spaced array; and each cell of each drug package includes at least one edible wall fixedly mounted to the frame, the at least one edible wall including or enclosing the respective single dose of the respective, first or second drug; and the end user package includes the frame, and the at least one edible wall of each of the cells, of each of the first and second drug packages.
FC15. An assembly system according to FC14, wherein each enclosure of the end user package includes a pair of outer walls spaced apart in opposed relation; and

the assembly apparatus is configured to create a vacuum, and to sealingly enclose the edible walls of each single, orally ingestible body, in said vacuum, between the outer walls of the respective enclosure; and

the outer walls of each enclosure are configured to be moveable together by external ambient atmospheric pressure to compress the edible walls of the respective single, orally ingestible body between the respective pair of outer walls.

FC16. An assembly system according to FC7, wherein the first drug package includes first label indicia identifying the first drug, and the second drug package includes second label indicia identifying the second drug;

and the assembly apparatus is arranged to combine together the first and second label indicia from the first and second drug packages as received in the assembly apparatus to form a composite label of the end user package identifying each of the first and second drugs.

FC17. An assembly system according to FC16, wherein the composite label is a label assembly including first and second label elements, the first label element bearing the first label indicia, the second label element bearing the second label indicia;

the first label element forming part of the first drug package as received in the assembly apparatus,

the second label element forming part of the second drug package as received in the assembly apparatus.

FC18. An assembly system according to FC1, wherein each of the first and second drug packages includes machine readable indicia, and the assembly apparatus is arranged:
to read the machine readable indicia from the first and second drug packages,
to receive information from a database, and,
based on the indicia and the received information, to identify and authenticate the first and second drugs in the first and second drug packages.
   FC19. An assembly system according to FC18, wherein the assembly apparatus is further arranged to upload information based on the machine readable indicia to the database.
   FC20. An assembly system according to FC18, wherein the assembly apparatus is further arranged to output information from the database to a printer to print an information leaflet identifying the first and second drugs.
   FC21. An assembly system according to FC1, wherein the assembly apparatus is configured to receive the first and second drug packages in a stacked configuration in which corresponding cells of the first and second drug packages are axially aligned; and

each of the first and second drug packages includes at least one shape profile, and

the assembly apparatus includes at least one corresponding shape profile, the shape profile of the assembly apparatus being adjustable to selectively fit or obstruct the shape profile of each of the first and second drug packages so as to selectively permit or prevent reception of each of the first and second drug packages in the assembly apparatus.

FC22. An assembly system according to FC1, wherein each drug package includes a frame, the respective plurality of cells being separated by the frame to form a spaced array; and

each cell of each drug package defines an enclosure, and the respective single dose is enclosed within the enclosure.

FC23. An assembly system according to FC22, wherein each drug package includes at least one movable closure element which closes at least one respective end of each cell;

and the assembly apparatus is arranged to displace the at least one movable closure element, relative to the frame, to open the at least one respective end of each cell while the plurality of packages are stacked in the machine.

FC24. An assembly system according to FC22, wherein each cell is closed by a frangible foil.
FC25. An assembly system according to FC22, wherein each single dose is enclosed in spheroidal form within the respective enclosure.
FC26. An assembly system according to FC25, wherein each single dose comprises one or more spheroids having a diameter of at least 2 mm.
FC27. An assembly system according to FC22, wherein each of the plurality of single, orally ingestible bodies includes a capsule formed from at least first and second capsule parts, the assembly apparatus being configured:

to receive the at least first and second capsule parts of a plurality of said capsules,

to collect together between the at least first and second capsule parts of each capsule the single dose of the first drug of a respective one of the cells of the first drug package, and the single dose of the second drug of a respective one of the cells of the second drug package, and

to close together the at least first and second capsule parts to enclose within each capsule the respective single dose of the first drug and the respective single dose of the second drug.

FC28. An assembly system according to FC27, wherein the first and second capsule parts comprise a cap and a body, the body being received in the cap; and each single dose comprises at least one spheroid, the spheroids of all of said single doses being of equal diameter; and a diameter of each spheroid is less than one half, and greater than one third, of an internal diameter of the body of the capsule.
FC29. An assembly system according to FC27, wherein the plurality of packages further includes an end user package and a plug package;
each cell of the plug package defining an enclosure containing a plug;
the assembly machine being configured:
to receive the end user package together with the plug package and the first and second drug packages; and
to slidingly displace each plug through respective axially aligned cells of the plurality of packages, to close an open end of a respective one of the cells of the end user package, to sealingly enclose a respective capsule containing said single doses of the first and second drugs in said respective one of the cells of the end user package;
wherein each cell of the end user package is openable by an end user to remove the respective capsule containing said single doses of the first and second drugs for use.
FC30. An assembly system according to FC29, wherein the first capsule part is a capsule cap, and the second capsule part is a capsule body; and each cell of the plug package defines an enclosure containing the plug together with a respective capsule body.
FC31. An assembly system according to FC30, wherein the plug defines a carrier, and the capsule body is arranged in the carrier.
FC32. An assembly system according to FC29, wherein the first capsule part is a capsule cap, and the second capsule part is a capsule body; and each cell of the end user package, as received in the assembly machine, contains a respective said capsule cap.
FC33. An assembly system according to FC32, wherein each cell of the end user package contains a locating structure, the locating structure supporting the capsule cap in spaced relation to a cell wall of the cell.
FC34. An assembly system according to FC29, wherein the end user package includes an assembly of sub-packages, each sub-package including a plurality of said cells, the sub-packages being separable for use by an end user.
FC35. An assembly system according to FC29, wherein the first capsule part is a capsule cap, and the second capsule part is a capsule body; and the plurality of packages further includes a cap package, each cell of the cap package including a respective capsule cap, and a collar; the collar defining an aperture and being configured to guide the respective capsule body to enter telescopically into the respective capsule cap via the aperture; the assembly machine being configured:

to displace the collar slidingly into the open end of the respective cell of the end user package; and

to slidingly displace the plug into the aperture of the collar, so that the plug and the collar together close the open end of the respective cell of the end user package containing the capsule.

FC36. An assembly system according to FC35, wherein the cap package and the end user package are connected together to form an end user package assembly, the machine being configured to receive the end user package assembly.
FC37. An assembly system according to FC27, wherein the assembly apparatus includes at least one vibration source, the at least one vibration source being configured to transfer vibrational energy to the capsule parts or the single doses during assembly.
FC38. An assembly system according to FC27, wherein each of the first and second drug packages has a different thickness in a thickness dimension, the thickness of each of the packages being proportionate to a volume of the respective single dose of the first or second drug of each of the cells of the respective package; and

the assembly apparatus is configured:
to receive the first and second drug packages in a stacked configuration in which corresponding cells of the first and second drug packages are axially aligned in the thickness dimension, and
to receive an indication corresponding to a maximum volume capacity of each of the capsules, and
to prevent operation of the assembly apparatus to combine together the first and second drugs of the first and second drug packages where a combined thickness of the first and second drug packages is greater than a maximum combined thickness corresponding to the maximum volume capacity of each of the capsules.
   FC39. An assembly system according to FC27, wherein the plurality of packages further includes a first capsule package, each cell of the first capsule package defining an enclosure containing the first capsule part of a respective one of the capsules; the assembly machine being configured:

to receive the first capsule package together with the first and second drug packages; and

to sealingly enclose each capsule containing said single doses of the first and second drugs in a respective one of the cells of the first capsule package to form an end user package;

wherein each cell of the first capsule package forms a cell of the end user package which is openable by an end user to remove the capsule containing said single doses of the first and second drugs for use.

FC40. An assembly system according to FC39, wherein the plurality of packages further includes a second capsule package, each cell of the second capsule package forming an enclosure containing the second capsule part of a respective one of the capsules; the assembly apparatus being configured:

to receive the first and second capsule packages and the first and second drug packages in a stacked configuration in which the first and second drug packages are arranged between the first and second capsule packages, and corresponding cells of the first and second capsule packages and the first and second drug packages are axially aligned; and

to urge the second capsule part of each cell of the second capsule package through the respective, axially aligned cells of the first and second drug packages into the respective, axially aligned cell of the first capsule package.

FC41. An assembly system according to FC40, wherein each cell of the second capsule package contains a carrier, each second capsule part is arranged in the respective carrier, and the assembly apparatus is arranged to urge the carrier containing the second capsule part through the respective, axially aligned cells of the first and second drug packages into the respective, axially aligned cell of the first capsule package, such that the carrier forms a part of the end user package.
FC42. An assembly system according to FC41, wherein each cell of each drug package is closed by a frangible foil, and the carrier is arranged to penetrate the foil.
FC43. An assembly system according to FC42, wherein the foil is patterned to define regions, each region of the foil closing a respective one of the cells and including a first layer and a second layer;

wherein the first layer is undivided, and the second layer is divided into sub-regions which extend radially inwardly away from a wall of the cell and towards a central axis of the cell.

FC44. An assembly system according to FC43, wherein the first layer is a metal, and the second layer is a polymer.
FC45. An assembly system according to FC41, wherein the first capsule package includes a flap, the flap having an adhesive surface and being movable to position its adhesive surface to cover a side of the first capsule package through which the carriers are admitted during assembly.
FC46. An assembly system according to FC45, wherein the first drug package includes first label indicia identifying the first drug, and the second drug package includes second label indicia identifying the second drug;

and the assembly apparatus is arranged to combine together the first and second label indicia from the first and second drug packages as received in the assembly apparatus to form a composite label of the end user package identifying each of the first and second drugs;

and the composite label is arranged to form a front surface of the flap facing away from the adhesive surface.

FC47. An assembly system according to FC41, wherein the first drug package includes first label indicia identifying the first drug, and the second drug package includes second label indicia identifying the second drug;

and the assembly apparatus is arranged to combine together the first and second label indicia from the first and second drug packages as received in the assembly apparatus to form a composite label of the end user package identifying each of the first and second drugs;

and the composite label is fixed by adhesive to cover a side of the first capsule package through which the carriers are admitted during assembly.

FC48. A method for forming drugs into orally ingestible bodies, including:

providing first and second drugs;

providing a plurality of packages, the plurality of packages including at least first and second drug packages, each package of the plurality of packages including a plurality of cells;

packaging the first drug in the first drug package so that each cell of the first drug package includes a single dose of the first drug;

packaging the second drug in the second drug package so that each cell of the second drug package includes a single dose of the second drug; and then

combining together the first and second drugs to form a plurality of single, orally ingestible bodies, wherein each single, orally ingestible body of the plurality of single, orally ingestible bodies includes the single dose of the first drug of a respective one of the cells of the first drug package, and the single dose of the second drug of a respective one of the cells of the second drug package;

characterised in that only one said single dose is packaged in each respective cell of each respective drug package, and the first and second drugs are combined together within the cells of the plurality of packages.
FC49. A method according to FC48, wherein each drug package is a single use drug package and is disposed of after forming the plurality of single, orally ingestible bodies.
FC50. A method according to FC48, wherein each drug package is a single use drug package and includes at least one re-usable, monolithic, foraminous block; and after using the drug package to form the plurality of single, orally ingestible bodies, the block is cleaned and re-used to form part of another single use drug package.
FC51. A method according to FC48, wherein each drug package includes a frame, the respective plurality of cells being separated by the frame to form a spaced array; and

each cell of each drug package defines an enclosure, and the respective single dose is enclosed within the enclosure.

FC52. A method according to FC51, wherein each drug package includes at least one movable closure element which closes at least one respective end of each cell;

and the at least one movable closure element is displaced, relative to the frame, to open the at least one respective end of each cell when combining together the first and second drugs.

FC53. A method according to FC51, wherein each enclosure is sealed by a frangible film, and the film is broken when combining together the first and second drugs.
FC54. A method according to FC48, further including: sealingly enclosing each of the plurality of single, orally ingestible bodies in a respective one of a plurality of cells of an end user package, wherein each cell of the end user package defines an enclosure which is openable by an end user to remove the respective single, orally ingestible body for use.
FC55. A method according to FC48, wherein each of the plurality of single, orally ingestible bodies includes a capsule formed from at least first and second capsule parts; the method further including:

receiving in the assembly apparatus the at least first and second capsule parts of a plurality of said capsules,

collecting together between the at least first and second capsule parts of each capsule the single dose of the first drug of a respective one of the cells of the first drug package, and the single dose of the second drug of a respective one of the cells of the second drug package, and

closing together the at least first and second capsule parts to enclose within each capsule the respective single dose of the first drug and the respective single dose of the second drug.

FC56. A method according to FC55, wherein the plurality of packages further includes an end user package and a plug package;
each cell of the plug package defining an enclosure containing a plug;
the method further including:
receiving the end user package together with the plug package and the first and second drug packages in the assembly machine, and
slidingly displacing each plug through respective axially aligned cells of the plurality of packages, to close an open end of a respective one of the cells of the end user package, to sealingly enclose a respective capsule containing said single doses of the first and second drugs in said respective one of the cells of the end user package;
wherein each cell of the end user package is openable by an end user to remove the respective capsule containing said single doses of the first and second drugs for use.
FC57. A method according to FC54, further including:

providing the first drug package with first label indicia identifying the first drug;

providing the second drug package with second label indicia identifying the second drug; and

combining together the first and second label indicia from the first and second drug packages as received in the assembly apparatus to form a composite label of the end user package identifying each of the first and second drugs.

FC58. A method according to FC48, further including:

providing each of the first and second drug packages with machine readable indicia, and

introducing the plurality of packages including at least the first and second drug packages into an assembly apparatus, and operating the assembly apparatus:
to combine together the first and second drugs to form said plurality of single, orally ingestible bodies,
to read the machine readable indicia from the first and second drug packages,
to receive information from a database, and,
based on the indicia and the received information, to identify and authenticate the first and second drugs in the first and second drug packages.
   FC59. A method according to FC58, further including: uploading information based on the machine readable indicia to the database.
   FC60. A method according to FC58, further including: outputting information from the database to a printer to print an information leaflet identifying the first and second drugs.
   FC61. A package for use in an assembly system, the package including a frame, the frame defining a plurality of cells separated by the frame to form a spaced array; wherein either:

(a) each cell includes at least one edible wall fixedly mounted in the frame, the at least one edible wall being configured to be detached from the frame and consumed in normal use by an end user; or

(b) each cell includes a cell wall, the cell wall extending along a cell axis between opposite, first and second ends of the cell at opposite, first and second sides of the frame; and each of the first and second ends of the cell is closed by a frangible foil or a movable closure element; and each cell contains a single dose of a drug; or

(c) each cell includes a cell wall, the cell wall extending along a cell axis between opposite, first and second ends of the cell at opposite, first and second sides of the frame; and each cell contains a part of an empty capsule, but without a complementary part required to complete the capsule.

FC62. A package according to FC61, wherein the package is a single use package.
FC63. A package according to FC61, wherein (a) each cell includes at least one edible wall fixedly mounted in the frame, the at least one edible wall being configured to be detached from the frame and consumed in normal use by an end user.
FC64. A package according to FC63, wherein each cell is closed on one side of the frame by an outer wall, the outer wall sealingly attached to the frame and configured to be ruptured or torn away from the frame in normal use by an end user to expose the at least one edible wall.
FC65. A plurality of packages according to FC63, wherein the frames of the packages are configured to be stacked and bonded together in an assembled configuration with the plurality of cells of each package in axial alignment with the plurality of cells of an adjacent one of the packages; and wherein a respective edible wall of each cell is configured to adhere to a respective edible wall of a corresponding one of the cells of an adjacent one of the packages when the frames are stacked and bonded together in the assembled configuration.
FC66. A plurality of packages according to FC65, wherein the at least one edible wall includes or encloses a single dose of a drug.
FC67. A plurality of packages according to FC66, wherein the at least one edible wall is exposed at each of two oppositely facing sides of the frame.
FC68. A plurality of packages according to FC67, wherein the frame is coated with a tacky adhesive on each of the two oppositely facing sides of the frame.
FC69. A plurality of packages according to FC68, wherein the frame is enclosed between two removable covers, each cover being arranged to protect a respective one of the two oppositely facing sides of the frame.
FC70. A plurality of packages according to FC63, wherein the frames of the packages are configured to be stacked and bonded together in an assembled configuration with the plurality of cells of each package in axial alignment with the plurality of cells of an adjacent one of the packages; and wherein each frame includes at least one label portion, each of the label portions bearing label indicia identifying the respective drug contained in the respective package; the label portions being configured to form a composite label displaying a respective label portion of each of the packages when the frames are stacked and bonded together in the assembled configuration.
FC71. A plurality of packages according to FC70, wherein each frame includes a plurality of label portions bearing identical label indicia.
FC72. A package according to FC61, wherein (b) each cell includes a cell wall, the cell wall extending along a cell axis between opposite, first and second ends of the cell at opposite, first and second sides of the frame; and each of the first and second ends of the cell is closed by a frangible foil or a movable closure element; and each cell contains a single dose of a drug.
FC73. A package according to FC72, wherein each of the first and second ends of the cell is closed by a frangible foil, and the foil is patterned to define regions, each region of the foil closing a respective one of the cells and including a first layer and a second layer; wherein the first layer is undivided, and the second layer is divided into sub-regions which extend radially inwardly away from the cell wall and towards the cell axis.
FC74. A package according to FC73, wherein the first layer is a metal, and the second layer is a polymer.
FC75. A package according to FC72, wherein each single dose comprises at least one spheroid.
FC76. A package according to FC75, wherein the at least one spheroid has a diameter of at least 2 mm.
FC77. A package according to FC72, wherein the package includes a label portion bearing label indicia identifying the drug, the label portion having an adhesive surface separate or separable from the frame.
FC78. A package according to FC61, wherein (c) each cell includes a cell wall, the cell wall extending along a cell axis between opposite, first and second ends of the cell at opposite, first and second sides of the frame; and each cell contains a part of an empty capsule, but without a complementary part required to complete the capsule.
FC79. A package according to FC78, wherein the part is arranged in a carrier, the carrier being received in the cell and slidable out of the cell along the cell axis.
FC80. A package according to FC79, wherein at least the first end of the cell is closed by a frangible foil, and the carrier is configured to rupture the foil by sliding movement of the carrier out of the cell along the cell axis.
FC81. A package according to FC78, FC79 or FC80, wherein at least the first end of the cell is closed by a frangible foil which is patterned to define regions, each region of the foil closing a respective one of the cells and including a first layer and a second layer;

wherein the first layer is undivided, and the second layer is divided into sub-regions which extend radially inwardly away from a wall of the cell and towards a central axis of the cell.

FC82. A package according to FC81, wherein the first layer is a metal, and the second layer is a polymer.
FC83. A package according to FC78, wherein the cell includes a locating structure supporting the part in spaced relation to the cell wall.
FC84. A package according to FC83, wherein the locating structure is a lining of flock within the cell.
FC85. A package according to FC83 or FC84, wherein the second end of the cell is closed by a removable outer wall, the outer wall configured to be lifted and torn outwardly away from the frame by a user to open the second end of the cell to permit the capsule to be removed from the cell.
FC86. An assembly apparatus for filling capsules with drugs, including:

a plurality of pushrods spaced apart in parallel relation, each pushrod having an end surface;

a pressure plate having a pressure surface arranged in opposed, spaced relation to the end surfaces of the pushrods to define a receiving space between the pressure plate and the end surfaces of the pushrods;

an actuation mechanism configured to cause relative movement between the pressure plate and the pushrods, in a compression stroke, along a displacement axis parallel with the pushrods; and

an alignment structure configured to maintain a stack of packages in alignment with the pushrods when, in use, during the compression stroke, the pushrods are urged through the stack of packages positioned in the receiving space.

FC87. An assembly apparatus according to FC86, including an assembly surface extending between the pushrods, the assembly surface being positionable in a start position proximate the end surfaces of the pushrods, and movable along the displacement axis during the compression stroke.
FC88. An assembly apparatus according to FC87, wherein the assembly surface is biased towards the pressure surface to compress the stack of packages between the assembly surface and the pressure surface, in use, during the compression stroke.
FC89. An assembly apparatus according to FC87, including a flat, front surface extending in a plane parallel with the displacement axis and normal to the assembly surface; the front surface and the assembly surface being fixed together to move together during the compression stroke.
FC90. An assembly apparatus according to FC86, including at least one cutter, the at least one cutter being arranged to cut through a portion of the stacked packages, in use, during the compression stroke.
FC91. An assembly apparatus according to FC86, wherein the alignment structure includes a plurality of alignment rods, the alignment rods extending in parallel with the pushrods beyond the end surfaces of the pushrods and into the receiving space.
FC92. An assembly apparatus according to FC86, including at least one shape profile, and a profile adjustment mechanism for adjusting the at least one shape profile to selectively fit or obstruct a corresponding shape profile of each of the packages so as to selectively permit or prevent each of the packages from being received in the receiving space.
FC93. An assembly apparatus according to FC92, wherein the at least one shape profile is an axially continuous, non-circular cross-section of at least one profile rod; the at least one profile rod extending along a profile rod axis in parallel with the pushrods beyond the end surfaces of the pushrods and into the receiving space; the profile adjustment mechanism being configured to rotate the at least one profile rod about the profile rod axis.
FC94. An assembly apparatus according to FC86, further including:
a controller for controlling the actuation mechanism, and
a reader for reading machine readable indicia in use from the stacked packages;

the controller being arranged to retrieve information from a database to identify and authenticate the stacked packages.

FC95. An assembly apparatus according to FC94, wherein the controller is arranged to upload information based on the machine readable indicia to the database.
FC96. An assembly apparatus according to FC94, wherein the controller is arranged to output information from the database to a printer to print an information leaflet identifying first and second drugs contained in the stacked packages.
FC97. An assembly apparatus according to FC86, wherein the apparatus is arranged to combine together indicia from respective ones of the packages as received in the machine to form a composite label of an end user package.
FC98. An assembly apparatus according to FC86, wherein the apparatus is arranged to displace movable closure elements of the packages while the packages are stacked in the apparatus.
FC99. An assembly apparatus according to FC86, including at least one vibration source, the at least one vibration source being configured to transfer vibrational energy to the drugs or respective parts of the capsules during assembly.
FC100. An assembly apparatus for use in assembling together a plurality of packages, each of the packages including a frame, the frame defining a plurality of cells separated by the frame to form a spaced array; the apparatus including:

an alignment structure for guiding the plurality of packages in a stacked configuration with the cells of each package in axial alignment with the cells of each other package of the plurality of packages; and

a compression mechanism for compressing together the frames of the plurality of packages in the stacked configuration to form an assembly defining an end user package.

FC101. An assembly apparatus according to FC100, wherein the assembly apparatus is arranged to create a vacuum, and to compress together the frames of the plurality of packages within the vacuum.
FC102. An assembly apparatus according to FC100, wherein the assembly apparatus is arranged, before compressing together the frames of the plurality of packages, to detach and separate the frame of each of two or more of the packages from a cover or covers removably connected to the frame.
FC103. An assembly apparatus according to FC100, wherein the assembly apparatus includes a plurality of elongate slots, each slot being configured to receive a respective one of the packages.
FC104. An assembly apparatus according to FC103, wherein each slot includes a shape profile configured to selectively fit or obstruct a corresponding shape profile of a respective one of the packages introduced into the slot.
FC105. An assembly apparatus according to FC104, wherein different ones of the slots have different shape profiles.
FC106. An assembly apparatus according to FC100, wherein the frame of each package includes a plurality of label portions; and

the assembly apparatus includes a cutting mechanism arranged to cut away different respective ones of the label portions of the plurality of packages, to leave each of the plurality of packages, after said cutting away, with a different one or different ones of its respective label portions when compared with each other one of the plurality of packages; and

the assembly apparatus is configured to assemble together the packages in the stacked configuration to display a remaining one of the label portions of each package of the plurality of packages, said remaining ones of the label portions being displayed in juxtaposed relation to form together a composite label of the end user package.

FC107. An assembly apparatus according to FC100, wherein the alignment structure includes alignment elements configured to engage the packages to maintain the packages in parallel relation, and the machine is configured to slide the packages together along the alignment elements.
FC108. An assembly apparatus according to FC107, wherein the alignment elements form part of a package transfer assembly, and the package transfer assembly is arranged to move the packages from an initial, receiving position to an assembly position in which the packages are compressed together by the compression mechanism.
FC109. An assembly apparatus according to FC100, wherein the assembly apparatus is arranged to read machine readable indicia from the packages, and to retrieve information from a database to identify and authenticate each of the packages.
FC110. An assembly apparatus according to FC109, wherein the assembly apparatus is further arranged to upload information based on the machine readable indicia to the database.
FC111. An assembly apparatus according to FC109, wherein the assembly apparatus is further arranged to output information from the database to a printer to print an information leaflet identifying drugs contained in the packages.
FC112. An end user package including a plurality of cells and a plurality of single, orally ingestible bodies;

each of the single, orally ingestible bodies being sealingly enclosed within a respective cell of the plurality of cells, each cell defining an enclosure which is openable by an end user to remove the respective single, orally ingestible body for use;

wherein each single, orally ingestible body includes a single dose of a first drug, and a single dose of a different, second drug; and either:

(a) each single, orally ingestible body includes a capsule containing a plurality of granules, a first one or first ones of the granules containing the first drug but not the second drug, a second one or second ones of the granules containing the second drug but not the first drug; or

(b) the end user package includes at least first and second drug packages, each of the drug packages including a respective frame, the frame defining a plurality of drug package cells separated by the respective frame to form a spaced array, each of the drug package cells including at least one edible wall fixedly mounted in the respective frame;

the at least one edible wall of each of the drug package cells of the first drug package including or enclosing the single dose of the first drug but not the second drug;

the at least one edible wall of each of the drug package cells of the second drug package including or enclosing the single dose of the second drug but not the first drug;

the frames being connected together, each of the drug package cells of the first drug package being in axial alignment with a respective one of the drug package cells of the second drug package, to form together a respective one of the cells of the end user package;

the edible walls of the respective drug package cells forming each cell of the end user package being connected together to form together the respective single, orally ingestible body contained within the respective cell of the end user package.

FC113. An end user package according to FC112, wherein (a) each single, orally ingestible body includes a capsule containing a plurality of granules, a first one or first ones of the granules containing the first drug but not the second drug, a second one or second ones of the granules containing the second drug but not the first drug.
FC114. An end user package according to FC113, wherein the first and second ones of the granules are spheroids having a mean diameter of at least 2 mm.
FC115. An end user package according to FC114, wherein the capsule includes a body and a cap, the body being received in the cap, and a diameter of each spheroid is less than one half, and greater than one third, of an internal diameter of the body of the capsule.
FC116. An end user package according to FC113, FC114, or FC115, wherein all of the capsules contain an equal number of said first one or first ones of the granules, and all of the capsules contain an equal number of said second one or second ones of the granules.
FC117. An end user package according to FC113, wherein each capsule is arranged in a carrier, the carrier being fittingly received within the cell and having an open end through which the capsule may be removed from the cell, and a closed end opposite the open end.
FC118. An end user package according to FC117, wherein each cell is internally flocked.
FC119. An end user package according to FC114 or FC115, wherein the first and second ones of the spheroids are substantially identical in appearance.
FC120. An end user package according to FC112, wherein (b) the end user package includes at least first and second drug packages, each of the drug packages including a respective frame, the frame defining a plurality of drug package cells separated by the respective frame to form a spaced array, each of the drug package cells including at least one edible wall fixedly mounted in the respective frame;

the at least one edible wall of each of the drug package cells of the first drug package including or enclosing the single dose of the first drug but not the second drug;

the at least one edible wall of each of the drug package cells of the second drug package including or enclosing the single dose of the second drug but not the first drug;

the frames being connected together, each of the drug package cells of the first drug package being in axial alignment with a respective one of the drug package cells of the second drug package, to form together a respective one of the cells of the end user package;

the edible walls of the respective drug package cells forming each cell of the end user package being connected together to form together the respective single, orally ingestible body contained within the respective cell of the end user package.

FC121. An end user package according to FC120, wherein each enclosure of the end user package includes a pair of outer walls spaced apart in opposed relation; and the edible walls of each single, orally ingestible body are bonded together by adhesion, and are compressed together between the outer walls of the respective enclosure by external ambient atmospheric pressure acting on the outer walls.
FC122. An end user package according to FC120, wherein each frame includes a label portion bearing label indicia identifying the respective drug contained in the respective drug package; the label portions being juxtaposed to form a composite label of the end user package.
FC123. An assembly system comprising:

an assembly apparatus, and

a plurality of packages, the plurality of packages including at least first and second drug packages; each package of the plurality of packages including a plurality of cells;

each cell of the first drug package including a single dose of a first drug, each cell of the second drug package including a single dose of a second drug;
each single dose comprising one or more particles, the particles of all said single doses being of equal size and shape, each particle having a dimension of at least 1.5 mm;
wherein each cell of each drug package includes only one single dose of the respective drug,
and each of the cells of the first drug package includes an equal number of particles,
and each of the cells of the second drug package includes an equal number of particles;
and the assembly apparatus is configured:

to receive the plurality of packages including at least the first and second drug packages,

to receive a plurality of capsule caps and capsule bodies, and

to combine together the first and second drugs of the first and second drug packages to form a plurality of capsules, wherein for each capsule of the plurality of capsules, the single dose of the first drug of a respective one of the cells of the first drug package is encapsulated, together with the single dose of the second drug of a respective one of the cells of the second drug package, between a respective said capsule cap and capsule body.

FC124. An assembly system according to FC1, wherein each of said particles is a spheroid having a diameter of at least 1.5 mm.
FC125. A method for filling drugs into capsules, including:

providing first and second drugs;

providing a plurality of packages, the plurality of packages including at least first and second drug packages, each package of the plurality of packages including a plurality of cells;

packaging the first drug in the first drug package so that each cell of the first drug package includes a single dose of the first drug; and

packaging the second drug in the second drug package so that each cell of the second drug package includes a single dose of the second drug;

wherein each single dose comprises one or more particles, the particles of all said single doses being of equal size and shape, each particle having a dimension of at least 1.5 mm;
and only one said single dose is packaged in each respective cell of each respective drug package,
and each of the cells of the first drug package includes an equal number of particles,
and each of the cells of the second drug package includes an equal number of particles;
the method further including:

receiving in an assembly apparatus the plurality of packages including at least the first and second drug packages together with a plurality of capsule caps and capsule bodies; and

operating the assembly apparatus to combine together the first and second drugs of the first and second drug packages to form a plurality of capsules, wherein for each capsule of the plurality of capsules, the single dose of the first drug of a respective one of the cells of the first drug package is encapsulated, together with the single dose of the second drug of a respective one of the cells of the second drug package, between a respective said capsule cap and capsule body.

FC126. An end user package including a plurality of capsules;
each capsule including a single dose of a first drug, and a single dose of a different, second drug;
each capsule containing a plurality of particles, a first one or first ones of the particles containing the first drug but not the second drug, a second one or second ones of the particles containing the second drug but not the first drug;
wherein the first and second ones of the particles are spheroids having a mean diameter of at least 1.5 mm, and all of the capsules contain an equal number of said first one or first ones of the particles, and all of the capsules contain an equal number of said second one or second ones of the particles.
FC127. An end user package according to FC126, wherein each capsule comprises a cap and a body, the body being received in the cap; and each single dose comprises at least one spheroid, the spheroids of all of said single doses being of equal diameter; and a diameter of each spheroid is less than one half, and greater than one third, of an internal diameter of the body of the capsule.
FC128. A kit of medicaments for use in the treatment of psychosis in an individual with a history of non-compliance with oral medication, including:
a plurality of crisis oral dosage units, and
a plurality of maintenance oral dosage units;
each of the crisis oral dosage units including a single dose of a first drug and a single dose of a different, second drug, said single doses being combined together as a single, orally ingestible body;
each of the maintenance oral dosage units including a single dose of the first drug without the second drug;
wherein the first drug is an antipsychotic, and the second drug is a psychoactive selected to alleviate an affective symptom associated with the psychosis.
FC129. A kit of medicaments according to FC128, wherein the first drug is selected to alleviate a first symptom of the psychosis, the first symptom being one of hallucinations and delusions; and the second drug is selected to alleviate the affective symptom without alleviating the first symptom.
FC130. A kit of medicaments according to FC128, wherein, when taken at a prescribed frequency: the first drug is selected to alleviate a first symptom of the psychosis within a first time period from initial administration, the first symptom being one of hallucinations and delusions, and the second drug is selected to alleviate the affective symptom within a second time period from initial administration; the second time period being shorter than the first time period.
FC131. The use of a kit of medicaments as defined in any of FC128, FC129 or FC130 for the treatment of psychosis in an individual with a history of non-compliance with oral medication.
FC132. The use of a kit of medicaments according to FC131, wherein the treatment of psychosis comprises the treatment of schizophrenia.
FC133. The use of a kit of medicaments according to FC131, wherein both the crisis oral dosage units and the maintenance oral dosage units are provided to the individual for alternative self-administration at the discretion of the individual, wherein the maintenance oral dosage units are provided for self-administration when the individual feels well, and the crisis oral dosage units are provided for self-administration when the individual feels unwell.
FC134. A method for treating psychosis in an individual with a history of non-compliance with oral medication, comprising:

providing different, first and second drugs, wherein the first drug is an antipsychotic, and the second drug is a psychoactive selected to alleviate an affective symptom associated with the psychosis;

combining together the first and second drugs to form a plurality of crisis oral dosage units, each of the crisis oral dosage units including a single dose of the first drug and a single dose of the second drug, said single doses being combined together as a single, orally ingestible body;

forming a plurality of maintenance oral dosage units, each of the maintenance oral dosage units including a single dose of the first drug without the second drug; and

providing both the crisis oral dosage units and the maintenance oral dosage units to the individual for alternative self-administration at the discretion of the individual, wherein the maintenance oral dosage units are provided for self-administration when the individual feels well, and the crisis oral dosage units are provided for self-administration when the individual feels unwell.

FC135. A method according to FC134, wherein the first drug is selected to alleviate a first symptom of the psychosis, the first symptom being one of hallucinations and delusions; and the second drug is selected to alleviate the affective symptom without alleviating the first symptom.
FC136. A method according to FC134, wherein, when taken at a prescribed frequency: the first drug is selected to alleviate a first symptom of the psychosis within a first time period from initial administration, the first symptom being one of hallucinations and delusions, and the second drug is selected to alleviate the affective symptom within a second time period from initial administration; the second time period being shorter than the first time period.
FC137. A method according to FC134, for the treatment of schizophrenia.
FC138. A method for manufacturing medicaments for treating psychosis in an individual with a history of non-compliance with oral medication, comprising:

identifying the individual;

identifying different, first and second drugs prescribed for the identified individual, wherein the first drug is an antipsychotic, and the second drug is a psychoactive selected to alleviate an affective symptom associated with the psychosis in the identified individual;

combining together the first and second drugs to form a plurality of crisis oral dosage units customised for the identified individual, each of the crisis oral dosage units including a single dose of the first drug and a single dose of the second drug, said single doses being combined together as a single, orally ingestible body;

forming a plurality of maintenance oral dosage units, each of the maintenance oral dosage units including a single dose of the first drug without the second drug; and packaging the plurality of crisis oral dosage units and the plurality of maintenance oral dosage units, respectively as two separate components in a kit of medicaments.

In the claims, reference numerals or characters may be inserted in parentheses, purely for ease of reference, and when so inserted should not be construed as limiting features.

1. An assembly system comprising:
an assembly apparatus, and
a plurality of packages, the plurality of packages including at least first and second drug packages;
each package of the plurality of packages including a plurality of cells;
each cell of the first drug package including a single dose of a first drug, each cell of the second drug package including a single dose of a second drug;
the assembly apparatus being configured:
to receive the plurality of packages including at least the first and second drug packages, and
to combine together the first and second drugs of the first and second drug packages to form a plurality of single, orally ingestible bodies, wherein each single, orally ingestible body of the plurality of single, orally ingestible bodies includes the single dose of the first drug of a respective one of the cells of the first drug package, and the single dose of the second drug of a respective one of the cells of the second drug package;
characterised in that each cell of each drug package includes only one single dose of the respective drug, and the assembly apparatus is configured to combine together the first and second drugs within the cells of the packages received in the assembly apparatus.

2. A method for forming drugs into orally ingestible bodies, including:
providing first and second drugs;
providing a plurality of packages, the plurality of packages including at least first and second drug packages, each package of the plurality of packages including a plurality of cells;
packaging the first drug in the first drug package so that each cell of the first drug package includes a single dose of the first drug;
packaging the second drug in the second drug package so that each cell of the second drug package includes a single dose of the second drug; and then
combining together the first and second drugs to form a plurality of single, orally ingestible bodies, wherein each single, orally ingestible body of the plurality of single, orally ingestible bodies includes the single dose of the first drug of a respective one of the cells of the first drug package, and the single dose of the second drug of a respective one of the cells of the second drug package;
characterised in that only one said single dose is packaged in each respective cell of each respective drug package, and the first and second drugs are combined together within the cells of the plurality of packages.

3. A package for use in an assembly system, the package including a frame, the frame defining a plurality of cells separated by the frame to form a spaced array; wherein either:
(a) each cell includes at least one edible wall fixedly mounted in the frame, the at least one edible wall being configured to be detached from the frame and consumed in normal use by an end user; or
(b) each cell includes a cell wall, the cell wall extending along a cell axis between opposite, first and second ends of the cell at opposite, first and second sides of the frame; and each of the first and second ends of the cell is closed by a frangible foil or a movable closure element; and each cell contains a single dose of a drug; or
(c) each cell includes a cell wall, the cell wall extending along a cell axis between opposite, first and second ends of the cell at opposite, first and second sides of the frame; and each cell contains a part of an empty capsule, but without a complementary part required to complete the capsule.

4. An assembly apparatus for filling capsules with drugs, including:
a plurality of pushrods spaced apart in parallel relation, each pushrod having an end surface;
a pressure plate having a pressure surface arranged in opposed, spaced relation to the end surfaces of the pushrods to define a receiving space between the pressure plate and the end surfaces of the pushrods;
an actuation mechanism configured to cause relative movement between the pressure plate and the pushrods, in a compression stroke, along a displacement axis parallel with the pushrods; and
an alignment structure configured to maintain a stack of packages in alignment with the pushrods when, in use, during the compression stroke, the pushrods are urged through the stack of packages positioned in the receiving space.

5. An assembly apparatus for use in assembling together a plurality of packages, each of the packages including a frame, the frame defining a plurality of cells separated by the frame to form a spaced array; the apparatus including:
an alignment structure for guiding the plurality of packages in a stacked configuration with the cells of each package in axial alignment with the cells of each other package of the plurality of packages; and
a compression mechanism for compressing together the frames of the plurality of packages in the stacked configuration to form an assembly defining an end user package.

6-14. (canceled)

15. An assembly system according to claim 1, wherein the assembly apparatus is configured to sealingly enclose each of the plurality of single, orally ingestible bodies in a respective one of a plurality of cells of an end user package, each cell of the end user package defining an enclosure which is openable by an end user to remove the respective single, orally ingestible body for use.

16. An assembly system according to claim 15, wherein:
each package of the plurality of packages includes a frame, the respective plurality of cells being separated by the frame to form a spaced array; and
the end user package incorporates the frame of at least one of the plurality of packages received in the assembly apparatus; and
each cell of the end user package includes a cell of said at least one of the plurality of packages received in the assembly apparatus; and
the end user package includes at least a part of another respective one of the plurality of packages received in the assembly apparatus, wherein the assembly apparatus is arranged to close each cell of the end user package with said at least a part; and
each cell of each drug package includes at least one edible wall fixedly mounted to the frame of the respective drug package, the at least one edible wall including or enclosing the respective single dose of the respective, first or second drug; and
the end user package includes the frame, and the at least one edible wall of each of the cells, of each of the first and second drug packages.

17. An assembly system according to claim 15, wherein the first drug package includes first label indicia identifying the first drug, and the second drug package includes second label indicia identifying the second drug;
and the assembly apparatus is arranged to combine together the first and second label indicia from the first and second drug packages as received in the assembly apparatus to form a composite label of the end user package identifying each of the first and second drugs.

18. An assembly system according to claim 1, wherein each of the first and second drug packages includes machine readable indicia, and the assembly apparatus is arranged:
to read the machine readable indicia from the first and second drug packages,
to receive information from a database, and,
based on the indicia and the received information, to identify and authenticate the first and second drugs in the first and second drug packages.

19. An assembly system according to claim 1, wherein each drug package includes a frame, the respective plurality of cells being separated by the frame to form a spaced array; and
each cell of each drug package defines an enclosure, and the respective single dose is enclosed within the enclosure; and wherein
either
(a) each drug package includes at least one movable closure element which closes at least one respective end of each cell,
and the assembly apparatus is arranged to displace the at least one movable closure element, relative to the frame, to open the at least one respective end of each cell while the plurality of packages are stacked in the machine;
or
(b) each cell is closed by a frangible foil.

20. An assembly system according to claim 1, wherein each drug package includes a frame, the respective plurality of cells being separated by the frame to form a spaced array; and
each cell of each drug package defines an enclosure, and the respective single dose is enclosed within the enclosure; and
each single dose is enclosed in spheroidal form within the respective enclosure;
wherein each single dose comprises one or more spheroids having a diameter of at least 2 mm.

21. An assembly system according to claim 1, wherein each drug package includes a frame, the respective plurality of cells being separated by the frame to form a spaced array; and
each cell of each drug package defines an enclosure, and the respective single dose is enclosed within the enclosure;
and each of the plurality of single, orally ingestible bodies includes a capsule formed from at least first and second capsule parts, the assembly apparatus being configured:
to receive the at least first and second capsule parts of a plurality of said capsules,
to collect together between the at least first and second capsule parts of each capsule the single dose of the first drug of a respective one of the cells of the first drug package, and the single dose of the second drug of a respective one of the cells of the second drug package, and
to close together the at least first and second capsule parts to enclose within each capsule the respective single dose of the first drug and the respective single dose of the second drug.

22. An assembly system according to claim 21, wherein the plurality of packages further includes an end user package and a plug package;
each cell of the plug package defining an enclosure containing a plug;
the assembly machine being configured:
to receive the end user package together with the plug package and the first and second drug packages; and
to slidingly displace each plug through respective axially aligned cells of the plurality of packages, to close an open end of a respective one of the cells of the end user package, to sealingly enclose a respective capsule containing said single doses of the first and second drugs in said respective one of the cells of the end user package;
wherein each cell of the end user package is openable by an end user to remove the respective capsule containing said single doses of the first and second drugs for use.

23. An assembly system according to claim 22, wherein the first capsule part is a capsule cap, and the second capsule part is a capsule body; and each cell of the plug package defines an enclosure containing the plug together with a respective capsule body;
wherein the plug defines a carrier, and the capsule body is arranged in the carrier.

24. An assembly system according to claim 22, wherein the first capsule part is a capsule cap, and the second capsule part is a capsule body; and each cell of the end user package, as received in the assembly machine, contains a respective said capsule cap.

25. An assembly system according to claim 24, wherein each cell of the end user package contains a locating structure, the locating structure supporting the capsule cap in spaced relation to a cell wall of the cell.

26. An assembly system according to claim 21, wherein the assembly apparatus includes at least one vibration source, the at least one vibration source being configured to transfer vibrational energy to the capsule parts or the single doses during assembly.

27. An assembly system according to claim 21, wherein the plurality of packages further includes a first capsule package, each cell of the first capsule package defining an enclosure containing the first capsule part of a respective one of the capsules; the assembly machine being configured:
to receive the first capsule package together with the first and second drug packages; and
to sealingly enclose each capsule containing said single doses of the first and second drugs in a respective one of the cells of the first capsule package to form an end user package;
wherein each cell of the first capsule package forms a cell of the end user package which is openable by an end user to remove the capsule containing said single doses of the first and second drugs for use; and
wherein the plurality of packages further includes a second capsule package, each cell of the second capsule package forming an enclosure containing the second capsule part of a respective one of the capsules; the assembly apparatus being configured:
to receive the first and second capsule packages and the first and second drug packages in a stacked configuration in which the first and second drug packages are arranged between the first and second capsule packages, and corresponding cells of the first and second capsule packages and the first and second drug packages are axially aligned; and
to urge the second capsule part of each cell of the second capsule package through the respective, axially aligned cells of the first and second drug packages into the respective, axially aligned cell of the first capsule package; and
wherein each cell of the second capsule package contains a carrier, each second capsule part is arranged in the respective carrier, and the assembly apparatus is arranged to urge the carrier containing the second capsule part through the respective, axially aligned cells of the first and second drug packages into the respective, axially aligned cell of the first capsule package, such that the carrier forms a part of the end user package.

28. A plurality of packages according to claim 3, wherein (a) each cell includes at least one edible wall fixedly mounted in the frame, the at least one edible wall being configured to be detached from the frame and consumed in normal use by an end user; and
wherein the frames of the packages are configured to be stacked and bonded together in an assembled configuration with the plurality of cells of each package in axial alignment with the plurality of cells of an adjacent one of the packages; and
wherein a respective edible wall of each cell is configured to adhere to a respective edible wall of a corresponding one of the cells of an adjacent one of the packages when the frames are stacked and bonded together in the assembled configuration.

29. A package according to claim 3, wherein (b) each cell includes a cell wall, the cell wall extending along a cell axis between opposite, first and second ends of the cell at opposite, first and second sides of the frame; and each of the first and second ends of the cell is closed by a frangible foil or a movable closure element; and each cell contains a single dose of a drug; and
wherein each single dose comprises at least one spheroid,
wherein the at least one spheroid has a diameter of at least 2 mm.

30. A package according to claim 3, wherein (c) each cell includes a cell wall, the cell wall extending along a cell axis between opposite, first and second ends of the cell at opposite, first and second sides of the frame; and each cell contains a part of an empty capsule, but without a complementary part required to complete the capsule; and
wherein the part is arranged in a carrier, the carrier being received in the cell and slidable out of the cell along the cell axis.

31. A package according to claim 3, wherein (c) each cell includes a cell wall, the cell wall extending along a cell axis between opposite, first and second ends of the cell at opposite, first and second sides of the frame; and each cell contains a part of an empty capsule, but without a complementary part required to complete the capsule; and
wherein the cell includes a locating structure supporting the part in spaced relation to the cell wall; and
wherein the locating structure is a lining of flock within the cell.

32. An assembly apparatus according to claim 4, further including:
a controller for controlling the actuation mechanism, and
a reader for reading machine readable indicia in use from the stacked packages;
the controller being arranged to retrieve information from a database to identify and authenticate the stacked packages.

33. An assembly apparatus according to claim 4, wherein the apparatus is arranged to combine together indicia from respective ones of the packages as received in the machine to form a composite label of an end user package.

34. An assembly apparatus according to claim 4, including at least one vibration source, the at least one vibration source being configured to transfer vibrational energy to the drugs or respective parts of the capsules during assembly.

35. An assembly apparatus according to claim 5, wherein the assembly apparatus is arranged to create a vacuum, and to compress together the frames of the plurality of packages within the vacuum.

36. An assembly apparatus according to claim 5, wherein the assembly apparatus is arranged to read machine readable indicia from the packages, and to retrieve information from a database to identify and authenticate each of the packages.
